(19) United States
(12) Patent Application Publication
(10) Pub. No.: US 2021/0102197 A1
(43) Pub. Date: Apr. 8, 2021

(54) DESIGNING SENSITIVE, SPECIFIC, AND OPTIMALLY ACTIVE BINDING MOLECULES FOR DIAGNOSTICS AND THERAPEUTICS

(71) Applicants: THE BROAD INSTITUTE, INC., Cambridge, MA (US); PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Pardis SABETI, Cambridge, MA (US); Hayden METSKY, Cambridge, MA (US); Cameron MYHRVOLD, Cambridge, MA (US); Nicholas HARADHVALA, Cambridge, MA (US)

(21) Appl. No.: 17/065,504

(22) Filed: Oct. 7, 2020

Related U.S. Application Data
(60) Provisional application No. 62/912,021, filed on Oct. 7, 2019.
(60) Provisional application No. 62/982,731, filed on Feb. 27, 2020.
(60) Provisional application No. 63/074,307, filed on Sep. 3, 2020.

(51) Int. Cl. C12N 15/10; C12N 15/113; C12N 9/22; G16B 35/10

(52) U.S. Cl. CPC C12N 15/1089; C12N 15/113; C12N 2310/20; G16B 35/10; C12N 2310/141; C12N 9/22

(57) ABSTRACT

The invention provides for methods for designing sensitive, specific, and optimally active binding molecules. Systems, methods and compositions utilizing the designed molecules in diagnostics and therapeutics are also provided.

### CROSS-REFERENCE TO RELATED APPLICATIONS

This application claims the benefit of U.S. Provisional Application No. 62/912,021 filed Oct. 7, 2019, U.S. Provisional Application 62/982,731 filed Feb. 27, 2020, and U.S. Provisional Application 63/074,307 filed Sep. 3, 2020. The entire contents of the above-identified applications are hereby fully incorporated herein by reference.

### REFERENCE TO AN ELECTRONIC SEQUENCE LISTING

The contents of the electronic sequence listing (“BROD-4950US ST25.txt”; Size is 12,800 bytes (16 KB on disk) and it was created on Oct. 1, 2020) is herein incorporated by reference in its entirety.

### TECHNICAL FIELD

The subject matter disclosed herein is generally directed to designing sensitive, specific, and optimally active binding molecules. Particular examples relate to using machine learning and software algorithms to design binding molecules to target and eliminate viruses in a sample.

### BACKGROUND

Emerging, evolving targets in viral outbreaks and other molecules with changing diversity present a challenge for diagnostic and therapeutic applications. The ability to optimize diagnostic and therapeutic molecules for that can keep pace with evolution is a continuing challenge.

Novel Severe acute respiratory syndrome-related coronavirus, SARS-CoV-2 (family: Coronaviridae), is the virus behind a severe outbreak originating in China. SARS-CoV-2 surveillance is essential to slowing widespread transmission. There are several diagnostic challenges associated with the current SARS-CoV-2 outbreak. First, high case counts overwhelm diagnostic capacity, underscoring the need for a rapid pipeline for sample processing and diagnosis. Second, SARS-CoV-2 is closely related to other important coronavirus subspecies and species, so diagnostic assays can yield false positives if they are not exquisitely specific to SARS-CoV-2. Third, suspected SARS-CoV-2 patients sometimes have a different respiratory viral infection or have co-infections with SARS-CoV-2 and other respiratory viruses. Therefore, it is important to characterize these other pathogens, for both patient diagnostics and outbreak response.

Citation or identification of any document in this application is not an admission that such a document is available as prior art to the present invention.

### SUMMARY

In certain example embodiments, a computer-implemented method to design viral diagnostics could rapidly render highly effective assays using the latest genomic diversity, including from novel species or strains. The method comprises designing a set of diagnostic molecules to be sensitive across sequence diversity for a set of target sequences. The system solves for sets of diagnostic molecules that with maximal detection activity across extensive genomic diversity. The system then constructs a realistic activity function by developing a dataset and training models to predict activity. Then the system performs a query algorithm that indexes k-mers from all inputs and splits the k-mers across many small tries according to a hash function. The query algorithm determines the specificity of the binding molecules, tolerating both high divergence and G-U wobble base pairing. The system performs a branch and bound search over the genome of a sample virus using the binding molecules to create a ranked set of assay options. The ranked binding molecule options may be used to detect desired viruses in a sample.

In an aspect, the computer-implemented method designs a set of diagnostic molecules to be sensitive across sequence diversity for a set of target sequences. The system identified a set of known sequences within a region and a function that quantifies detection activity between a binding molecule and a targeted sequence. The system constructs a ground set of possible binding molecules by finding representative subsequences across the set using locality sensitive hashing. The system identifies a desired binding molecule set, a subset of the ground set, that maximizes a function of the expected activity between the binding molecules and sequences in the set of target sequences. The system identifies the desired binding molecule set by approaching the function as a non-monetone submodular maximization problem that is solved with a fast randomized combinatorial algorithm.

To construct a realistic activity function, the system develops a dataset and training models to predict activity. The system creates a database of unique guide-target pairs having sequence composition representative of viral genomes. The system then classifies all guide-target pairs as inactive or active and trains a classifier on all pairs. The system creates a regressing model for active pairs with a convolutional neural network to create a model that predicts the activity of active guide-target pairs and classifies the pairs based on activity.

To develop an exact query algorithm to enforce specificity across viral taxa, the system indexes k-mers from all input taxonomies, in which the k-mers are split across many small tries according to a hash function. For example, the system performs a method comprising: selecting K-mer of a target sequence to index; splitting a sequence of each K-mer of the target sequence into a configured number of components; hashing each component to a bit vector; fetching corresponding tries; and inserting each K-mer of the target sequence into each of the corresponding tries. The result finds any non-specificity of a query and thus identifies binding molecules with high specificity to the target set.

The system then performs a branch and bound search across a viral genome to identify regions to target, scored according to both their amplification potential (e.g., presence of conserved endpoints) and the optimal activity of a probe set within the region. The system may connect directly with viral genome databases to download and curate sequences from the targeted taxa, as well as for building the index that enforces specificity. The system finds relatively likely combinations of substitutions where a binding molecule binds to estimate a probability that a binding molecule's activity will degrade over time.

The binding molecule system may perform this method regularly or even continuously for thousands of viruses. Doing so could make it more likely to identify highly effective binding molecules for many viruses at the start of an outbreak. The binding molecules for many strains could even be preemptively validated. Routinely performing the method would also provide binding molecule designs that always best reflect the latest diversity.

Nucleic acid detection systems for detecting the presence of a target molecule in a sample are provided comprising: one or more binding molecules generated according to the methods described herein. In certain embodiments, the binding molecule is an amplification primer, hybridization probe, toehold switch, or guide molecule.

In an aspect, the nucleic acid detection system comprises a target molecule for a virus. The virus can be a coronavirus, in an embodiment, Severe acute respiratory syndrome-related coronavirus, SARS-CoV-2 (family: Coronaviridae)(also referred to as 2019-nCoV, COVID 2019). In some embodiments, the nucleic acid detection system comprises one or more CRISPR systems, the CRISPR systems comprising one or more Cas proteins and one or more guide molecules made according to the methods disclosed herein and designed to bind to one or more corresponding target sequences of one or more viral species or subspecies; and a detection construct.

In an aspect, the system comprises one or more Cas proteins, the Cas proteins are a Class 1 or Class 2 CRISPR protein. The one or more Cas proteins may comprise one or more Type II, one or more Type V Cas protein, one or more Type VI Cas proteins, or a combination of Type V and Type VI proteins. In embodiments, the one or more Cas proteins is Cas13, optionally selected from Cas13a, Cas13b, or Cas13c.

In embodiments, the one or more Cas proteins comprises one or more HEPN domains, optionally wherein the one or more HEPN domains comprise a RxxxxH motif sequence. In an aspect, the RxxxxH motif comprises a R[N/H/K]X1X2X3H (SEQ ID NO: 1-3)

sequence, wherein X1 is R, S, D, E, Q, N, G, or Y, and X2 is independently I, S, T, V, or L, and X3 is independently L, F, N, Y, V, I, S, D, E, or A.

The system in one aspect, comprises a detection construct that suppresses generation of a detectable positive signal until cleaved or deactivated, or masks a detectable positive signal, or generates a detectable negative signal until the detection construct is deactivated or cleaved. The detection construct may comprise: a silencing RNA that suppresses generation of a gene product encoded by a reporting construct, wherein the gene product generates the detectable positive signal when expressed; a ribozyme that generates the negative detectable signal, and wherein the positive detectable signal is generated when the ribozyme is deactivated; or a ribozyme that converts a substrate to a first color and wherein the substrate converts to a second color when the ribozyme is deactivated; an aptamer and/or comprises a polynucleotide-tethered inhibitor; a polynucleotide to which a detectable ligand and a masking component are attached; a nanoparticle held in aggregate by bridge molecules, wherein at least a portion of the bridge molecules comprises a polynucleotide, and wherein the solution undergoes a color shift when the nanoparticle is disbursed in solution; a quantum dot linked to one or more quencher molecules by a linking molecule, wherein at least a portion of the linking molecule comprises a polynucleotide; a polynucleotide in complex with an intercalating agent, wherein the intercalating agent changes absorbance upon cleavage of the polynucleotide; or two fluorophores tethered by a polynucleotide that undergo a shift in fluorescence when released from the polynucleotide.

The system can comprise an aptamer that is a polynucleotide-tethered inhibitor that sequesters an enzyme, wherein the enzyme generates a detectable signal upon release from the aptamer or polynucleotide-tethered inhibitor by acting upon a substrate; or is an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or sequesters a pair of agents that when released from the aptamers combine to generate a detectable signal. In an aspect, the nanoparticle is a colloidal metal, or the detectable ligand is a fluorophore or quantum dot and the masking component is a quencher molecule.

The system may further comprise reagents to amplify target sequences comprising reagents for nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).

The system may further comprise tris(2-carboxyethyl)phosphine, EDTA, and nuclease inhibitors.

Diagnostic devices are provided comprising one or more individual discrete volumes, each individual discrete volume comprising a CRISPR system as disclosed herein. In embodiments, the individual discrete volumes are droplets, are defined on a solid substrate, are microwells, or are spots defined on a substrate. Diagnostic devices can be configured to allow a mobile phone readout of the detectable signal, allowing for a smartphone application quantification of in-tube fluorescence.

Kits for detecting viral nucleic acids in a sample are provided, comprising nucleic acid amplification reagents; and one or more molecules designed according to the methods disclosed herein. In particular embodiments, the kits comprise a CRISPR system and one or more of the guide molecules designed according to the methods herein. Kits for detecting viral nucleic acids in a sample are provided comprising nucleic acid amplification reagents; and a CRIPSR system as disclosed herein.

Methods for developing or designing a therapy or therapeutic are provided, comprising optimizing a molecule for the therapy or therapeutic according to the methods disclosed herein, wherein specificity and sensitivity are optimized. Methods of modifying a target locus of interest are provided, comprising delivering to the target a molecule designed according to the methods herein. In certain embodiments, the designed molecule is an amplification primer, hybridization probe, toehold switch, or guide molecule. Methods of therapy may comprise delivering to the target a CRISPR system comprising one or more Cas proteins and wherein the molecule designed according to the methods is one or more guide molecules.

Compositions for modifying a target molecule are provided, the composition comprising a molecule designed according to the methods disclosed herein. The composition may comprise a designed molecule that is an amplification primer, hybridization probe, toehold switch, or guide molecule; in an aspect, the composition comprises a CRISPR system, the CRISPR system comprising one or more Cas proteins and one or more designed guide molecules.

Methods for detecting target nucleic acids in samples comprise contacting one or more samples with the systems disclosed herein, the system further comprising a polynucleotide-based masking construct comprising a non-target sequence, heating the sample for 5 to 10 minutes, wherein the Cas protein exhibits collateral nuclease activity and cleaves the non-target sequence of the nuclease-based masking construct once activated by the target sequence; and detecting a signal from cleavage of the non-target sequence, thereby detecting the one or more target sequences in the sample.

Methods of compositions may comprise the one or guide molecules is about 27 to about 29 nucleotides in length and/or the target locus of interest is provided via a nucleic acid molecule in vitro and/or the target locus of interest is provided via a nucleic acid molecule within a cell and/or the target locus of interest is provided via a nucleic acid molecule within a cell wherein the cell comprises a prokaryotic cell and/or the target locus of interest is provided via a nucleic acid molecule within a cell wherein the cell comprises a eukaryotic cell and/or the modification of the target locus of interest comprises a nucleotide strand break and/or the Cas protein is expressed from a nucleic acid molecule codon optimized for expression in eukaryotic cell and/or the effector protein is associated with one or more functional domains and/or the complex delivers an epigenetic modifier or a transcriptional or translational activation or repression signal and/or the complex delivers a functional domain that modifies transcription or translation of the target locus and/or the effector protein comprises at least one or more nuclear localization signals and/or when in complex with the Cas protein the guide molecule(s) is capable of effecting sequence specific binding of the complex to a target sequence of the target locus of interest and/or the guide molecules comprise a dual direct repeat sequence and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules comprise one or more regulatory elements operably configured to express the polypeptides and/or the guide molecule(s), optionally wherein the one or more regulatory elements comprise a promoter(s) or inducible promotor(s) and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules are comprised within one or more vector(s) and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and the one or more polynucleotide molecules are comprised within one vector and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules are comprised within one or more or one vector and the one or more vectors comprise viral vector(s) and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and the one or more polynucleotide molecules are comprised within one or more or one vector and the one or more vectors comprise viral vector(s) and the one or more viral vector(s) comprise one or more retroviral, lentiviral, adenoviral, adeno-associated or herpes simplex viral vector(s) and/or the effector protein and guide molecule(s) are provided via one or more polynucleotide molecules encoding the polypeptides and/or the nucleic acid component(s), and wherein the one or more polynucleotide molecules are operably configured to express the polypeptides and/or the nucleic acid component(s) and are comprised in a delivery system or the complex or its components or a component of the complex is comprised in a delivery system and/or delivering comprises a delivery vehicle comprising liposome(s), particle(s), exosome(s), microvesicle(s), a gene-gun or one or more viral vector(s) and/or the one or more polynucleotide molecules comprise one or more regulatory elements operably configured to express the polypeptides and/or the nucleic acid component(s), and the one or more regulatory elements comprise a promoter(s) or inducible promotor(s).

The therapeutic methods and compositions may comprise Cas proteins that are a Class 1 or Class 2 CRISPR protein, in an aspect the Cas protein is a Type II, Type V or Type VI protein, a Cas9, Cas12 or Cas13 protein. In embodiments, the target is associated with a disease, virus, is expressed in cancer cells, or is expressed in pathogen-infected cells.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is a block diagram depicting a portion of a simplified communications and processing architecture of a typical system to design sensitive, specific, and optimally active binding molecules, in accordance with certain examples of the technology disclosed herein.

FIG. 2 is a block diagram illustrating methods to design sensitive, specific, and optimally active binding molecules, in accordance with certain examples of the technology disclosed herein.

FIG. 3 is a block diagram illustrating methods to formulate and implement an approach to identify binding molecules with maximal activity across extensive genomic diversity, in accordance with certain examples of the technology disclosed herein.

FIG. 4 is a block diagram illustrating methods to construct a realistic activity function by developing a dataset and training models to predict activity, in accordance with certain examples of the technology disclosed herein.

FIG. 5 is a block diagram illustrating methods to develop an exact query algorithm to enforce specificity across viral taxa, in accordance with certain examples of the technology disclosed herein.

FIG. 6 is a block diagram illustrating methods to integrate methods with a branch and bound search over the genome to identify optimal assays, in accordance with certain examples of the technology disclosed herein.

FIG. 7 is a block diagram depicting a computing machine and modules, in accordance with certain examples.

FIG. 8 is an overview of Agile Design with Automated Patrolling of Targets (ADAPT). Steps in the design include 1) designing the target to be sensitive across sequence diversity, 2) designing the binding molecule to be specific to the target molecule, 3) predicting activity, and 4) generating optimally active sequence.

FIG. 9 depicts the searching for amplicons and guides in aligned genomes when using the model for a CRISPR system. Memoizing computations across amplicons is an extremely effective approach. Cost is calculated as β1*(# primers)+β2*log(amplicon length)+β3*(# guides). While O(L²) amplicons. Easy to prune with the best N.

FIG. 10 includes graphs that show ADAPT enables few guide anywhere along the diverse Lassa virus segment S. Vaseline approaches using 1 guide, mostly 60-80% detected. With ADAPT, 2-5 guides typically provide >99% detected everywhere, providing more design options.

FIG. 11 is a violin plot showing cross-validation providing >90% coverage on validation sequence data.

FIG. 12 is a violin plot showing G-U pairing enlarges space of hits. In an experiment sampling about 1M unique viral 28-mers from 570 species; querying each 28-mer against the other 569 species, the plots show the fractions with a hit querying with and without G-U pairing (left) and the number of hits per query, querying without and without G-U pairing (right). Non-specific hits are common for all species at 4 or more mismatches.

FIG. 13 depicts the pre-process and query approach to shard k-mer across tries.

FIG. 14 shows the approach as described in FIG. 13 lowers runtime and has promise for further improvement, with runtime decreased by 10-100× and the total nodes visited across tries either with shard (p=1 or p=2) or without sharding (right) suggesting parallelization may provide further speedup.

FIG. 15 depicts creation of datasets to model activity. As an example, the approach can be applied to Cas13-droplet technology for high-throughput screening, focusing on mismatches, with about 5000 guide-target pairs (94 guides) with 0 or 1 mismatch. In another example about 20,000 guide-target pairs with 2-6 mismatches will be created to model activity.

FIG. 16 provides modeling activity and approach for evaluation. Convolutional neural networking (CNN) with similarities to state of the art Cas9 (see, Lin et al., Bioinformatics 2018) and Cas12 (See Kim et al. Nature Biotechnology 2018) models can be utilized, making splits by guide position with no guide overlap across splits. Nested CV can be used to compar models; extensive CNN hyperparameter search using CV, with testing on held-out set.

FIG. 17 compares no locally connected layers with locally connected layers (no weight sharing) for mean validation MSE, with each point a choice of hyperparameters. Locally connected layers seem to help, which is surprising because this is a rare or nonexistent occurrence in other Cas9/12 models. It is believed possible it is a position-specific dimensionality reduction.

FIG. 18A charts guides across latent samples and across targets. Generated guides on test set show varied distances from their targets, helpful for exploring a space of guides. FIG. 18B includes interpretation of the latent space of variation. A heatmap (left) shows most latent samples alter multiple positions. The hamming distance averages over a target is provided (right) with distance of latent sample from 0 correlated with Hamming distance.

FIG. 19A-19B Testing an assay for SARS-CoV-2 using synthetic RNA targets. Shown are data from both fluorescent (19A) and lateral flow detection (19B). Target concentrations (in cp/μl) are indicated. RPA NTC: water input into RPA; Det NTC: water input into Cas13 detection reaction. In (19A), error bars indicate one standard deviation based on n=3 technical replicates.

FIG. 20A-20D Initial assay development for SHERLOCK-based SARS-CoV-2 detection. (20A) Schematic of two- and single-step SHERLOCK assays using RNA extracted from patient samples with a fluorescent or colorimetric readout. Times, range of suggested incubation times; pipette, step involving user manipulation. RT-RPA, reverse transcriptase-recombinase polymerase amplification; C, control line; T, test line. (20B) Schematic of the SARS-CoV-2 genome and SHERLOCK assay location. Sequence conservation across the primer and crRNA binding sites for publicly available SARS-CoV-2 genomes (see Methods for details). Text denotes nucleotide position with lowest percent conservation across the assay location. ORF, open reading frame; narrow rectangles, untranslated regions; dashed border, unlikely to be expressed (Kim et al. Cell 181:914-821 (2020)). (20C) Colorimetric detection of synthetic RNA using two-step SHERLOCK after 30 min. NTC r, non-template control introduced in RPA, NTC d, non-template control introduced in detection; T, test line; C, control line. (20D) Background-subtracted fluorescence of the two-step and original single-step SHERLOCK protocols using synthetic SARS-CoV-2 RNA after 3 h. The 1 h timepoint from this experiment is shown in FIG. 21E. NTC, non-template control introduced in RPA. Error bars, s.d. for 2-3 technical replicates.

FIG. 21A-21H Optimization of the single-step SHERLOCK reaction. (21A) Background-subtracted fluorescence of Cas13-based detection with synthetic RNA, reverse transcriptase, and RPA primers (but no RPA enzymes) after 3 h. (21B) Single-step SHERLOCK normalized fluorescence using various buffering conditions after 3 h. (21C) Background-subtracted fluorescence of single-step SHERLOCK with synthetic RNA and variable RPA forward and reverse primer concentrations after 3 h. (21D) Single-step SHERLOCK normalized fluorescence over time using two different fluorescent reporters (left) and two different reverse transcriptases (right). (21E) Background-subtracted fluorescence of the original single-step and optimized single-step SHERLOCK with synthetic RNA after 1 h. Data from the 3 h timepoint from this experiment is shown in FIG. 20D. (21F) Colorimetric detection of synthetic RNA input using optimized single-step SHERLOCK after 3 h. (21G) Optimized single-step SHERLOCK background-subtracted fluorescence using RNA extracted from patient samples after 1 h. (21H) Concordance between SHERLOCK and RT-qPCR for 7 patient samples and 4 controls. For (21C and 21E), see methods for details about normalized fluorescence calculations. For (21B, 21D, 21F, and 21G), NTC, non-template control. For (21B, 21D, 21E, and 21F), error bars, s.d. for 2-3 technical replicates. For (21B and 21D) RNA input at 10⁴ cp/82 L.

FIG. 22A-22C Additional two-step SHERLOCK testing. (22A) Colorimetric detection of synthetic DNA using two-step SHERLOCK after 3 h. NTC, non-template control; T, test line; C, control line. (22B) Colorimetric detection of HUDSON-treated SARS-CoV-2 viral seedstock using two-step SHERLOCK after 3 h. NTC, non-template control; T, test line; C, control line. (22C) Ct values of real-time RT-qPCR for extracted RNA from SARS-CoV-2 seedstock at various concentrations labelled by the result of Applicants' two-step SHERLOCK assay, performed side-by-side. The vertical line demarcates 1 cp/μl The horizontal line demarcates samples with non-quantifiable Ct values (i.e. no amplification), imputed as a Ct of 40.

FIG. 23A-23E Optimization of single-step for improved sensitivity. (23A) Background-subtracted fluorescence detected after the single-step SHERLOCK reaction was incubated for 3 h with DNA as input. (23B) Background-subtracted fluorescence of the Cas13-detection reaction (no RPA enzymes) with 3 h incubation. RNase H+, final concentration of 0.1 U/μl (23C) Background-subtracted fluorescence of the Cas13-detection reaction (no RPA) with 3 h incubation. (23D) Background-subtracted fluorescence detected after the single-step reaction was incubated for 3 h with varying magnesium concentrations. (23E) Background-subtracted fluorescence detected after the single-step reaction was incubated for 3 h with varying RPA primer concentrations. For (23A-23E), NTC, non-template controls; error bars, s.d. for 2-3 technical replicates. All listed concentrations refer to concentration within the reaction mixture before addition of the oligonucleotide template.

FIG. 24A-24B Optimization of fluorescent reporter. (24A) Single-step SHERLOCK normalized fluorescence (see methods for details) over time using quenched poly-uracil FAM reporters of varying lengths or RNaseAlert with RNA input at 104 cp/μl. (24B) Baseline fluorescence of poly-uracil FAM reporters or RNaseAlert in single-step SHERLOCK after 3 h.

FIG. 25 Single-step SHERLOCK time course. Optimized single-step SHERLOCK assay fluorescence over time at varying RNA input concentrations. Background-subtracted fluorescence at 1 h is shown in FIG. 21E. NTC, non-template control; error bars, s.d. for 3 technical replicates. Note: error bars for NTC are present but very small.

FIG. 26A-26I SARS-CoV-2 detection from unextracted samples using SHINE. (26A) Schematic of SHINE, which is HUDSON paired with single-step SHERLOCK using an in-tube fluorescent or colorimetric readout. Times, range of suggested incubation times; C, control line; T, test line. (26B) RNaseAlert fluorescence measured after 30 min at room temperature from universal viral transport medium (UTM), saliva, and phosphate buffered saline (PBS) after heat and chemical treatment. (26C) SARS-CoV-2 RNA detection in HUDSON-treated UTM as measured by single-step SHERLOCK and the in-tube fluorescence readout after 1 h. (26D) SARS-CoV-2 RNA detection in HUDSON-treated saliva as measured by single-step SHERLOCK and the in-tube fluorescence readout after 1 h. (26E) Schematic of the companion smartphone application for quantitatively analyzing in-tube fluorescence and reporting binary outcomes of SARS-CoV-2 detection. (26F) Colorimetric detection of SARS-CoV-2 RNA in unextracted patient NP swabs using the SHINE after 1 h. (26G) SARS-CoV-2 detection from unextracted patient samples using SHINE and smartphone application quantification of in-tube fluorescence after 40 min. Threshold line determined as average readout value for controls plus 3 standard deviations. (26H) Concordance table between SHINE and RT-qPCR for 50 patient samples.

FIG. 27A-27C HUDSON optimization experiments. (27A) Samples were treated with 100 mM TCEP and 1 mM EDTA and subjected to a 20 min heating step at 50° C. RNase inhibitor, 4 U/μl. (27B) Samples were treated with 100 mM TCEP, 1mM EDTA, and 4 U/μl ARNase inhibitor. (27C) Samples were treated with 100 mM TCEP and 1 mM EDTA and subjected to a 5 min heating step at 50° C. (27A-27C) Positive and negative controls undergo no treatment. RNaseAlert (final concentration: 200 nM) was added immediately after the heating step.

FIG. 28 SHINE for UTM and saliva with colorimetric detection. HUDSON-treated UTM (left) and saliva (right) with synthetic RNA template added after initial heating step. Treated samples were used as input into single-step SHERLOCK with the colorimetric readout.

FIG. 29A-29B SHINE for UTM and saliva with in-tube fluorescent detection. (29A-29B) HUDSON-treated UTM (29A) and saliva (29B) with synthetic RNA template added after initial heating step. Samples were used as input in the single-step SHERLOCK assay. Transilluminator images were captured using a smartphone camera. NTC, non-template control.

FIG. 30A-30B Limit of detection of SHINE on UTM and saliva. (30A-30B) HUDSON-treated UTM (30A) and saliva (30B) with synthetic RNA added after the initial heating step when nucleases are inactivated. Samples were used as input in the single-step SHERLOCK assay incubated for 1 h. Transilluminator images were captured using a smartphone camera and analyzed by the companion smartphone application (App).

FIG. 31 SHINE on unextracted patient samples. HUDSON-treated NP swabs in UTM were used as input in the single-step SHERLOCK assay. Transilluminator images were captured using a smartphone camera after 40 minutes.

FIG. 32 SHINE's ability to detect viral RNA is significantly associated with the RT-qPCR threshold cycle. Viral Ct values measured by SARS-CoV-2 RT-qPCR of extracted RNA from 30 patient NP samples grouped by the result of SARS-CoV-2 SHINE. The association between viral Ct and SHINE outcome was assessed using a one-sided Wilcoxon rank sum test. **, p=0.0084.

FIG. 33—ADAPT's approach to comprehensive design. (a) Diagnostic performance for influenza A virus subtyping may degrade over time, even considering the most conserved sites. At each year, Applicants select the 15 most conserved 30-mers from recent sequences for segment 6 (N) for all N1 subtypes; each point represents a 30-mer. Plotted value is the fraction of sequences in subsequent years (colored) that contain the 30-mer; bars indicate the mean. To aid visualization, only odd years are shown. 2007 N1 30-mers are absent following 2007 owing to shift during the 2009 H1N1 pandemic. (b) Variants along the SARS-CoV-2 genome emerging over time. Bottom row (Combined') shows all 315 variants, against a reference genome, that cross a 0.1% or 1% frequency threshold between Feb. 7 and May 9, 2020i.e., variants either (1) at <0.1% frequency in genomes collected through February 7 and at 0.1% to 1% frequency in the 17,712 genomes collected through May 9 (low; light purple); or (2) at <1% frequency in genomes collected through February 7 and at _1% frequency in genomes collected through May 9 (high; dark purple). Labeled rows indicate the week in which each variant crosses the frequency threshold. (c) ADAPT's approach for designing maximally active probe sets. ADAPT finds representative subsequences in a genomic region that span known diversity, which are possible probes (colored). ADAPT calculates an activity (shaded) between each of these probes and each target sequence, forming the ground set of probes. ADAPT finds a probe set P, a subset of the ground set, maximizing an objective function of the expected value of A(P, s), the activity between P and a sequence s, subject to soft and hard constraints on P. (d) Fraction of Lassa virus (LASV; segment S) genomes detected, with different design strategies in a 200 nt sliding window, using a model in which 30 nt probes detect a target if they are within 1 mismatch, counting G-U pairs at matches. ‘Consensus’, probe-length consensus subsequence that detects the most number of genomes; ‘Mode’, most common probe-length subsequence. ADAPT, with hard constraints of 1-3 probes, maximizes activity. (e) ADAPT with a dual objective: minimal number of guides to detect >90%, >95%, and >99% of LASV genomes using the model in (d). In (d) and (e), shaded regions are 95% pointwise confidence bands calculated across randomly sampled input genomes.

FIG. 34 Enforcing taxon-specificity Predicting detection activity of CRISPR-Cas13a. (a) The library consists of a 865 nt long wildtype target sequence and 91 guide RNAs complementary to it, along with 225 unique targets containing mismatches and varying PFS alleles relative to the wildtype. Applicants measure fluorescence every _20 minutes for each pair and use the growth rate to quantify activity. The dataset contains 19,209 unique guide-target pairs. (b) Applicants predict activity for a guide-target pair in two parts, which requires training a classifier on all pairs and a regression model on the active pairs. (c) Results of model selection, for classification, with nested cross-validation. For each model and input type (color) on five outer folds, Applicants performed a five-fold cross-validated hyperparameter search. Plotted value is the mean auROC of the five models, and error bar indicates the 95% confidence interval. L1 LR and L2 LR, logistic regression; L1L2 LR, elastic net; GBT, gradient-boosted classification tree; RF, random forest; SVM, support vector machine; MLP, multilayer perceptron; LSTM, long short-term memory recurrent neural network; CNN, neural network with parallel convolutional filters and a locally connected layer. One-hot (1D), one-hot encoding of target and guide sequence without explicit pairing; One-hot MM, one-hot encoding of target sequence and of mismatches in guides relative to the target; Handcrafted, curated features of hypothesized importance (details in Methods); One-hot (2D), one-hot encoding of target and guide sequence with encoded guide-target pairing. Supplementary FIG. 8 shows auPR and regression models. (d) ROC curve of CNN, which is used in ADAPT, classifying pairs as inactive or active on a held-out test set. Points indicate sensitivity and FPR for a baseline classifier: choosing a guide-target pair to be active if it has a non-G PFS and the guide-target Hamming distance is less than a specified threshold (color). Inset plot shows comparison of FPR between CNN (black) and baseline classifiers at equivalent sensitivity. Red ‘+’ indicates decision threshold in ADAPT. (e) Regression results of CNN for predicting activity values from active guide-target pairs in the test set. Color, point density. _, Spearman correlation. Not shown: points with predicted activity <4 (0.23% of all). (f) Sample data as (e). Each row contains one quartile of pairs according to predicted activity (top row is predicted most active), with the bottom row showing all active pairs. Smoothed density estimates and interquartile ranges show the distribution of true activity for the pairs from each quartile. P-values are computed from Mann-Whitney U tests (one-sided).

FIG. 35—End-to-end design with ADAPT. (a) Sketch of ADAPT's steps. ADAPT accepts a list of taxonomic identifiers for which to design assays and fetches and curates their sequences from NCBI's viral genome databases. It then performs a branch and bound search to find suitable genomic regions—typically, each is an amplicon—that contain a maximally active probe set, while enforcing taxon-specificity. ADAPT outputs the top non-overlapping design options, ranked by an objective function. (b) Cross-validated evaluation of detection. For each species, Applicants ran ADAPT on 80% of available genomes and estimated performance, averaged over the top 5 design options, on the remaining 20%. Distributions are across 20 random splits and dots indicate mean. Purple, fraction of genomes detected by primers and for which Cas13a guides are classified as active. Green, same except Cas13a guides also have regressed activity in the top 25% of Applicants' dataset. NIPV, Nipah virus; EBOV, Zaire ebolavirus; ZIKV, Zika virus; LASV S/L, Lassa virus segment S/L; EVA, Enterovirus A; RVA, Rhinovirus A. (c) Number of Cas13a guides in the highest-ranked design option for each species. Color indicates the length of the targeted region (amplicon) in the design. (d) Activity of each guide set with two summary statistics: median and the 5th-percentile taken across each species's sequences. For the latter, value a indicates that 95% of sequences are detected with activity_a. Dashed line indicates the “high activity” threshold from (b). Sequences at 0 activity are classified as not being detected. (e) End-to-end elapsed real time running ADAPT. In (c-e), each point is a species.

FIG. 36—Growing number of viral genomes and diversity. Growth of data over time for 573 viral species known to infect humans. Each species is a color. (a) Cumulative number of genome sequences, counted from NCBI5 viral genome neighbors and influenza databases, for each species that were available up to each year. For genomes with multiple segments, this counts only the number of sequences of the segment that has the most sequences. 5 species with the most number of genomes are labeled. IAV, influenza A virus; RVA, rotavirus A; HBV, hepatitis B virus; IBV, influenza B virus; DENY, dengue virus. (b) Number of unique 31-mers for the genomes in (a), a simple measure of diversity. In both panels, year indicates the year of the entry creation data in the database. HIV-1, human immunodeficiency virus 1; HCV, hepatitis C virus; HHV-5, human betaherpesvirus 5.

FIG. 37—Comprehensiveness of conserved influenza A virus 30-mers over time. Even when considering the most conserved sequences, diagnostic performance of probes can degrade over time owing to genomic changes. At each year, Applicants select the 15 most conserved non-overlapping 30-mers according to recent sequence data up to that year—a simple model for designing diagnostic probes at different years, without any consideration to other constraints such as specificity or activity. Each point represents a 30-mer from the year in which it was designed. Applicants then measure the fraction of all sequences in subsequent years (colored) that contain each 30-mer—a simple test of comprehensiveness. Bars indicate the mean fraction of sequences containing the 15 30-mers at each combination of design and test year. To aid visualization, only odd years are shown. (a) Segment 6 (N) sequences from all N2 subtypes. (b) Segment 4 (H) sequences from all H1 subtypes. (c) Segment 4 (H) sequences from all H3 subtypes. FIG. 1a shows segment 6 (N) sequences from N1 subtypes.

FIG. 38—Comparison of algorithms for submodular maximization. Objective values of the optimal solutions identified by two algorithms for submodular maximization: the canonical greedy algorithm for monotone functions36 and a randomized algorithm with provable guarantees on non-monotone functions35. The function here is non-monotone, but results are comparable. Three viral species are shown. Each was evaluated for two choices of the weight on the soft constraint/penalty, indicated by_, as well as different choices of the soft cardinality constraint (h) and hard constraint (H) with h_H. Supplementary Note 1 contains a definition of the objective function, including the penalty weight and cardinality constraints. Each point indicates the result of one of 5 runs; differences account for randomness both in the randomized greedy algorithm and in constructing the ground set.

FIG. 39—Comprehensiveness of probe design. (a) Same as FIG. 1d but with additional viruses. (b) Same as FIG. 1e but with additional viruses. Gaps at a site are present when it is not possible to construct a probe set that reaches the desired coverage, owing to gaps or missing data.

FIG. 40—Guide-target library design. (a) Top, depiction of the wildtype target. The wildtype contains a T7 promoter on the 5′ end for transcription, four positive control regions, and three experimental regions. Each positive control region contains a unique guide that matches perfectly all targets, except negative control targets (not shown). Bottom, zoom of one experimental region from the wildtype target. Guide sequence comes from tiling along this region—29 guides per experimental region. There are also negative control guides (not shown) that only match the negative control targets. Other targets contain mismatches relative to the wildtype target, and thus contain mismatches relative to the guide sequences. (b) Distribution of the Hamming distance between guide and target across guide-target pairs. Color represents the number of pairs with each protospacer flanking site (PFS) at each Hamming distance. The 19,209 unique guide-target pairs included in Applicants' final, curated dataset (Methods) are shown. (c) Same as (b), but the distribution of PFS across the guide-target pairs. Color represents the number of pairs with each nucleotide immediately following the PFS (3′ end of protospacer).

FIG. 41—Assessing activity through CRISPR-Cas13a reaction kinetics. (a) Cas13 activity for a series of target concentrations, using two control targets and guides from Applicants' Cas13 library. Applicants model fluorescence for each guide-target pair over time (FIG. 2a; Methods), fitting a curve of the form C(1 e kt)+B where t is time and e kt represents remaining reporter presence over time. Applicants take log(k) to be the measure of Cas13 activity. (b) Theoretical fluorescence saturation over time—namely, the term 1 e ktfor five activity values. Over the time scale of Applicants' experiment (t up to_120 minutes), Applicants cannot observe reporter activation when k is small, motivating the use of an activity cutoff. Therefore, Applicants label guide-target pairs with log(k)_4 as inactive and those with log(k)>4 as active.

FIG. 42—Dataset of CRISPR-Cas13a guide-target pairs. All panels show the 18,508 unique guide-target pairs in the dataset for training and testing. Activity is defined in Methods. (a) Distribution of number of replicate activity measurements for each pair. (b) Distribution of standard deviation across replicate activity measurements for each pair. (c) Activity of each guide against the wildtype target (matching exactly), shown by their position along the target. Dot indicates the mean activity across the wildtype targets, shown with a 95% confidence interval. (d) Variation in activity across guide-target pairs and among replicate measurements. Each row represents a guide-target pair. Purple dot indicates the mean activity across replicate measurements; pairs are sorted vertically by this value. Bars indicate the 95% confidence interval for the mean. (e) Variation in activity between guides and across targets for each guide. Each row represents a guide. Black dot indicates the median activity across all targets and bars span the 20th and 80th percentiles of activity across all targets. Purple dot indicates the mean activity across the wildtype targets (matching the guide exactly). (f) Distribution of activity across all guide-target pairs and only pairs with the wildtype target. (g) Distribution of activity across all guide-target pairs in the training data and the pairs in the test data (the two sets do not overlap along the target or contain the same guides; Methods). In (dg), there are 10 resampled replicate activity measurements for each guide-target pair.

FIG. 43—Nested cross-validation for classification and regression. For each model and input type (color) on each of five outer folds, Applicants performed a five-fold cross-validated hyperparameter search. The plotted value is the mean of a statistic across the five outer folds, and the error bar indicates the 95% confidence interval. (a) Area under precision-recall curve (auPR) for different classification models. auROC is in FIG. 2c. L1 LR and L2 LR, logistic regression; L1L2 LR, elastic net; GBT, gradient-boosted classification trees; RF, random forest; SVM, support vector machines; MLP, multilayer perceptron; LSTM, long short-term memory recurrent neural network; CNN, convolutional neural network including parallel convolution filters of different widths and a locally-connected layer. One-hot (1D) is one-hot encoding of both target and guide sequence without explicit pairing; One-hot MM is one-hot encoding of target sequence and of mismatches in guides relative to the target; Handcrafted is curated features of hypothesized importance (Methods); One-hot (2D) is one-hot encoding of both target and guide sequence with encoded guide-target pairing. (b) Mean squared error (MSE) for different regression models (lower is better). L1 and L2 LR, regularized linear regression; L1L2 LR, elastic net; GBT, gradient-boosted regression trees; RF, MLP, LSTM, and CNN are as in (a) except constructed for regression. Input types are as in (a). (c) Same as (b) but the statistic is Spearman correlation.

FIG. 44—Architecture of convolutional neural network for guide-target activity prediction. Convolutional neural network (CNN) architecture for classifying and regressing activity; hyperparameter search and training is separate for each task. The inputs are one-hot encoded for the target and guides sequences (8 channels in total). There multiple convolutional filters of different widths processing the input in parallel, as well as multiple locally connected filters of different widths; outputs of these different filters are concatenated in the merge layer. Pooling includes maximum, average, and both. ‘BN’ is batch normalization and ‘FC’ is fully connected. There are N fully connected layers. The dropout layers are in front of each fully connected layer.

FIG. 45—Hyperparameter search for convolutional neural networks. Applicants used a random search over the hyperparameter space (200 draws) to select each convolutional neural network (CNN) model. Each plot corresponds to a hyperparameter and shows choices of that hyperparameter; see Methods for all hyperparameters. The evaluations are cross-validated: each dot indicates the mean of a metric, computed across five folds, for a draw of hyperparameters. ‘LC’, locally connnected. The ‘+’ in LC and convolutional widths separates different widths of parallel filters; ‘None’ indicates that the model does not use an LC or convolutional layer. P-values are computed from Mann-Whitney U tests (one-sided). (a) Results of hyperparameter search for classification. BCE, binary cross-entropy. (b) Results of hyperparameter search for regression. MSE, mean squared error.

FIG. 46—Precision-recall curve of classifier. (a) Precision-recall (PR) curve of CNN model, which is used in ADAPT, classifying pairs as inactive or active on a held-out test set. ROC curve is in FIG. 2d. Points indicate precision and recall for a baseline classifier: choosing a guide-target pair to be active if and only if it has a non-G PFS and the Hamming distance between the guide and target is less than the specified threshold (color). Red ‘+’ indicates the decision threshold in ADAPT. Dashed line is precision of random classifier (equivalently, the fraction of guide-target pairs that are active). (b) Comparison of precision between CNN (black) and baseline classifiers (color as in (a)) at equivalent recall.

FIG. 47—Regression results on guide-target pairs classified as active. (a) Same as FIG. 34e, except on guide-target pairs that are classified to be active by the CNN reported in FIG. 34d (other data show regression results on pairs that are true active). Color, point density. _, Spearman correlation. (b) Same as FIG. 34f, except on guide-target pairs that are classified to be active. Each row contains one quartile based on their predicted activity (top row is predicted most active), with the bottom row showing all pairs classified to be active. Smoothed density estimates and interquartile ranges show the distribution of true activity for the pairs from each quartile. P-values are computed from Mann-Whitney U tests (one-sided).

FIG. 48—Importance of features in linear models. (a) Feature coefficients in linear models for classification. Plotted value is the mean of the coefficient across training on five folds, and error bar is the 95% confidence interval. Input type for all models is ‘One-hot MM+Handcrafted’ as defined in FIG. 34c and FIG. 43. Coefficients are sorted by absolute value and the top 20 are shown. ‘L1+L2’ is elastic net. PFS, protospacer flanking site (3′ end) with two positions. All positions are defined based on the protospacer target sequence and are 0-based. Mismatch alleles are in the guide spacer sequence and are mismatches relative to the target. (b) Same as (a) but for regression models. Note that positive coefficients for mismatch features do not necessarily imply that mismatches improve activity compared to a matching guide-target pair.

FIG. 49—Classification performance on subsets of test data. Evaluations of classification on different subsets of the held-out test data corresponding to variables that may considerably affect activity. Here, the model is the same for all evaluations and only tested (not trained) on different subsets. (a) ROC curves computed from guide-target pairs with the different protospacer flanking sites (PFS). (b) Precision-recall (PR) curves computed from pairs with the different PFS. Dashed lines are precision of random classifiers for each PFS (equivalently, the fraction of guide-target pairs that are active with each PFS). (c) ROC curves computed from pairs with different Hamming distances between guide and target. (d) PR curves computed from pairs with different Hamming distances between guide and target. Dashed lines are precision of random classifiers for each choice of Hamming distance (equivalently, the fraction of guide-target pairs that are active at each Hamming distance). In all panels, yellow curve is for all test data.

FIG. 50—Regression performance on subsets of test data. Evaluations of regression on different subsets of active guide-target pairs in the held-out test data, where the subsets correspond to variables that may considerably affect activity. Here, the model is the same for all evaluations and only tested (not trained) on different subsets. (a) Each plot corresponds to one guide, with the points shown representing guide-target pairs across the different targets the guide detects. Number above each plot is the position of the guide along the wildtype target. (b) Pairs separated by the different protospacer flanking sites (PFS), indicated above each plot. Each point is a guide-target pair. (c) Pairs separated by the different PFS. Each row contains one quartile based on their predicted activity (top row is predicted most active), with the bottom row showing all active pairs with the PFS. Numbers indicate the number of pairs in each quartile; the quartile for each pair is based on its predicted activity across all PFS, not only the PFS for each plot. (d) Guide-target pairs colored by PFS. Same data as in FIG. 2e. (e) Same as (b), except separated by different Hamming distances between guide and target. (f) Same as (c), except separated by Hamming distance. (g) Same as (d), except colored by Hamming distance. In (a), (b), and (e), is Spearman correlation.

FIG. 51—Learning curves. Learning curves for the convolutional neural networks used in ADAPT. At each number of input training data points, Applicants perform nested cross-validation to select models: on each of five outer folds, Applicants perform a five-fold cross-validated hyperparameter search to select a model. Line indicates the mean of a statistic on the validation data across the five selected models and error bars give a 95% confidence interval. (a) Learning curve selecting models for classification. (b) Learning curve selecting models for regression.

FIG. 52—CRISPR-Cas13a guide-target activity. (a) Fraction of guide-target pairs that are active for each 2-nt protospacer flanking site (PFS; i.e., the canonical Cas13 PFS together with the nucleotide adjacent on the 3′ side of the protospacer). This analysis considers only matching guide-target pairs (i.e., no mismatches) and determines a pair to be active if the median log(k) value across replicates is >2. Error bars represent 95% exact binomial confidence intervals. (b) Density of activity for different numbers of mismatches between guides and targets. Here, the number of mismatches is equivalent to Hamming distance. (c) Profile of mismatches among guide-target pairs with similar activity. Each row in the heatmap represents a guide-target pair, ordered by activity, with those having the least activity on top; values on the left indicate activity. For each row y, Applicants consider the 1,000 guide-target pairs with activity closest to the pair represented by y. Then, at each position x in the protospacer, Applicants consider all mismatches at x across Applicants' dataset and calculate the fraction of them to which the 1,000 guide-target pairs, centered at y, contribute. Applicants plot this fraction; higher values at a row indicate a preponderance of mismatches among the guide-target pairs with the activity represented by that row. (d) Density of guide-target pairs that have no mismatches (purple) compared to those that have at least one mismatch in the first four positions of the protospacer and no mismatch elsewhere (yellow). As in (b), here a G-U pair is counted as a mismatch.

FIG. 53—Potential hits with tolerance of G-U base pairing. Being tolerant of G-U base pairing increases the potential for non-specific hits of a k-mer. Applicants built an index of 1 million 28-mers from 570 human-associated viral species. For each of 100 randomly selected species, Applicants queried 28-mers for hits against the other 569 species (details in Methods). Applicants performed this for each choice of m mismatches, counting a non-specific hit as one within m mismatches of the query, both being sensitive to G-U base pairing (purple; counting it as a match) and not being sensitive to it (green; counting it as a mismatch). Violin plots show the distribution, across the selected species, of the mean of the measured value. (a) Fraction of queries that yield a non-specific hit. The measured value for a query is 0 (no hit) or 1 (_1 hit), so the mean represents the fraction with a hit. (b) Number of non-specific hits per query.

FIG. 54—Sharding k-mers across tries for specificity queries. (a) Constructing a bit signature after transforming a string to a two-letter alphabet, described in Supplementary Note 2b. Two strings that match up to G-U base pairing (shown here as G-T) have the same bit signature. (b) Inserting a k-mer into the data structure of tries. Each k-mer is inserted into p tries, and there are p_2k=p tries in total. (c) Querying a k-mer for near neighbors (within m mismatches, sensitive to G-U base pairing as a match).

FIG. 55—Benchmarking of specificity queries. (a) The runtime of querying using an index of _1 million 28-mers across 570 human-associated viral species. For each of 100 randomly selected species, Applicants queried 28-mers for hits against the other 569 species. Violin plots show the distribution, across the selected species, of the mean runtime for each query. Green shows results on a single, large trie of 28-mers; purple (p=1) and yellow (p=2) show results on the approach described in Supplementary Note 2d, with two choices of the partition number p. (b) Same as (a), but showing total number of nodes visited across the trie(s). The decrease in this value using Applicants' approach suggests that parallelizing the approachby searching within multiple tries in parallel—may provide a further speedup.

FIG. 56—Dispersion in ADAPT's designs. For each species, Applicants ran ADAPT 20 times. For each pair of runs, Applicants calculate the Jaccard similarity comparing the top 5 design options from each. Violin plots show a smoothed density estimate of the pairwise Jaccard similarities; dot indicates the mean and bars show 1 standard deviation around the mean. (a) Using resampled input genomes for each run and considering two design options to be equal if they have exactly the same primers and probes. (b) Using the same input genomes for each run and considering two design options to be equal if they have exactly the same primers and probes. (c) Using resampled input genomes for each run and considering two design options to be equal if their endpoints are within 40 nt of each other. (d) Using the same input genomes for each run and considering two design options to be equal if their endpoints are within 40 nt of each other. When using resampled input genomes, the comparisons account for algorithmic randomness and input sampling. When using the same input genomes, the comparisons account only for algorithmic randomness. EBOV, Zaire ebolavirus; EVA, Enterovirus A; LASV L/S, Lassa virus segment L/S; NIPV, Nipah virus; ZIKV, Zika virus.

FIG. 57—Results of ADAPT's designs for 1,926 vertebrate-infecting viruses. Running ADAPT on 1,933 vertebrate-infecting species produced designs on 1,926 (Methods). (a) Length of each target region (amplicon) in the highest-ranked design output by ADAPT for each species. As part of the design Applicants restricted the length to _250-nt for all species except two (Methods). Horizontal axis is the number of input sequences for design. (b) Number of Cas13a guides in the highest-ranked design option for each species, produced using the objective function in which Applicants minimize the number of guides subject to detecting >98% of sequences with high activity. Color indicates the length of the targeted region (amplicon) in the design. (c) Maximum resident set size (RSS), in MB, of the process running ADAPT on each species. (d) Distribution, across species, of the fraction of input sequences passing curation. (e) Fraction of input sequences passing curation for each species compared the number of input sequences for that species. (f) Number of clusters for each species compared to the number of input sequences for that species.

FIG. 58—Effects of enforcing specificity on ADAPT's designs for 1,926 vertebrate-infecting viruses. In each panel, each point is a species and comparisons are with and without enforcing species-level specificity within each family. (a) End-to-end elapsed real time running ADAPT. (b) Maximum resident set size (RSS), in MB, of the process running ADAPT. (c) Mean activity of the guide set, from the highest-ranked design option, across input sequences. (d) Objective value of the highest-ranked design option, which incorporates expected activity of the guide set, the number of primers, and the target region length. Not shown, 9 species with objective value <0. In all panels, 1,926 species are shown (7 of the 1,933 vertebrate-infecting species did not produce designs; Methods).

FIG. 59—Searching for genomic regions. ADAPT searches for a region of the genome, bound by conserved sequence to use for primers, that contains probes that can collectively detect the region. The requirement that a region be bound by conserved sequence and represent an amplicon is optional.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M. J. MacPherson, B.D. Hames, and G. R. Taylor eds.): Antibodies, A Laboratory Manual (1988) (Harlow and Lane, eds.): Antibodies A Lab oraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011) .

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +1-10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and systems for designing binding molecules, and methods of using the designed binding molecules and systems to provide end-to end design and use of highly sensitive, specific and optimally active binding molecules for targets that find use in therapeutic and diagnostic applications. In embodiments, the binding molecules utilize nucleic acid or protein technologies. The methods are applicable to design of molecules for high-throughput systems, which in embodiments, can comprise a CRISPR system diagnostic.

The methods disclosed herein have broad applications in diagnostics, serologic tests, for use in therapies and drug resistance, allowing for agile design in the detection of targets. The end-to-end design provided herein includes designing molecules to be sensitive across target sequence diversity, designing the molecules to be specific to the target, predicting of activity of designed molecules, and generating optimally active binding molecule sequences.

Exemplary development is provided to help address the challenge of testing for SARS-CoV-2 and numerous other respiratory viral pathogens providing a set of comprehensive design options for 67 species and subspecies for CRISPR-based detection assays.

### Design of Binding Molecules

The technology described herein includes computer implemented methods, computer program products, and systems to design viral diagnostics could rapidly render highly effective assays using the latest genomic diversity, including from novel species or strains. The method comprises designing a set of diagnostic molecules to be sensitive across sequence diversity for a set of target sequences. Diagnostic molecules and binding molecules are used herein interchangeably. The system solves for sets of diagnostic molecules that with maximal detection activity across extensive genomic diversity. The system then constructs a realistic activity function by developing a dataset and training models to predict activity. Then the system performs a query algorithm that indexes k-mers from all inputs and splits the k-mers across many small tries according to a hash function. The query algorithm determines the specificity of the binding molecule, tolerating both high divergence and G-U wobble base pairing. The system performs a branch and bound search over the genome of a sample virus using the binding molecule to create a ranked set of assay options. The ranked binding molecule options may be used to detect desired viruses in a sample.

### Example System Architectures

Turning now to the drawings, in which like numerals represent like (but not necessarily identical) elements throughout the figures, example embodiments are described in detail.

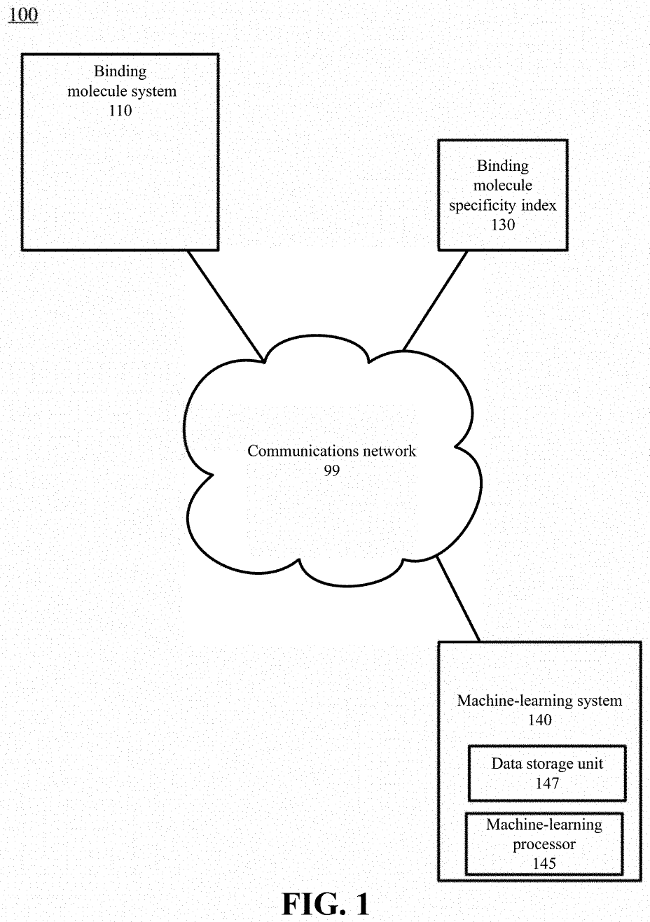

FIG. 1 is a block diagram depicting a portion of a simplified communications and processing architecture of a typical system 100 to design sensitive, specific, and optimally active binding molecules, in accordance with certain examples. As used herein, the term “binding molecules” means sequence-specific molecules designed to hybridize to a target or set of target molecules. In some embodiments, a user 101 associated with a user computing device 110 must install an application and/or make a feature selection to obtain the benefits of the techniques described herein.

As depicted in FIG. 1, the system 100 includes network computing devices 110, 130, and 140 that are configured to communicate with one another via one or more networks 105 or via any suitable communication technology.

Each network 99 includes a wired or wireless telecommunication means by which network devices (including devices 110, 130, and 140) can exchange data. For example, each network 99 can include a local area network (“LAN”), a wide area network (“WAN”), an intranet, an Internet, a mobile telephone network, storage area network (“SAN”), personal area network (“PAN”), a metropolitan area network (“MAN”), a wireless local area network (“WLAN”), a virtual private network (“VPN”), a cellular or other mobile communication network, Bluetooth, NFC, or any combination thereof or any other appropriate architecture or system that facilitates the communication of signals, data. Throughout the discussion of example embodiments, it should be understood that the terms “data” and “information” are used interchangeably herein to refer to text, images, audio, video, or any other form of information that can exist in a computer-based environment. The communication technology utilized by the devices 110, 130, and 140 may be similar networks to network 99 or an alternative communication technology.

Each network computing device 110, 130, and 140 includes a computing device having a communication module capable of transmitting and receiving data over the network 99 or a similar network. For example, each network device 110, 130, and 140 can include a server, desktop computer, laptop computer, tablet computer, a television with one or more processors embedded therein and/or coupled thereto, smart phone, handheld or wearable computer, personal digital assistant (“PDA”), wearable devices such as smart watches or glasses, or any other wired or wireless, processor-driven device. In the example embodiment depicted in FIG. 1, the network devices 110, 130, and 140 are operated by end-users or operators to design binding molecules.

A user can use a device or application on a binding molecule system 110, which may be, for example, a web browser application, a stand-alone application, a server based device, or any other type of device to perform the operations herein via a distributed network 99. The binding molecule system 110 can interact with web servers or other computing devices connected to the network 99, including a Binding molecule specificity index 130 or a machine-learning system 140. In another example, the binding molecule system 110 communicates with other devices via near field communication (“NFC”) or other wireless communication technology, such as Bluetooth, WiFi, infrared, or any other suitable technology.

The binding molecule specificity index 130 includes a database or index of stored or created data related to the specificity of a particular binding molecule. The binding molecule specificity index 130 may use data provided by the methods described herein.

The machine-learning system 140 may include a data storage unit 147 and a machine-learning processor 145. The example data storage unit 147 can include one or more tangible computer-readable storage devices, or the data storage unit may be a separate system, such as, a different physical or virtual machine, or a cloud-based storage service. The machine-learning system 140 represents any type of neural network computing system or other computing system that employs any machine-learning process or algorithm that operates on a machine-learning processor 145. The machine-learning system 140 is able to receive data from many varied sources and use the data to interpret patterns and characterize features of users 101, instruments, issuers systems 130, and others involved in the transaction process. The machine-learning system 140 is able to continually or periodically update the received information in a manner that allows the data presented by the digital wallet system 140 to become more useful and accurate as more data is received and stored. The G machine-learning system 140 may be a function or computing device of binding molecule system 110 or any other suitable system. Alternatively, the machine-learning system 140 may be hosted by a third party system or any other suitable host. The machine-learning system 140 represents an example of a machine-learning process or algorithm. Any other suitable process may be used, such as a different supervised learning process, an unsupervised learning process, or reinforcement learning. The machine-learning system 140 may be based on a convolutional neural network, support vector machines, linear regressions, decision trees, or any other type of common algorithms.

It will be appreciated that the network connections shown are examples and other means of establishing a communications link between the computers and devices can be used. Moreover, those having ordinary skill in the art having the benefit of the present disclosure will appreciate that the binding molecule specificity index system 130, machine-learning system140, and the binding molecule system 110 illustrated in FIG. 1 can have any of several other suitable computer system configurations. For example, a user computing device 110 embodied as a mobile phone or handheld computer may not include all the components described above.

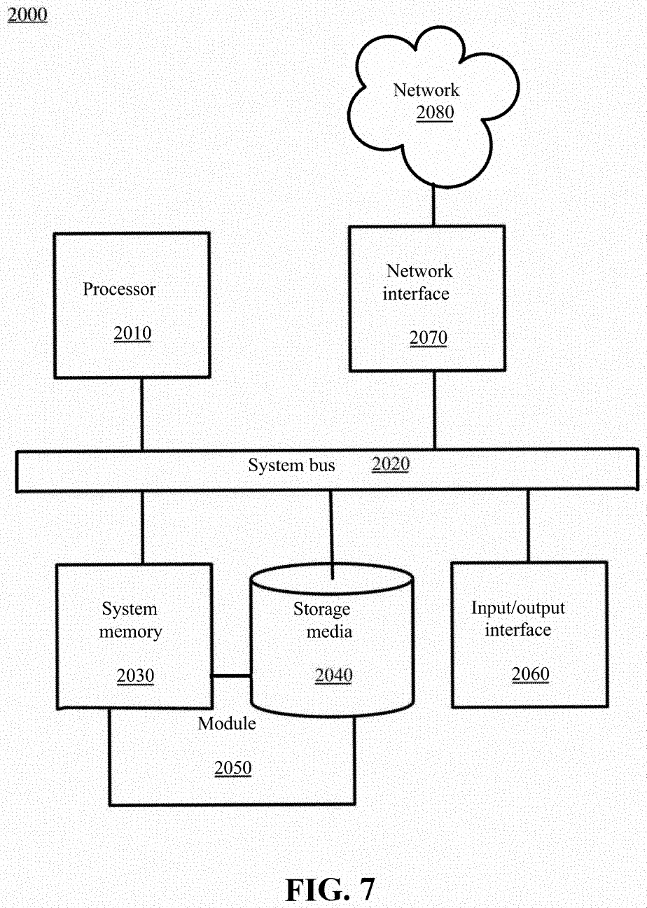

In example embodiments, the network computing devices and any other computing machines associated with the technology presented herein may be any type of computing machine such as, but not limited to, those discussed in more detail with respect to FIG. 7. Furthermore, any modules associated with any of these computing machines, such as modules described herein or any other modules (scripts, web content, software, firmware, or hardware) associated with the technology presented herein may by any of the modules discussed in more detail with respect to FIG. 7. The computing machines discussed herein may communicate with one another as well as other computer machines or communication systems over one or more networks, such as network 99. The network 99 may include any type of data or communications network, including any of the network technology discussed with respect to FIG. 7.

### Example Processes

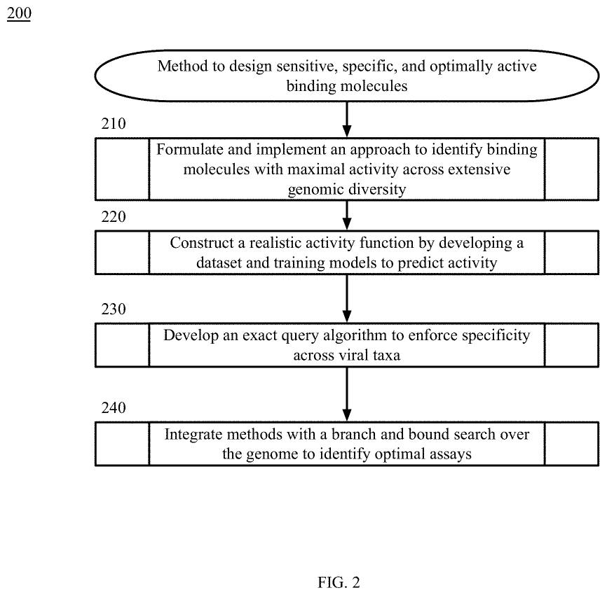

The example methods illustrated in FIGS. 2-6 are described hereinafter with respect to the components of the example architecture 100. The example methods also can be performed with other systems and in other architectures including similar elements.

Referring to FIG. 2, and continuing to refer to FIG. 1 for context, a block diagram illustrates methods 200 to use aggregated health data to provide health assessments, in accordance with certain examples of the technology disclosed herein.

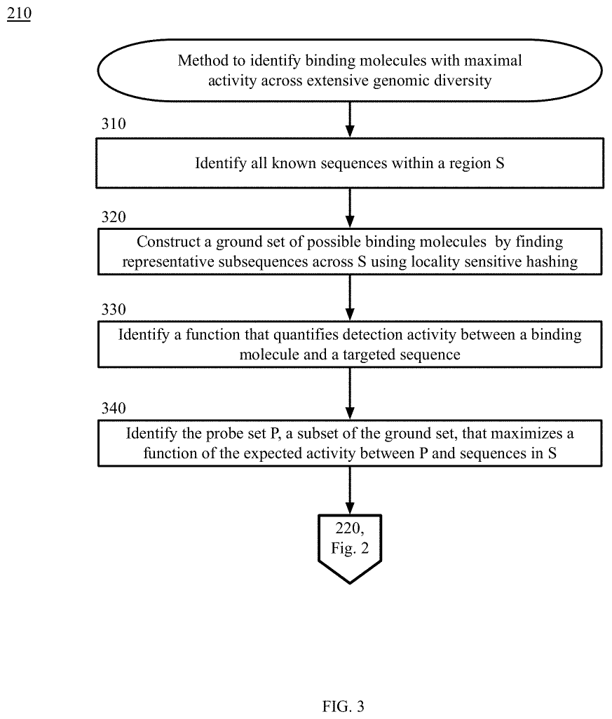

In block 210, the binding molecule system 110 designs a set of binding molecules to be sensitive across sequence diversity for a set of target sequences. Block 210 is described in greater detail with respect to FIG. 3.

In block 210, the binding molecule system 110 formulates and implements an approach to identify binding molecules with maximal activity across extensive genomic diversity. Block 210 is described in greater detail with respect to FIG. 3.

FIG. 3 is a block diagram illustrating methods 210 to formulate and implement an approach to identify binding molecules with maximal activity across extensive genomic diversity, in accordance with certain examples of the technology disclosed herein.

In block 310, the binding molecule system 110 identifies all known sequences within a region S. For example, the binding molecule system 110 may select the most conserved K-mers from recent sequences for a particular virus.

In block 320, the binding molecule system 110 constructs a ground set of possible binding molecules by finding representative subsequences across the region S using locality sensitive hashing. The binding molecule system 110 finds representative subsequences in a genomic region that span known diversity. These representative subsequences are possible binding molecules. The binding molecule system 110 calculates an activity between each of these binding molecules and each target sequence to form the ground set of binding molecules.

In block 330, the binding molecule system 110 identifies a function that quantifies detection activity between a binding molecule and a targeted sequence. The function goal is to find the set of binding molecules, a subset of the ground set, that maximizes a function of the expected activity between the binding molecules and sequences in S.

In block 340, the binding molecule system 110 solves the function by identifying the binding molecule set P, a subset of the ground set, that maximizes a function of the expected activity between P and sequences in S. The binding molecule system 110 implements a fast randomized combinatorial algorithm for maximizing a non-negative and non-monotone submodular function under a cardinality constraint, which provides a binding molecule set whose objective value is within a factor 1/e of the optimal.

Simple strategies constructing binding molecules using the consensus or the most common sequence in a region fail to capture a desired amount of the sequence diversity. However, maximizing expected activity detects more sequences—even when constrained to a single binding molecule—across the genome. The amount detected further increases as the binding molecule system 110 permits more binding molecules. Similarly, using a related objective function that minimizes the number of binding molecules subject to constraints on activity and comprehensiveness reaches near-complete comprehensiveness with few binding molecules.

From block 340, the method 210 returns to block 220 of FIG. 2.

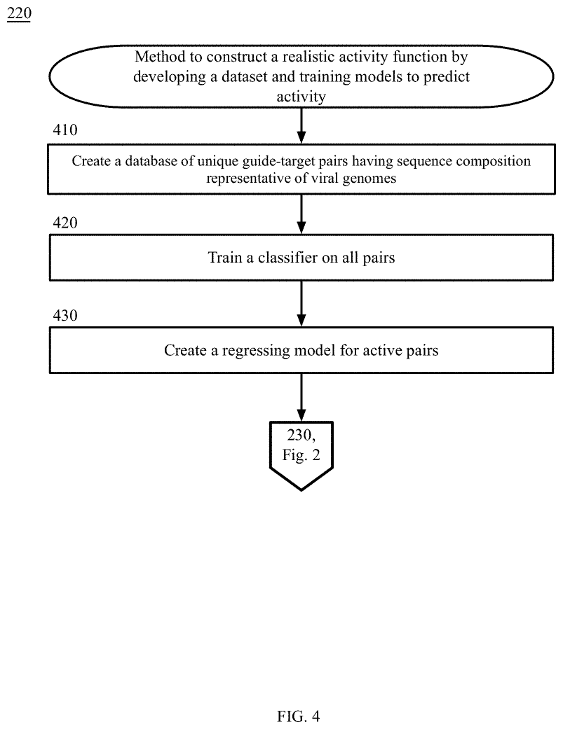

Returning to FIG. 2, in block 220, the binding molecule system 110 construct a realistic activity function by developing a dataset and training models to predict activity. Block 220 is described in greater detail with respect to FIG. 4.

FIG. 4 is a block diagram illustrating methods to construct a realistic activity function by developing a dataset and training models to predict activity, in accordance with certain examples of the technology disclosed herein.

In block 410, the binding molecule system 110 creates a database of unique guide-target pairs having sequence composition representative of viral genomes. The binding molecule system 110 develops an activity function informed by detection reaction kinetics. The database includes a set of unique guide-target pairs having sequence composition representative of viral genomes. The binding molecule system 110 uses a two-step hurdle model that involves classifying a pair from the database as inactive or active, and then regressing activity for active pairs.

In block 420, the binding molecule system 110 classifies all guide-target pairs as inactive or active. The binding molecule system 110 trains a classifier based on the data from the database. In an example, a convolutional neural network (“CNN”) is used to classify the guide target pairs. Other algorithms, programs, or processes may be used for the classification.

In block 430, the binding molecule system 110 creates a regressing model for active pairs. In an example, a CNN is used to create a model. Other algorithms, programs, or processes may be used to create the regressing model. The CNN model allows for multiple parallel convolutional and locally-connected filters of different widths. Using a locally-connected layer significantly improves model performance because the locally-connected layer helps the model to learn strong spatial dependencies in the data—for example, a mismatch-sensitive seed region—that may be missed by convolutional layers and difficult for fully connected layers to ascertain.

From block 430, the method 220 returns to block 230 of FIG. 2.

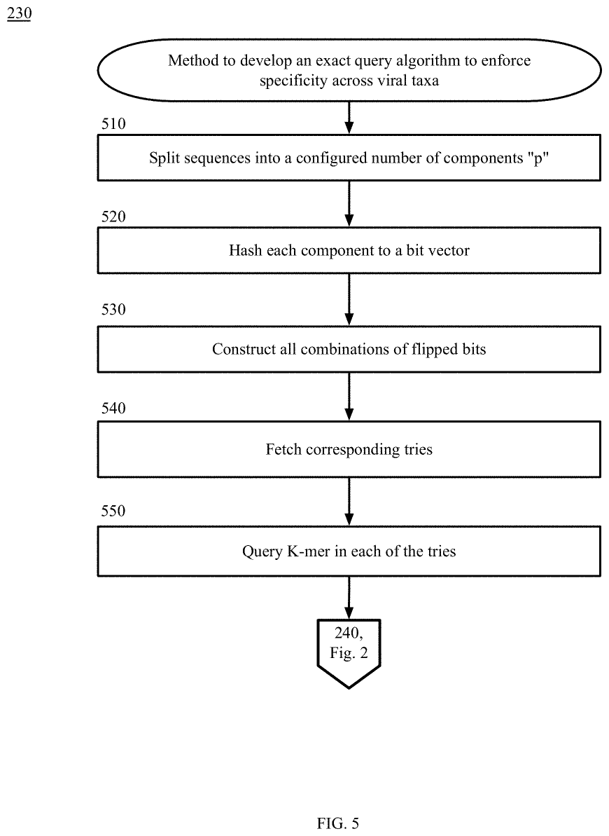

Returning to FIG. 2, in block 230, the binding molecule system 110 develops an exact query algorithm to enforce specificity across viral taxa. Block 230 is described in greater detail with respect to FIG. 5.

FIG. 5 is a block diagram illustrating methods to develop an exact query algorithm to enforce specificity across viral taxa, in accordance with certain examples of the technology disclosed herein. In design a binding molecule, the operator will desire to avoid binding molecules that are cross-reactive. The goal of the method 230 is to query binding molecules (such as 28-mers) to determine if the binding molecules specific to a non-targeted species. The method 230 compensates for the challenges of design a binding molecule to be specific to a targeted sequence when the sequences, such as 28-mers, are short. The method needs to tolerate high divergence (such as a sequence that has up to 5 mismatches).

In one approach, the binding molecule system 110 builds compressed tries of 28-mers and queries with branching for G-U pairs and mismatches. The query algorithm is suited to determining the specificity of a probe, tolerating both high divergence and G-U wobble base pairing.

In block 510, the binding molecule system 110 splits the sequences of the K-mer of the binding molecule into a configured number of components “p.” Sharding of the binding molecule into higher number of components p allows fewer bit flips but yields larger tries, and vice-versa.

In block 520, the binding molecule system 110 hashes each component to a bit vector.

In block 530, the binding molecule system 110 constructs all combinations of flipped bits corresponding to a specified divergence.

In block 540, the binding molecule system 110 fetches corresponding tries.

In block 550, the binding molecule system 110 queries K-mer in each of the tries. The binding molecule system 110 finds all valid hits. By the pigeonhole principle, at least one partition has less than or equal to [m/p] mismatches against each valid hit. The binding molecule system 110 may employ a loose bound on query runtime.

From block 550, the method 230 returns to block 240 of FIG. 2.

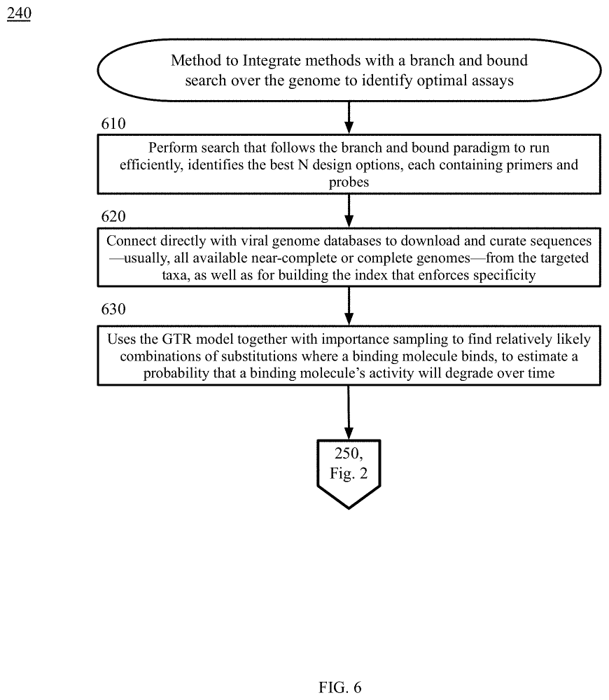

Returning to FIG. 2, in block 240, the binding molecule system 110 integrates methods with a branch and bound search over the genome to identify optimal assays. Block 240 is described in greater detail with respect to FIG. 6.

FIG. 6 is a block diagram illustrating methods to integrate methods with a branch and bound search over the genome to identify optimal assays, in accordance with certain examples of the technology disclosed herein.

In block 610, the binding molecule system 110 connects directly with viral genome databases to download and curate sequences—usually, all available near-complete or complete genomes—from the targeted taxa, as well as for building the index that enforces specificity.

In block 620, the binding molecule system 110 performs a search that follows the branch and bound paradigm to run efficiently and identifies the best N design options, each containing primers and binding molecules. The binding molecule system 110 performs a search across a viral genome to identify regions to target, scored according to both their amplification potential (e.g., presence of conserved endpoints) and the optimal activity of a probe set within the region.

In an alternate example, the binding molecule system 110 uses the GTR model to find relatively likely combinations of substitutions where a probe binds, to estimate a probability that a probe's activity will degrade over time.

In an alternate embodiment, the binding molecule system 110 generates models without the need for actual sample genomes. The binding molecule system 110 does not only rely on testing provided and identified binding molecules, but instead uses the databases and other data to generate potential binding molecules. The potential binding molecules are tested and modeled as described in the method 200 herein.

From block 460, the method 220 returns to block 230.

In block 230, the binding molecule system 110 predicts diagnostic-target activity of a binding molecule. Block 230 is described in greater detail with respect to FIG. 5.

FIG. 5 is a block diagram illustrating methods 230 to predict guide-target activity of a binding molecule, in accordance with certain examples of the technology disclosed herein.

In block 510, the binding molecule system 110 compares target sequence to a database of diagnostic-target pairs. In block 520, the binding molecule system 110 identifies binding molecules with a smaller number of mismatches.

From block 520, the method 230 returns to block 240. In block 240, the binding molecule system 110 generates an optimally active binding molecule. Block 240 is described in greater detail with respect to FIG. 6.

FIG. 6 is a block diagram illustrating methods 240 to generate an optimally active guide sequence, in accordance with certain examples of the technology disclosed herein. This method 240 allows the binding molecule system 110 to 1) detect one target, 2) detect across a set of targets, 3) differentiate strains (several mismatches between targets), and 4) differentiate SNP (one mismatch between targets).

In an example, a Wasserstein Generative Adversarial Nets with Active Maximization are used to train the machine-learning algorithm. In an example, the Generative Adversarial Nets us modified to be conditional on a target sequence.

In block 610, the binding molecule system 110 identifies a set of mismatched binding molecules. In block 620, the binding molecule system 110 identifies a set of targets sequences. In block 630, the binding molecule system 110 inputs a set of mismatched guides and set of targets sequences into a machine-learning algorithm. In block 640, the binding molecule system 110 using machine-learning algorithm to generate a binding molecule that would match an input of a target sequence.

### Additional Exemplary Systems and Processes

FIG. 7 depicts a computing machine 2000 and a module 2050 in accordance with certain examples. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the examples presented previously are illustrative, and, in alternative examples, certain acts can be performed in a different order, in parallel with one another, omitted entirely, and/or combined between different examples, and/or certain additional acts can be performed, without departing from the scope and spirit of various examples. Accordingly, such alternative examples are included in the scope of the following claims, which are to be accorded the broadest interpretation to encompass such alternate examples.

### Binding Molecules

Molecules designed by the methods described herein can account for extensive and changing diversity in emerging, evolving targets that can be used for diagnostics and therapeutics. The methods provide broad application to a variety of nucleic acid and protein technologies, and eliminate laborious experimental testing currently required to keep pace with evolution of targets. In general, the binding molecules designed are sequence-specific molecules designed for a target or set of target molecules or sequences. Because the designs of the molecules should be routinely checked or updated, especially around outbreaks, the ADAPT approach advantageously provides the needed agility to respond to highly evolving target molecules.

In an aspect, the methods for designing sequence-specific molecules includes predicting the designed molecule-target activity, which can reduce experimental testing and make more effective guides. In embodiments, utilizing a CRISPR-Cas system, predicting activity includes context around the guide molecule, including the proto-flanking spacer, guide sequence composition, target molecule structure (e.g. DNA, RNA) and mismatches between the guide and the target. In certain embodiments, regression of activity for positive guide-target pairs is a goal in the design approach with regard to activity prediction.

The molecules can be designed to provide an optimal guide to: bind one target, bind across a set of targets, differentiate mismatches between targets, and differentiate SNPs with one mismatch between targets.

Exemplary molecules for design include nucleic acid and polypeptide molecules for a variety of diagnostic and therapeutic uses.

In certain example embodiments, the binding molecules are designed for diagnostic purposes. The methods allow comprehensive accounting for diversity and designing optimal molecules for nucleic acid diagnostics, such as PCR, RNA toehold switches (see, e.g., Pardee et al., 2016, Cell 165 1255-1266), antibodies, and CRISPR-based diagnostics, serologic tests, vaccines, therapies and drugs, including modeling resistance. Binding molecules are sequence specific molecules, typically a polypeptide or nucleic acid, and in embodiments include amplification primers, probes, and guide molecules. Because of the broad range of molecules that can be designed according to the current methods, the source of the sequences that are evaluated can also vary. In example embodiments, optimizing binding molecules in near real-time is performed with the disclosed methodologies so that highly evolving diseases can be identified during outbreaks, evolving drug resistance, and other applications that benefit from an agile, highly adaptive approach to design of binding molecules. In an aspect, the optimization of binding molecules includes enabling nearreal-time design i.e., fetching current sequences from sequencing databases, curating the sequences, clustering and aligning the sequences, see.e.g FIG. 9 for exemplary aligning of genomes. Viral sequencing data in near real time can be accessed, for example, at ncbi.nlm.nih.gov/genome/viruses and viprbrc.org/brc/home.spg?decorator=vipr. Viral sequences can be aligned by species, or clusters, clades or other classifications depending on the end use. In this way, the methodology can be tailored for target specificity, which may, for example, be to a species or to a Glade, or to a newly evolved mutation during an outbreak.

Similarly, binding molecules can comprise detection of drug resistance genes, for example, antibiotic resistance genes, which are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Binding molecules according to the present invention can be designed for maximal activity. Maximal activity may be determined based, for example, on detection reaction kinetics. (See, e.g. FIG. 4). In an embodiment, the binding molecules can be designed for maximal activity for viral detection. By way of example only, in an approach to design of probes for viral activity, for example guide sequences for use with CRISPR-Cas systems, probe set design for maximal activity may be detection of the full scope of known sequences, variation in a genomic region, for all vertebrates or particular vertebrate, and/or all known sequences. In an embodiment, quantification of best activity between probe and target can be performed, with weighting depending on desired outcome (e.g. detection of emerging infections, geographic region-specific sequences).

### Hybridization Probes

In certain example embodiments, the binding molecule may be a hybridization probe. A probe typically refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention can be labeled with any reporter molecule, so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

Hybridization assays in which a nucleic acid that displays “probe” nucleic acids for each of the genes to be assayed/profiled in the profile to be generated can be employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with designed probes under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### RNA Toehold Switches

In certain example embodiments, the binding molecule may be a RNA toehold switch,. Green, et al., 2014; Green et al., 2017. Toehold switches are riboregulators that can activate gene expression in response to cognate RNAs, with a cis-repressing switch RNA hairpin and a transacting trigger RNA. Two hairpin switches can also be designed according to current methods, with optimization of spacing from hairpin-to hairpin, as well as binding sites of triggers. The toehold switches can be unfolded upon binding a trigger RNA, and are useful in detecting a variety of targets including viral targets such as Zika and Ebola (See, e.g. Pardee et al., 2016). Toehold switches are sensitive to sequence variations between it and the trigger RNA, with design requiring sequence properties, structures and specificities to be considered, and particularly suited for the methods disclosed herein.

### Amplification Primers

In certain example embodiments, binding molecules may be amplification primers. Amplfication primers can include amplification or preamplification molecules that can be target specific, optionally selected from PCR, RPA, or RCA, or can comprise preferential amplification of microbial DNA.

In some embodiments, the amplification is target-specific and probes comprise proximity dependent probes, such as molecular inversion probes. In particular embodiments, molecular inversion probe amplification comprises hybridizing probes to a target of interest; circularizing the hybridized probes; digesting non-hybridized linear probes; and adding a primer pair and amplifying the circularized probes.

The approach of utilizing primers in proximity or adjacent to a sequence of interest, which may include particular pathways implicated in an infection or disease can enrich for samples in larger drug scale drug screening, including for either host and/or microbial, e.g., bacterial treatments. Accordingly, primers for drug resistant strains, pathways of interest, families, classes or other groupings of interest can be utilized for enrichment, large scale drug studies, and other high throughput applications can be utilized. Primers adjacent to, or within about 100, about 90, about 80, about 70, about 60, about 50, about 40, about 30, about 20, about 10, about 9, about 8, about 7, about 6, about 5, about 4, about 3, about 2, or about 1 nucleotide(s) of the gene of interest can be used.

Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). Amplification approaches can be used with reverse transcriptase where appropriate. In particular embodiments, the reverse transcriptase used is SuperScript IV reverse transcriptase.

In embodiments, the sequence specific primer is designed for a NASBA amplification. RNA or DNA amplification by NASBA is initiated with reverse transcription of target RNA by a sequence-specific reverse primer which can be designed according to the methods and systems described herein to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence.

In certain other example embodiments, a recombinase polymerase amplification (RPA) specific primers can be designed in accordance with the methods and systems disclosed herein. The RPA reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by a CRISPR Cas system as described elsewhere herein. Optionally the guide molecules of the CRISPR system can also be designed in accordance with the methods and systems described here. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

### Polypeptides

The systems, devices, and methods disclosed herein may also be adapted for detection of polypeptides (or other molecules) in addition to detection of nucleic acids, via incorporation of a specifically configured polypeptide detection aptamer. The polypeptide detection aptamers are distinct from masking construct aptamers. First, the aptamers are designed to specifically bind to one or more target molecules using the methods and systems as described herein. In one example embodiment, the target molecule is a target polypeptide. In another example embodiment, the target molecule is a target chemical compound, such as a target therapeutic molecule. Methods for designing and selecting aptamers with specificity for a given target are as described elsewhere herein. In addition to specificity to a given target the aptamers can be further designed to incorporate a RNA polymerase promoter binding site.

In certain example embodiments, the RNA polymerase promoter is a T7 promoter. Prior to binding the aptamer binding to a target, the RNA polymerase site is not accessible or otherwise recognizable to a RNA polymerase. However, the aptamer is configured so that upon binding of a target the structure of the aptamer undergoes a conformational change such that the RNA polymerase promoter is then exposed. An aptamer sequence downstream of the RNA polymerase promoter acts as a template for generation of a trigger RNA oligonucleotide by a RNA polymerase. Thus, the template portion of the aptamer may further incorporate a barcode or other identifying sequence that identifies a given aptamer and its target. The aptamers can be designed to work with guide molecules as described herein, such that when used with CRISPR systems, the guide molecules can be designed to recognize the specific trigger oligonucleotide sequences. Binding of guide molecules to the trigger oligonucleotides activates the Cas polypeptides of the CRISPR systems to generate detectable signals. In another example embodiment, binding of the aptamer may expose a primer binding site upon binding of the aptamer to a target polypeptide. For example, the aptamer may expose an RPA primer binding site. Thus, the addition or inclusion of the primer will then feed into an amplification reaction, such as the RPA reaction.

In certain example embodiments, the aptamer may be a conformation-switching aptamer, which upon binding to the target of interest may change secondary structure and expose new regions of single-stranded DNA. In certain example embodiments, these new-regions of single-stranded DNA may be used as substrates for ligation, extending the aptamers and creating longer ssDNA molecules which can be specifically detected using the embodiments disclosed herein. The aptamer design could be further combined with ternary complexes for detection of low-epitope targets, such as glucose (Yang et al. 2015: pubs.acs.org/doi/abs/10.1021/acs.analchem.5b01634). Example conformation shifting aptamers and corresponding guide RNAs (crRNAs) are disclosed as in International Patent Publication WO/2018/10712 at [0260]-[0261], incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. Antisense RNA is single stranded RNA that is complementary to a protein coding messenger RNA (mRNA) with which it hybridizes, and can block mRNA translation.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Guide Molecules Optimized for Use in CRISPR Systems

In certain example embodiments the binding molecule may be a guide molecule for use with a CRISPR-Cas system. CRISPR systems for diagnostic and therapeutic purposes can be used with guide molecule that have been designed according to the methods disclosed herein. The binding molecules can include use of designed amplification primers with the CRISPR systems, guide molecules designed by the methods disclosed herein, or both. In particular embodiments, the CRISPR system comprises a Cas protein and one or more guide molecules designed according to the methods disclosed herein. In certain embodiments, the guide molecules detect or are diagnostic for the presence of a virus or viral infection in a sample. In embodiments, the optimization of binding molecules includes enabling nearreal-time design i.e., fetching current sequences from sequencing databases, curating the sequences, clustering and aligning the sequences, see, e.g., FIG. 9 for exemplary aligning of genomes. Viral sequencing data in near real time can be accessed, for example, at ncbi.nlm.nih.gov/genome/viruses and atviprbrc.org/brc/home.spg?decorator=vipr. In certain embodiments, the guide molecules are designed with CRISPR-Cas systems designed to modify a target sequence for therapeutic purposes.

### CRISPR Systems

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). Cas proteins can be Class 1 or Class 2 proteins. Class 1 proteins comprise Type I, III and IV proteins. Class 2 proteins comprise Type II, V and VI proteins.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molce1.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1 , n5, Figure

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAIVIPs) family subunits (e.g., Cas 5, Cas6, and/or Cas7). RAMP proteins are characterized by having one or more RNA recognition motif domains. In some embodiments, multiple copies of RAIVIPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin EV, Makarova KS. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin EV, Makarova KS. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasY(Cas12d), CasX (Cas12e), Cas14, and/or Cas(I).

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

In certain embodiment, two or more CRISPR effector systems are utilized, which may be RNA-targeting effector proteins, DNA-targeting effector proteins, or a combination thereof. The RNA-targeting effector proteins may be a Cas13 protein, such as Cas13a, Cas13b, Cas13c, or Cas13d. The DNA-targeting effector protein may be a Cas12 protein such as Cas12a (Cpf1) and Cas12b (C2c1).

Non-specific RNase activity can be leveraged to cleave reporters upon target recognition, allowing for the design of sensitive and specific diagnostics using Cas12 and Cas13 proteins, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems”and PCT/US18/67307 filed Dec. 21, 2018 entitled “Novel CRISPR Enzymes and Systems”, US 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26, 2018 entitled “Novel Cas12b Enzymes and Systems”, U.S. 715,640 filed Aug. 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 November 24;358(6366):1019-1027; Gootenberg J S, et al., Multiplexed and portable nucleic acid detection platform with Cas13, Cas12a, and Csm6., Science. 2018 April 27;360(6387):439-444; Gootenberg J S, et al., Nucleic acid detection with CRISPR-Cas13a/C2c2., Science. 2017 April 28;356(6336):438-442; Abudayyeh OO, et al., RNA targeting with CRISPR-Cas13, Nature. 2017 October 12;550(7675):280-284; Smargon AA, et al., Cas13b Is a Type VI-B CRISPR-Associated RNA-Guided RNase Differentially Regulated by Accessory Proteins Csx27 and Csx28. Mol Cell. 2017 February 16;65(4):618-630.e7; Abudayyeh OO, et al., C2c2 is a single-component programmable RNA-guided RNA-targeting CRISPR effector, Science. 2016 August 5;353(6299):aaf5573; Yang L, et al., Engineering and optimising deaminase fusions for genome editing. Nat Commun. 2016 November 2;7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

Cas12a Orthologs

The present invention encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”) and Cas12a. Presently, the subtype V-A loci encompasses cas1, cas2, a distinct gene denoted cpf1 and a CRISPR array. Cas12a, or Cpf1(CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cas12a lacks the HNH nuclease domain that is present in all Cas9 proteins, and the RuvC-like domain is contiguous in the Cas12a sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cas12a also make it an ideal switchable nuclease for non-specific cleavage of nucleic acids. In one embodiment, a Cas12a system is engineered to provide and take advantage of collateral non-specific cleavage of RNA. In another embodiment, a Cas12a system is engineered to provide and take advantage of collateral non-specific cleavage of ssDNA. Accordingly, engineered Cas12a systems provide platforms for nucleic acid detection and transcriptome manipulation. Cas12a is developed for use as a mammalian transcript knockdown and binding tool. Cas12a is capable of robust collateral cleavage of RNA and ssDNA when activated by sequence-specific targeted DNA binding.

The Cas12a gene is found in several diverse bacterial genomes, typically in the same locus with cas1, cas2, and cas4 genes and a CRISPR cassette (for example, FNFX1 1431-FNFX1 1428 of Francisella cf . novicida Fx1). Thus, the layout of this putative novel CRISPR-Cas system appears to be similar to that of type II-B. Furthermore, similar to Cas9, the Cas12a protein contains a readily identifiable C-terminal region that is homologous to the transposon ORF-B and includes an active RuvC-like nuclease, an arginine-rich region, and a Zn finger (absent in Cas9). However, unlike Cas9, Cas12a is also present in several genomes without a CRISPR-Cas context and its relatively high similarity with ORF-B suggests that it might be a transposon component. It was suggested that if this was a genuine CRISPR-Cas system and Cas12a is a functional analog of Cas9 it would be a novel CRISPR-Cas type, namely type V (See Annotation and Classification of CRISPR-Cas Systems. Makarova KS, Koonin EV. Methods Mol Biol. 2015;1311:47-75). However, as described herein, Cas12a is denoted to be in subtype V-A to distinguish it from C2c1p which does not have an identical domain structure and is hence denoted to be in subtype V-B.

In particular embodiments, the effector protein is a Cas12a effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiandium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cas12a effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

In a more preferred embodiment, the Cas12ap is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cas12a protein derived from an organism from the bacterial species of Eubacterium rectale. In some embodiments, the amino acid sequence of the Cas12a effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cas12a effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cas12a protein whereby the sequence identity is determined over the length of the truncated form. In some embodiments, the Cas12a effector recognizes the PAM sequence of TTTN or CTTN.

Cas12b Orthologs

The present invention encompasses the use of a C2c1 effector proteins, derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”), or Cas12b. Presently, the subtype V-B loci encompasses cas1-Cas4 fusion, cas2, a distinct gene denoted Cas12b and a CRISPR array. Cas12b (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, Cas12b lacks the HNH nuclease domain that is present in all Cas9 proteins, and the RuvC-like domain is contiguous in the Cas12b sequence, in contrast to Cas9 where it contains long inserts including the HNH domain. Accordingly, in particular embodiments, the CRISPR-Cas enzyme comprises only a RuvC-like nuclease domain.

C2c1 (also known as Cas12b) proteins are RNA guided nucleases. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, Cas12b nuclease activity also requires relies on recognition of PAM sequence. Cas12b PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum.

Cas12b creates a staggered cut at the target locus, with a 5′ overhang, or a “sticky end” at the PAM distal side of the target sequence. In some embodiments, the 5′ overhang is 7 nt. See Lewis and Ke, Mol Cell. 2017 February 2;65(3):377-379.

The Cas12b gene is found in several diverse bacterial genomes, typically in the same locus with cas1, cas2, and cas4 genes and a CRISPR cassette. Thus, the layout of this putative novel CRISPR-Cas system appears to be similar to that of type II-B. Furthermore, similar to Cas9, the Cas12b protein contains an active RuvC-like nuclease, an arginine-rich region, and a Zn finger (absent in Cas9).

In particular embodiments, the effector protein is a Cas12b effector protein from an organism from a genus comprising Alicyclobacillus, Desulfovibrio, Desulfonatronum, Opitutaceae, Tuberibacillus, Bacillus, Brevibacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the Cas12b effector protein is from a species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG _13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

In a more preferred embodiment, the Cas12bp is derived from a bacterial species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In certain embodiments, the Cas12bp is derived from a bacterial species selected from Alicyclobacillus acidoterrestris (e.g., ATCC 49025), Alicyclobacillus contaminans (e.g., DSM 17975).

In an embodiment, the Cas12b protein may be an ortholog of an organism of a genus which includes, but is not limited to Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG 13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In particular embodiments, the homologue or orthologue of Cas12b as referred to herein has a sequence homology or identity of at least 80%, more preferably at least 85%, even more preferably at least 90%, such as for instance at least 95% with one or more of the Cas12b sequences disclosed herein. In further embodiments, the homologue or orthologue of Cas12b as referred to herein has a sequence identity of at least 80%, more preferably at least 85%, even more preferably at least 90%, such as for instance at least 95% with the wild type AacCas12b or BthCas12b.

In certain embodiments, the Cas12b protein is a catalytically inactive Cas12b which comprises a mutation in the RuvC domain. In some embodiments, the catalytically inactive Cas12b protein comprises a mutation corresponding to amion acid positions D570, E848, or D977 in Alicyclobacillus acidoterrestris Cas12b. In some embodiments, the catalytically inactive Cas12b protein comprises a mutation corresponding to D570A, E848A, or D977A in Alicyclobacillus acidoterrestris Cas12b.

The programmability, specificity, and collateral activity of the RNA-guided Cas12b also make it an ideal switchable nuclease for non-specific cleavage of nucleic acids. In one embodiment, a Cas12b system is engineered to provide and take advantage of collateral non-specific cleavage of RNA. In another embodiment, a Cas12b system is engineered to provide and take advantage of collateral non-specific cleavage of ssDNA. Accordingly, engineered Cas12b systems provide platforms for nucleic acid detection and transcriptome manipulation, and inducing cell death. Cas12b is developed for use as a mammalian transcript knockdown and binding tool. Cas12b is capable of robust collateral cleavage of RNA and ssDNA when activated by sequence-specific targeted DNA binding.

According to the invention, engineered Cas12b systems are optimized for DNA or RNA endonuclease activity and can be expressed in mammalian cells and targeted to effectively knock down reporter molecules or transcripts in cells.

### Cas13 Orthologs

In embodiments, the proteins for use with optimized guides in CRISPR-Cas systems are a Type VI CRISPR protein. In particular embodiment, the Type VI protein is a Type VI-A (Cas13a) or Type VI-B (Cas13b) or Type VI-C (Cas 13c) or Type VI-D (Cas13d) protein. In particular embodiments, the Type VI RNA-targeting Cas enzyme is C2c2, or Cas13a. In other example embodiments, the Type VI RNA-targeting Cas enzyme is Cas 13b. In certain example embodiments, the CRISPR system the effector protein is a Type VI nuclease. The activity of Type VI nucleases depends on the presence of two HEPN domains, and is a defining feature of Type VI CRISPR-Cas proteins. These have been shown to be RNase domains, i.e. nuclease (in particular an endonuclease) cutting RNA. On the basis that the HEPN domains of Cas13 are at least capable of binding to and, in their wild-type form, cutting RNA, then it is preferred that the Cas13 effector protein has RNase function. Regarding Cas13a CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in PCT/US2017/038154 entitled “Novel Type VI CRISPR Orthologs and Systems,” at, for example, pages 256-264 and 285-336, U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application 62/484,786 entitled “Novel Type VI CRISPR Orthologs and Systems,” filed on Apr. 12, 2017.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains. The skilled person will understand that truncated forms of the C2c2 proteins can be utilized, whereby the sequence identity is determined over the length of the truncated form.

In an embodiment of the invention, a HEPN domain comprises at least one RxxxxH motif comprising the sequence of R{N/H/K}X1X2X3H (SEQ ID NO: 4-6). In an embodiment of the invention, a HEPN domain comprises a RxxxxH motif comprising the sequence of R{N/H}X1X2X3H (SEQ ID NO: 7-8). In an embodiment of the invention, a HEPN domain comprises the sequence of R{N/K}X1X2X3H (SEQ ID NO: 9-10). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

The RNA targeting effector protein can, in some embodiments, comprise one or more HEPN domains, which can optionally comprise a RxxxxH motif sequence. In some instances, the RxxxH motif comprises a R{N/H/K]X1X2X3H (SEQ ID NO: 1-3) sequence, which in some embodiments X1 is R, S, D, E, Q, N, G, or Y, and X2 is independently I, S, T, V, or L, and X3 is independently L, F, N, Y, V, I, S, D, E, or A.

In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira. Species of organism of such a genus can be as otherwise herein discussed.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium] aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37 ; Butyrivibrio sp. YAB3001; Blautia sp. Marseille-P2398; Leptotrichia sp. Marseille-P3007 ; Bacteroides ihuae; Porphyromonadaceae bacterium KH3CP3RA; Listeria riparia; and Insolitispirillum peregrinum.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

Some methods of identifying orthologues of CRISPR-Cas system enzymes may involve identifying tracr sequences in genomes of interest. Identification of tracr sequences may relate to the following steps: Search for the direct repeats or tracr mate sequences in a database to identify a CRISPR region comprising a CRISPR enzyme. Search for homologous sequences in the CRISPR region flanking the CRISPR enzyme in both the sense and antisense directions. Look for transcriptional terminators and secondary structures. Identify any sequence that is not a direct repeat or a tracr mate sequence but has more than 50% identity to the direct repeat or tracr mate sequence as a potential tracr sequence. Take the potential tracr sequence and analyze for transcriptional terminator sequences associated therewith.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In certain example embodiments, different orthologues from a same class of CRISPR effector protein may be used, such as two Cas13a orthologues, two Cas13b orthologues, or two Cas13c orthologues, which is described in International Application No. PCT/US2017/065477, Tables 1-6, pages 40-52, and incorporated herein by reference. On certain other example embodiments, different orthologues with different nucleotide editing preferences may be used such as a Cas13a and Cas13b orthologs, or a Cas13a and a Cas13c orthologs, or a Cas13b orthologs and a Cas13c orthologs etc.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 September 12; 154(6):1380-1389), Cas12 (Liu et al. Nature Communications, 8, 2095 (2017) , and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, Gly Ser linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423 , the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a CG base pair into a TA base pair (Komor et al. 2016. Nature. 533:420-424; Nishida et al. 2016. Science. 353; and Li et al. Nat. Biotech. 36:324-327) and ABEs convert an AT base pair to a GC base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi . org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO2019/005884, WO 2019/005886, WO 2019/071048, PCT/U520018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 posible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Classl or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference. CRISPR effector proteins described in International Application No. PCT/US2017/065477, Tables 1-6, pages 40-52, can be used in the presently disclosed methods, systems and devices, and are specifically incorporated herein by reference.

CRISPR Guides that may be used in the Present Invention

Guide molecules used herein can be designed according to the methods disclosed. In embodiments, guide molecules are designed for optimal activity, e.g. predicted to be highly active at a target, specific to its species, optimum activity at a particular reaction condition, e.g. temperature, or a combination of design parameters, and as described herein in the methods for design of binding molecules such as guide molecules.

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

As used herein, the term “guide molecule,” “guide sequence,” “crRNA,” “guide RNA,” or “single guide RNA,” or “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the guide sequence and a CRISPR protein to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq. sourceforge.net).

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In general, the CRISPR-Cas, CRISPR-Cas9 or CRISPR system may be as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell. In some embodiments, especially for non-nuclear uses, NLSs are not preferred. In some embodiments, a CRISPR system comprises one or more nuclear exports signals (NESs). In some embodiments, a CRISPR system comprises one or more NLSs and one or more NESs. In some embodiments, direct repeats may be identified in silico by searching for repetitive motifs that fulfill any or all of the following criteria: 1. found in a 2Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specififity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D 10A mutation) can be delivered with a pair of guide RNAs targeting a site of interest. Guide sequences and strategies to minimize toxicity and off-target effects can be as in WO2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

CRISPR enzymes as defined herein can employ more than one RNA guide without losing activity. Optimized guides as disclosed herein can be designed for use in tandem approaches. This enables the use of the CRISPR enzymes, systems or complexes as defined herein for targeting multiple DNA targets, genes or gene loci, with a single enzyme, system or complex as defined herein. The guide molecules may be tandemly arranged, optionally separated by a nucleotide sequence such as a direct repeat as defined herein. The guides are preferably designed according to the methods disclosed herein, and tandem guides can be similarly designed in accordance with the methods disclosed. The position of the different guide RNAs is the tandem does not influence the activity. It is noted that the terms “CRISPR-Cas system”, “CRISPR-Cas complex” “CRISPR complex” and “CRISPR system” are used interchangeably. Also the terms “CRISPR enzyme”, “Cas enzyme”, or “CRISPR-Cas enzyme”, can be used interchangeably. In preferred embodiments, said CRISPR enzyme, CRISP-Cas enzyme or Cas enzyme is Cas9, or any one of the modified or mutated variants thereof described herein elsewhere.

In one aspect, the invention provides a non-naturally occurring or engineered CRISPR enzyme, preferably a class 2 CRISPR enzyme, preferably a Type V or VI CRISPR enzyme as described herein, used for tandem or multiplex targeting. It is to be understood that any of the CRISPR (or CRISPR-Cas or Cas) enzymes, complexes, or systems according to the invention as described herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a Cas enzyme, complex or system as defined herein for targeting multiple gene loci. In one embodiment, this can be established by using multiple (tandem or multiplex) guide RNA (gRNA) sequences.

In one aspect, the invention provides methods for using one or more elements of a Cas enzyme, complex or system as defined herein for tandem or multiplex targeting, wherein said CRISP system comprises multiple guide RNA sequences. Preferably, said gRNA sequences are separated by a nucleotide sequence, such as a direct repeat as defined herein elsewhere.

The Cas enzyme, system or complex as defined herein provides an effective means for modifying multiple target polynucleotides. The Cas enzyme, system or complex as defined herein has a wide variety of utility including modifying (e.g., deleting, inserting, translocating, inactivating, activating) one or more target polynucleotides in a multiplicity of cell types. As such the CRISPR-Cas systems or complex as defined herein of the invention has a broad spectrum of applications in, e.g., gene therapy, drug screening, disease diagnosis, and prognosis, including targeting multiple gene loci within a single CRISPR system.

In one aspect, the invention provides a Cas enzyme, system or complex as defined herein, i.e. a CRISPR-Cas complex having a Cas9 protein having at least one destabilization domain associated therewith, and multiple guide RNAs that target multiple nucleic acid molecules such as DNA molecules, whereby each of said multiple guide RNAs specifically targets its corresponding nucleic acid molecule, e.g., DNA molecule. Each nucleic acid molecule target, e.g., DNA molecule can encode a gene product or encompass a gene locus. Using multiple guide RNAs hence enables the targeting of multiple gene loci or multiple genes. In some embodiments the Cas9 enzyme may cleave the DNA molecule encoding the gene product. In some embodiments expression of the gene product is altered. The Cas9 protein and the guide RNAs do not naturally occur together. The invention comprehends the guide RNAs comprising tandemly arranged guide sequences. The invention further comprehends coding sequences for the Cas9 protein being codon optimized for expression in a eukaryotic cell. In a preferred embodiment the eukaryotic cell is a mammalian cell, a plant cell or a yeast cell and in a more preferred embodiment the mammalian cell is a human cell. Expression of the gene product may be decreased. The Cas9 enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas9 CRISPR system or complex binds to the multiple target sequences. In some embodiments, the functional CRISPR system or complex may edit the multiple target sequences, e.g., the target sequences may comprise a genomic locus, and in some embodiments there may be an alteration of gene expression. In some embodiments, the functional CRISPR system or complex may comprise further functional domains. In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In a further aspect, the present invention provides compositions comprising the CRISPR enzyme, system and complex as defined herein or the polynucleotides or vectors described herein. Also provides are CRISPR systems or complexes comprising multiple guide RNAs, preferably in a tandemly arranged format. Said different guide RNAs may be separated by nucleotide sequences such as direct repeats.

Also provided is a method of treating a subject, e.g., a subject in need thereof, comprising inducing gene editing by transforming the subject with the polynucleotide encoding the Cas9 CRISPR system or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises the Cas9 enzyme, complex or system comprising multiple guide RNAs, preferably tandemly arranged. Where any treatment is occurring ex vivo, for example in a cell culture, then it will be appreciated that the term ‘subject’ may be replaced by the phrase “cell or cell culture.”

Compositions comprising Cas9 enzyme, complex or system comprising multiple guide RNAs, preferably tandemly arranged, or the polynucleotide or vector encoding or comprising said Cas9 enzyme, complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. Use of a Cas9 CRISPR system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible Cas9 activator allows one to induce transcription right before the screen and therefore minimizes the chance of false negative hits. Accordingly, by use of the instant invention in screening, e.g., gain of function screens, the chance of false negative results may be minimized.

In one aspect, the invention provides an engineered, non-naturally occurring CRISPR system comprising a Cas9 protein and multiple guide RNAs that each specifically target a DNA molecule encoding a gene product in a cell, whereby the multiple guide RNAs each target their specific DNA molecule encoding the gene product and the Cas9 protein cleaves the target DNA molecule encoding the gene product, whereby expression of the gene product is altered; and, wherein the CRISPR protein and the guide RNAs do not naturally occur together. The invention comprehends the multiple guide RNAs comprising multiple guide sequences, preferably separated by a nucleotide sequence such as a direct repeat and optionally fused to a tracr sequence. In an embodiment of the invention the CRISPR protein is a type V or VI CRISPR-Cas protein and in a more preferred embodiment the CRISPR protein is a Cas9 protein. The invention further comprehends a Cas9 protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides an engineered, non-naturally occurring vector system comprising one or more vectors comprising a first regulatory element operably linked to the multiple Cas9 CRISPR system guide RNAs that each specifically target a DNA molecule encoding a gene product and a second regulatory element operably linked coding for a CRISPR protein. Both regulatory elements may be located on the same vector or on different vectors of the system. The multiple guide RNAs target the multiple DNA molecules encoding the multiple gene products in a cell and the CRISPR protein may cleave the multiple DNA molecules encoding the gene products (it may cleave one or both strands or have substantially no nuclease activity), whereby expression of the multiple gene products is altered; and, wherein the CRISPR protein and the multiple guide RNAs do not naturally occur together. In a preferred embodiment the CRISPR protein is codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of each of the multiple gene products is altered, preferably decreased.

In one aspect, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas9 enzyme complexed with the one or more guide sequence(s) that is hybridized to the one or more target sequence(s); and (b) a second regulatory element operably linked to an enzyme-coding sequence encoding said Cas9 enzyme, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a Cas CRISPR complex to a different target sequence in a eukaryotic cell. In some embodiments, the CRISPR complex comprises one or more nuclear localization sequences and/or one or more NES of sufficient strength to drive accumulation of said Cas CRISPR complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the Cas enzyme, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the Cas9 CRISPR complex to the respective target sequence(s) in a eukaryotic cell, wherein the Cas CRISPR complex comprises a Cas9 enzyme complexed with the one or more guide sequence(s) that is hybridized to the respective target sequence(s); and/or (b) a second regulatory element operably linked to an enzyme-coding sequence encoding said Cas enzyme comprising preferably at least one nuclear localization sequence and/or NES. In some embodiments, the host cell comprises components (a) and (b). Where applicable, a tracr sequence may also be provided. In some embodiments, component (a), component (b), or components (a) and (b) are stably integrated into a genome of the host eukaryotic cell. In some embodiments, component (a) further comprises two or more guide sequences operably linked to the first regulatory element, and optionally separated by a direct repeat, wherein when expressed, each of the two or more guide sequences direct sequence specific binding of a Cas CRISPR complex to a different target sequence in a eukaryotic cell. In some embodiments, the Cas enzyme comprises one or more nuclear localization sequences and/or nuclear export sequences or NES of sufficient strength to drive accumulation of said CRISPR enzyme in a detectable amount in and/or out of the nucleus of a eukaryotic cell.

An aspect of the invention encompasses methods of modifying a genomic locus of interest to change gene expression in a cell by introducing into the cell any of the compositions described herein.

An aspect of the invention is that the above elements are comprised in a single composition or comprised in individual compositions. These compositions may advantageously be applied to a host to elicit a functional effect on the genomic level.

As used herein, the term “guide RNA” or “gRNA” has the leaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. Each gRNA may be designed to include multiple binding recognition sites (e.g., aptamers) specific to the same or different adapter protein. Each gRNA may be designed to bind to the promoter region −1000-+1 nucleic acids upstream of the transcription start site (i.e. TSS), preferably −200 nucleic acids. This positioning improves functional domains which affect gene activation (e.g., transcription activators) or gene inhibition (e.g., transcription repressors). The modified gRNA may be one or more modified gRNAs targeted to one or more target loci (e.g., at least 1 gRNA, at least 2 gRNA, at least 5 gRNA, at least 10 gRNA, at least 20 gRNA, at least 30 g RNA, at least 50 gRNA) comprised in a composition. Said multiple gRNA sequences can be tandemly arranged and are preferably separated by a direct repeat.

Thus, gRNA, the CRISPR enzyme as defined herein may each individually be comprised in a composition and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral sgRNA selection) and concentration of gRNA (e.g., dependent on whether multiple gRNAs are used) may be advantageous for eliciting an improved effect. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR transgenic cell /animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses Cas9 akin to Platt et al. The target cell or animal thus comprises the CRISPR enzyme (e.g., Cas9) conditionally or inducibly (e.g., in the form of Cre dependent constructs), on expression of a vector introduced into the target cell, the vector expresses that which induces or gives rise to the condition of the CRISPR enzyme (e.g., Cas9) expression in the target cell. By applying the teaching and compositions as defined herein with the known method of creating a CRISPR complex, inducible genomic events are also an aspect of the current invention. Examples of such inducible events have been described herein elsewhere.

In some embodiments, phenotypic alteration is preferably the result of genome modification when a genetic disease is targeted, especially in methods of therapy and preferably where a repair template is provided to correct or alter the phenotype.

In some embodiments delivery methods include: Cationic Lipid Mediated “direct” delivery of Enzyme-Guide complex (RiboNucleoProtein) and electroporation of plasmid DNA.

Methods, products and uses described herein may be used for non-therapeutic purposes. Furthermore, any of the methods described herein may be applied in vitro and ex vivo.

In an aspect, provided is a non-naturally occurring or engineered composition comprising:

I. two or more CRISPR-Cas system polynucleotide sequences comprising

(a) a first guide sequence capable of hybridizing to a first target sequence in a polynucleotide locus,

(b) a second guide sequence capable of hybridizing to a second target sequence in a polynucleotide locus,

(c) a direct repeat sequence, and

II. a Cas9 enzyme or a second polynucleotide sequence encoding it,

wherein when transcribed, the first and the second guide sequences direct sequence-specific binding of a first and a second Cas CRISPR complex to the first and second target sequences respectively,

wherein the first CRISPR complex comprises the Cas enzyme complexed with the first guide sequence that is hybridizable to the first target sequence,

wherein the second CRISPR complex comprises the Cas enzyme complexed with the second guide sequence that is hybridizable to the second target sequence, and

wherein the first guide sequence directs cleavage of one strand of the DNA duplex near the first target sequence and the second guide sequence directs cleavage of the other strand near the second target sequence inducing a double strand break, thereby modifying the organism or the non-human or non-animal organism. Similarly, compositions comprising more than two guide RNAs can be envisaged e.g. each specific for one target, and arranged tandemly in the composition or CRISPR system or complex as described herein.

In another embodiment, the Cas is delivered into the cell as a protein. In another and particularly preferred embodiment, the Cas is delivered into the cell as a protein or as a nucleotide sequence encoding it. Delivery to the cell as a protein may include delivery of a Ribonucleoprotein (RNP) complex, where the protein is complexed with the multiple guides.

In an aspect, host cells and cell lines modified by or comprising the compositions, systems or modified enzymes of present invention are provided, including stem cells, and progeny thereof.

In an aspect, methods of cellular therapy are provided, where, for example, a single cell or a population of cells is sampled or cultured, wherein that cell or cells is or has been modified ex vivo as described herein, and is then re-introduced (sampled cells) or introduced (cultured cells) into the organism. Stem cells, whether embryonic or induce pluripotent or totipotent stem cells, are also particularly preferred in this regard. But, of course, in vivo embodiments are also envisaged.

Inventive methods can further comprise delivery of templates, such as repair templates, which may be dsODN or ssODN, see below. Delivery of templates may be via the cotemporaneous or separate from delivery of any or all the CRISPR enzyme or guide RNAs and via the same delivery mechanism or different. In some embodiments, it is preferred that the template is delivered together with the guide RNAs and, preferably, also the CRISPR enzyme. An example may be an AAV vector where the CRISPR enzyme is AsCas9 or LbCas9.

Inventive methods can further comprise: (a) delivering to the cell a double-stranded oligodeoxynucleotide (dsODN) comprising overhangs complimentary to the overhangs created by said double strand break, wherein said dsODN is integrated into the locus of interest; or (b) delivering to the cell a single-stranded oligodeoxynucleotide (ssODN), wherein said ssODN acts as a template for homology directed repair of said double strand break. Inventive methods can be for the prevention or treatment of disease in an individual, optionally wherein said disease is caused by a defect in said locus of interest. Inventive methods can be conducted in vivo in the individual or ex vivo on a cell taken from the individual, optionally wherein said cell is returned to the individual.

The invention also comprehends products obtained from using CRISPR enzyme or Cas enzyme or CRISPR-Cas system for use in tandem or multiple targeting as defined herein.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT (SEQ ID NO: 11) or |
| | NGRRN (SEQ ID NO: 12) |
| NmeCas9 | NNNNGATT (SEQ ID NO: 13) |
| CjCas9 | NNNNRYAC (SEQ ID NO: 14) |
| StCas9 | NNAGAAW (SEQ ID NO: 15) |
| Cas12a (Cpf1) (including | TTTV (SEQ ID NO: 16) |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ (SEQ ID NO: 17) |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver BP et al. Engineered CRISPR-Cas9 nucleases with altered PAM specificities. Nature. 2015 July 23;523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Target Molecules

The systems, devices, and methods, disclosed herein are directed to detecting the presence of one or more target molecules, in embodiments, one or more nucleic acids or polypeptides. The optimally active binding molecules may be generated for detecting one target, detecting across a set of targets, differentiating strains where there may be one or several mismatches between targets, and differentiating SNPs with one mismatch between targets. In particular embodiments, the generation of the binding molecule is modified to be conditional on a target sequence. Modification of the generator can include identifying a representative target, for example from a sequence database or other identified target, that is optimized for maximal activity, differential identification and/or other desired properties. Binding molecules include primers for enrichment can be designed for a variety of target sequences, including microbial infections, including for example, bacterial infections. Primers can be designed for viral, bacterial and other infectious diseases, strains, or groups of strains. Other examples of interest can include enrichment with primers for immune checkpoints, gene expression in tumors, cancers—including loss of heterozygosity, and cancer drug resistance detection, and epigenetic modifications.

Target molecules may be one or more microbial agents in a sample, such as a biological sample obtained from a subject. In certain example embodiments, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g. bacterial resistance), monitoring of disease progression and/or outbreak, and antibiotic screening.

### Viruses

Primers may be used to enrich for a viral infection (e.g. of a subject or plant), including a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2.

By way of example only, several clinically important viruses have evolved ribavirin resistance including Foot and Mouth Disease Virus doi:10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistent RNA viruses, such as hepatitis and HIV, have evolved resistance to existing antiviral drugs: hepatitis B virus (lamivudine, tenofovir, entecavir) doi:10/1002/hep22900; hepatitis C virus (telaprevir, BILN2061, ITMN-191, SCh6, boceprevir, AG-021541, ACH-806) doi:10.1002/hep.22549; and HIV (many drug resistance mutations) hivb.standford.edu. The embodiments disclosed herein may be used to detect such variants among others.

In certain example embodiments, the systems, devices, and methods, disclosed herein are directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan Ebola virus, Bundibugyo virus, Tai Forest Ebola virus, Reston Ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanj a virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or human parainfluenza virus (HPIV-1, HPIV-2, HPIV-3, HPIV-4)Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1,-2, and -3, (GLRaV-1,-2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e. after infection has occurred or prophylactically, i.e. before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

In embodiments, the virus is associated with a respiratory illness. In an aspect, the virus is a coronavirus. In certain embodiments, the systems, methods and compositions comprise two or more binding molecules to one or more viruses or subtypes. Multiplex design of guide molecules for the detection of coronaviruses and/or other respiratory viruses in a sample to identify the cause of a respiratory infection is envisioned, and design can be according to the methods disclosed herein. Regarding detection of coronavirus, guide design can be predicated on genome sequences disclosed in Tian et al, “Potent binding of 2019 novel coronavirus spike protein by a SARS coronavirus-specific human monoclonal antibody”; doi: 10.1101/2020.01.28.923011, incorporated by reference, which details human monoclonal antibody, CR3022 binding of the 2019-nCoV RBD (KD of 6.3 nM) or Sequences of the 2019-nCoV are available at GISAID accession no. EPI ISL 402124 and EPI ISL 402127-402130, and described in doi:10.1101/2020.01.22.914952, or EP ISL 402119-402121 and EP ISL 402123-402124; see also GenBank Accession No. MN908947.3. Guide design can target unique viral genomic regions of SARS-CoV-2 (also referred to as 2019-nCoV) or conserved genomic regions across one or more viruses of the coronavirus family. The coronavirus is a positive-sense single stranded RNA family of viruses, infecting a variety of animals and humans. SARS-CoV is one type of coronavirus infection, as well as MERS-CoV. In an aspect, one may use known SARS and SARS-related coronaviruses or other viruses from one or more hosts to generate a non-redundant alignment. Related viruses can be found, for example in bats.

Design can include species level Severe acute respiratory syndrome-related coronavirus species. Includes SARS-CoV-2, SARS-CoV-1, and SARS-like CoV. Gene targets may comprise ORF lab, N protein, RNA-dependent RNA polymerase (RdRP), E protein, ORF1b-nsp14, Spike glycoprotein (S), or pancorona targets. Molecular assays have been under development and can be used as a starting point to develop guide molecules for the methods and systems described herein. See, “Diagnostic detection of 2019-nCoV by real-time RT-PCR” Charite, Berlin Germany (17 Jan. 2020)' Detection of 2019 novel coronavirus (2019-nCoV) in suspected human cases by RT-PCR Hong Kong University (23 Jan. 2020); PCR and sequencing protocol for 2019-nCoV - Department of Medical Sciences, Ministry of Public Health, Thailand (updated 28 Jan. 2020); PCR and sequencing protocols for 2019-nCoV- National Institute of Infectious Diseases Japan (24 Jan. 2020); US CDC panel primer and probes U.S. CDC, USAV U.S. CDC, USA (28 Jan. 2020); China CDC Primers and probes for detection 2019-nCoV (24 Jan. 2020), incorporated in their entirety by reference. Further, the guide molecule design may exploit differences or similarities with SARS-CoV. In an aexample, the assay is set for subspecies-level, identifying the cause of the COVID-19 outbreak, and may exclude detection of highly related RaTG13 genome and other bat and pangolin SARS-like CoVs.

Design can include subspecies-level detection of SARS-like CoV, including most known bat and pangolin SARS-like CoVs, optionally excluding detection of SARS-CoV-2 and SARS-CoV-1. Other human coronaviruses can be detected, including for example, HCoV-229E, HCoV-HKU1, HCoV-NL63, Betacoronivirus 1. Orthomyxyxoviruses panels can also be designed, including all known subtypes of influenza A virus, segment 2; all H1 subtypes (e.g. H1N1), segment 4; all H3 subtypes (E.g. H3N2), segment 4; N1 subtypes (e.g. H1N1) segment 6; all N2 subtypes (e.g. H3N2), segment 6; or all known lineages of influenza B virus, segment 1. Similar design for paramyxoviruses, including HPIV-1, HPIV-2, HPIV-3, or FPIV-4. Design of Picornaviruses panel, including Rhinovirus, A, B, C or a combination thereof, Enterovirus, A, B, C, D or a combination thereof, Phenumoviruses, including HRSV (Human orthopneumovirus) and HMPV (Human metapneumovirus). Other coronaviruses can be detected, including in other species, such as hedgehogs, rabbits, mice, pangolin and bats. Exemplary coronaviruses can include Bat Hp-betacoronavirus Zhejiang2013, pipistrellus bat coronavirus HKU5, rabbit coronavirus HKU14, Rousettus bat coronavirus GCCDC1, Rousettus bat coronavirus HKU9, Tylonycteris bat coronavirus HKU4, coronavirus HKU15, Byulbul coronavirus HKU11, common moorhen coronavirus HKU21, murine coronavirus, China Rattus coronavirus HKU24, Rhinolpophus ferrumequinum alphacoronavirus HuB-2013, Scotophilus bat coronavirus, 512, Wencheng Sm shreq coronavirus, Rhinolophus bat coronavirus HKU2, Nyctalus velutinum alphacoronavirus SC-2103, Porcine epidemic diarrhea virus, NL63-related bat coronavirus strain BtKYNL63-9b, Myotis ricketti alphacoronavirus Sax-2011, Mink coronavirus 1, Ferret coronavirus, Miniopterus bat coronavirus HKU8, Alphacoronavirusl, BtRf-AlphaCoV/YN2012, Coronavirus AcCoV-JC34, Bat coronavirus CDPHE15, Lucheng Rn rat coronavirus, Batr coronavirus CDPHE15, Magpie-robin coronavirus HKU18, Munia coronavirus HKU13, Night heron coronavirus HKU19, Sparrow coronavirus HKU17, Thrush coronavirus HKU12, White-eye coronavirus HKU16, Wigeon Coronavirus HKU20, Avian coronavirus, Beluga whale coronavirus SW1. Binding molecules have been designed according to the methods disclosed herein and can be accessed at adapt. sabetilab .org, including at adapt.sabetilab.org/covid-19/, incorporated herein by reference in its entirety.

Researchers have recently identified similarities and differences between SARS-CoV-2 and SARS-CoV. “Coronavirus Genome Annotation Reveals Amino Acid Differences with Other SARS Viruses,” genomeweb, Feb. 10, 2020. For example, guide molecules based on the 8a protein, which was present in SARS-CoV but absent in 2019-nCoV, can be utilized to differentiate between the viruses. Similarly, the 8b and 3b proteins have different lengths in SARS-CoV and 2019-nCoV and can be utilized to design guide molecules to detect non-overlapping proteins of nucleotides encoding in the two viruses. Wu et al., Genome Composition and Divergence of the Novel Coronavirus (2019-nCoV) Originating in China, Cell Host & Microbe (2020), DOI: 10.1016/j.chom.2020.02.001, incorporated herein by reference, including all supplemental information, in particular Table S1.

The binding molecules herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen. The rapid ability to design binding molecules according to the evolution of a pathogen may further identify a pattern of transmission, including, for example, superinfection, contamination, deleterious or adaptive mutations, and mechanisms responsible for the severity of an epidemic episode. Such methods of monitoring outbreaks can be as described, for example in WO2018/107129 [0306]-[0326], incorporated herein by reference.

The pattern of pathogen transmission may comprise continued new transmissions from the natural reservoir of the pathogen or subject-to-subject transmissions (e.g. human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e. at the beginning of the pathogen outbreak. Determining the pattern of the pathogen transmission at the beginning of the outbreak increases likelihood of stopping the outbreak at the earliest possible time thereby reducing the possibility of local and international dissemination.

### Methods of Using the Designed Molecules

In an aspect, the embodiments disclosed herein are directed to methods for detecting target nucleic acids in a sample using the systems described herein. The present invention also relates to methods for increasing specificity of the designed molecules for both therapeutics and diagnostics, particularly in instances where therapies may require highly specific, sensitive, or rapidly evolving targets. The invention provides methods of modifying a polynucleotide, which may comprise deletion, insertion or other editing of a target polynucleotide. In embodiments, the expression of a polynucleotide is modified, in one aspect, in a cell. Methods of generating a model eukaryotic cell comprising a mutated disease gene are also provided. Such methods are described, for example, in PCT/US2017/047459 and PCT/US2017/047458, incorporated herein by reference.

In embodiments, the methods may use a CRISPR-Cas system based therapy or therapeutics, detection or diagnostics. In a further aspect, the invention relates to methods for increasing safety of CRISPR-Cas systems, such as CRISPR-Cas system-based therapy or therapeutics. In a further aspect, the present invention relates to methods for increasing specificity, efficacy, and/or safety, preferably all, of CRISPR-Cas systems, such as CRISPR-Cas system based therapy or therapeutics and/or diagnostics. The invention thus provides a methods of treating a disease, disorder or infection in an individual in need thereof comprising identifying suitable treatment conditions and administering an effective amount of the compositions, systems provided herein, or assaying for effective treatments based on the screening disclosed. In an aspect, the disease, disorder, or infection may comprise a viral infection.

In certain embodiments the detection methods can comprise Combinatorial Arrayed Reactions for Multiplexed Evaluation of Nucleic acids (CARMEN) for diagnosis of a particular disease or infection, for example viral infections. After amplification (with optional reverse transcription), detection is performed with Cas13, using in vitro transcription to convert amplified DNA into RNA. The resulting RNA is detected with exquisite sequence specificity by Cas13-crRNA complexes, and collateral cleavage produces a signal using a cleavage reporter RNA; Briefly, the steps of CARMEN comprise (Step 1) Samples are amplified, color coded, and emulsified. In parallel, detection mixes are assembled, color coded and emulsified. (Step 2) Droplets from each emulsion are pooled into a single tube and mixed by pipetting. (Step 3) The droplets are loaded into the chip in a single pipetting step. SIDE VIEW: The droplets are deposited through the loading slot into the flow space between the chip and glass. Tilting the loader moves the pool of droplets around the flow space, allowing the droplets to float up into the microwells. (Step 4) The chip is clamped against glass, isolating the contents of each microwell, and imaged by fluorescence microscopy to identify the color code and position of each droplet. (Step 5) Droplets are merged, initiating the detection reaction. (Step 6) The detection reactions in each microwell are monitored over time (a few minutes 3 hours) by fluorescence microscopy. CARMEN is as described for example in U.S. Provisional 62/767,070 filed Nov. 14, 2018, 62/841,812 filed May 1, 2019 and 62/871,056 filed Jul. 5, 2019, incorporated herein by reference.

The methods disclosed herein can, in some embodiments, comprise the steps of generating a first set of droplets, each droplet in the first set of droplets comprising at least one target molecule and an optical barcode; generating a second set of droplets, each droplet in the second set of droplets comprising a detection CRISPR system comprising an RNA targeting effector protein and one or more guide RNAs designed to bind to corresponding target molecules, an masking construct and an optical barcode. The first and second set of droplets are typically combined into a pool of droplets by mixing or agitating the first and second set of droplets. The pool of droplets can then be flooded onto a microfluidic device comprising an array of microwells and at least one flow channel beneath the microwells, the microwells sized to capture at least two droplets; detecting the optical barcodes of the droplets captured in each microwell; merging the droplets captured in each microwell to formed merged droplets in each microwell, at least a subset of the merged droplets comprising a detection CRISPR system and a target sequence; initiating the detection reaction; and measuring a detectable signal of each merged droplet at one or more time periods.

In another aspect, the embodiments disclosed herein are directed to a diagnostic device comprising a plurality of individual discrete volumes. Each individual discrete volume comprises a CRISPR system comprising CRISPR effector protein, one or more guide RNAs designed to bind to a corresponding target molecule, and a masking construct. Individual discrete volumes may also comprise optical barcodes, target molecules, and/or amplification reagents. Individual discrete volumes may be provided that comprise a CRISPR system with an optical barcode; other individual discrete volumes that may be provided that comprises optical barcodes, optionally with target molecules and/or amplification reagents. In certain example embodiments, RNA amplification reagents may be pre-loaded into the individual discrete volumes or be added to the individual discrete volumes concurrently with, prior to, or subsequent to addition of a sample or target molecule to an individual discrete volume. In one aspect, merging of individual discrete volumes such as droplets effects the addition of particular reagents to a merged individual discrete volume. The device may be a microfluidic based device, a wearable device, or device comprising a flexible material substrate on which the individual discrete volumes are defined or provided.

In certain example embodiments, a single guide sequences specific to a single target is placed in separate volumes. Each volume may then receive a different sample or aliquot of the same sample. In certain example embodiments, multiple guide sequences each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, G, U/ T. Accordingly, masking constructs completely comprising, or comprised of a substantial portion, of a single nucleotide may be generated, each with a different fluorophore that can be detected at differing wavelengths. In this way up to four different targets may be screened in a single individual discrete volume. In certain example embodiments, different orthologues from a same class of CRISPR effector protein may be used, such as two Cas13a orthologues, two Cas13b orthologues, or two Cas13c orthologues, or a Cas12 and Cas13 orthologue.

In another aspect, the embodiments disclosed herein are directed to a method for detecting target nucleic acids in a sample comprising distributing a sample or set of samples, that may be comprised in their own individual discrete volumes, to a set of individual discrete volumes, each individual discrete volume comprising a CRISPR effector protein, one or more guide RNAs designed to bind to one target oligonucleotides, and a masking construct. Such distribution in particularly preferred embodiments is preferably by random droplet distribution. The set of samples are then maintained under conditions sufficient to allow binding of the one or more guide RNAs to one or more target molecules. Binding of the one or more guide RNAs to a target nucleic acid in turn activates the CRISPR effector protein. Once activated, the CRISPR effector protein then deactivates the masking construct, for example, by cleaving the masking construct such that a detectable positive signal is unmasked, released, or generated. Detection of the positive detectable signal in an individual discrete volume indicates the presence of the target molecules.

In yet another aspect, the embodiments disclosed herein are directed to a method for detecting polypeptides. The method for detecting polypeptides is similar to the method for detecting target nucleic acids described above. However, a peptide detection aptamer is also included. The peptide detection aptamers function as described above and facilitate generation of a trigger oligonucleotide upon binding to a target polypeptide. The guide RNAs are designed to recognize the trigger oligonucleotides thereby activating the CRISPR effector protein. Deactivation of the masking construct by the activated CRISPR effector protein leads to unmasking, release, or generation of a detectable positive signal.

Methods of compositions may comprise one or more guide molecules is about 27 to about 29 nucleotides in length and/or the target locus of interest is provided via a nucleic acid molecule in vitro and/or the target locus of interest is provided via a nucleic acid molecule within a cell and/or the target locus of interest is provided via a nucleic acid molecule within a cell wherein the cell comprises a prokaryotic cell and/or the target locus of interest is provided via a nucleic acid molecule within a cell wherein the cell comprises a eukaryotic cell and/or the modification of the target locus of interest comprises a nucleotide strand break and/or the Cas protein is expressed from a nucleic acid molecule codon optimized for expression in eukaryotic cell and/or the effector protein is associated with one or more functional domains and/or the complex delivers an epigenetic modifier or a transcriptional or translational activation or repression signal and/or the complex delivers a functional domain that modifies transcription or translation of the target locus and/or the effector protein comprises at least one or more nuclear localization signals and/or when in complex with the Cas protein the guide molecule(s) is capable of effecting sequence specific binding of the complex to a target sequence of the target locus of interest and/or the guide molecules comprise a dual direct repeat sequence and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules comprise one or more regulatory elements operably configured to express the polypeptides and/or the guide molecule(s), optionally wherein the one or more regulatory elements comprise a promoter(s) or inducible promotor(s) and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules are comprised within one or more vector(s) and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and the one or more polynucleotide molecules are comprised within one vector and/or the Cas protein and guide molecule(s) are provided via one or more polynucleotide molecules encoding the polypeptides and/or the nucleic acid component(s), and wherein the one or more polynucleotide molecules are operably configured to express the polypeptides and/or the guide molecule(s) and the one or more polynucleotide molecules are comprised within one or more or one vector and the one or more vectors comprise viral vector(s) and/or the Cas protein and guide molecule(s) are provided via one or more polynucleotide molecules encoding the polypeptides and/or the guide molecule(s), and wherein the one or more polynucleotide molecules are operably configured to express the polypeptides and/or the guide molecule(s) and the one or more polynucleotide molecules are comprised within one or more or one vector and the one or more vectors comprise viral vector(s) and the one or more viral vector(s) comprise one or more retroviral, lentiviral, adenoviral, adeno-associated or herpes simplex viral vector(s) and/or the effector protein and guide molecule(s) are provided via one or more polynucleotide molecules encoding the polypeptides and/or the nucleic acid component(s), and wherein the one or more polynucleotide molecules are operably configured to express the polypeptides and/or the nucleic acid component(s) and are comprised in a delivery system or the complex or its components or a component of the complex is comprised in a delivery system and/or delivering comprises a delivery vehicle comprising liposome(s), particle(s), exosome(s), microvesicle(s), a gene-gun or one or more viral vector(s) and/or the one or more polynucleotide molecules comprise one or more regulatory elements operably configured to express the polypeptides and/or the nucleic acid component(s), and the one or more regulatory elements comprise a promoter(s) or inducible promotor(s).

Methods for detecting target nucleic acids in a sample are provided that comprise distributing a sample or set of samples into one or more individual discrete volumes, the individual discrete volumes comprising the nucleic acid detection system. Methods can comprise treating the sample with heat. In some embodiments, the method may further comprise the steps of i) incubating the sample at 37-50° C. for 5-20 minutes; ii) incubating the sample at 64-95° C. for 5 minutes; iii) performing RT-RPA; iv) performing T7 transcription; and v) detecting the target nucleic acids.

In embodiments, the reaction can be performed as an extraction-free detection, combining extraction and detection together. In particular aspects, nucleic acid extraction is eliminated by using heat and chemical deduction to both destroy RNA-degrading nucleases and lyse, for example, viral particles, in a sample. The reaction conditions can be optimized in addition to or in concert with the guide optimization. For example, additional reaction condition optimization of different pHs, monovalent salt, magnesium, and primer concentrations on assay sensitivity can be evaluated as described in the examples herein.

In some embodiments, the target nucleic acid may be DNA and the method may further comprise the step of extracting DNA from cells in the sample. In certain embodiments, the present invention provides steps of obtaining a sample of biological fluid (e.g., urine, blood plasma or serum, sputum, cerebral spinal fluid), and extracting the DNA. The mutant nucleotide sequence to be detected, may be a fraction of a larger molecule or can be present initially as a discrete molecule. In certain embodiments, blood samples are collected and plasma immediately separated from the blood cells by centrifugation. Serum may be filtered and stored frozen until DNA extraction. In some embodiments, the sample may be collected on a Whatman FTA card, as described in the Examples. The method may further comprise eluting the sample from the FTA card. In particular embodiments, the detection of extraction and heat treatment can be performed in a variety of sample types, including plasma. See, e.g., PCT/US2020/022776, Examples 6-7 for exemplary methods of use; see also, HUDSON Methods: Myhrvold,et al., (2018). Field-deployable viral diagnostics using CRISPR-Cas13. Science, 360(6387), 444-448. doi:10.1126/science.aas8836, each of which is incorporated herein by reference.

In some embodiments, the method may further comprise treating the sample with heat, optionally at 99° C. for 10 minutes. In certain embodiments, the sample can be heated in more than one step. In embodiments, the first step of heating is performed at about 35° C. to about 55° C., in particular embodiments, at 40° C., on other embodiments, at 50° C. In certain methods, the initial heating step is performed for about 5 minutes to about 25 minutes, in embodiments, the initiation step comprises heating for 20 minutes at 50° C., or for about 5 minutes at 40° C. A subsequent step of heating may be performed at a different temperature, and may be dependent on sample type, e.g. saliva, blood, plasma. In an aspect, the sample is heated in a second step for about 5 minutes to about 15 minutes. The temperature range for the second heating step may be performed at a temperature of about 50° C. to about 110° C., about 50, 55, 60, 65, 70, 75, 80, 90, 95, 100, 105 or 110° C. In certain embodiments, the heating step can be performed for 5 minutes at 95° C., 10 minutes at 95° C., or 5 minutes at 70° C.

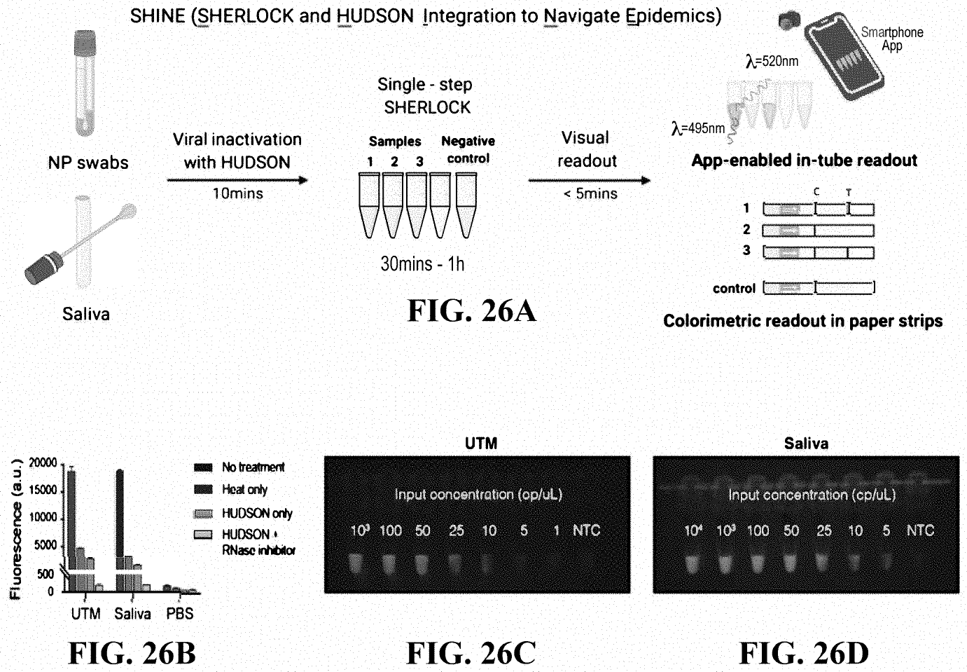

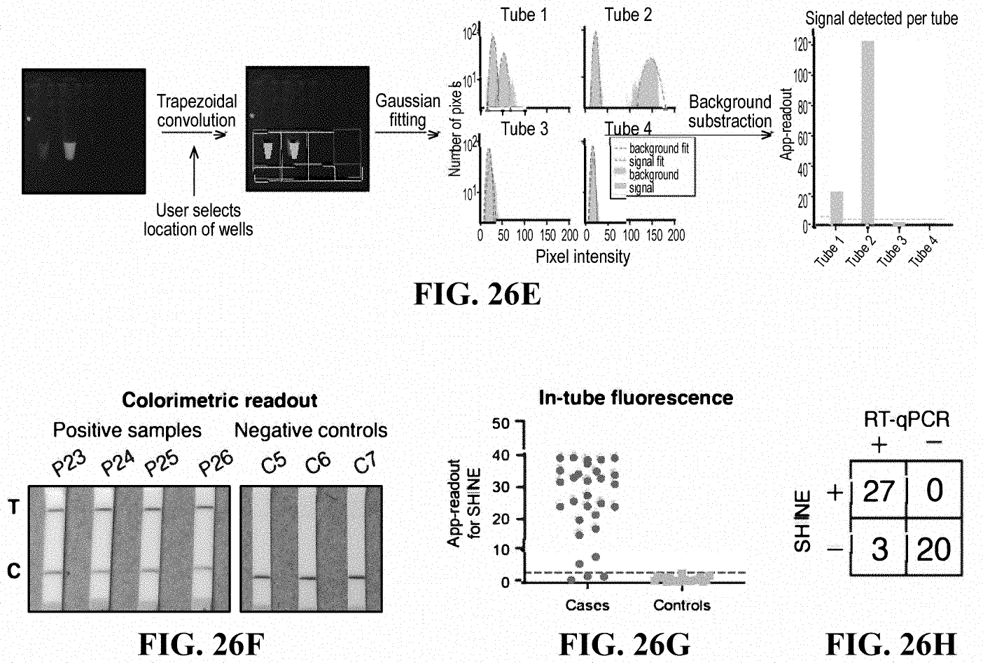

In an aspect, the detection assay produces a colorimetric readout or a fluorometric readout. Therefore, detection can comprise detection of a fluorescent or colorimetric readout, which may be comprise detection in an individual discrete volume, e.g., via a tube, lateral flow readout on a substrate, or droplets on a substrate. In certain embodiments, in-tube readout can be performed. In an aspect, a companion smartphone application can be used the utilizes a smartphone camera to image reaction tubes. In an aspect, upon capture of images of reaction tubes, the application then calculates the distance of the experimental tube's pixel intensity distribution from that of a user-selected negative control tube, and returns a binary result indicating the presence or absence of viral RNA in the sample (see, e.g., FIG. 26A and 26E; and Example 2 for further details). Without being bound by theory, the optimization of guide molecule, reaction conditions, and smartphone application allows for minimized equipment requirements and user interpretation bias when implemented.

### Detection Constructs

Detection constructs used in the methods and systems described herein may be selected based upon end use, and can preferably generate a fluorescent of colorimetric readout. A “detection construct” refers to a molecule that can be cleaved or otherwise deactivated by an activated CRISPR system effector protein described herein. The term “detection construct” may also be referred to in the alternative as a “masking construct.” Depending on the nuclease activity of the CRISPR effector protein, the masking construct may be a RNA-based masking construct or a DNA-based masking construct. The Nucleic Acid-based masking constructs comprises a nucleic acid element that is cleavable by a CRISPR effector protein. Cleavage of the nucleic acid element releases agents or produces conformational changes that allow a detectable signal to be produced. Example constructs demonstrating how the nucleic acid element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. It will be understood that in certain example embodiments a minimal background signal may be produced in the presence of an active masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in certain embodiments a first signal may be detected when the masking agent is present or when a CRISPR system has not been activated (i.e. a negative detectable signal), which then converts to a second signal (e.g. the positive detectable signal) upon detection of the target molecules and cleavage or deactivation of the masking agent, or upon activation of the CRISPR effector protein. The positive detectable signal, then, is a signal detected upon activation of the CRISPR effector protein, and may be, in a colorimetric or fluorescent assay, a decrease in fluorescence or color relative to a control or an increase in fluorescence or color relative to a control. In certain embodiments, RNAse or DNAse activity is detected colorimetrically via cleavage of enzyme-inhibiting aptamers. One potential mode of converting DNAse or RNAse activity into a colorimetric signal is to couple the cleavage of a DNA or RNA aptamer with the re-activation of an enzyme that is capable of producing a colorimetric output. In the absence of RNA or DNA cleavage, the intact aptamer will bind to the enzyme target and inhibit its activity. The advantage of this readout system is that the enzyme provides an additional amplification step: once liberated from an aptamer via collateral activity (e.g. Cpf1 collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, the masking construct may be a DNA or RNA aptamer and/or may comprise a DNA or RNA-tethered inhibitor.

In certain embodiments, the masking construct may comprise a DNA or RNA oligonucleotide to which a detectable ligand and a masking component are attached.

In certain embodiments, RNAse or DNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to DNase RNAse activity, colorimetric enzyme and inhibitor pairs can be engineered into DNase and RNAse sensors. The colorimetric DNase or RNAse sensor based upon small-molecule inhibitors involves three components: the colorimetric enzyme, the inhibitor, and a bridging RNA or DNA that is covalently linked to both the inhibitor and enzyme, tethering the inhibitor to the enzyme. In the uncleaved configuration, the enzyme is inhibited by the increased local concentration of the small molecule; when the DNA or RNA is cleaved (e.g. by Cas13 or Cas12 collateral cleavage), the inhibitor will be released and the colorimetric enzyme will be activated.

In certain embodiments, the aptamer or DNA- or RNA-tethered inhibitor may sequester an enzyme, wherein the enzyme generates a detectable signal upon release from the aptamer or DNA or RNA tethered inhibitor by acting upon a substrate. In some embodiments, the aptamer may be an inhibitor aptamer that inhibits an enzyme and prevents the enzyme from catalyzing generation of a detectable signal from a substance. In some embodiments, the DNA- or RNA-tethered inhibitor may inhibit an enzyme and may prevent the enzyme from catalyzing generation of a detectable signal from a substrate.

Particular constructs for visual readout in tubes or liquid individual discrete volumes may comprise a quenched FAM reporter. Such a reporter may be designed according to cleavage preferences of the enzyme utilized in the assay, for example, a poly-U FAM quenched reporter when used with LwaCas13a. Cleavage preferences have been explored and detailed in International Patent Publication No. WO/2019/126577, incorporated by reference in its entirety. In brief, cleavage motifs of Cas proteins have been interrogated and nucleic acid based reporters utilized based on those cleavage preferences allow for optimized diagnostic readouts and multiplexed reactions when using Cas proteins with orthogonal base preferences. See, e.g. WO/2019/126577, FIGS. 125A-132, incorporated specifically by reference.

Further discussion of detection constructs that can be used herein are described, for example, in PCT/US2020/022795, and U.S. Provisional Applications 62/818,702, and 62/890,555, all of which are incorporated by reference in their entirety. In particular, exemplary detection constructs, detectable ligands, and masking constructs are detailed in [0050]-[0086], specifically incorporated herein by reference.

### Screening Microbial Genetic Perturbations

In certain example embodiments, the CRISPR systems disclosed herein may be used to screen microbial genetic perturbations. Such methods may be useful, for example to map out microbial pathways and functional networks. Microbial cells may be genetically modified and then screened under different experimental conditions. As described above, the embodiments disclosed herein can screen for multiple target molecules in a single sample, or a single target in a single individual discrete volume in a multiplex fashion. Genetically modified microbes may be modified to include a nucleic acid barcode sequence that identifies the particular genetic modification carried by a particular microbial cell or population of microbial cells. A barcode is s short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the CRISPR effector systems described herein may be used to detect the barcode. Detection of the positive detectable signal indicates the presence of a particular genetic modification in the sample. The methods disclosed herein may be combined with other methods for detecting complimentary genotype or phenotypic readouts indicating the effect of the genetic modification under the experimental conditions tested. Genetic modifications to be screened may include, but are not limited to a gene knock-in, a gene knock-out, inversions, translocations, transpositions, or one or more nucleotide insertions, deletions, substitutions, mutations, or addition of nucleic acids encoding an epitope with a functional consequence such as altering protein stability or detection. In a similar fashion, the methods described herein may be used in synthetic biology application to screen the functionality of specific arrangements of gene regulatory elecments and gene expression modules.

In certain example embodiments, the methods may be used to screen hypomorphs. Generation of hypomorphs and their use in identifying key bacterial functional genes and identification of new antibiotic therapeutics as disclosed in PCT/US2016/060730 entitled “Multiplex High-Resolution Detection of Micro-organism Strains, Related Kits, Diagnostic Methods and Screening Assays” filed Nov. 4, 2016, which is incorporated herein by reference.

The different experimental conditions may comprise exposure of the microbial cells to different chemical agents, combinations of chemical agents, different concentrations of chemical agents or combinations of chemical agents, different durations of exposure to chemical agents or combinations of chemical agents, different physical parameters, or both. In certain example embodiments the chemical agent is an antibiotic or antiviral. Different physical parameters to be screened may include different temperatures, atmospheric pressures, different atmospheric and non-atmospheric gas concentrations, different pH levels, different culture media compositions, or a combination thereof.

### Detecting Gene Edits And/Or Off-Target Effects

The embodiments disclosed herein may be used in combination with other gene editing tools to confirm that a desired genetic edit or edits were successful and/or to detect the presence of any off-target effects. Cells that have been edited may be screened using one or more guides to one or more target loci. As the embodiments disclosed herein utlize CRISPR systems, theranostic applications are also envisioned. For example, genotyping embodiments disclosed herein may be used to select appropriate target loci or identify cells or populations of cells in needed of the target edit. The same or separate system may then be used to determine editing efficiency.

### Immunotherapy Applications

The embodiments disclosed herein can also be useful in further immunotherapy contexts. For instance, in some embodiments, methods of diagnosing, prognosing and/or staging an immune response in a subject comprise detecting a first level of expression, activity and/or function of one or more biomarker and comparing the detected level to a control level wherein a difference in the detected level and the control level indicates that the presence of an immune response in the subject. In some embodiments, the binding molecules can be used to evaluate the state of immune cells, such as T cells, including T cell activation and/or dysfunction. Additionally, the binding molecules can be used in assays to determine whether a patient is suitable for administering an immunotherapy of other type of therapy.

In certain embodiments, the present invention may be used to determine dysfunction or activation of tumor infiltrating lymphocytes (TIL). TILs may be isolated from a tumor using known methods. The TILs may be analyzed to determine whether they should be used in adoptive cell transfer therapies. Additionally, chimeric antigen receptor T cells (CAR T cells) may be analyzed for a signature of dysfunction or activation before administering them to a subject. Exemplary signatures for dysfunctional and activated T cell have been described (see e.g., Singer M, et al., A Distinct Gene Module for Dysfunction Uncoupled from Activation in Tumor-Infiltrating T Cells. Cell. 2016 September 8;166(6):1500-1511.e9. doi: 10.1016/j .ce11.2016.08.052).

In some embodiments, the systems and assays disclosed herein may allow clinicians to identify whether a patient's response to a therapy (e.g., an adoptive cell transfer (ACT) therapy) is due to cell dysfunction, and if it is, levels of up-regulation and down-regulation across the biomarker signature will allow problems to be addressed. For example, if a patient receiving ACT is non-responsive, the cells administered as part of the ACT may be assayed by an assay disclosed herein to determine the relative level of expression of a biomarker signature known to be associated with cell activation and/or dysfunction states. If a particular inhibitory receptor or molecule is up-regulated in the ACT cells, the patient may be treated with an inhibitor of that receptor or molecule. If a particular stimulatory receptor or molecule is down-regulated in the ACT cells, the patient may be treated with an agonist of that receptor or molecule.

### Cancer and Cancer Drug Resistance Detection

In certain embodiments, the present invention may be used to detect genes and mutations associated with cancer. In certain embodiments, mutations associated with resistance are detected. The amplification of resistant tumor cells or appearance of resistant mutations in clonal populations of tumor cells may arise during treatment (see, e.g., Burger J A, et al., Clonal evolution in patients with chronic lymphocytic leukaemia developing resistance to BTK inhibition. Nat Commun. 2016 May 20;7:11589; Landau D A, et al., Mutations driving CLL and their evolution in progression and relapse. Nature. 2015 October 22;526(7574):525-30; Landau D A, et al.,

Clonal evolution in hematological malignancies and therapeutic implications. Leukemia. 2014 January;28(1):34-43; and Landau D A, et al., Evolution and impact of subclonal mutations in chronic lymphocytic leukemia. Cell. 2013 February 14;152(4):714-26). Accordingly, detecting such mutations requires highly sensitive assays and monitoring requires repeated biopsy. Repeated biopsies are inconvenient, invasive and costly. Resistant mutations can be difficult to detect in a blood sample or other noninvasively collected biological sample (e.g., blood, saliva, urine) using the prior methods known in the art. Resistant mutations may refer to mutations associated with resistance to a chemotherapy, targeted therapy, or immunotherapy.

In certain embodiments, mutations occur in individual cancers that may be used to detect cancer progression. In one embodiment, mutations related to T cell cytolytic activity against tumors have been characterized and may be detected by the present invention (see e.g., Rooney et al., Molecular and genetic properties of tumors associated with local immune cytolytic activity, Cell. 2015 January 15; 160(1-2): 48-61). Personalized therapies may be developed for a patient based on detection of these mutations (see e.g., WO2016100975A1). In certain embodiments, cancer specific mutations associated with cytolytic activity may be a mutation in a gene selected from the group consisting of CASP8, B2M, PIK3CA, SMC1A, ARID5B, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, IDO2, ALOX12B and ALOX15B, or copy number gain, excluding whole-chromosome events, impacting any of the following chromosomal bands: 6q16.1-q21, 6q22.31-q24.1, 6q25.1-q26, 7p11.2-q11.1, 8p23.1, 8p11.23-p11.21 (containing IDOL IDO2), 9p24.2-p23 (containing PDL1, PDL2), 10p15.3, 10p15.1-p13, 11p14.1, 12p13.32-p13.2, 17p13.1 (containing ALOX12B, ALOX15B), and 22q11.1-q11.21.

In certain embodiments, the present invention is used to detect a cancer mutation (e.g., resistance mutation) during the course of a treatment and after treatment is completed. The sensitivity of the present invention may allow for noninvasive detection of clonal mutations arising during treatment and can be used to detect a recurrence in the disease.

In certain example embodiments, detection of microRNAs (miRNA) and/or miRNA signatures of differentially expressed miRNA, may be used to detect or monitor progression of a cancer and/or detect drug resistance to a cancer therapy. As an example, Nadal et al. (Nature Scientific Reports, (2015) doi:10.1038/srep12464) describe mRNA signatures that may be used to detect non-small cell lung cancer (NSCLC).

In certain example embodiments, the presence of resistance mutations in clonal subpopulations of cells may be used in determining a treatment regimen. In other embodiments, personalized therapies for treating a patient may be administered based on common tumor mutations. In certain embodiments, common mutations arise in response to treatment and lead to drug resistance. In certain embodiments, the present invention may be used in monitoring patients for cells acquiring a mutation or amplification of cells harboring such drug resistant mutations.

Treatment with various chemotherapeutic agents, particularly with targeted therapies such as tyrosine kinase inhibitors, frequently leads to new mutations in the target molecules that resist the activity of the therapeutic. Multiple strategies to overcome this resistance are being evaluated, including development of second generation therapies that are not affected by these mutations and treatment with multiple agents including those that act downstream of the resistance mutation. In an exemplary embodiment, a common mutation to ibrutinib, a molecule targeting Bruton's Tyrosine Kinase (BTK) and used for CLL and certain lymphomas, is a Cysteine to Serine change at position 481 (BTK/C481S). Erlotinib, which targets the tyrosine kinase domain of the Epidermal Growth Factor Receptor (EGFR), is commonly used in the treatment of lung cancer and resistant tumors invariably develop following therapy. A common mutation found in resistant clones is a threonine to methionine mutation at position 790.

Non-silent mutations shared between populations of cancer patients and common resistant mutations that may be detected with the present invention are known in the art (see e.g., WO/2016/187508). In certain embodiments, drug resistance mutations may be induced by treatment with ibrutinib, erlotinib, imatinib, gefitinib, crizotinib, trastuzumab, vemurafenib, RAF/MEK, check point blockade therapy, or antiestrogen therapy. In certain embodiments, the cancer specific mutations are present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Recently, gene expression in tumors and their microenvironments have been characterized at the single cell level (see e.g., Tirosh, et al. Dissecting the multicellular ecosystem of metastatic melanoma by single cell RNA-seq. Science 352, 189-196, doi:10.1126/science.aad0501 (2016)); Tirosh et al., Single-cell RNA-seq supports a developmental hierarchy in human oligodendroglioma. Nature. 2016 Nov 10;539(7628):309-313. doi: 10.1038/nature20123. Epub 2016 Nov. 2; and International patent publication serial number WO2017004153 A1). In certain embodiments, gene signatures may be detected using the present invention. In one embodiment complement genes are monitored or detected in a tumor microenvironment. In one embodiment MITF and AXL programs are monitored or detected. In one embodiment, a tumor specific stem cell or progenitor cell signature is detected. Such signatures indicate the state of an immune response and state of a tumor. In certain embodiments, the state of a tumor in terms of proliferation, resistance to treatment and abundance of immune cells may be detected.

Thus, in certain embodiments, the invention provides low-cost, rapid, multiplexed cancer detection panels for circulating DNA, such as tumor DNA, particularly for monitoring disease recurrence or the development of common resistance mutations.

### Therapeutics

It will be understood that the systems according to the invention as described herein, such as the CRISPR-Cas systems for use in the methods according to the invention as described herein, may be suitably used for any type of application known for CRISPR-Cas systems, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described herein elsewhere.

Any given genome editing application may comprise combinations of proteins, small RNA molecules, and/or repair templates, making delivery of these multiple parts substantially more challenging than small molecule therapeutics. Two main strategies for delivery of genome editing tools have been developed: ex vivo and in vivo. In ex vivo treatments, diseased cells are removed from the body, edited and then transplanted back into the patient. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

There may be drawbacks with ex vivo approaches that limit application to a small number of diseases. For instance, target cells must be capable of surviving manipulation outside the body. For many tissues, like the brain, culturing cells outside the body is a major challenge, because cells either fail to survive, or lose properties necessary for their function in vivo. Thus, in view of this disclosure and the knowledge in the art, ex vivo therapy as to tissues with adult stem cell populations amenable to ex vivo culture and manipulation, such as the hematopoietic system, by the CRISPR-Cas system are enabled. [Bunn, H. F. & Aster, J. Pathophysiology of blood disorders, (McGraw-Hill, New York, 2011)]

In vivo genome editing involves direct delivery of editing systems to cell types in their native tissues. In vivo editing allows diseases in which the affected cell population is not amenable to ex vivo manipulation to be treated. Furthermore, delivering nucleases to cells in situ allows for the treatment of multiple tissue and cell types. These properties probably allow in vivo treatment to be applied to a wider range of diseases than ex vivo therapies.

To date, in vivo editing has largely been achieved through the use of viral vectors with defined, tissue-specific tropism. Such vectors are currently limited in terms of cargo carrying capacity and tropism, restricting this mode of therapy to organ systems where transduction with clinically useful vectors is efficient, such as the liver, muscle and eye [Kotterman, M. A. & Schaffer, D. V. Nature reviews. Genetics 15, 445-451 (2014); Nguyen, T. H. & Ferry, N. Gene therapy 11 Suppl 1, S76-84 (2004); Boye, S. E., et al. Molecular therapy : the journal of the American Society of Gene Therapy 21, 509-519 (2013)].

A potential barrier for in vivo delivery is the immune response that may be created in response to the large amounts of virus necessary for treatment, but this phenomenon is not unique to genome editing and is observed with other virus based gene therapies [Bessis, N., et al. Gene therapy 11 Suppl 1, S10-17 (2004)]. It is also possible that peptides from editing nucleases themselves are presented on MHC Class I molecules to stimulate an immune response, although there is little evidence to support this happening at the preclinical level. Another major difficulty with this mode of therapy is controlling the distribution and consequently the dosage of genome editing nucleases in vivo, leading to off-target mutation profiles that may be difficult to predict. However, in view of this disclosure and the knowledge in the art, including the use of virus- and particle-based therapies being used in the treatment of cancers, in vivo modification of HSCs, for instance by delivery by either particle or virus, is within the ambit of the skilled person.

Ex Vivo Editing Therapy: The long standing clinical expertise with the purification, culture and transplantation of hematopoietic cells has made diseases affecting the blood system such as SCID, Fanconi anemia, Wiskott-Aldrich syndrome and sickle cell anemia the focus of ex vivo editing therapy. Another reason to focus on hematopoietic cells is that, thanks to previous efforts to design gene therapy for blood disorders, delivery systems of relatively high efficiency already exist. With these advantages, this mode of therapy can be applied to diseases where edited cells possess a fitness advantage, so that a small number of engrafted, edited cells can expand and treat disease. One such disease is HIV, where infection results in a fitness disadvantage to CD4+ T cells.

Ex vivo editing therapy can comprise gene correction strategies, including gene correction of a mutated IL2RG gene in hematopoietic stem cells (HSCs) obtained from a patient suffering from SCID-X1 [Genovese, P., et al. Nature 510, 235-240 (2014)]. Genovese et. al. accomplished gene correction in HSCs using a multimodal strategy. First, HSCs were transduced using integration-deficient lentivirus containing an HDR template encoding a therapeutic cDNA for IL2RG. Following transduction, cells were electroporated with mRNA encoding ZFNs targeting a mutational hotspot in IL2RG to stimulate HDR based gene correction. To increase HDR rates, culture conditions were optimized with small molecules to encourage HSC division. With optimized culture conditions, nucleases and HDR templates, gene corrected HSCs from the SCID-X1 patient were obtained in culture at therapeutically relevant rates. HSCs from unaffected individuals that underwent the same gene correction procedure could sustain long-term hematopoiesis in mice, the gold standard for HSC function. HSCs are capable of giving rise to all hematopoietic cell types and can be autologously transplanted, making them an extremely valuable cell population for all hematopoietic genetic disorders [Weissman, I. L. & Shizuru, J. A. Blood 112, 3543-3553 (2008)]. Gene corrected HSCs could, in principle, be used to treat a wide range of genetic blood disorders making this study an exciting breakthrough for therapeutic genome editing.

In Vivo Editing Therapy: In vivo editing can be used advantageously from this disclosure and the knowledge in the art.

Targeted deletion, therapeutic applications: Targeted deletion of genes may be preferred. Preferred are, therefore, genes involved in immunodeficiency disorder, hematologic condition, or genetic lysosomal storage disease, e.g., Hemophilia B, SCID, SCID-X1, ADA-SCID, Hereditary tyrosinemia, β-thalassemia, X-linked CGD, Wiskott-Aldrich syndrome, Fanconi anemia, adrenoleukodystrophy (ALD), metachromatic leukodystrophy (MLD), HIV/AIDS, other metabolic disorders, genes encoding mis-folded proteins involved in diseases, genes leading to loss-of-function involved in diseases; generally, mutations that can be targeted in an HSC, using any herein-discussed delivery systems and designed molecules are envisioned.

Genome editing: The CRISPR/Cas systems of the present invention can be used to correct genetic mutations including as herein discussed; see also WO2013163628.

The present invention, contemplates correction of hematopoietic disorders. For example, Severe Combined Immune Deficiency (SCID) results from a defect in lymphocytes T maturation, always associated with a functional defect in lymphocytes B (Cavazzana-Calvo et al., Annu. Rev. Med., 2005, 56, 585-602; Fischer et al., Immunol. Rev., 2005, 203, 98-109). In the case of Adenosine Deaminase (ADA) deficiency, one of the SCID forms, patients can be treated by injection of recombinant Adenosine Deaminase enzyme. Since the ADA gene has been shown to be mutated in SCID patients (Giblett et al., Lancet, 1972, 2, 1067-1069), several other genes involved in SCID have been identified (Cavazzana-Calvo et al., Annu. Rev. Med., 2005, 56, 585-602; Fischer et al., Immunol. Rev., 2005, 203, 98-109). There are four major causes for SCID: (i) the most frequent form of SCID, SCID-X1 (X-linked SCID or X-SCID), is caused by mutation in the IL2RG gene, resulting in the absence of mature T lymphocytes and NK cells. IL2RG encodes the gamma C protein (Noguchi, et al., Cell, 1993, 73, 147-157), a common component of at least five interleukin receptor complexes. These receptors activate several targets through the JAK3 kinase (Macchi et al., Nature, 1995, 377, 65-68), which inactivation results in the same syndrome as gamma C inactivation; (ii) mutation in the ADA gene results in a defect in purine metabolism that is lethal for lymphocyte precursors, which in turn results in the quasi absence of B, T and NK cells; (iii) V(D)J recombination is an essential step in the maturation of immunoglobulins and T lymphocytes receptors (TCRs). Mutations in Recombination Activating Gene 1 and 2 (RAG1 and RAG2) and Artemis, three genes involved in this process, result in the absence of mature T and B lymphocytes; and (iv) Mutations in other genes such as CD45, involved in T cell specific signaling have also been reported, although they represent a minority of cases (Cavazzana-Calvo et al., Annu. Rev. Med., 2005, 56, 585-602; Fischer et al., Immunol. Rev., 2005, 203, 98-109). In aspect of the invention, relating to CRISPR or CRISPR-Cas complexes contemplates system, the invention contemplates that it may be used to correct ocular defects that arise from several genetic mutations further described in Genetic Diseases of the Eye, Second Edition, edited by Elias I. Traboulsi, Oxford University Press, 2012. Non-limiting examples of ocular defects to be corrected include macular degeneration (MD), retinitis pigmentosa (RP). Non-limiting examples of genes and proteins associated with ocular defects include but are not limited to the following proteins: (ABCA4) ATP-binding cassette, sub-family A (ABC1), member 4 ACHM1 achromatopsia (rod monochromacy) 1 ApoE Apolipoprotein E (ApoE) C1QTNF5 (CTRPS) C1q and tumor necrosis factor related protein 5 (C1QTNF5) C2 Complement component 2 (C2) C3 Complement components (C3) CCL2 Chemokine (C-C motif) Ligand 2 (CCL2) CCR2 Chemokine (C-C motif) receptor 2 (CCR2) CD36 Cluster of Differentiation 36 CFB Complement factor B CFH Complement factor CFH H CFHR1 complement factor H-related 1 CFHR3 complement factor H-related 3 CNGB3 cyclic nucleotide gated channel beta 3 CP ceruloplasmin (CP) CRP C reactive protein (CRP) CST3 cystatin C or cystatin 3 (CST3) CTSD Cathepsin D (CTSD) CX3CR1 chemokine (C-X3-C motif) receptor 1 ELOVL4 Elongation of very long chain fatty acids 4 ERCC6 excision repair cross-complementing rodent repair deficiency, complementation group 6 FBLN5 Fibulin-5 FBLN5 Fibulin 5 FBLN6 Fibulin 6 FSCN2 fascin (FSCN2) HMCN1 Hemicentrin 1 HMCN1 hemicentin 1 HTRA1 HtrA serine peptidase 1 (HTRA1) HTRA1 HtrA serine peptidase 1 IL-6 Interleukin 6 IL-8 Interleukin 8 LOC387715 Hypothetical protein PLEKHA1 Pleckstrin homology domain-containing family A member 1 (PLEKHA1) PROM1 Prominin 1(PROM1 or CD133) PRPH2 Peripherin-2 RPGR retinitis pigmentosa GTPase regulator SERPING1 serpin peptidase inhibitor, Glade G, member 1 (C1- inhibitor) TCOF1 Treacle TIMP3 Metalloproteinase inhibitor 3 (TIMP3) TLR3 Toll-like receptor 3 The present invention, with regard to CRISPR or CRISPR-Cas complexes contemplates also contemplates delivering to the heart. For the heart, a myocardium tropic adena-associated virus (AAVM) is preferred, in particular AAVM41 which showed preferential gene transfer in the heart (see, e.g., Lin-Yanga et al., PNAS, Mar. 10, 2009, vol. 106, no. 10). For example, US Patent Publication No. 20110023139, describes use of zinc finger nucleases to genetically modify cells, animals and proteins associated with cardiovascular disease. Cardiovascular diseases generally include high blood pressure, heart attacks, heart failure, and stroke and TIA. By way of example, the chromosomal sequence may comprise, but is not limited to, IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJS (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCGS (ATP-binding cassette, sub-family G (WHITE), member 5), PRDX2 (peroxiredoxin 2), CAPNS (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, Glade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, Glade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, Glade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD4OLG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), M1VIP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, Glade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F 13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac 1)), LMNA (lamin NC), CD59 (CD59 molecule, complement regulatory protein), SCNSA (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, Glade A (alpha-1 antiproteinase, antitrypsin), member 3), CASR (calcium-sensing receptor), GJAS (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMPI (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-.beta.-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (B321 placental growth factor), THPO (thrombopoietin), GP6 (glycoprotein VI (platelet)), TACR1 (tachykinin receptor 1), NTS (neurotensin), HNF1 A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOXSAP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, Glade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, Glade F (alpha-2 antiplasmin, pigment epithelium derived factor), member 1), EDN3 (endothelin 3), DHFR (dihydrofolate reductase), GAS6 (growth arrest-specific 6), S1VIPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, Glade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDXS (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetygalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysacchande binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LEVI and senescent cell antigen-like domains 1), RHOB (ras homolog gene family, member B), LY96 (lymphocyte antigen 96), FOXO1 (forkhead box O1), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep(15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene). In an additional embodiment, the chromosomal sequence may further be selected from Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), and combinations thereof. In one iteration, the chromosomal sequences and proteins encoded by chromosomal sequences involved in cardiovascular disease may be chosen from Cacnal C, Sodl, Pten, Ppar(alpha), Apo E, Leptin, and combinations thereof. The text herein accordingly provides exemplary targets as to CRISPR or CRISPR-Cas systems or complexes.

### Delivery

Delivery of the designed molecules may be in vivo, ex vivo, or in vitro. Nucleic acid delivery is a useful method of in vivo delivery.

The drug delivery system optionally and preferably is designed to shield the nucleotide based therapeutic agent from degradation, whether chemical in nature or due to attack from enzymes and other factors in the body of the subject.

The drug delivery system of US Patent Publication 20110195123 is optionally associated with sensing and/or activation appliances that are operated at and/or after implantation of the device, by non and/or minimally invasive methods of activation and/or acceleration/deceleration, for example optionally including but not limited to thermal heating and cooling, laser beams, and ultrasonic, including focused ultrasound and/or RF (radiofrequency) methods or devices.

According to some embodiments of US Patent Publication 20110195123, the site for local delivery may optionally include target sites characterized by high abnormal proliferation of cells, and suppressed apoptosis, including tumors, active and or chronic inflammation and infection including autoimmune diseases states, degenerating tissue including muscle and nervous tissue, chronic pain, degenerative sites, and location of bone fractures and other wound locations for enhancement of regeneration of tissue, and injured cardiac, smooth and striated muscle.

The site for implantation of the composition, or target site, preferably features a radius, area and/or volume that is sufficiently small for targeted local delivery. For example, the target site optionally has a diameter in a range of from about 0.1 mm to about 5 cm.

The location of the target site is preferably selected for maximum therapeutic efficacy. For example, the composition of the drug delivery system (optionally with a device for implantation as described above) is optionally and preferably implanted within or in the proximity of a tumor environment, or the blood supply associated thereof.

For example the composition (optionally with the device) is optionally implanted within or in the proximity to pancreas, prostate, breast, liver, via the nipple, within the vascular system and so forth.

The target location is optionally selected from the group comprising, consisting essentially of, or consisting of (as non-limiting examples only, as optionally any site within the body may be suitable for implanting a Loder): 1. brain at degenerative sites like in Parkinson or Alzheimer disease at the basal ganglia, white and gray matter; 2. spine as in the case of amyotrophic lateral sclerosis (ALS); 3. uterine cervix to prevent HPV infection; 4. active and chronic inflammatory joints; 5. dermis as in the case of psoriasis; 6. sympathetic and sensoric nervous sites for analgesic effect; 7. Intra osseous implantation; 8. acute and chronic infection sites; 9. Intra vaginal; 10. Inner ear-auditory system, labyrinth of the inner ear, vestibular system; 11. Intra tracheal; 12. Intra-cardiac; coronary, epicardiac; 13. urinary bladder; 14. biliary system; 15. parenchymal tissue including and not limited to the kidney, liver, spleen; 16. lymph nodes; 17. salivary glands; 18. dental gums; 19. Intra-articular (into joints); 20. Intra-ocular; 21. Brain tissue; 22. Brain ventricles; 23. Cavities, including abdominal cavity (for example but without limitation, for ovary cancer); 24. Intra esophageal and 25. Intra rectal.

Optionally insertion of the system (for example a device containing the composition) is associated with injection of material to the ECM at the target site and the vicinity of that site to affect local pH and/or temperature and/or other biological factors affecting the diffusion of the drug and/or drug kinetics in the ECM, of the target site and the vicinity of such a site.

Optionally, according to some embodiments, the release of said agent could be associated with sensing and/or activation appliances that are operated prior and/or at and/or after insertion, by non and/or minimally invasive and/or else methods of activation and/or acceleration/deceleration, including laser beam, radiation, thermal heating and cooling, and ultrasonic, including focused ultrasound and/or RF (radiofrequency) methods or devices, and chemical activators.

According to other embodiments of US Patent Publication 20110195123, the drug preferably comprises a RNA, for example for localized cancer cases in breast, pancreas, brain, kidney, bladder, lung, and prostate as described below. Although exemplified with RNAi, many drugs are applicable to be encapsulated in Loder, and can be used in association with this invention, as long as such drugs can be encapsulated with the Loder substrate, such as a matrix for example, and this system may be used and/or adapted to deliver the CRISPR Cas system of the present invention.

As another example of a specific application, neuro and muscular degenerative diseases develop due to abnormal gene expression. Local delivery of RNAs may have therapeutic properties for interfering with such abnormal gene expression. Local delivery of anti apoptotic, anti-inflammatory and anti-degenerative drugs including small drugs and macromolecules may also optionally be therapeutic. In such cases the Loder is applied for prolonged release at constant rate and/or through a dedicated device that is implanted separately. All of this may be used and/or adapted to the CRISPR Cas system of the present invention.

As yet another example of a specific application, psychiatric and cognitive disorders are treated with gene modifiers. Gene knockdown is a treatment option. Loders locally delivering agents to central nervous system sites are therapeutic options for psychiatric and cognitive disorders including but not limited to psychosis, bi-polar diseases, neurotic disorders and behavioral maladies. The Loders could also deliver locally drugs including small drugs and macromolecules upon implantation at specific brain sites. All of this may be used and/or adapted to the CRISPR Cas system of the present invention.

As another example of a specific application, silencing of innate and/or adaptive immune mediators at local sites enables the prevention of organ transplant rejection. Local delivery of RNAs and immunomodulating reagents with the Loder implanted into the transplanted organ and/or the implanted site renders local immune suppression by repelling immune cells such as CD8 activated against the transplanted organ. All of this may be used/and or adapted to the CRISPR Cas system of the present invention.

As another example of a specific application, vascular growth factors including VEGFs and angiogenin and others are essential for neovascularization. Local delivery of the factors, peptides, peptidomimetics, or suppressing their repressors is an important therapeutic modality; silencing the repressors and local delivery of the factors, peptides, macromolecules and small drugs stimulating angiogenesis with the Loder is therapeutic for peripheral, systemic and cardiac vascular disease.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as ERCP, stereotactic methods into the brain tissue, Laparoscopy, including implantation with a laparoscope into joints, abdominal organs, the bladder wall and body cavities.

Implantable devices may also include cells, such as epidermal progenitor cells that have been edited or modified to express the CRISPR-Cas systems disclosed herein. See. Yue et al. “Engineered Epidermal Progenitor Cells Can Correct Diet-Induced Obesity and Diabetes” Cell Stem Cell (2017) 21(2):256-263.

Implantable device technology herein discussed can be employed with herein teachings and hence by this disclosure and the knowledge in the art, CRISPR-Cas system or components thereof or nucleic acid molecules thereof or encoding or providing components may be delivered via an implantable device.

### Aerosol Delivery

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector.

### Viral Capsid Particles

In an aspect, the invention provides a particle delivery system comprising a hybrid virus capsid protein or hybrid viral outer protein, wherein the hybrid virus capsid or outer protein comprises a virus capsid or outer protein attached to at least a portion of a non-capsid protein or peptide. The genetic material of a virus is stored within a viral structure called the capsid. The capsid of certain viruses are enclosed in a membrane called the viral envelope. The viral envelope is made up of a lipid bilayer embedded with viral proteins including viral glycoproteins. As used herein, an “envelope protein” or “outer protein” means a protein exposed at the surface of a viral particle that is not a capsid protein. For example envelope or outer proteins typically comprise proteins embedded in the envelope of the virus. Non-limiting examples of outer or envelope proteins include, without limit, gp41 and gp120 of HIV, hemagglutinin, neuraminidase and M2 proteins of influenza virus.

In an embodiment of the delivery system, the non-capsid protein or peptide has a molecular weight of up to a megadalton, or has a molecular weight in the range of 110 to 160 kDa, 160 to 200 kDa, 200 to 250 kDa, 250 to 300 kDa, 300 to 400 kDa, or 400 to 500 kDa, the non-capsid protein or peptide comprises a CRISPR protein.

The present application provides a vector for delivering an effector protein and at least one CRISPR guide RNA to a cell comprising a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the virus is an adeno-associated virus (AAV) or an adenovirus. In another embodiment, the effector protein is a CRISPR anzyme. In a further embodiment, the CRISPR enzyme is Cas9.

In a related aspect, the invention provides a lentiviral vector for delivering an effector protein and at least one CRISPR guide RNA to a cell comprising a promoter operably linked to a polynucleotide sequence encoding Cas9 and a second promoter operably linked to a polynucleotide sequence encoding at least one guide RNA, wherein the polynucleotide sequences are in reverse orientation.

In an embodiment of the delivery system, the virus is lentivirus or murine leukemia virus (MuMLV).

In an embodiment of the delivery system, the virus is an Adenoviridae or a Parvoviridae or a retrovirus or a Rhabdoviridae or an enveloped virus having a glycoprotein protein (G protein).

In an embodiment of the delivery system, the virus is VSV or rabies virus.

In an embodiment of the delivery system, the capsid or outer protein comprises a capsid protein having VP1, VP2 or VP3.

In an embodiment of the delivery system, the capsid protein is VP3, and the non-capsid protein is inserted into or attached to VP3 loop 3 or loop 6.

In an embodiment of the delivery system, the virus is delivered to the interior of a cell.

In an embodiment of the delivery system, the capsid or outer protein and the non-capsid protein can dissociate after delivery into a cell.

In an embodiment of the delivery system, the capsid or outer protein is attached to the protein by a linker.

In an embodiment of the delivery system, the linker comprises amino acids.

In an embodiment of the delivery system, the linker is a chemical linker.

In an embodiment of the delivery system, the linker is cleavable.

In an embodiment of the delivery system, the linker is biodegradable.

In an embodiment of the delivery system, the linker comprises (GGGGS)1-3, ENLYFQG (SEQ ID NO:18), or a disulfide.

In an embodiment, the delivery system comprises a protease or nucleic acid molecule(s) encoding a protease that is expressed, said protease being capable of cleaving the linker, whereby there can be cleavage of the linker. In an embodiment of the invention, a protease is delivered with a particle component of the system, for example packaged, mixed with, or enclosed by lipid and or capsid. Entry of the particle into a cell is thereby accompanied or followed by cleavage and dissociation of payload from particle. In certain embodiments, an expressible nucleic acid encoding a protease is delivered, whereby at entry or following entry of the particle into a cell, there is protease expression, linker cleavage, and dissociation of payload from capsid. In certain embodiments, dissociation of payload occurs with viral replication. In certain embodiments, dissociation of payload occurs in the absence of productive virus replication.

In an embodiment of the delivery system, each terminus of a CRISPR protein is attached to the capsid or outer protein by a linker.

In an embodiment of the delivery system, the non-capsid protein is attached to the exterior portion of the capsid or outer protein.

In an embodiment of the delivery system, the non-capsid protein is attached to the interior portion of the capsid or outer protein.

In an embodiment of the delivery system, the capsid or outer protein and the non-capsid protein are a fusion protein.

In an embodiment of the delivery system, the non-capsid protein is encapsulated by the capsid or outer protein.

In an embodiment of the delivery system, the non-capsid protein is attached to a component of the capsid protein or a component of the outer protein prior to formation of the capsid or the outer protein.

In an embodiment of the delivery system, the protein is attached to the capsid or outer protein after formation of the capsid or outer protein.

In an embodiment, the delivery system comprises a targeting moiety, such as active targeting of a lipid entity of the invention, e.g., lipid particle or nanoparticle or liposome or lipid bylayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference. Mention is also made of WO/2016/027264, and the documents it cites, all of which are incorporated herein by reference. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference.

An actively targeting lipid particle or nanoparticle or liposome or lipid bylayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bylayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the bloodbrain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody(or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer- lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bylayer). The microenvironment of a cell mass such as a tumor microenvironment can be targeted; for instance, it may be advantageous to target cell mass vasculature, such as the the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation. Matrix metalloproteases (M1VIPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be been utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydro phobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO:19)) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the delivery system comprising an actively targeting lipid particle or nanoparticle or liposome or lipid bylayer delivery system; or comprising a lipid particle or nanoparticle or liposome or lipid bylayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety. Likewise, the following table provides exemplary targeting moieties that can be used in the practice of the invention an as to each an aspect of the invention provides a delivery system that comprises such a targeting moiety.

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent | | ovarian carcinoma |
| fusogenic peptide | | |
| diINF-7 | | |
| anti-VEGFR | VEGF Receptor | tumor vasculature |
| anti-CD19 | CD19 (B cell marker) | leukemia, lymphoma |
| cell-penetrating | | blood-brain barrier |
| peptide | | |
| cyclic arginine-glycine- | avβ3 | glioblastoma cells, human |
| aspartic acid-tyrosine- | | umbilical vein endothelial |
| cysteine peptide | | cells, tumor angiogenesis |
| (c(RGDyC)-LP) (SEQ | | |
| ID NO: 20) | | |
| ASSHN (SEQ ID NO: | | endothelial progenitor cells; |
| 21) peptide | | anti-cancer |
| PR_b peptide | α₅β₁ integrin | cancer cells |
| AG86 peptide | α₆β₄ integrin | cancer cells |
| KCCYSL (SEQ ID NO: | HER-2 receptor | cancer cells |
| 22) (P6.1 peptide) | | |
| affinity peptide LN | Aminopeptidase N | APN-positive tumor |
| (YEVGHRC) (SEQ ID | (APN/CD13) | |
| NO: 23) | | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the delivery system comprises a lipid entity having a targeting moiety.

With regard to CRISPR-Cas systems, it is possible to deliver Cas9 and gRNA (and, for instance, HR repair template) into cells using liposomes or particles. Thus delivery of the CRISPR enzyme, such as a Cas9 and/or delivery of the RNAs of the invention may be in RNA form and via microvesicles, liposomes or particles . For example, Cas9 mRNA and gRNA can be packaged into liposomal particles for delivery in vivo. Liposomal transfection reagents such as lipofectamine from Life Technologies and other reagents on the market can effectively deliver RNA molecules into the liver.

Means of delivery of RNA also preferred include delivery of RNA via nanoparticles (Cho, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder, A., Levins, C., Cortez, C., Langer, R., and Anderson, D., Lipid-based nanotherapeutics for siRNA delivery, Journal of Internal Medicine, 267: 9-21, 2010, PMID: 20059641). Indeed, exosomes have been shown to be particularly useful in delivery siRNA, a system with some parallels to the CRISPR system. For instance, El-Andaloussi S, et al. (“Exosome-mediated delivery of siRNA in vitro and in vivo.” Nat Protoc. 2012 December;7(12):2112-26. doi: 10.1038/nprot.2012.131. Epub 2012 Nov. 15.) describe how exosomes are promising tools for drug delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purify and characterized from transfected cell supernatant, then RNA is loaded into the exosomes. Delivery or administration according to the invention can be performed with exosomes, in particular but not limited to the brain. Vitamin E (a-tocopherol) may be conjugated with CRISPR Cas and delivered to the brain along with high density lipoprotein (HDL), for example in a similar manner as was done by Uno et al. (HUMAN GENE THERAPY 22:711-719 (June 2011)) for delivering short-interfering RNA (siRNA) to the brain. Mice were infused via Osmotic minipumps (model 1007D; Alzet, Cupertino, Calif.) filled with phosphate-buffered saline (PBS) or free TocsiBACE or Toc-siBACE/HDL and connected with Brain Infusion Kit 3 (Alzet). A brain-infusion cannula was placed about 0.5mm posterior to the bregma at midline for infusion into the dorsal third ventricle. Uno et al. found that as little as 3 nmol of Toc-siRNA with HDL could induce a target reduction in comparable degree by the same ICV infusion method. A similar dosage of CRISPR Cas conjugated to a-tocopherol and co-administered with HDL targeted to the brain may be contemplated for humans in the present invention, for example, about 3 nmol to about 3 μmol of CRISPR Cas targeted to the brain may be contemplated.

Zou et al. ((HUMAN GENE THERAPY 22:465-475 (April 2011)) describes a method of lentiviral-mediated delivery of short-hairpin RNAs targeting PKCy for in vivo gene silencing in the spinal cord of rats. Zou et al. administered about 10 μl of a recombinant lentivirus having a titer of 1×10⁹ transducing units (TU)/ml by an intrathecal catheter. A similar dosage of CRISPR Cas expressed in a lentiviral vector may be contemplated for humans in the present invention, for example, about 10-50 ml of CRISPR Cas in a lentivirus having a titer of 1×10⁹ transducing units (TU)/ml may be contemplated. A similar dosage of CRISPR Cas expressed in a lentiviral vector targeted to the brain may be contemplated for humans in the present invention, for example, about 10-50 ml of CRISPR Cas targeted to the brain in a lentivirus having a titer of 1×10⁹ transducing units (TU)/ml may be contemplated.

Anderson et al. (US 20170079916) provides a modified dendrimer nanoparticle for the delivery of therapeutic, prophylactic and/or diagnostic agents to a subject, comprising: one or more zero to seven generation alkylated dendrimers; one or more amphiphilic polymers; and one or more therapeutic, prophylactic and/or diagnostic agents encapsulated therein. Anderson et al. (US 20160367686) provides compounds and compositions for the delivery of an agent to a subject or cell comprising the compound , or a salt thereof; an agent; and optionally, an excipient. The agent may be an organic molecule, inorganic molecule, nucleic acid, protein, peptide, polynucleotide, targeting agent, an isotopically labeled chemical compound, vaccine, an immunological agent, or an agent useful in bioprocessing. The composition may further comprise cholesterol, a PEGylated lipid, a phospholipid, or an apolipoprotein. Anderson et al. (US20150232883) provides a delivery particle formulations and/or systems, preferably nanoparticle delivery formulations and/or systems that can deliver one or more components of the CRISPR-Cas systems disclosed herein.The delivery particle formulations may further comprise a surfactant, lipid or protein, wherein the surfactant may comprise a cationic lipid. Anderson et al. (US20050123596) provides examples of microparticles that are designed to release their payload when exposed to acidic conditions, wherein the microparticles comprise at least one agent to be delivered, a pH triggering agent, and a polymer, wherein the polymer is selected from the group of polymethacrylates and polyacrylates. Anderson et al (US 20020150626) provides lipid-protein-sugar particles for delivery of nucleic acids, wherein the polynucleotide is encapsulated in a lipid-protein-sugar matrix by contacting the polynucleotide with a lipid, a protein, and a sugar; and spray drying mixture of the polynucleotide, the lipid, the protein, and the sugar to make microparticles.

### Sample Types

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

In other embodiments, a sample may be an environmental sample, such as water, soil, or a surface such as industrial or medical surface. In some embodiments, methods such as disclosed in US patent publication No. 2013/0190196 may be applied for detection of nucleic acid signatures, specifically RNA levels, directly from crude cellular samples with a high degree of sensitivity and specificity. Sequences specific to each pathogen of interest may be identified or selected by comparing the coding sequences from the pathogen of interest to all coding sequences in other organisms by BLAST software.

### Devices

The present disclosure encompasses devices comprising the designed binding molecules, or that can be utilized with the designed binding molecules in method and systems disclosed herein. In embodiments, the device is a microfluidic device that generates and/or merges different droplets (i.e. individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout to allow sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiator) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hour et al. “Direct Detection and drug-resistance profiling of bacteremias using inertial microfluidics” Lap Chip. 15(10):2297-2307 (2016).

In systems described herein, may further be incorporated into wearable medical devices that assess biological samples, such as biological fluids, of a subject outside the clinic setting and report the outcome of the assay remotely to a central server accessible by a medical care professional. The device may include the ability to self-sample blood, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresis” to Andrew Conrad.

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried, therefore embodiments that do not require a supporting device are also contemplated, i.e. the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In certain embodiments, the CRISPR effector protein is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

The present invention may be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader); Portable CRISPR-based diagnostics, Nat Bio 37, 832 (2019), DOI: 10.1038/s41587-019-0220-1.

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based masking constructs, use of a hand held UV light, or other suitable device, may be successfully used to detect a signal owing to the near complete quantum yield provided by quantum dots.

### Lateral Flow Devices

Lateral flow detection devices that comprise SHERLOCK systems are contemplated for use with the optimized binding molecules disclosed herein. The device may comprise a lateral flow substrate for detecting a SHERLOCK reaction. Substrates suitable for use in lateral flow assays are known in the art. These may include, but are not necessarily limited to membranes or pads made of cellulose and/or glass fiber, polyesters, nitrocellulose, or absorbent pads (J Saudi Chem Soc 19(6):689-705; 2015). The SHERLOCK system, i.e. one or more CRISPR systems and corresponding reporter constructs are added to the lateral flow substrate at a defined reagent portion of the lateral flow substrate, typically on one end of the lateral flow substrate.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The SHERLOCK system may be freeze-dried to the lateral flow substrate and packaged as a ready to use device, or the SHERLOCK system may be added to the reagent portion of the lateral flow substrate at the time of using the device. Samples to be screened are loaded at the sample loading portion of the lateral flow substrate. The samples must be liquid samples or samples dissolved in an appropriate solvent, usually aqueous. The liquid sample reconstitutes the SHERLOCK reagents such that a SHERLOCK reaction can occur. The liquid sample begins to flow from the sample portion of the substrate towards the first and second capture regions. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the CRISPR effector protein collateral effect is activated. As activated CRISPR effector protein comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

Oligonucleotide linkers having molecules on either end may comprise DNA if the CRISPR effector protein has DNA collateral activity (Cpf1 and C2c1) or RNA if the CRISPR effector protein has RNA collateral activity. Oligonucleotide linkers may be single stranded or double stranded, and in certain embodiments, they could contain both RNA and DNA regions. Oligonucleotide linkers may be of varying lengths, such as 5-10 nucleotides, 10-20 nucleotides, 20-50 nucleotides, or more.

In some embodiments, the polypeptide identifier elements include affinity tags, such as hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof, FLAG tags, Myc tags, AUl tags, T7 tags, OLLAS tags, Glu-Glu tags, VSV tags, or a combination thereof. Other Affinity tags are well known in the art. Such labels can be detected and/or isolated using methods known in the art (for example, by using specific binding agents, such as antibodies, that recognize a particular affinity tag). Such specific binding agents (for example, antibodies) can further contain, for example, detectable labels, such as isotope labels and/or nucleic acid barcodes such as those described herein.

For instance, a lateral flow strip allows for RNAse (e.g. Cas13a) detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g. anti-FITC) antibodies hybridized at the first line and anti-second molecule (e.g. anti-biotin) antibodies at the second downstream line. As the SHERLOCK reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g. color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined herein, e.g. (lateral) flow tests or (lateral) flow immunochromatographic assays.

### Kits

Kits are provided herein, and can comprise one or more optimized binding molecules, instructions, software for design of binding molecules, solid substrates, reagents and/or systems for us with the diagnostic methods disclosed herein, including, for example, ampllfication reagents, or any combination thereof.

The kits can include instructions for the treatment regime, reagents, equipment (test tubes, reaction vessels, needles, syringes, etc.) and standards for calibrating or conducting the diagnostic methods, optimization of binding molecules, and/or treatment. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

The container means of the kits will generally include at least one vial, test tube, solid substrate, microwells, flask, bottle, or other container means, into which a component may be placed, and preferably, suitably aliquoted. The container means may contain freeze dried or other materials provided with the kit. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments are discussed herein.

### EXAMPLES

### Example 1

The emergence and outbreak of SARS-CoV-2, the causative agent of COVID-19, has rapidly become a global concern and has highlighted the need for fast, sensitive, and specific diagnostics. Here assay designs and experimental resources are provided for use with CRISPR-based nucleic acid detection, that could be valuable for ongoing surveillance. Utilizing the methods described herein, assay designs for detection of 67 viral species and subspecies, including: SARS-CoV-2, phylogenetically-related viruses, and viruses with similar clinical presentation are detailed. The designs are outputs of algorithms as described herein for rapidly designing nucleic acid detection assays that are comprehensive across genomic diversity and predicted to be highly sensitive and specific. Of the design set, 4 SARS-CoV-2 designs with a CRISPR-Cas13 detection system were experimentally screened and then the highest-performing SARS-CoV-2 assay was extensively tested. The sensitivity and speed of this assay using synthetic targets with fluorescent and lateral flow detection is demonstrated. Moreover, the provided protocol can be extended for testing the other 66 provided designs.

The novel Severe acute respiratory syndrome-related coronavirus, SARS-CoV-2 (family: Coronaviridae), is the virus behind a severe outbreak originating in China [1]. SARS-CoV-2 surveillance is essential to slowing widespread transmission. In January 2020, Applicants quickly made available a capture enrichment panel [2] using CATCH [3] that is aimed at enhancing sequencing of SARS-CoV-2 and other respiratory viruses. Capture has also been important for ongoing SARS-like coronavirus surveillance [4], and the panel's inclusion of SARS-like bat and pangolin coronaviruses can aid surveillance efforts.

Testing has focused on extensively testing a point-of-care assay for SARS-CoV-2 using the Cas13-based SHERLOCK technology [6,8,9]. Using this assay, Applicants have demonstrated sensitive detection of synthetic SARS-CoV-2 RNA at 10 copies per microliter.

### Results

### Designs for Single Assay and Multiplex Panels

We have been developing algorithms and machine learning models for rapidly designing nucleic acid detection assays, linked in a system called ADAPT (manuscript in preparation). The designs satisfy several constraints, including on:

Comprehensiveness: Assays account for a high fraction of known sequence diversity in their species or subspecies (>97% for most assays), and are meant to be effective against variable targets.

Predicted sensitivity: Assays are predicted by Applicants' machine learning model to have high detection activity against the full scope of targeted genomic diversity (here, based on LwaCas13a activity only).

Predicted specificity: Assays have high predicted specificity to their species or subspecies, factoring in the full extent of known strain diversity, allowing them to be grouped into panels that are accurate in differentiating between related taxa.

Comprehensiveness and—to some extent—specificity of the designs can be verified in silico .

Using ADAPT 67 assays were designed, satisfying the above constraints, to identify: the SARS-related coronavirus species; SARS-CoV-2; two other subspecies in that species with high similarity to SARS-CoV-2; all other known Coronaviridae species, including 4 other species that commonly cause human illness; and other common respiratory viral species and subspecies (Table 1). Each assay targets a single species or subspecies and can be used individually (e.g., point-of-care detection); additionally, owing to how they are designed, multiple assays can be grouped together to test for multiple targets and differentiate them with high specificity.

Sequences for single assays and multiplex panels are available at adapt.sabetilab.org, incorporated herein by reference.

TABLE 1
A summary of the species and subspecies assayed

| Taxonomic rank | Assay Target |
|---|---|
| Species | Severe acute respiratory |
| | syndrome-related coronavirus |
| | (all known strain diversity) |
| Subspecies | SARS-CoV-2 |
| Subspecies | SARS-CoV-1 |
| Subspecies | SARS-like CoV |
| Species | Human coronavirus 229E |
| Species | Human coronavirus NL63 |
| Species | Betacoronavirus 1 (including |
| | Human coronavirus OC43) |
| Species | Human coronavirus HKU1 |
| Species | Middle East respiratory |
| | syndrome-related coronavirus |
| Species | Influenza A virus (all subtypes) |
| Subspecies | H1 (e.g., H1N1 subtype) |
| Subspecies | H3 (e.g., H3N2 subtype) |
| Subspecies | N1 (e.g., H1N1 subtype) |
| Subspecies | N2 (e.g., H3N2 subtype) |
| Species | Influenza B virus |
| Species | Human respirovirus 1 (HPIV-1) |
| Species | Human rubulavirus 2 (HPIV-2) |
| Species | Human respirovirus 3 (HPIV-3) |
| Species | Human rubulavirus 4 (HPIV-4) |
| Species | Rhinovirus A |
| Species | Rhinovirus B |
| Species | Rhinovirus C |
| Species | Enterovirus A |
| Species | Enterovirus B |
| Species | Enterovirus C |
| Species | Enterovirus D |
| Species | Human orthopneumovirus (HRSV) |
| Species | Human metapneumovirus (HMPV) |
| Species (39) | All additional species in |
| | Coronaviridae family |

Table 1. A summary of the species and subspecies constituting the 67 designs at adapt.sabetilab.org. SARS-CoV-2 is designed to exclude detection of the highly similar RaTG13 sequence, and other similar bat and pangolin SARS-like coronaviruses; the SARS-like subspecies includes most bat and pangolin SARS-like coronaviruses.

### SARS-CoV-2 SHERLOCK Assay Testing

Applicants initially screened a set of 4 designs for SHERLOCK [6,8,9] assays, output by ADAPT to detect SARS-CoV-2, and identified an assay, which was the best-performing and also Applicants'the highest ranked design a priori. This assay was extensively tested using a synthetic RNA target and determined the limit of detection to be 10 copies/μl using both fluorescent and lateral flow detection (FIG. 19A-19B). This assay performs well in comparison to the recently disclosed DETECTR [10] assay (sensitivity: 70-300 cp/μl) [11] and SHERLOCK assay (10-100 cp/μl) [12] for SARS-CoV-2. A protocol for performing this assay is provided in the Methods section, and can be used for testing any of the other designs provided.

### Methods

### SHERLOCK Protocol for SARS-CoV-2

### Isothermal Amplification (RT-RPA)

List of equipment and materials:
Heat block, water bath, or thermocycler, prewarmed to 41° C.
RevertAid Reverse Transcriptase (Thermo)
RNase inhibitor (NEB Murine)
Primer mix @ 5μM of each primer (see Table 4 for primer sequences)
Synthetic DNA or RNA target (see Table 4 for sequences), or extracted RNA from a viral seedstock or patient sample
Rehydration buffer, lyophilized RPA pellets, MgAc @ 280 mM from RPA kit (TwistAmp Basic kit, TwistDx)
Nuclease-free water

TABLE 2
Reagents for isothermal amplification (RT-RPA)

| Reagent | Initial concentration | | Amount to add for N RPA pellets | |
|---|---|---|---|---|
| Rehydration buffer | N/A | | 29.5 × N | μl |
| RPA primer mix | 5 μM of each primer | | 4.8 × N | μl |
| Nuclease-free water | N/A | | 2.1 × N | μl |
| RNase inhibitors | 40 | U/μl | 5 × N | μl |
| RevertAid RT | 200 | U/μl | N | μl |
| Magnesium Acetate (MgAc) | 280 | mM | 3.04 × N | μl |

Step by Step Protocol:
1. Determine the number of pellets needed based on the size of the experiment (2 samples per pellet, if doing 20 μl RPA reactions).
2. Make a master mix for N pellets, consisting of Rehydration buffer, RPA primer mix, water, RNase inhibitors, and RevertAid RT. Do not include MgAc at this step.
3. Resuspend each RPA pellet using the total volume for 1 RPA pellet (−30 μl) of master mix.
4. Add the MgAc to the master mix. Keep the master mix on ice.
5. Aliquot the master mix (containing MgAc) into wells of a 96-well plate or strip tubes pre-chilled on ice. For 20 μl reactions, aliquot 18 μl of master mix.
6. Add sample or a control target to each aliquot of master mix (2 μl, if using 20 μl reactions), mix thoroughly, and incubate at 41° C. for 20 minutes.

### Cas13 Detection

List of Equipment and Materials
Heat block, water bath, or thermocycler, prewarmed to 37° C.
For visual readout: camera or cell phone with camera; for fluorescent readout: qPCR machine/plate reader capable of detecting FAM
For fluorescent readout: optical 96-well plate, optical strip-tubes, or black 96-well plate with clear bottom
10× Cleavage buffer (1× CB is 40 mM Tris pH 7.5, 1 mM DTT)
RNase inhibitors (NEB Murine)
Cleavage reporter, for visual readout: IDT for lateral flow (sequences in Table 4); for fluorescent readout: RNase Alert v2 (Thermo)
LwaCas13 protein @ 0.5 mg/ml, in 16 ul aliquots, diluted in 1× Storage buffer (50 mM Tris pH 7.5, 600 mM NaCl, 5% glycerol, 2 mM DTT) [15]
Cas13 crRNA @ 2 μM (see Table 4 for sequences)
T7 RNA polymerase (Lucigen N×Gen)
rNTP mix @25 mM each (NEB)
MgCl2@ 100 mM
Nuclease-free water

TABLE 3
Reagents for Cas13 detection

| Reagent | Initial concentration | | Amount to add for N reactions | |
|---|---|---|---|---|
| Cleavage buffer | 10X | | 2.4 × N | μl |
| Nuclease-free water | N/A | | 11.22 × N | μl |
| rNTP mix | 25 mM of each nucleotide | | 0.96 × N | μl |
| RNase inhibitors | 40 | U/μl | 1.2 × N | μl |
| Cleavage reporter | 16 μM (visual readout) or | | 1.5 × N | μl |
| | 2 μM (fluorescent readout) | | | |
| Lwa/Cas13 protein | Diluted in 1X SB | | 2.4 × N | μl |
| T7 RNA Polymerase | 50 | U/μl | 0.72 × N | μl |
| Cas13 crRNA | 2 | μM | 0.24 × N | μl |
| MgCl₂ | 100 | mM | 2.16 × N | μl |

| | | |
|---|---|---|
| RPA product | N/A | 1 μl per reaction |

TABLE 4
Primer, target, and crRNA sequences for the SARS-CoV-2 assay.

| Name | Sequence |
|---|---|
| RPA forward primer | gaaatTAATACGACTCACTATAgggCCAAGGTAAACCTTTGG |
| | AATTTGGTGCCAC (SEQ ID NO: 24) |
| RPA reverse primer | Actatcatcatctaaccaatcttcttcttg (SEQ ID NO: 25) |
| Synthetic DNA target | gaaatTAATACGACTCACTATAgggGTGAGTTTAAATTGGCTT |
| | CACATATGTATTGTTCTTTCTACCCTCCAGATGAGGATGA |
| | AGAAGAAGGTGATTGTGAAGAAGAAGAGTTTGAGCCATC |
| | AACTCAATATGAGTATGGTACTGAAGATGATTACCAAGG |
| | TAAACCTTTGGAATTTGGTGCCACTTCTGCTGCTCTTCAAC |
| | CTGAAGAAGAGCAAGAAGAAGATTGGTTAGATGATGATA |
| | GTCAACAAACTGTTGGTCAACAAGACGGCAGTGAGGACA |
| | ATCAGACAACTACTATTCAAACAATTGTTGAGGTTCAACC |
| | TCAATTAGAGATGGAACTTACACCAGTTGTTCAGACTATT |
| | GAAGTGAATAGTTTTAGTGGTTATTTAAAACTTACTGACA |
| | ATGTATACATTAAAAATGCAGACATTGTGGAAGAAGCTA |
| | AAAAGGTAAAACCAACAGTGGTTGTTAATGCAGCCAATG |
| | TTTACCTTAAACATGGAGGAGG (SEQ ID NO: 26) |
| Cas13 crRNA | GAUUUAGACUACCCCAAAAACGAAGGGGACUAAAACcuc |
| | uucuucagguugaagagcagcagaa (SEQ ID NO: 27) |
| Cleavage reporter (lateral flow) | /56-FAM/rUrU rUrUrU rUrUrU rUrUrU rUrUrU /3Bio/ |
| | (SEQ ID NO: 28) |

Step by Step Protocol of Cas Detection
1. Dilute LwaCas13a by adding 110.5 μl of 1× Storage buffer to a 16 μl aliquot of Cas13 protein (prior to dilution @ 0.5 mg/ml).
2. Prepare a master mix based on the table above, adding the components in the order they are listed in the table. Do not add RPA product at this step.
3. Aliquot 19 μl of master mix into wells of a 96-well plate or strip tubes pre-chilled on ice. If using a fluorescent readout and depending on the instrument used, an optical PCR or black plate with a clear base can be used.
4. Add 1 μL of RPA product to each master mix aliquot. Seal the plate and incubate at 37° C. for 30 minutes to 3 hours. For fluorescent readout, measure fluorescence every 5 minutes. For visual readout, see additional details below.

Visual Readout

List of Equipment and Materials
HybriDetect 1 lateral flow strips (Milenia)
Hybridetect Assay Buffer (Milenia)

Step by Step Protocol
1. After incubation at 37° C. for 30 minutes to 3 hours, add 80 μl of Hybridetect Assay Buffer to the total volume of each detection reaction.
2. Add a lateral flow strip to each well and incubate for 2-5 minutes at room temperature.
   a. Take care to avoid contamination of the strips, by using tweezers to remove individual strips and place in buffer.
3. Remove strips, place on flat, well-lit surface, and analyze or acquire images of the strips.

### Discussion

Ongoing SARS-CoV-2 sequencing is key to developing and monitoring diagnostics. In the case of the SARS-CoV-2 outbreak, genomes have been generated and shared at a remarkable pace, and hank those who have contributed their data through GISAID [13]. Relying on this data [14], it has been shown that it is possible to rapidly design CRISPR-based diagnostics during an outbreak and to test them with multiple detection methods.

Among other goals for this work, planned evaluation includes: (1) sensitivity of the SARS-CoV-2 assay against clinical isolates and patient samplesincluding sputum, throat, and nasal swabs—some of which may be challenging sample types to test; (2) specificity at both the species and subspecies levels against highly related viruses. For the latter, use of a mixture of synthetic targets is intended, reflecting different viral sequences, and patient samples or viral seedstocks when available. The goals is that the comprehensiveness and high predicted sensitivity and specificity of the designs will enable many groups to proceed rapidly and successfully from testing through deployment.

The following References pertain to this Example.

1. Zhou P, Yang X-L, Wang X-G, Hu B, Zhang L, Zhang W, et al. A pneumonia outbreak associated with a new coronavirus of probable bat origin. Nature. 2020. doi:10.1038/s41586-020-2012-7

2. V-Respiratory probe set (2020-01). Available: https://github.com/broadinstitute/catch/tree/master/probe-designs

3. Metsky H C, Siddle K J, Gladden-Young A, Qu J, Yang D K, Brehio P, et al. Capturing sequence diversity in metagenomes with comprehensive and scalable probe design. Nat Biotechnol. 2019;37: 160-168. doi:10.1038/s41587-018-0006-x

4. Li B, Si H-R, Zhu Y, Yang X-L, Anderson DE, Shi Z-L, et al. Discovery of Bat Coronaviruses through Surveillance and Probe Capture-Based Next-Generation Sequencing. mSphere. 2020;5. doi:10.1128/mSphere.00807-19

5. Wee S-L. As Deaths Mount, China Tries to Speed Up Coronavirus Testing. The New York Times. 9 Feb. 2020. Available: https://www.nytimes.com/2020/02/09/world/asia/china-coronavirus-tests.html. Accessed 24 Feb. 2020.

6. Myhrvold C, Freije C A, Gootenberg J S, Abudayyeh O O, Metsky H C, Durbin A F, et al. Field-deployable viral diagnostics using CRISPR-Cas13. Science. 2018;360: 444-448. doi: 10.1126/science. aas8836

7. Wang M, Wu Q, Xu W, Qiao B, Wang J, Zheng H, et al. Clinical diagnosis of 8274 samples with 2019-novel coronavirus in Wuhan. Infectious Diseases (except HIV/AIDS). medRxiv; 2020. doi:10.1101/2020.02.12.20022327

8. Gootenberg J S, Abudayyeh O O, Lee J W, Essletzbichler P, Dy A J, Joung J, et al. Nucleic acid detection with CRISPR-Cas13a/C2c2. Science. 2017;356: 438-442. doi: 10.1126/science. aam9321

9. Gootenberg J S, Abudayyeh O O, Kellner M J, Joung J, Collins J J, Zhang F. Multiplexed and portable nucleic acid detection platform with Cas13, Cas12a, and Csm6. Science. 2018;360: 439-444. doi:10.1126/science.aaq0179

10. Chen J S, Ma E, Harrington L B, Da Costa M, Tian X, Palefsky J M, et al. CRISPR-Cas12a target binding unleashes indiscriminate single-stranded DNase activity. Science. 2018;360: 436-439. doi:10.1126/science.aar6245

11. James P. Broughton, Wayne Deng, Clare L. Fasching, Jasmeet Singh, Charles Y. Chiu, Janice S. Chen. A protocol for rapid detection of the 2019 novel coronavirus SARS-CoV-2 using CRISPR diagnostics: SARS-CoV-2 DETECTR. 2020. Available: https://mammoth.bio/wp-content/upl oads/2020/02/A-protocol-for-rapi d-detecti on-of-the-2019-novel-coronavirus-SARS-CoV-2-using-CRISPR-diagnostics-SARS-CoV-2-DETECTR.pdf

12. Feng Zhang, Omar O. Abudayyeh, Jonathan S. Gootenberg. A protocol for detection of COVID-19 using CRISPR diagnostics. Available: https://www.broadinstitute.org/files/publications/special/COVID-19%20detection%20(updated).pdf

13. Shu Y, McCauley J. GISAID: Global initiative on sharing all influenza data—from vision to reality. Euro Surveill. 2017;22. doi:10.2807/1560-7917.ES.2017.22.13.30494

14. Hou T, Zeng W, Yang M, Chen W, Ren L, Ai J, et al. Development and Evaluation of A CRISPR-based Diagnostic For 2019-novel Coronavirus. Infectious Diseases (except HIV/AIDS). medRxiv; 2020. doi:10.1101/2020.02.22.20025460

15. Kellner M J, Koob J G, Gootenberg J S, Abudayyeh O O, Zhang F. SHERLOCK: nucleic acid detection with CRISPR nucleases. Nat Protoc. 2019;14: 2986-3012. doi:10.1038/s41596-019-0210-2

### Example 2

### Integrated Sample Inactivation, Amplification, and Cas13-based Detection of SARS-CoV-2 using ADAPT Designed Diagnostic Molecules

Point-of-care diagnostic testing is essential to prevent and effectively respond to infectious disease outbreaks. Insufficient nucleic acid diagnostic testing infrastructure (1) and the prevalence of asymptomatic transmission (2, 3) have accelerated the global spread of severe acute respiratory syndrome coronavirus 2 (SARS-CoV-2) (4-6), with confirmed case counts surpassing 5 million (7). Ubiquitous nucleic acid testing whether in doctor's offices, pharmacies, or mobile/drive-thru/pop-up testing sites would increase diagnostic access and is essential for safely reopening businesses, schools, and country borders. Easy-to-use, scalable diagnostics with a quick turnaround time and limited equipment requirements would fulfill this major need and have the potential to alter the trajectory of this global pandemic.

The paradigm for nucleic acid diagnostic testing is a centralized model where patient samples are sent to large clinical laboratories for processing and analysis. RT-qPCR, the highly specific and sensitive current gold-standard for SARS-CoV-2 diagnosis (8), requires laboratory infrastructure for nucleic acid extraction, thermal cycling, and analysis of assay results. The need for thermocyclers can be eliminated through the use of isothermal (i.e., single temperature) amplification methods, such as loop-mediated isothermal amplification (LAMP) and recombinase polymerase amplification (RPA) (9-14). However, isothermal amplification methods still require technological advances (Qian, Boswell, Lu, Chidley, et al. co-submitted to Science) to increase sensitivity on unextracted RNA samples and to reduce non-specific amplification (15, 16), which would enable testing at scale outside of laboratories.

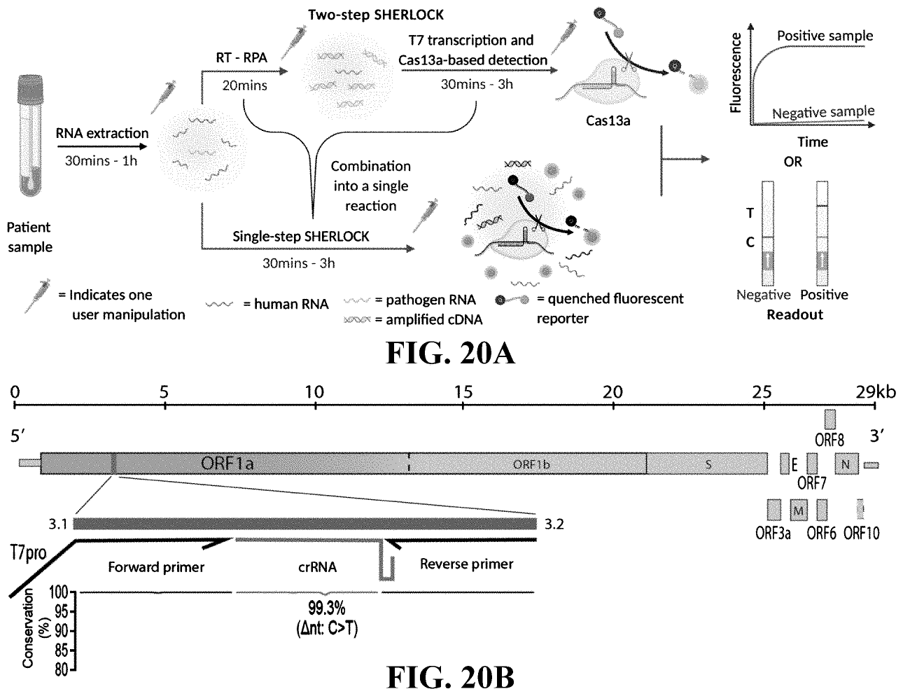

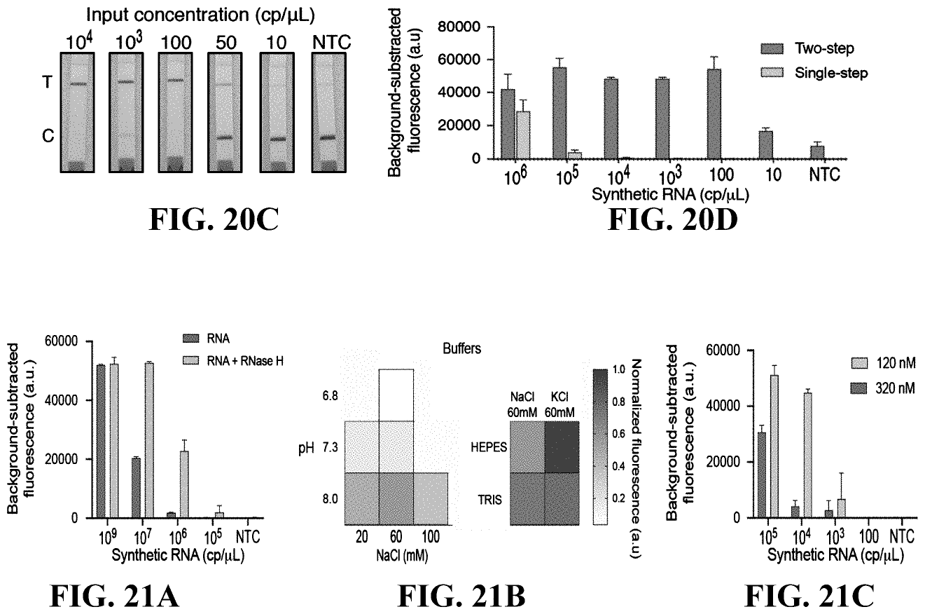

Recently developed CRISPR-based diagnostics have the potential to transform infectious disease diagnosis. Both CRISPR-Cas13- and Cas12-based assays have been developed for SARS-CoV-2 detection using extracted nucleic acids as input (17-22). One such CRISPR-based diagnostic, SHERLOCK (Specific High-sensitivity Enzymatic Reporter unLOCKing), involves two separate steps, starting with extracted nucleic acid: (1) isothermal RPA and (2) T7 transcription and Cas13-mediated collateral cleavage of a single-stranded RNA reporter (23) (FIG. 20A). Cas13-based detection is highly programmable and specific, as it relies on complementary base pairing between the target RNA and the CRISPR RNA (crRNA) sequence (23, 24). However, in their current state, these technologies require nucleic acid extraction (often using kits that are in short supply) and multiple sample transfer steps, limiting their widespread use. SHERLOCK can be paired with HUDSON (Heating Unextracted Diagnostic Samples to Obliterate Nucleases), which eliminates the need for nucleic acid extraction by using heat and chemical reduction to both destroy RNA-degrading nucleases and lyse viral particles (25). Together, SHERLOCK and HUDSON can be performed with limited laboratory infrastructure, solely requiring a heating element. However, the scalability of these methods is currently limited by the need to prepare multiple reaction mixtures and transfer samples between them.

To address the current limitations of nucleic acid diagnostics, Applicants developed SHINE (SHERLOCK and HUDSON Integration to Navigate Epidemics) for extraction-free, rapid, and sensitive detection of SARS-CoV-2 RNA. Applicants established a SARS-CoV-2 assay (18), then combined SHERLOCK's amplification and Cas13-based detection steps, decreasing user manipulations and assay time (FIG. 20A). Applicants demonstrated that SHINE can detect SARS-CoV-2 RNA in HUDSON-treated patient samples with either a paper-based colorimetric readout, or an in-tube fluorescent readout which can be performed with portable equipment and with reduced risk of sample contamination.

Applicants first developed a two-step SHERLOCK assay which sensitively detected SARS-CoV-2 RNA at 10 copies per microliter (cp/μL). Using ADAPT, a computational design tool for nucleic acid diagnostics (Metsky et al. in prep), and as described herein, Applicants identified a region within open reading frame 1a (ORF1a) of SARS-CoV-2 that comprehensively captures known sequence diversity, with high predicted Cas13 targeting activity and SARS-CoV-2 specificity (FIG. 20B) (18). Using both colorimetric and fluorescent readouts, Applicants detected 10 cp/μL of synthetic RNA after incubating samples for 1 h or less, but preparing the reactions required 45-90 minutes of hands-on time depending on the number of samples (FIG. 20C and 20D and FIG. 22A). Applicants tested this assay on HUDSON-treated SARS-CoV-2 viral seedstocks, detecting down to 1.31e5 PFU/ml via colorimetric readout (FIG. 22B). Finally, in a side-by-side comparison of the two-step SHERLOCK assay and the CDC RT-qPCR assay, Applicants demonstrated similar limits of detection, reliably identifying 1-10 cp/μL with stochasticity evident at lower viral titers (FIG. 22C).

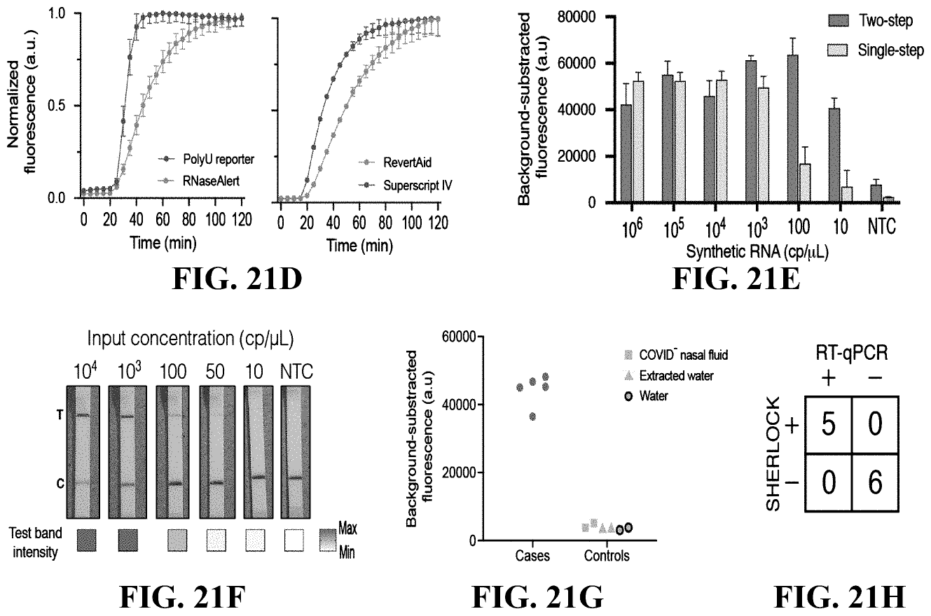

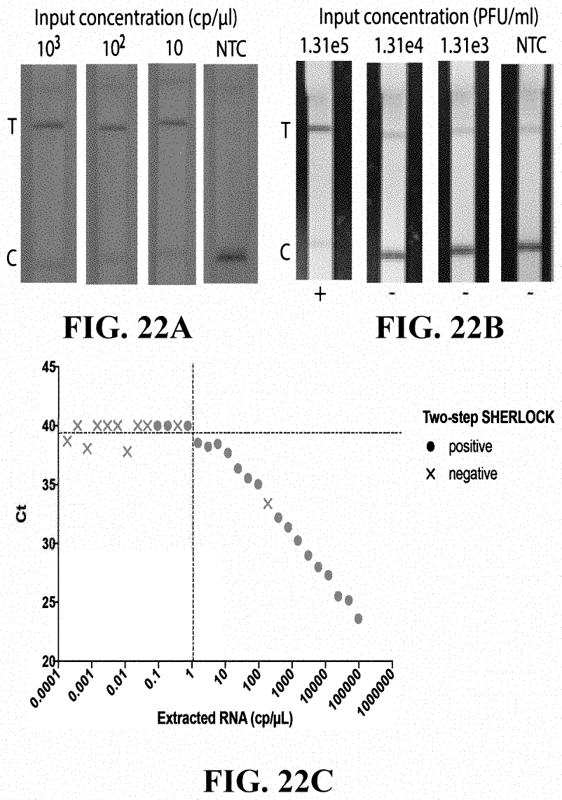

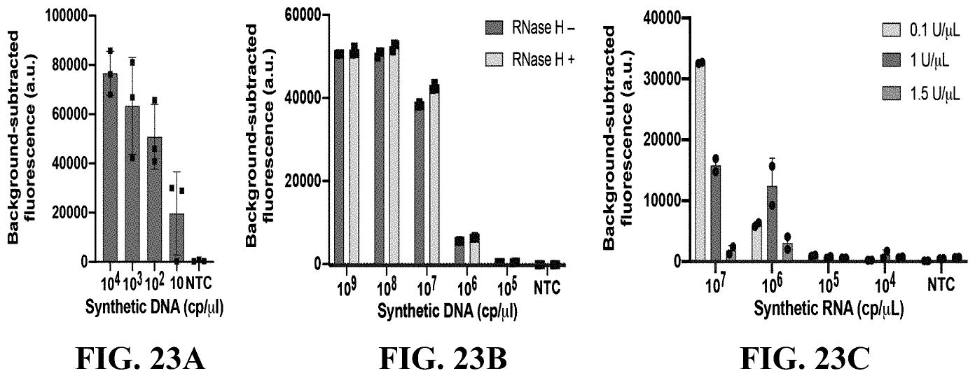

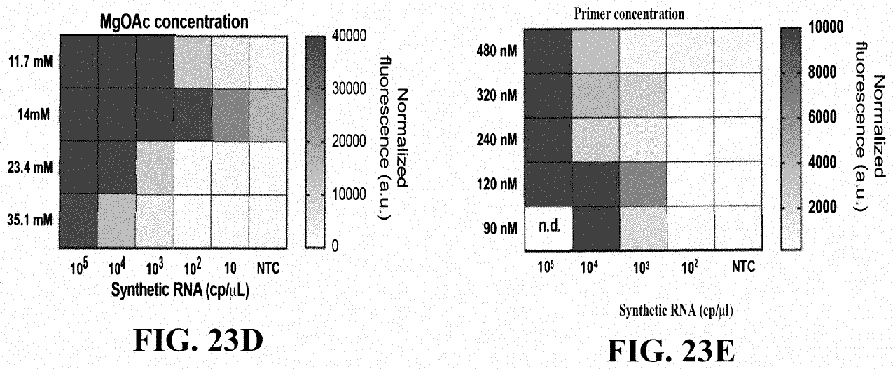

Applicants sought to develop an integrated, streamlined assay that was significantly less time- and labor-intensive than two-step SHERLOCK. However, when Applicants combined RT-RPA (step 1), T7 transcription, and Cas13-based detection (step 2) into a single step (i.e., single-step SHERLOCK), the sensitivity of the assay decreased dramatically. This decrease was specific for RNA input, and likely due to incompatibility of enzymatic reactions with the given conditions (limit of detection (LOD) 106 cp/μL; FIG. 20D and FIG. 23A). As a result, Applicants evaluated whether additional reaction components and optimized reaction conditions could increase the sensitivity and speed of the assay. Addition of RNase H, in the presence of reverse transcriptase, improved the sensitivity of Cas13-based detection of RNA 10-fold (LOD 10⁵ cp/μL; FIG. 21A and FIG. 23B and 23C). RNase H likely enhanced the sensitivity by increasing the efficiency of RT through degradation of DNA:RNA hybrid intermediates (Qian, Boswell, Lu, Chidley, et al. co-submitted to Science).

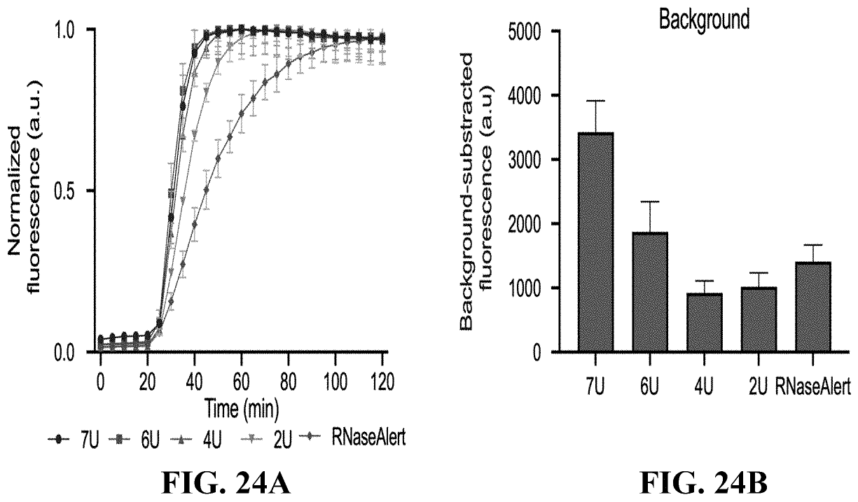

Given that each enzyme involved has optimal activity at distinct reaction conditions, Applicants evaluated the role of different pHs, monovalent salt, magnesium, and primer concentrations on assay sensitivity. Optimized buffer, magnesium, and primer conditions resulted in an LOD of 1,000 cp/μL (FIG. 21B and 21C and FIG. 23D and 23E). Applicants then improved the speed of Cas13 cleavage and RT to reduce the sample-to-answer time. Given the uracil-cleavage preference of Cas13a (24, 26, 27), detection of RNA in the single-step SHERLOCK assay reached half-maximal fluorescence in 67% of the time when RNaseAlert was substituted for a polyU reporter (FIG. 21D, left and FIG. 24A, 24B). In addition, reactions containing SuperScript IV reverse transcriptase reached half-maximal fluorescence two times faster than RevertAid (FIG. 21D, right).

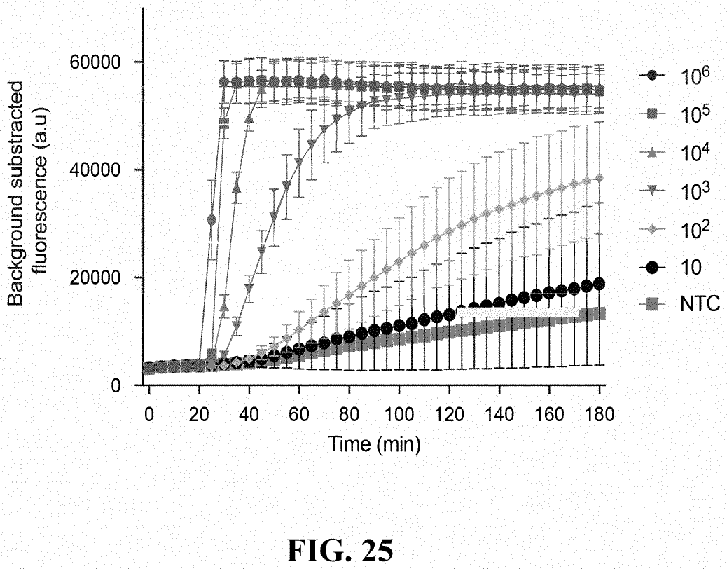

Together, these improvements resulted in an optimized single-step SHERLOCK assay that could detect SARS-CoV-2 RNA with reduced sample-to-answer time and equal sensitivity compared to Applicants' two-step assay. Applicants quantified the LOD of their optimized single-step SHERLOCK assay on synthetic RNA, detecting as few as 10 cp/μL using a fluorescent readout—100,000 times more sensitive than the initial assay—and 100 cp/μL using the lateral-flow-based colorimetric readout (FIG. 21E and 21F and FIG. 25).

Applicants then evaluated their assay's performance on SARS-CoV-2 RNA extracted from patient nasopharyngeal (NP) swabs. Applicants compared their fluorescent single-step SHERLOCK assay to previously-performed RT-qPCR using a pilot set of 9 samples. Applicants detected SARS-CoV-2 from 5 of 5 SARS-CoV-2-positive patient samples tested, demonstrating 100% concordance with RT-qPCR, with no false positives for 4 SARS-CoV-2-negative extracted samples nor 2 non-template controls (FIG. 21H and Table 5).

TABLE 5
Patient Sample Information.
Viral quantity is adjusted as sample was diluted 1:3 for
input into in-house RT-qPCR assay. NP, nasopharyngeal
swab; UTM, universal viral transport medium.

| Sample | | Sample | Viral | Adjusted viral | |
|---|---|---|---|---|---|
| name | Sample ID | type | Ct | quantity (cp/μl) | FIGS. |
| E1 | MA_MGH_00155 | NP in UTM | 25.989 | 892.22 | 21G and 21H |
| E2 | MA_MGH_00156 | NP in UTM | 17.753 | 85,470.64 | 21G and 21H |
| E3 | MA_MGH_00157 | NP in UTM | 17.235 | 113,800.532 | 21G and 21H |
| E4 | MA_MGH_00159 | NP in UTM | 20.861 | 15,298.96 | 21G and 21H |
| E5 | MA_MGH_00160 | NP in UTM | 20.006 | 24,515.988 | 21G and 21H |
| E6 | MA_MGH_00166 | NP in UTM | 25.548 | 1,136.696 | 21G and 21H |
| P1 | MA_MGH_00441 | NP in UTM | 22.109 | 52787.064 | 26G and 26H |
| P2 | MA_MGH_00442 | NP in UTM | 25.764 | 4386.186 | 26G and 26H |
| P3 | MA_MGH_00443 | NP in UTM | 26.809 | 2154.246 | 26G and 26H |
| P4 | MA_MGH_00445 | NP in UTM | 16.429 | 3390658.2 | 26G and 26H |
| P5 | MA_MGH_00446 | NP in UTM | 17.136 | 2022960.6 | 26G and 26H |
| P6 | MA_MGH_00447 | NP in UTM | 16.39 | 3426433.56 | 26G and 26H |
| P7 | MA_MGH_00453 | NP in UTM | 21.935 | 68312.187 | 26G and 26H |
| P8 | MA_MGH_00454 | NP in UTM | 21.936 | 68293.593 | 26G and 26H |
| P9 | MA_MGH_00456 | NP in UTM | 15.678 | 5660253.54 | 26G and 26H |
| P10 | MA_MGH_00458 | NP in UTM | 23.331 | 25530.948 | 26G and 26H |
| P11 | MA_MGH_00459 | NP in UTM | 15.313 | 7329902.04 | 26G and 26H |
| P12 | MA_MGH_00460 | NP in UTM | 24.979 | 7977.141 | 26G and 26H |
| P13 | MA_MGH_00461 | NP in UTM | 16.2 | 3914305.92 | 26G and 26H |
| P14 | MA_MGH_00463 | NP in UTM | 17.05 | 2157841.26 | 26G and 26H |
| P15 | MA_MGH_00464 | NP in UTM | 20.234 | 227657.529 | 26G and 26H |
| P16 | MA_MGH_00465 | NP in UTM | 16.102 | 4201061.4 | 26G and 26H |
| P17 | MA_MGH_00466 | NP in UTM | 17.933 | 1151605.125 | 26G and 26H |
| P18 | MA_MGH_00467 | NP in UTM | 19.023 | 541607.094 | 26G and 26H |
| P19 | MA_MGH_00468 | NP in UTM | 18.661 | 689269.86 | 26G and 26H |
| P20 | MA_MGH_00469 | NP in UTM | 14.983 | 9240984 | 26G and 26H |
| P21 | MA_MGH_00471 | NP in UTM | 22.963 | 33130.494 | 26G and 26H |
| P22 | MA_MGH_00472 | NP in UTM | 13.271 | 30975183 | 26G and 26H |
| P23 | MA_MGH_00473 | NP in UTM | 18.757 | 645122.16 | 26F, 26G and 26H |
| P24 | MA_MGH_00474 | NP in UTM | 17.397 | 1682047.8 | 26F, 26G and 26H |
| P25 | MA_MGH_00475 | NP in UTM | 19.896 | 289419.75 | 26F, 26G and 26H |
| P26 | MA_MGH_00476 | NP in UTM | 16.181 | 3970140.3 | 26F, 26G and 26H |
| P27 | MA_MGH_00477 | NP in UTM | 19.812 | 305659.512 | 26F, 26G and 26H |
| P28 | MA_MGH_00479 | NP in UTM | 13.868 | 20324826 | 26F, 26G and 26H |
| P29 | MA_MGH_00480 | NP in UTM | 21.362 | 102417.507 | 26G and 26H |
| P30 | MA_MGH_00481 | NP in UTM | 22.187 | 57196.188 | 26G and 26H |

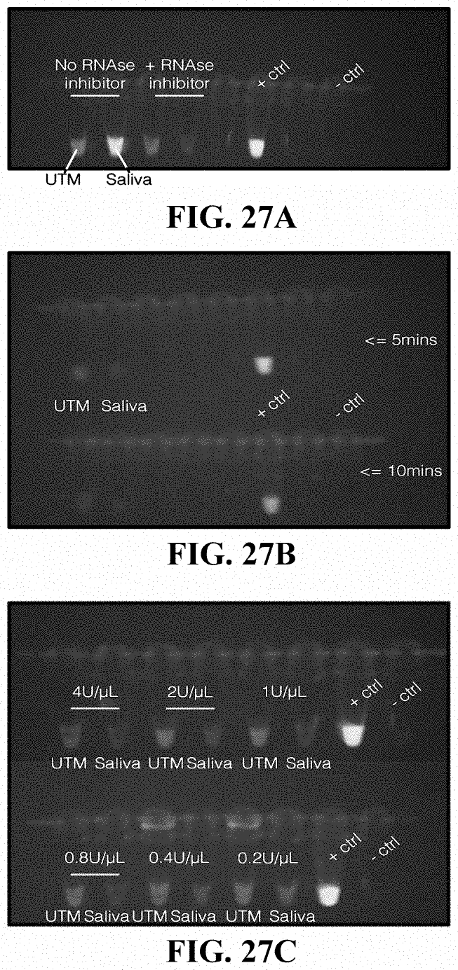

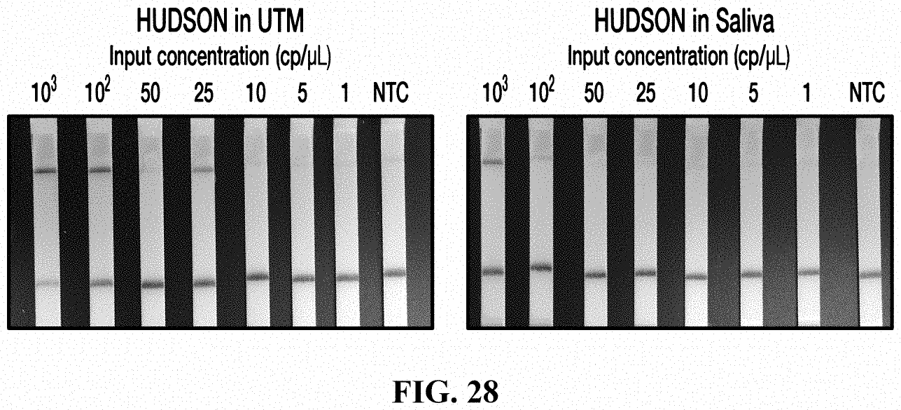

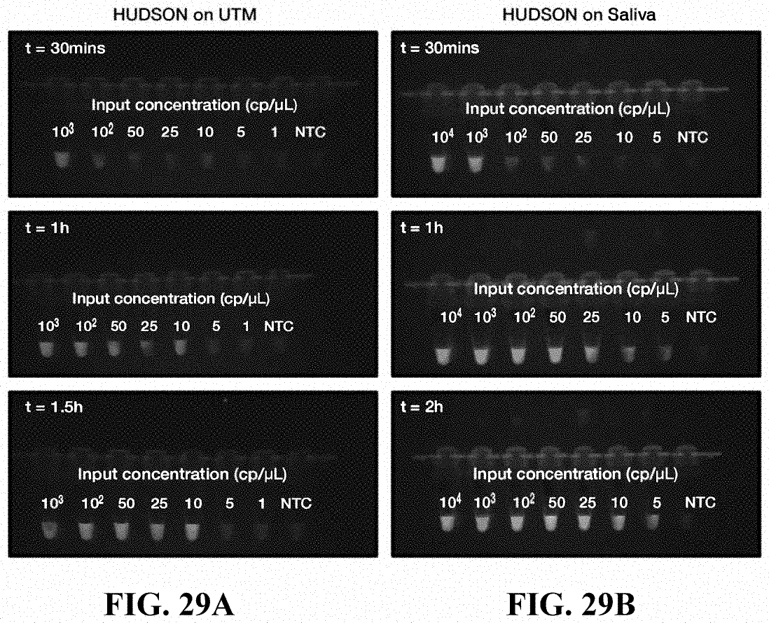

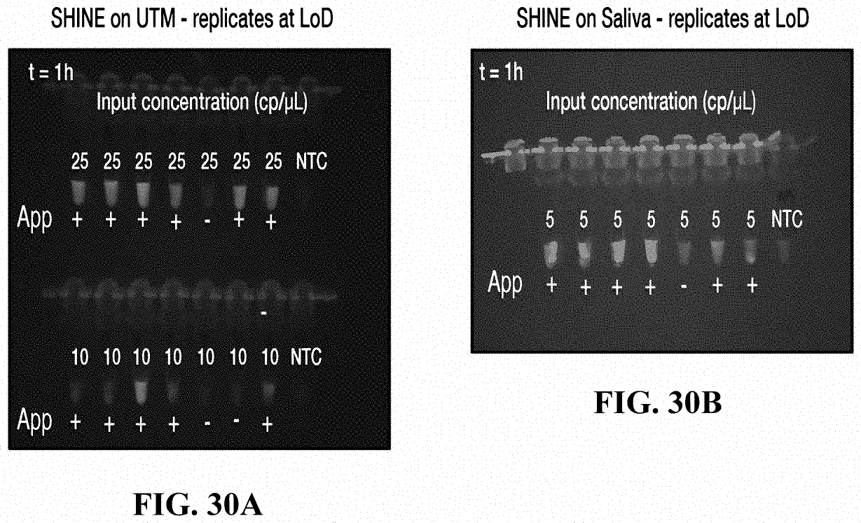

Finally, Applicants paired HUDSON and SHERLOCK with multiple visual readouts to create SHINE (SHERLOCK and HUDSON Integration to Navigate Epidemics), a platform whose results are interpretable by a companion smartphone application (FIG. 26A). In order to reduce total run time, Applicants reduced the incubation time of HUDSON from 30 min to 10 min for both universal viral transport medium (UTM), used for NP swab samples, and for saliva, through the addition of RNase inhibitors (25) (FIG. 26B and FIG. 27). With this faster HUDSON protocol, Applicants detected 50 cp/μL of synthetic RNA when spiked into UTM and 100 cp/μL when spiked into saliva, using a colorimetric readout (FIG. 28). However, the lateral flow readout requires opening of tubes containing amplified products and interpreting the test band by eye, which introduces risks of sample contamination and user bias, respectively. Thus, Applicants incorporated an in-tube fluorescent readout with SHINE. Within 1 hour, Applicants detected as few as 10 cp/μL of SARS-CoV-2 synthetic RNA in HUDSON-treated UTM and 5 cp/μL in HUDSON-treated saliva with the in-tube fluorescent readout (FIG. 26C and 26D and FIGS. 29 and 30). To reduce user-bias in interpreting results of this in-tube readout, Applicants developed a companion smartphone app which uses the built-in smartphone camera to image the reaction tubes. The application then calculates the distance of the experimental tube's pixel intensity distribution from that of a user-selected negative control tube, and returns a binary result indicating the presence or absence of viral RNA in the sample (FIG. 26A and 26E; see Methods for details). Thus, SHINE both minimized equipment requirements and user interpretation bias when implemented with this in-tube readout and the smartphone application.

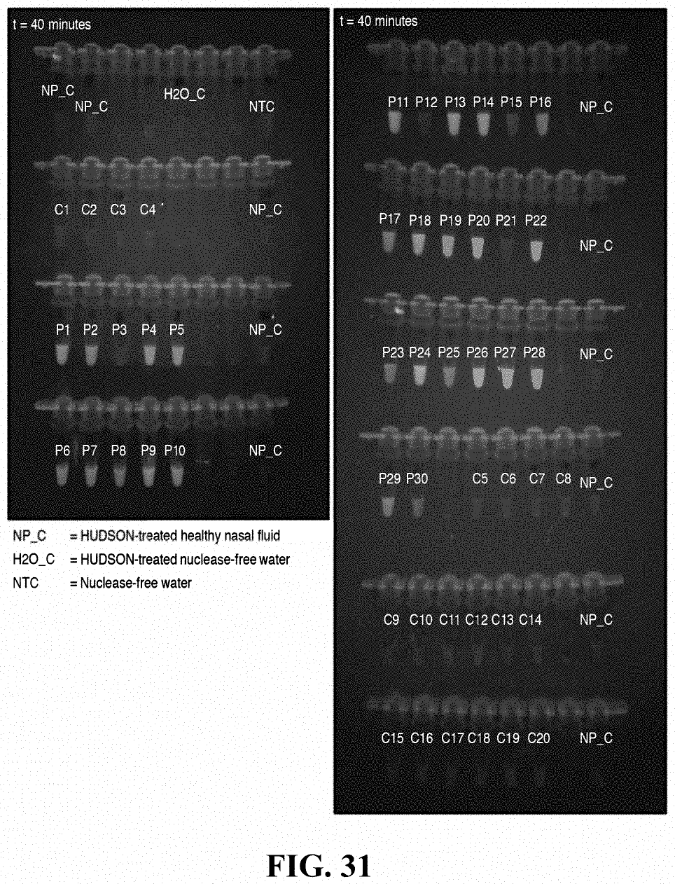

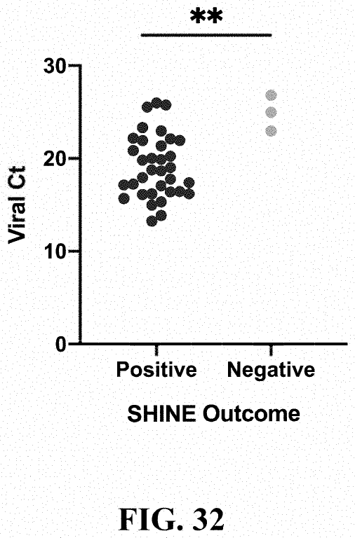

Applicants used SHINE to test a set of 50 unextracted, NP samples from 30 RT-qPCR-confirmed, COVID-19-positive patients and 20 COVID-19-negative patients. Applicants used SHINE with the paper-based colorimetric readout on 6 SARS-CoV-2-positive samples and detected SARS-CoV-2 RNA in all 6 positive samples, and in none of the negative controls (100% concordance, FIG. 26F). For all 50 samples, Applicants used SHINE with the in-tube fluorescence readout and companion smartphone application. Applicants detected SARS-CoV-2 RNA in 27 of 30 COVID-19-positive samples (90% sensitivity) and none of the COVID-19-negative samples (100% specificity) after a 10-minute HUDSON and a 40-minute single-step SHERLOCK incubation (FIG. 26G and 26H, FIG. 31, and Table 5 and 6). Thus, SHINE demonstrated 94% concordance using the in-tube readout with a total run time of 50 minutes. Notably, the RT-qPCR-positive patient NP swabs that SHINE failed to detect tended to have higher Ct values than those that SHINE detected as positive (p=0.0084 via one-sided Wilcoxon rank sum test; FIG. 32). Moreover, this observation could be related to sample degradation and differences in sample processing, as SHINE samples went through additional freeze-thaw cycles and RT-qPCR was performed on extracted and DNase-treated samples.

TABLE 6
COVID-19 Negative Patient Sample Information.
Control samples were collected prior to
the start of the coronavirus pandemic, and therefore
are assumed to not contain SARS-CoV-2 RNA.

| Sample | | Sample | Viral | Adjusted viral | |
|---|---|---|---|---|---|
| name | Sample ID | type | Ct | quantity (cp/μl) | FIGS. |
| C1 | MA_MGH_00411 | NP in UTM | — | — | 26G and 26H |
| C2 | MA_MGH_00412 | NP in UTM | — | — | 26G and 26H |
| C3 | MA_MGH_00413 | NP in UTM | — | — | 26G and 26H |
| C4 | MA_MGH_00414 | NP in UTM | — | — | 26G and 26H |
| C5 | MA_MGH_00320 | NP in UTM | — | — | 26F, 26G and 26H |
| C6 | MA_MGH_00321 | NP in UTM | — | — | 26F, 26G and 26H |
| C7 | MA_MGH_00322 | NP in UTM | — | — | 26F, 26G and 26H |
| C8 | MA_MGH_00323 | NP in UTM | — | — | 26F, 26G and 26H |
| C9 | MA_MGH_00324 | NP in UTM | — | — | 26G and 26H |
| C10 | MA_MGH_00325 | NP in UTM | — | — | 26G and 26H |
| C11 | MA_MGH_00326 | NP in UTM | — | — | 26G and 26H |
| C12 | MA_MGH_00327 | NP in UTM | — | — | 26G and 26H |
| C13 | MA_MGH_00328 | NP in UTM | — | — | 26G and 26H |
| C14 | MA_MGH_00329 | NP in UTM | — | — | 26G and 26H |
| C15 | MA_MGH_00338 | NP in UTM | — | — | 26G and 26H |
| C16 | MA_MGH_00353 | NP in UTM | — | — | 26G and 26H |
| C17 | MA_MGH_00354 | NP in UTM | — | — | 26G and 26H |
| C18 | MA_MGH_00355 | NP in UTM | — | — | 26G and 26H |
| C19 | MA_MGH_00356 | NP in UTM | — | — | 26G and 26H |
| C20 | MA_MGH_00357 | NP in UTM | — | — | 26G and 26H |

Here, Applicants have described SHINE, a simple method for detecting viral RNA from unextracted patient samples with minimal equipment requirements. SHINE's simplicity matches that of the most streamlined nucleic acid diagnostics. Furthermore, the in-tube fluorescence readout and companion smartphone application lend themselves to scalable, high-throughput testing and automated interpretation of results. SHINE's simplicity and CRISPR-based programmability underscore its potential to address diagnostic needs during the COVID-19 pandemic, and in outbreaks to come.

Additional advances are still required for diagnostic testing to occur in virtually any location. Ideally, all steps would be performed at ambient temperature (without heat), in 15 minutes or less, using a colorimetric readout that does not require tube opening. Existing nucleic acid diagnostics, as far as is known, are not capable of meeting all these requirements simultaneously. Sample collection without UTM (i.e., “dry swabs”) combined with spin-column-free extraction buffers, and incorporation of solution-based, colorimetric readouts could address these limitations (28-31). Together, these advances could greatly enhance the accessibility of diagnostic testing and provide an essential tool in the fight against infectious diseases. By reducing personnel time, equipment, and assay time-to-results without sacrificing sensitivity or specificity, Applicants have taken steps towards the development of such a tool.

### Materials and Methods

Detailed information about reagents, including the commercial vendors and stock concentrations, is provided in Table 7.

TABLE 7
Reagents used in either the optimized one-step assay or in the optimization process.

| | | | Stock | | |
|---|---|---|---|---|---|
| Reagent | Reaction | Source | Concentration | | Notes |
| EDTA | HUDSON | Thermo Fisher | 0.5M | | |
| | | Scientific ™ | | | |
| TCEP—HCl | HUDSON | Thermo Fisher | 0.5M | | |
| | | Scientific ™ | | | |
| Universal Viral | HUDSON | BD | N/A | | |
| Transport | | | | | |
| Medium (UTM) | | | | | |
| Saliva, Pooled | HUDSON | Lee Biosolutions, | N/A | | |
| Human Donors | | Inc. | | | |
| RNase Inhibitor | HUDSON; | NEB ® | 40 | U/μL | Murine |
| | RPA; | | | | |
| | SHERLOCK | | | | |
| HiScribe ™ T7 | IVT | NEB ® | N/A | | |
| High Yield RNA | | | | | |
| Synthesis Kit | | | | | |
| RNAClean XP | IVT | Beckman Coulter, | N/A | | |
| | | Inc. | | | |
| RNase-Free | IVT | QIAGEN | | | |
| DNase I | | | | | |
| T7 Promoter | IVT | Integrated DNA | | | Table 8 for sequences |
| ssDNA Primer | | Technologies ™ | | | |
| Ambion ® Linear | PCR | Thermo Fisher | 5 | mg/mL | |
| Acrylamide | | Scientific ™ | | | |
| AMPure XP | PCR | Beckman Coulter, | N/A | | |
| | | Inc. | | | |
| Inter-amplicon | PCR | Integrated DNA | 5 | μM | Table 8 for sequences |
| Primer | | Technologies ™ | | | |
| MagMAX ™ | PCR | Thermo Fisher | N/A | | |
| mirVana ™ Total | | Scientific ™ | | | |
| RNA Isolation | | | | | |
| Kit | | | | | |
| PCR Primers | PCR | Integrated DNA | | | Table 8 for sequences |
| (forward, reverse) | | Technologies ™ | | | |
| Power SYBR ® | PCR | Thermo Fisher | N/A | | |
| Green Master | | Scientific ™ | | | |
| Mix | | | | | |
| TaqPath ™ 1-Step | PCR | Thermo Fisher | 4X | | |
| RT-qPCR Master | | Scientific ™ | | | |
| Mix | | | | | |
| TURBO ™ | PCR | Thermo Fisher | 2 | U/μL | |
| DNase | | Scientific ™ | | | |
| Magnesium | RPA | TwistDx ™ | 280 | mM | TwistAmp ® Basic Kit |
| Acetate (MgOAc) | | | | | |
| RevertAid | RPA | Thermo Fisher | 200 | U/μL | |
| Reverse | | Scientific ™ | | | |
| Transcriptase | | | | | |
| RNase H | RPA | NEB ® | 5 | U/μL | |
| RPA Pellets | RPA | TwistDx ™ | N/A | | TwistAmp ® Basic Kit |
| (lyophilized) | | | | | |
| RPA Primers | RPA | Integrated DNA | 5 μM of each primer | | Table 8 for sequences |
| (forward, reverse) | | Technologies ™ | | | |
| SuperScript IV | RPA | Thermo Fisher | 200 | U/μL | Invitrogen |
| Reverse | | Scientific ™ | | | |
| Transcriptase | | | | | |
| Synthetic DNA | RPA | Integrated DNA | 10¹⁰ | copies/μL | Table 8 for sequences |
| Target | | Technologies ™ | | | |
| Synthetic RNA | RPA | N/A | 10¹⁰ | copies/μL | generated via in vitro |
| Target | | | | | transcription of |
| | | | | | synthetic DNA target |
| Nuclease-free | RPA; | Thermo Fisher | N/A | | Invitrogen |
| Water | SHERLOCK | Scientific ™ | | | |
| Reaction Buffer | RPA; | N/A | 5X | | 0.1M HEPES pH 8.0, |
| (Optimized) | SHERLOCK | | | | 300 mM KCl, 25% |
| | | | | | PEG-8000 |
| Reaction Buffer | RPA; | N/A | 5X | | 0.1M HEPES pH 6.8, |
| (Original) | SHERLOCK | | | | 300 mM NaCl, 25% |
| | | | | | PEG-8000, 25 uM DTT |
| Cas13a crRNA | SHERLOCK | Integrated DNA | 2 | μM | Table 8 for sequences |
| | | Technologies ™ | | | |
| Cleavage Buffer | SHERLOCK | N/A | 10X | | 400 mM Tris pH 7.5, |
| (CB) | | | | | 10 mM DTT |
| FAM Cleavage | SHERLOCK | Integrated DNA | 16 | μM | Table 8 for sequences |
| Reporter | | Technologies ™ | | | |
| (Biotinylated) | | | | | |
| polyU FAM | SHERLOCK | Integrated DNA | 2 | μM | Table 8 for sequences |
| Cleavage | | Technologies ™ | | | |
| Reporter | | | | | |
| (Quencher) | | | | | |
| LwaCas13a | SHERLOCK | GenScript ® | 0.5 | mg/mL | Custom protein |
| protein | | | | | purification |
| | | | | | as described |
| | | | | | previously (23) |
| Magnesium | SHERLOCK | Thermo Fisher | 1M | | Invitrogen |
| Chloride (MgCl2) | | Scientific ™ | | | |
| HybriDetect | SHERLOCK | Milenia ® Biotec | | | |
| Assay Buffer | | | | | |
| HybriDetect 1 | SHERLOCK | Milenia ® Biotec | N/A | | |
| Lateral Flow | | | | | |
| Strips | | | | | |
| RNaseAlert ® | SHERLOCK | Thermo Fisher | 2 | μM | |
| Substrate v2 | | Scientific ™ | | | |
| rNTPs | SHERLOCK | NEB ® | 25 mM of each nucleotide | | |
| Storage Buffer | SHERLOCK | N/A | 1X | | 50 mM Tris pH 7.5, |
| (SB) | | | | | 600 mM NaCl, 5% |
| | | | | | glycerol, 2 mM DTT |
| T7 RNA | SHERLOCK | Lucigen ® | 50 | U/μL | NextGen ® |
| Polymerase | | | | | |

HUDSON = Heating Unextracted Diagnostic Samples to Obliterate Nucleases.
IVT = In Vitro Transcription.
PCR = Polymerase Chain Reaction.
RPA = Recombinase Polymerase Amplification.
SHERLOCK = Specific High Sensitivity Enzymatic Reporter Unlocking

Clinical samples and ethics statement. Clinical samples were acquired from clinical studies evaluated and approved by the Institutional Review Board/Ethics Review Committee of the Massachusetts General Hospital and Massachusetts Institute of Technology (MIT). The Office of Research Subject Projection at the Broad Institute of MIT and Harvard University approved use of samples for the work performed in this study.

Extracted sample preparation and RT-qPCR testing. Nasal swabs were collected and stored in universal viral transport medium (UTM; BD) and stored at −80° C. prior to nucleic acid extraction. Nucleic acid extraction was performed using MagMAX™ mirVana™ Total RNA isolation kit. The starting volume for the extraction was 250 μl and extracted nucleic acid was eluted into 60 μl of nuclease-free water. Extracted nucleic acid was then immediately Turbo DNase-treated (Thermo Fisher Scientific), purified twice with RNACleanXP SPRI beads (Beckman Coulter), and eluted into 15 μl of Ambion Linear Acrylamide (Thermo Fisher Scientific) water (0.8%).

Turbo DNase-treated extracted RNA was then tested for the presence of SARS-CoV-2 RNA using a lab-developed, probe-based RT-qPCR assay based on the N1 target of the CDC assay. RT-qPCR was performed on a 1:3 dilution of the extracted RNA using TaqPath™ 1-Step RT-qPCR Master Mix (Thermo Fisher Scientific) with the following forward and reverse primer sequences, respectively: GACCCCAAAATCAGCGAAAT (SEQ ID NO: 29), TCTGGTTACTGCCAGTTGAATCTG (SEQ ID NO: 30). The RT-PCR assay was run with a double-quenched FAM probe with the following sequence: 5′-FAM-ACCCCGCATTACGTTTGGTGGACC-BHQ1-3′ (SEQ ID NO: 31). RT-qPCR was run on a

QuantStudio 6 (Applied Biosystems) with RT at 48° C. for 30 min and 45 cycles with a denaturing step at 95° C. for 10 s followed by annealing and elongation steps at 60° C. for 45 s. The data were analyzed using the Standard Curve (SC) module of the Applied Biosystems Analysis Software.

SARS-CoV-2 assay design and synthetic template information. SARS-CoV-2-specific forward and reverse RPA primers and Cas13-crRNAs were designed as previously described (18). In short, the designs were algorithmically selected, targeting 100% of 20 published SARS-CoV-2 genomes, and predicted by a machine learning model to be highly active (Metsky et al. in prep). Moreover, the crRNA was selected for its high predicted specificity towards detection of SARS-CoV-2, versus related viruses, including other bat and mammalian coronaviruses and other human respiratory viruses (https://adapt. sabetilab . org/covi d-19/).

Synthetic DNA targets with appended upstream T7 promoter sequences (5′-GAAATTAATACGACTCACTATAGGG-3′ (SEQ ID NO: 32)) were ordered as double-stranded DNA (dsDNA) gene fragments from IDT, and were in vitro transcribed to generate synthetic RNA targets. In vitro transcription was conducted using the Hi Scribe T7 High Yield RNA Synthesis Kit (New England Biolabs (NEB)) as previously described (23). In brief, a T7 promoter ssDNA primer (5′-GAAATTAATACGACTCACTATAGGG-3′ (SEQ ID NO: 33)) was annealed to the dsDNA template and the template was transcribed at 37° C. overnight. Transcribed RNA was then treated with RNase-free DNase I (QIAGEN) to remove any remaining DNA according to the manufacturer's instructions. Finally, purification occurred using RNAClean SPRI XP beads at 2× transcript volumes in 37.5% isopropanol.

Sequence information for the synthetic targets, RPA primers, and Cas13-crRNA is listed in Table 8.

TABLE 8
Oligonucleotides used in this study.

| | | SARS-CoV-2 |
|---|---|---|
| Reagent | Sequence | Gene Location |
| Cas13a crRNA | CUCUUCUUCAGGUUGAAGAGCAGCAGAA | Orf1ab |
| | (SEQ ID NO: 34) | |
| PCR Primer (forward) | GACCCCAAAATCAGCGAAAT (SEQ ID | N1 |
| | NO: 35) | |
| PCR Primer (reverse) | TCTGGTTACTGCCAGTTGAATCTG | N1 |
| | (SEQ ID NO: 36) | |
| PCR Probe | /FAM/ACCCCGCATTACGTTTGGTGGAC | |
| | C/BHQ1(SEQ ID NO: 37) | |
| RPA Primer (forward) | CCAAGGTAAACCTTTGGAATTTGGTGCC | Orf1ab |
| | AC (SEQ ID NO: 38) | |
| RPA Primer (reverse) | ACTATCATCATCTAACCAATCTTCTTCT | Orf1ab |
| | TG (SEQ ID NO: 39) | |
| Synthetic DNA Target | GTGAGTTTAAATTGGCTTCACATATGTA | Orf1ab |
| | TTGTTCTTTCTACCCTCCAGATGAGGAT | |
| | GAAGAAGAAGGTGATTGTGAAGAAGAAG | |
| | AGTTTGAGCCATCAACTCAATATGAGTA | |
| | TGGTACTGAAGATGATTACCAAGGTAAA | |
| | CCTTTGGAATTTGGTGCCACTTCTGCTG | |
| | CTCTTCAACCTGAAGAAGAGCAAGAAGA | |
| | AGATTGGTTAGATGATGATAGTCAACAA | |
| | ACTGTTGGTCAACAAGACGGCAGTGAGG | |
| | ACAATCAGACAACTACTATTCAAACAAT | |
| | TGTTGAGGTTCAACCTCAATTAGAGATG | |
| | GAACTTACACCAGTTGTTCAGACTATTG | |
| | AAGTGAATAGTTTTAGTGGTTATTTAAA | |
| | ACTTACTGACAATGTATACATTAAAAAT | |
| | GCAGACATTGTGGAAGAAGCTAAAAAGG | |
| | TAAAACCAACAGTGGTTGTTAATGCAGC | |
| | CAATGTTTACCTTAAACATGGAGGAGG | |
| | (SEQ ID NO: 40) | |
| T7 Promoter ssDNA | GAAATTAATACGACTCACTATAGGG | dsDNA appended |
| Primer | (SEQ ID NO: 41) | upstream of target |
| FAM Cleavage | /56- | |
| Reporter (Biotinylated) | FAM/rUrUrUrUrUrUrUrUrUrUrUrUrUrU/3Bio/ | |
| | (SEQ ID NO: 42) | |
| polyU (i.e., 6U or 7U) | /56- | |
| FAM Cleavage | FAM/rUrUrUrUrUrU(rU)/3IABkFQ/ | |
| Reporter (Quencher) | (SEQ ID NO: 43) | |

Two-step SARS-CoV-2 assay. The two-step SHERLOCK assay was performed as previously described (18, 23, 25). Briefly, the assay was performed in two steps: (1) isothermal amplification via recombinase polymerase amplification (RPA) and (2) LwaCas13a-based detection using a single-stranded RNA (ssRNA) fluorescent reporter. For RPA, the TwistAmp Basic Kit (TwistDx) was used as previously described (i.e., with RPA forward and reverse primer concentrations of 400 nM and a magnesium acetate concentration of 14 mM) (25) with the following modifications: RevertAid reverse transcriptase (Thermo Fisher Scientific) and murine RNase inhibitor (NEB) were added at final concentrations of 4 U/μl each, and synthetic RNAs or viral seedstocks were added at known input concentrations making up10% of the total reaction volume. The RPA reaction was then incubated on the thermocycler for 20 minutes at 41° C. For the detection step, 1 μl of RPA product was added to 19 μl detection master mix. The detection master mix consisted of the following reagents (final concentrations in master mix listed), with magnesium chloride added last: 45 nM LwaCas13a protein resuspended in 1× storage buffer (SB: 50 mM Tris pH 7.5, 600 mM NaCl, 5% glycerol, and 2 mM dithiothreitol (DTT); such that the resuspended protein is at 473.7 nM), 22.5 nM crRNA, 125 nM RNaseAlert substrate v2 (Thermo Fisher Scientific), 1× cleavage buffer (CB; 400 mM Tris pH 7.5 and 10 mMDTT), 2 U/μlL murine RNase inhibitor (NEB), 1.5 U/μl NextGen T7 RNA polymerase (Lucigen), 1 mM of each rNTP (NEB), and 9 mM magnesium chloride. Reporter fluorescence kinetics were measured at 37° C. on a Biotek Cytation 5 plate reader using a monochromator (excitation: 485 nm, emission: 520 nm) every 5 minutes for up to 3 hours.

Single-step SARS-CoV-2 assay optimization. The starting point for optimization of the single-step SHERLOCK assay was generated by combining the essential reaction components of both the RPA and the detection steps in the two-step assay, described above (23, 25). Briefly, a master mix was created with final concentrations of 1× original reaction buffer (20 mM HEPES pH 6.8 with 60 mM NaCl, 5% PEG, and 5 μM DTT), 45 nM LwaCas13a protein resuspended in 1× SB (such that the resuspended protein is at 2.26 μM), 136 nM RNaseAlert substrate v2, 1 U/μl murine RNase inhibitor, 2 mM of each rNTP, 1 U/μl NextGen T7 RNA polymerase, 4 U/μl RevertAid reverse transcriptase, 0.32 μM forward and reverse RPA primers, and 22.5 nM crRNA. The TwistAmp Basic Kit lyophilized reaction components (1 lyophilized pellet per 102 μl final master mix volume) were resuspended using the master mix. After pellet resuspension, cofactors magnesium chloride and magnesium acetate were added at final concentrations of 5 mM and 17 mM, respectively, to complete the reaction.

Master mix and synthetic RNA template were mixed and aliquoted into a 384-well plate in triplicate, with 20 μl per replicate at a ratio of 19:1 master mix: sample. Fluorescence kinetics were measured at 37° C. on a Biotek Cytation 5 or Biotek Synergy H1 plate reader every 5 minutes for 3 hours, as described above. Applicants observed no significant difference in performance between the two plate reader models.

Optimization occurred iteratively, with a single reagent modified in each experiment. The reagent condition (e.g., concentration, vendor, or sequence) that produced the most optimal results—defined as either a lower limit of detection (LOD) or improved reaction kinetics (i.e., reaction saturates faster)—was incorporated into the protocol. Thus, the protocol used for every future reagent optimization consisted of the most optimal reagent conditions for every reagent tested previously.

For all optimization experiments, the modulated reaction component is described in the figures, associated captions, or associated legends. Across all experiments, the following components of the master mix were held constant: 45 nM LwaCas13a protein resuspended in 1× SB (such that the resuspended protein is at 2.26 μM), 1 U/μl murine RNase inhibitor, 2 mM of each rNTP, 1 U/μl NextGen T7 RNA polymerase, and 22.5 nM crRNA, and TwistDx RPA TwistAmp Basic Kit lyophilized reaction components (1 lyophilized pellet per 102 μl final master mix volume). In all experiments, the master mix components except for the magnesium cofactor(s) were used to resuspend the lyophilized reaction components, and the magnesium cofactor(s) were added last. All other experimental conditions, which differ among the experiments due to real-time optimization, are detailed in Table 7.

Single-step SARS-CoV-2 optimized reaction. The optimized reaction (see Supplementary Protocol for exemplary implementation) consists of a master mix with final concentrations of 1× optimized reaction buffer (20 mM HEPES pH 8.0 with 60 mM KCl and 5% PEG), 45 nM LwaCas13a protein resuspended in 1× SB (such that the resuspended protein is at 2.26 μM), 125 nM polyU [i.e., 6 uracils (6U) or 7 uracils (7U) in length, unless otherwise stated] FAM quenched reporter, 1 U/μl murine RNase inhibitor, 2 mM of each rNTP, 1 U/μl NextGen T7 RNA polymerase, 2 U/μl Invitrogen SuperScript IV (SSIV) reverse transcriptase (Thermo Fisher Scientific), 0.1 U/μl RNase H (NEB), 120 nM forward and reverse RPA primers, and 22.5 nM crRNA. Once the master mix is created, it is used to resuspend the TwistAmp Basic Kit lyophilized reaction components (1 lyophilized pellet per 102 μl final master mix volume). Finally, magnesium acetate is the sole magnesium cofactor, and is added at a final concentration of 14 mM to generate the final master mix.

The sample is added to the complete master mix at a ratio of 1:19 and the fluorescence kinetics are measured at 37° C. using a Biotek Cytation 5 or Biotek Synergy H1 plate reader as described above.

Visual detection via in-tube fluorescence and via lateral flow strip. Minor modifications were made to the single-step SARS-CoV-2 optimized reaction to visualize the readout via in-tube fluorescence or lateral flow strip.

For in-tube fluorescence, Applicants generated the single-step master mix as described above, except the 7U FAM quenched reporter was used at a concentration of 62.5 nM. The sample was added to the complete master mix at a ratio of 1:19. Samples were incubated at 37° C. and images were collected after 30, 45, 60, 90, 120 and 180 minutes of incubation, with image collection terminating once experimental results were clear. A dark reader transilluminator (DR196 model, Clare Chemical Research) was used to illuminate the tubes.

For lateral-flow readout, Applicants generated the single-step master mix as described above, except Applicants used a biotinylated FAM reporter at a final concentration of 1 μM rather than the quenched polyU FAM reporters. The sample was added to the complete master mix at a ratio of 1:19. After 1-2 hours of incubation at 37° C., the detection reaction was diluted 1:4 in Milenia HybriDetect Assay Buffer, and the Milenia HybriDetect 1 (TwistDx) lateral flow strip was added. Sample images were collected 5 min following incubation of the strip.

In-tube fluorescence reader mobile phone application. To enable smartphone-based fluorescence analysis, Applicants designed a companion application. Using the application, the user captures an image of a set of strip tubes illuminated by a transilluminator. The user then identifies regions of interest in the captured image by overlaying a set of pre-drawn boxes onto experimental and control tubes. Image and sample information is then transmitted to a server for analysis. Within each of the user-selected squares, the server models the bottom of each tube as a trapezoid and uses a convolutional kernel to determine the location of maximal signal within each tube, using data from the green channel of the RGB image. The server then identifies the background signal proximal to each tube and fits a Gaussian distribution around the background signal and around the in-tube signal. The difference between the mean pixel intensity of the background signal and the mean pixel intensity of the in-tube signal is then calculated as the background-subtracted fluorescence signal for each tube. To identify experimentally significant fluorescent signals, a score is computed for each experimental tube; this score is equal to the distance between the experimental and control background-subtracted fluorescence divided by the standard deviation of pixel intensities in the control signal. Finally, positive or negative samples are determined based on whether the score is above (positive, +) or below (negative, −) 1.5, a threshold identified empirically.

HUDSON protocols. HUDSON nuclease and viral inactivation were performed on viral seedstock as previously described with minor modifications to the temperatures and incubation times (25). In short, 100 mM TCEP (Thermo Fisher Scientific) and 1 mM EDTA (Thermo Fisher Scientific) were added to non-extracted viral seedstock and incubated for 20 minutes at 50° C., followed by 10 minutes at 95° C. The resulting product was then used as input into the two-step SHERLOCK assay.

The improved HUDSON nuclease and viral inactivation protocol was performed as previously described, with minor modifications (25). Briefly, 100 mM TCEP, 1 mM EDTA, and 0.8 U/μl murine RNase inhibitor were added to clinical samples in universal viral transport medium or human saliva (Lee Biosolutions). These samples were incubated for 5 minutes at 40° C., followed by 5 minutes at 70° C. (or 5 minutes at 95° C., if saliva). The resulting product was used in the single-step detection assay. In cases where synthetic RNA targets were used, rather than clinical samples (e.g., during reaction optimization), targets were added after the initial heating step (40° C. at 5 minutes). This is meant to recapitulate patient samples, as RNA release occurs after the initial heating step when the temperature is increased, and viral particles lyse.

For optimization of nuclease inactivation using HUDSON, only the initial heating step was performed. The products were then mixed 1:1 with 400 mM RNaseAlert substrate v2 in nuclease-free water and incubated at room temperature for 30 minutes before imaging on a transilluminator or measuring reporter fluorescence on a Biotek Synergy H1 [at room temperature using a monochromator (excitation: 485 nm, emission: 520 nm) every 5 minutes for up to 30 minutes]. The specific HUDSON protocol parameters modified are indicated in the figure captions.

SHINE. The SHINE assay consists of the optimized HUDSON protocol (described above) with the resulting product used as the sample input into Applicants' optimized, one-step SHERLOCK protocol (described above).

Data analysis and schematic generation. Conservation of SARS-CoV-2 sequences across Applicants' SHERLOCK assay was determined using publicly available genome sequences via GISAID. Analysis was based on an alignment of 5376 SARS-CoV-2 genomic sequences. Percent conservation was measured at each nucleotide within the RPA primer and Cas13-crRNA binding sites and represents the percentage of genomes that have the consensus base at each nucleotide position.

As described above, fluorescence values are reported as background-subtracted, with the fluorescence value collected before reaction progression (i.e., the latest time at which no change in fluorescence is observed, usually time 0, 5, or 10 minutes) subtracted from the final fluorescence value (3 hours, unless otherwise indicated).

Normalized fluorescence values are calculated using data aggregated from multiple experiments with at least one condition in common. The maximal fluorescence value across all experiments is set to 1, with fluorescence values from the same experiment set as ratios of the maximal fluorescence value. Common conditions across experiments are set to the same normalized value, and that value is propagated to determine the normalized values within an experiment.

The Wilcoxon rank sum test was conducted in MATLAB (MathWorks). Schematics shown in FIG. 20A and FIG. 26A were created using BioRender.com. All other schematics were generated in Adobe Illustrator (v24.1.2). Data panels were primarily generated via Prism 8 (GraphPad), except FIG. 26E which was generated using Python (version 3.7.2), seaborn (version 0.10.1) and matplotlib (version 3.2.1) (33, 34).

Discussion: SHINE's simplicity and compatibility with multiple sample types uniquely highlight its utility for widespread use. Previously developed CRISPR-based detection methods, such as SHERLOCK and DETECTR, are highly sensitive and specific, but assay results require nucleic acid extraction and multiple sample-manipulation steps (18, 19, 23, 25, 26, 29, 35). Integration of HUDSON and single-step SHERLOCK eliminates these limitations. HUDSON rapidly inactivated nucleases in both UTM (used for NP swabs) and saliva, illustrating its utility for downstream SARS-CoV-2 detection. Furthermore, less invasive sample collection is essential for routine or daily testing. Therefore, SHINE's compatibility with saliva samples is particularly important as saliva reliably contains SARS-CoV-2 RNA and its collection is less invasive (36-38). Additionally, the SHERLOCK component of SHINE could be re-designed for detection of other respiratory or saliva-secreted viruses, as well as new viral strains or mutations, underscoring the technology's future potential in shining light on the SARS-CoV-2 pandemic and other outbreaks.

Applicants have shown that SHINE can be used to readily detect SARS-CoV-2 RNA from NP samples without extraction, but further advancements are required to improve sensitivity across varying sample conditions. Notably, SHINE demonstrates perfect concordance with RT-qPCR in samples with Ct values below 22.5, only exhibiting stochasticity among the lower-titer samples. The association of RT-qPCR Ct value with SHINE's performance suggests that some of the observed non-concordance in test results may be due to assay sensitivity, possibly combined with degradation of sample material in the time or storage conditions between the two assays as the assays were not performed side-by-side. Improvements in isothermal amplification or HUDSON could also make these methods more comparable in performance.

Further optimization of visual readouts is required to deploy SHINE widely. Current colorimetric readout using paper-based, lateral flow strips has reduced speed compared to the in-tube fluorescent readout. Alternative visual readouts that do not require inserting a paper strip into each sample or measuring specific wavelengths of light would further decrease user and equipment needs (30, 31). These closed-tube visual readouts significantly decrease the risk of contamination across samples of amplified products. Ultimately, Applicants hope to lyophilize SHINE, simplifying distribution and allowing tests to be shelf-stable.

The following references relate to Example 2, and are incorporated herein by reference:

1. S. Kaplan, K. Thomas, Despite Promises, Testing Delays Leave Americans “Flying Blind” (2020), (available at nytimes.com/2020/04/06/health/coronavirus-testing-us.html).

2. Y. Bai, L. Yao, T. Wei, F. Tian, D.-Y. Jin, L. Chen, M. Wang, Presumed Asymptomatic Carrier Transmission of COVID-19. JAMA (2020), doi:10.1001/j ama.2020.2565.

3. C. Rothe, M. Schunk, P. Sothmann, G. Bretzel, G. Froeschl, C. Wallrauch, T. Zimmer, V. Thiel, C. Janke, W. Guggemos, M. Seilmaier, C. Drosten, P. Vollmar, K. Zwirglmaier, S. Zange, R. Wolfe!, M. Hoelscher, Transmission of 2019-nCoV Infection from an Asymptomatic Contact in Germany. N. Engl. J. Med. 382, 970-971 (2020).

4. C. Wang, P. W. Horby, F. G. Hayden, G. F. Gao, A novel coronavirus outbreak of global health concern. Lancet. 395, 470-473 (2020).

5. N. Zhu, D. Zhang, W. Wang, X. Li, B. Yang, J. Song, X. Zhao, B. Huang, W. Shi, R. Lu, P. Niu, F. Zhan, X. Ma, D. Wang, W. Xu, G. Wu, G. F. Gao, W. Tan, China Novel Coronavirus Investigating and Research Team, A Novel Coronavirus from Patients with Pneumonia in China, 2019. N. Engl. J. Med. 382, 727-733 (2020).

6. P. Zhou, X.-L. Yang, X.-G. Wang, B. Hu, L. Zhang, W. Zhang, H.-R. Si, Y. Zhu, B. Li, C.-L. Huang, H.-D. Chen, J. Chen, Y. Luo, H. Guo, R.-D. Jiang, M.-Q. Liu, Y. Chen, X.-R. Shen, X. Wang, X.-S. Zheng, K. Zhao, Q.-J. Chen, F. Deng, L.-L. Liu, B. Yan, F.-X. Zhan, Y.-Y. Wang, G.-F. Xiao, Z.-L. Shi, A pneumonia outbreak associated with a new coronavirus of probable bat origin. Nature. 579, 270-273 (2020).

7. World Health Organization (WHO). COVID-19 Situation Report, May 25, 2020, (available at who.int/docs/default-source/coronaviruse/situation-reports/20200524-covid-19-sitrep-125.pdf?sfvrsn=80e7d7f0_2).

8. U.S. Food and Drug Administration, Policy for COVID-19 Tests During the Public Health Emergency (Revised) (2020), (available at fda.gov/regulatory-information/search-fda-guidance-documents/policy-coronavirus-disease-2019-tests-during-public-health-emergency-revised).

9. T. Notomi, H. Okayama, H. Masubuchi, T. Yonekawa, K. Watanabe, N. Amino, T. Hase, Loop-mediated isothermal amplification of DNA. Nucleic Acids Res. 28, E63 (2000).

10. G.-S. Park, K. Ku, S.-H. Baek, S.-J. Kim, S. I. Kim, B.-T. Kim, J.-S. Maeng, Development of Reverse Transcription Loop-Mediated Isothermal Amplification Assays Targeting SARS-CoV-2. J. Mol. Diagn. (2020), doi:10.1016/j .j moldx.2020. 03 .006.

11. Y. H. Baek, J. Um, K. J. C. Antigua, J.-H. Park, Y. Kim, S. Oh, Y.-I. Kim, W.-S. Choi, S. G. Kim, J. H. Jeong, B. S. Chin, H. D. G. Nicolas, J.-Y. Ahn, K. S. Shin, Y. K. Choi, J.-S. Park, M.-S. Song, Development of a reverse transcription-loop-mediated isothermal amplification as a rapid early-detection method for novel SARS-CoV-2. Emerg. Microbes Infect., 1-31 (2020).

12. A. Niemz, T. M. Ferguson, D. S. Boyle, Point-of-care nucleic acid testing for infectious diseases. Trends in Biotechnology. 29 (2011), pp. 240-250.

13. Abott ID NOW™ COVID-19- Alere is now, (available at alere.com/en/home/product-details/id-now-covid-19.html).

14. O. Piepenburg, C. H. Williams, D. L. Stemple, N. A. Armes, DNA detection using recombination proteins. PLoS Biol. 4, e204 (2006).

15. H. Zaghloul, M. El-Shahat, Recombinase polymerase amplification as a promising tool in hepatitis C virus diagnosis. World J. Hepatol. 6, 916-922 (2014).

16. L. Yan, J. Zhou, Y. Zheng, A. S. Gamson, B. T. Roembke, S. Nakayama, H. O. Sintim, Isothermal amplified detection of DNA and RNA. Molecular BioSystems. 10 (2014), p. 970.

17. Zhang F, Abudayyeh O O, Gootenberg J S, A protocol for detection of COVID-19 using CRISPR diagnostics, (available at broadinstitute.org/files/publications/special/COVID-19%20detection%20(updated).pdf).

18. H. C. Metsky, C. A. Freije, T.-S. F. Kosoko-Thoroddsen, P. C. Sabeti, C. Myhrvold, CRISPR-based surveillance for COVID-19 using genomically-comprehensive machine learning design. bioRxiv (2020).

19. J. P. Broughton, X. Deng, G. Yu, C. L. Fasching, V. Servellita, J. Singh, X. Miao, J. A. Streithorst, A. Granados, A. Sotomayor-Gonzalez, K. Zorn, A. Gopez, E. Hsu, W. Gu, S. Miller, C.-Y. Pan, H. Guevara, D. A. Wadford, J. S. Chen, C. Y. Chiu, CRISPR-Cas12-based detection of SARS-CoV-2. Nat. Biotechnol. (2020), doi:10.1038/s41587-020-0513-4.

20. L. Guo, X. Sun, X. Wang, C. Liang, H. Jiang, Q. Gao, M. Dai, B. Qu, S. Fang, Y. Mao, Y. Chen, G. Feng, Q. Gu, L. Wang, R. R. Wang, Q. Zhou, W. Li, SARS-CoV-2 detection with CRISPR diagnostics, medRxiv, doi:10.1101/2020.04.10.023358.

21. J. N. Rauch, E. Valois, S. C. Solley, F. Braig, R. S. Lach, N. J. Baxter, K. S. Kosik, C. Arias, D. Acosta-Alvear, M. Z. Wilson, A Scalable, Easy-to-Deploy, Protocol for Cas13-B ased Detection of SARS-CoV-2 Genetic Material, medRxiv, doi:10.1101/2020.04.20.052159.

22. U.S. Food and Drug Administration, Sherlock CRISPR SARS-CoV-2 Kit, (available at fda.gov/media/137747/download).

23. J. S. Gootenberg, O. O. Abudayyeh, J. W. Lee, P. Essletzbichler, A. J. Dy, J. Joung, V. Verdine, N. Donghia, N. M. Daringer, C. A. Freije, C. Myhrvold, R. P. Bhattacharyya, J. Livny, A. Regev, E. V. Koonin, D. T. Hung, P. C. Sabeti, J. J. Collins, F. Zhang, Nucleic acid detection with CRISPR-Cas13a/C2c2. Science. 356, 438-442 (2017).

24. O. O. Abudayyeh, J. S. Gootenberg, S. Konermann, J. Joung, I. M. Slaymaker, D. B. T. Cox, S. Shmakov, K. S. Makarova, E. Semenova, L. Minakhin, K. Severinov, A. Regev, E. S. Lander, E. V. Koonin, F. Zhang, C2c2 is a single-component programmable RNA-guided RNA-targeting CRISPR effector. Science. 353, aaf5573 (2016).

25. C. Myhrvold, C. A. Freije, J. S. Gootenberg, O. O. Abudayyeh, H. C. Metsky, A. F. Durbin, M. J. Kellner, A. L. Tan, L. M. Paul, L. A. Parham, K. F. Garcia, K. G. Barnes, B. Chak, A. Mondini, M. L. Nogueira, S. Isern, S. F. Michael, I. Lorenzana, N. L. Yozwiak, B. L. MacInnis, I. Bosch, L. Gehrke, F. Zhang, P. C. Sabeti, Field-deployable viral diagnostics using CRISPR-Cas13. Science. 360, 444-448 (2018).

26. J. S. Gootenberg, O. O. Abudayyeh, M. J. Kellner, J. Joung, J. J. Collins, F. Zhang, Multiplexed and portable nucleic acid detection platform with Cas13, Cas12a, and Csm6. Science. 360, 439-444 (2018).

27. A. East-Seletsky, M. R. O'Connell, D. Burstein, G. J. Knott, J. A. Doudna, RNA Targeting by Functionally Orthogonal Type VI-A CRISPR-Cas Enzymes. Mol. Cell. 66, 373-383.e3 (2017).

28. S. Srivatsan, P. D. Han, K. van Raay, C. R. Wolf, D. J. McCulloch, A. E. Kim, E. Brandstetter, B. Martin, J. Gehring, W. Chen, S. Kosuri, E. Q. Konnick, C. M. Lockwood, M. J. Rieder, D. A. Nickerson, H. Y. Chu, J. Shendure, L. M. Starita, Seattle Flu Study Investigators, Preliminary support for a “dry swab, extraction free” protocol for SARS-CoV-2 testing via RT-qPCR, bioRxiv, doi:10.1101/2020.04.22.056283.

29. J. Joung, A. Ladha, M. Saito, M. Segel, R. Bruneau, M.-L. W. Huang, N.-G. Kim, X. Yu, J. Li, B. D. Walker, A. L. Greninger, K. R. Jerome, J. S. Gootenberg, O. O. Abudayyeh, F. Zhang, Point-of-care testing for COVID-19 using SHERLOCK diagnostics. medRxiv, doi:10.1101/2020.05.04.20091231.

30. A. E. Calvert, B. J. Biggerstaff, N. A. Tanner, M. Lauterbach, R. S. Lanciotti, Rapid colorimetric detection of Zika virus from serum and urine specimens by reverse transcription loop-mediated isothermal amplification (RT-LAMP). PLoS One. 12, e0185340 (2017).

31. B. A. Rabe, C. Cepko, SARS-CoV-2 Detection Using an Isothermal Amplification Reaction and a Rapid, Inexpensive Protocol for Sample Inactivation and Purification, medRxiv, doi:10.1101/2020.04.23.20076877.

32. D. Kim, J.-Y. Lee, J.-S. Yang, J. W. Kim, V. N. Kim, H. Chang, The Architecture of SARS-CoV-2 Transcriptome. Cell. 181, 914-921.e10 (2020).

33. M. Waskom, O. Botvinnik, D. O'Kane, P. Hobson, S. Lukauskas, D. C. Gemperline, T. Augspurger, Y. Halchenko, J. B. Cole, J. Warmenhoven, J. de Ruiter, C. Pye, S. Hoyer, J. Vanderplas, S. Villalba, G. Kunter, E. Quintero, P. Bachant, M. Martin, K. Meyer, A. Miles, Y. Ram, T. Yarkoni, M. L. Williams, C. Evans, C. Fitzgerald, Brian, C. Fonnesbeck, A. Lee, A. Qalieh, mwaskom/seaborn: v0.8.1 (September 2017) (2017), doi:10.5281/zenodo.883859.

34. J. D. Hunter, Matplotlib: A 2D Graphics Environment. Comput. Sci. Eng. 9, 90-95 (2007).

35. J. S. Chen, E. Ma, L. B. Harrington, M. Da Costa, X. Tian, J. M. Palefsky, J. A. Doudna, CRISPR-Cas12a target binding unleashes indiscriminate single-stranded DNase activity. Science. 360, 436-439 (2018).

36. E. Williams, K. Bond, B. Zhang, M. Putland, D. A. Williamson, Saliva as a non-invasive specimen for detection of SARS-CoV-2. J. Clin. Microbiol. (2020), doi:10.1128/JCM.00776-20.

37. K. K.-W. To, O. T.-Y. Tsang, W.-S. Leung, A. R. Tam, T.-C. Wu, D. C. Lung, C. C.-Y. Yip, J.-P. Cai, J. M.-C. Chan, T. S.-H. Chik, D. P.-L. Lau, C. Y.-C. Choi, L.-L. Chen, W.-M. Chan, K.-H. Chan, J. D. Ip, A. C.-K. Ng, R. W.-S. Poon, C.-T. Luo, V. C.-C. Cheng, J. F.-W. Chan, I. F.-N. Hung, Z. Chen, H. Chen, K.-Y. Yuen, Temporal profiles of viral load in posterior oropharyngeal saliva samples and serum antibody responses during infection by SARS-CoV-2: an observational cohort study. Lancet Infect. Dis. 20, 565-574 (2020).

38. A. L. Wyllie, J. Fournier, A. Casanovas-Massana, M. Campbell, M. Tokuyama, P. Vijayakumar, B. Geng, M. Catherine Muenker, A. J. Moore, C. B. F. Vogels, M. E. Petrone, I. M. Ott, P. Lu, A. Lu-Culligan, J. Klein, A. Venkataraman, R. Earnest, M. Simonov, R. Datta, R. Handoko, N. Naushad, L. R. Sewanan, J. Valdez, E. B. White, S. Lapidus, C. C. Kalinich, X. Jiang, D. J. Kim, E. Kudo, M. Linehan, T. Mao, M. Moriyama, J. E. Oh, A. Park, J. Silva, E. Song, T. Takahashi, M. Taura, O.-E. Weizman, P. Wong, Y. Yang, S. Bermejo, C. Odio, S. B. Omer, C. S. Dela Cruz, S. Farhadian, R. A. Martinello, A. Iwasaki, N. D. Grubaugh, A. I. Ko, Saliva is more sensitive for SARS-CoV-2 detection in COVID-19 patients than nasopharyngeal swabs, medRxiv, doi:10.1101/2020.04.16.20067835.

### Example 3

### Rapid Design of Maximally Active and Specific Viral Diagnostics Abstract

Principled methods to design viral diagnostics could rapidly render highly effective assays using the latest genomic diversity, including from novel species or strains. Here Applicants give new algorithms and machine learning models, linked in a system named ADAPT, that design assays to use with molecular detection methods for which one can quantify activity between a probe and target sequence. ADAPT solves for probe sets with maximal detection activity in expectation across a taxon's full genomic diversity. Applicants formulate this goal as a non-monotone submodular maximization problem and apply an algorithm that yields provably good designs. To integrate realistic activities, Applicants focus on CRISPR-Cas13a detection, and develop and test a library of 19,000 guide-target pairs on which Applicants train a convolutional neural network that outperforms other models Applicants test at predicting Cas13a detection activity. Applicants develop, and incorporate into ADAPT, a data structure and exact query algorithm to enforce high specificity across viral taxa, down to the lineage level, while being perfectly tolerant of wobble base pairing present during RNA detection. ADAPT connects directly with publicly available sequence databases to provide designs through an end-to-end system. Applicants used ADAPT to design maximally active and species-specific Cas13a detection assays for the 1,933 vertebrate-infecting viral species, comprehensively accounting for their 145,000 near-complete or complete genomes, in under 24 hours for all but 3 species. Experimental applications. The results show that ADAPT can continually design highly active and specific diagnostic assays, thereby expanding Applicants' toolkit for viral surveillance.

### Main Text

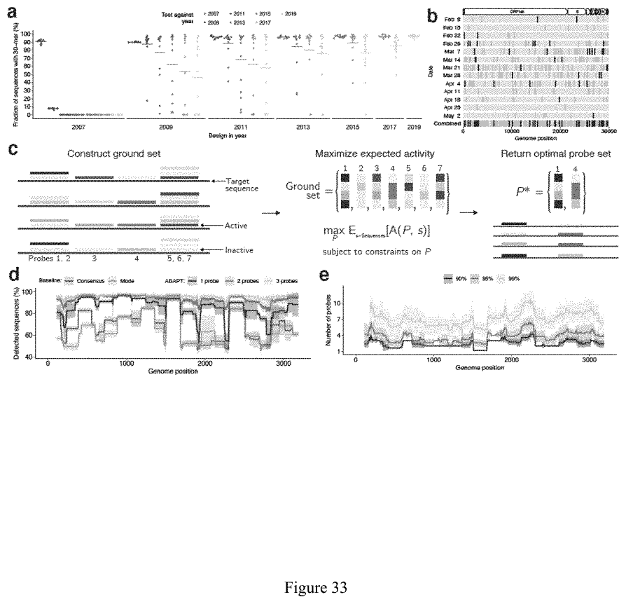

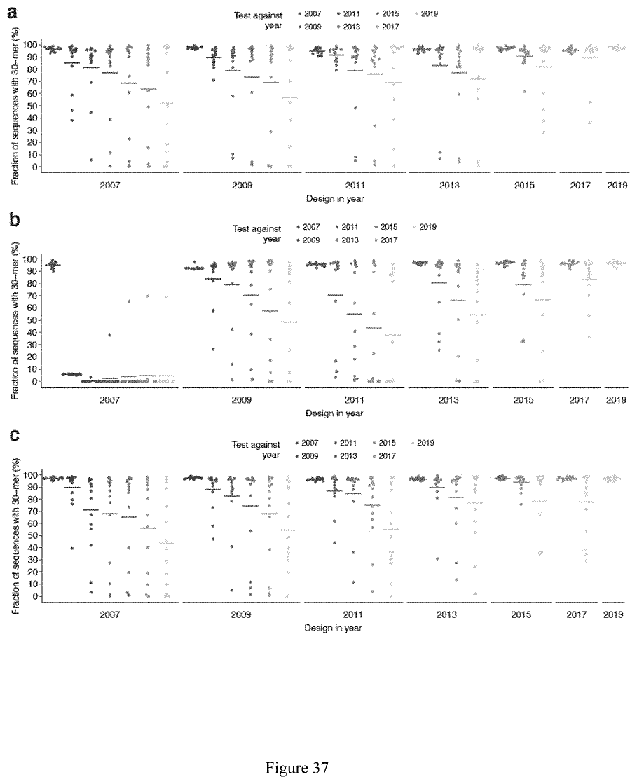

Viruses exhibit extensive and ever-changing genomic diversity 1, and new or understudied strains can quickly lead to epidemics. Sequencing of viral genomes illuminates this diversity and is growing exponentially. Even among only the human-infecting viral species, the number of sequenced genomes and their diversity is climbing steadily (FIG. 11). For the SARS-CoV-2 subspecies, databases provided 10,000 sequenced genomes within just 3 months6. This vast data underlie the development of tools for surveillance, diagnostics, vaccines, and therapies. Inadequate consideration to genomic diversity impacts surveillance and diagnostics in practice. Studies find significantly reduced sensitivities of commercially-available influenza A virus (IAV) RT-qPCR tests owing to rapid evolution, with false-negative rates commonly over 10% and, for some circulating strains, nearly 100%7-9. The same issue bedevils nucleic acid assays for many other viruses. Yet it is not trivial for assays to be genomically-comprehensive: for example, if Applicants were to select a collection of the most conserved 30-mers from which to develop an assay for IAV subtyping, the fraction of genomes that contains many of them falls quickly year-to-year, especially profoundly during the 2009 H1N1 pandemic, owing to antigenic drift and segment reassortment (FIG. 33a, FIG. 37). Within an epidemic, mutations quickly accumulate on top of genome sequences made available early on (FIG. 33b), which may degrade diagnostic sensitivity. Beyond a diagnostic's immediate use, fully accounting for current sequence variation is helpful for the long-term because current allele frequency is the best predictor of fixation under neutral evolution and even sometimes under strong selective pressure.

There are many technologies for nucleic acid diagnostics united by their dependence on sequence data. Alongside RT-qPCR and other amplification techniques, newly developed technologies with enzyme-activated signal enable detection integrated into low-cost platforms with little equipment. Recent examples include CRISPR-based systems, such as SHERLOCK16-18 or DETECTR19, that couple an RNA-activated RNase (Cas13) or DNA-activated DNase (Cas12) with a reporter; enzyme activation, owing to the presence of a target sequence, leads to a readout. Another is toehold switch RNA sensors20, 21, in which a sensor's binding to a target causes detectable protein translation. These assays incorporate probes to detect the target, such as RNA guides for CRISPR-based diagnostics. Some probes better reflect genomic diversity and some yield better sensitivity owing to factors like sequence composition and complementarity to the target.

Despite a growing molecular diagnostic toolkit, Applicants lack computational methods to rapidly translate vast sequence data into effective assay designs. The programmability of such tools18, 22 offers a chance to quickly produce assays for new species or strains, update assays to reflect continual evolution, and multiplex across many taxa. While there is a rich history of methods for designing PCR-based diagnostics23-33, several shortcomings limit their general use. Many do not account for sequence variation or extract long, highly conserved regions to mine that must be an exact match to all genomes within a species and not a match to other species; this does not suffice for most viruses. Also, all PCR design methods are tailored to detecting DNA and thus avoid RNA challenges (e.g., G-U pairing), use well-studied hybridization models, and usually binarize decisions about detection. Recent detection technologies, in which the signal is a product of enzyme activation, likely require more sophisticated models of activity. Ideally, Applicants want design methods that are compatible with high sequence heterogeneity and models that make quantitative predictions of detection activity, and that can be integrated in a system to apply rapidly and continually at scale.

Here Applicants develop ADAPT (Activity-informed Design with All-inclusive Patrolling of Targets), a system for the end-to-end design of maximally active and specific viral nucleic acid diagnostic assays. There are four problems Applicants address. First, Applicants formulate and implement an approach to identify probes with maximal activity across extensive genomic diversity. Second, Applicants construct a realistic activity function by developing a dataset and training models to predict CRISPR-Cas13a activity. Then, Applicants develop an exact query algorithm to enforce specificity across viral taxa even at the lineage-level. Finally, Applicants integrate Applicants' methods within a branch and bound search over the genome to identify optimal assays, and connect directly with genome databases to use their sequences. The output of ADAPT are assay options, ranked according to an objective function. ADAPT is available under the MIT license at https://github.com/broadinstitute/adapt.

Applicants applied ADAPT to design maximally active, species-specific Cas13a detection assays for the 1,933 species known to infect vertebrates. Applicants experimentally benchmarked the activity and specificity of several designs. Results, 1-2 sentences

### Designing Genomically-Comprehensive Probe Sets

At the core of ADAPT, Applicants design a probe set that best detects the full scope of known sequence variation in a genomic region. For this task, imagine Applicants have all known sequences within this region, represented by S, and a function that quantifies detection activity between a probe and targeted sequence such that higher activities are more desirable. Later, Applicants will describe how to search for appropriate regions and develop the activity function. ADAPT constructs a ground set of possible probes by finding representative subsequences across S using locality-sensitive hashing. Then, Applicants establish the following goal: find the probe set P, a subset of the ground set, that maximizes a function of the expected activity between P and sequences in S (FIG. 33c). In this objective function, the expectation is taken over the sequences in S and the probability for each reflects a prior on applying the probes toward a particular sequence; for example, it could weight highly sequences that are recent or from a particular geographic region, though in practice Applicants usually weight them uniformly. Larger numbers of probes might require more detection reactions or interfere if multiplexed in a single reaction, and thus Applicants impose a penalty in the objective function on the number of probes (details in Supplementary Note la). Applicants also impose a hard constraint on the number of probes.

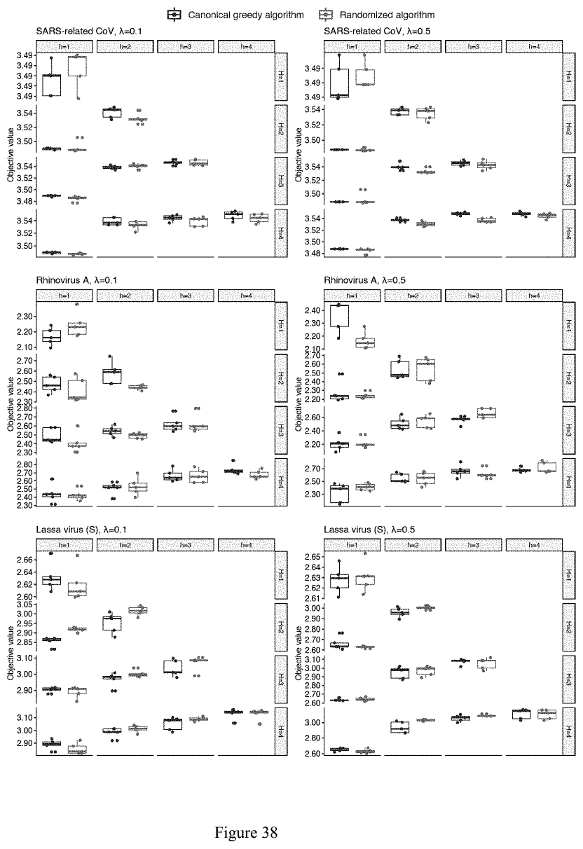

Having formulated an objective function, Applicants then sought to develop an approach to solve it. Applicants' objective function is non-monotone submodular and, under reasonable activity constraints on probes permitted in the ground set, it is non-negative. ADAPT implements a fast randomized combinatorial algorithm from ref. 35 for maximizing a non-negative and non-monotone submodular function under a cardinality constraint, which provides a probe set whose objective value is within a factor 1/e of the optimal. Supplementary Note la contains the proofs and algorithmic details. Applicants also evaluated the canonical greedy algorithm36 for constrained submodular maximization, finding that it returns similar results in practice (FIG. 38), though does not offer provable guarantees in this case because of its monotonicity requirement.

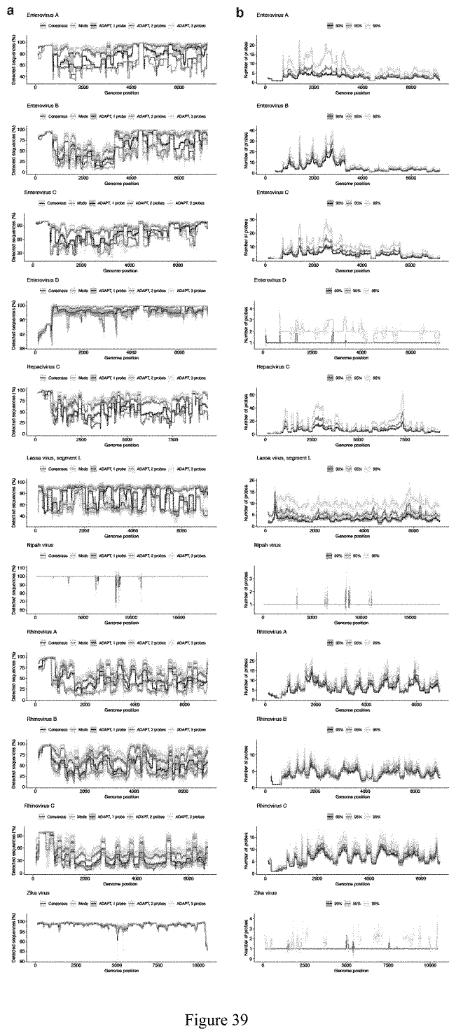

This approach yields probes with greater comprehensiveness across sequence diversity than do two less sophisticated strategies. For benchmarking, Applicants chose an activity function that equals 1 if a probe is within 1 mismatch of a target (detected), counting G-U wobble pairs as matching, and equals 0 otherwise (not detected); this function has the interpretable property that expected activity is equivalent to the fraction of genomes that are detected. Simple strategies constructing probes using the consensus or the most common sequence in a region fail to capture much of the sequence diversity for Lassa virus and other highly diverse viruses (FIG. 33d, FIG. 39a). On the other hand, maximizing expected activity detects more sequenceseven when constrained to a single probe—across the genome, and the amount detected further increases as Applicants permit more probes (FIG. 33d, FIG. 39a). Similarly, using a related objective function that minimizes the number of probes subject to constraints on activity and comprehensiveness (Supplementary Note lb), Applicants find that, throughout the genome, it is possible to reach near-complete comprehensiveness with few probes (FIG. 33e, FIG. 39b). On species with less extensive diversity, such as Zika virus (FIG. 39a), simple strategies perform well, indicating that ADAPT's more involved approach is not always necessary. Nevertheless, having options to target many different regions of a genome with high comprehensiveness, including for diverse taxa, enables ADAPT to incorporate true predictions of activity and enforce taxon-specificity, each of which will constrain the design options.

### Predicting Detection Activity of CRISPR-Cas13a

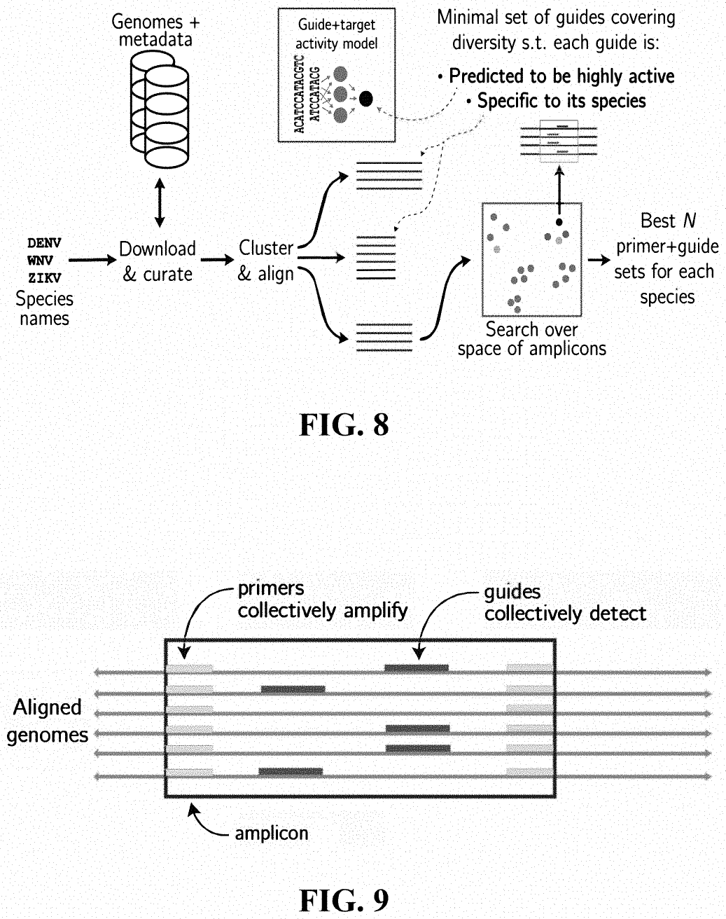

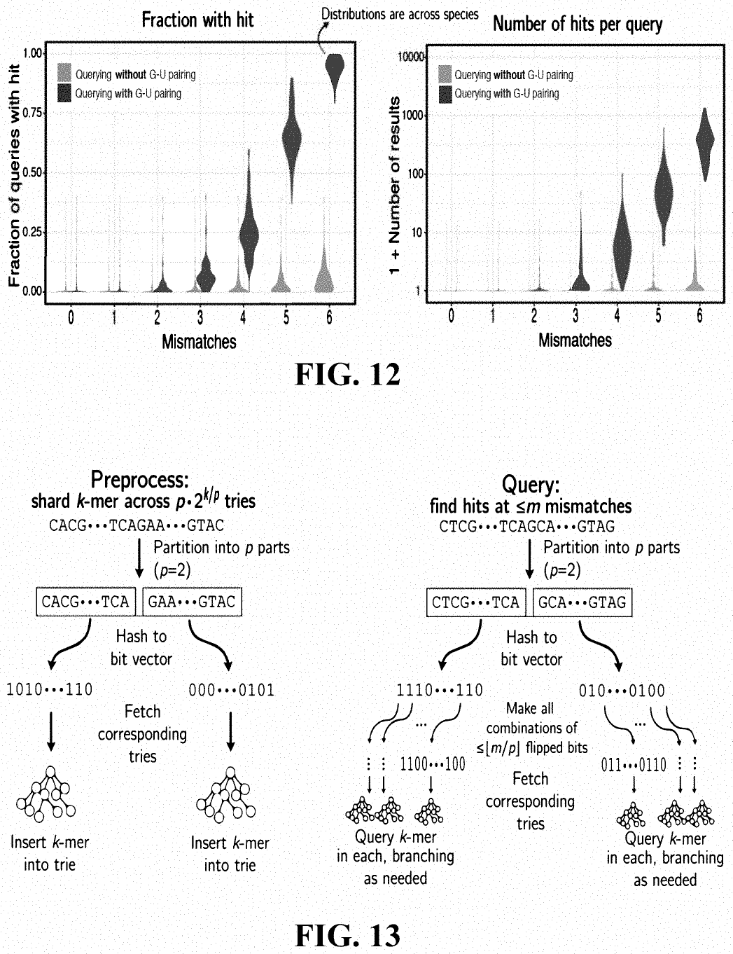

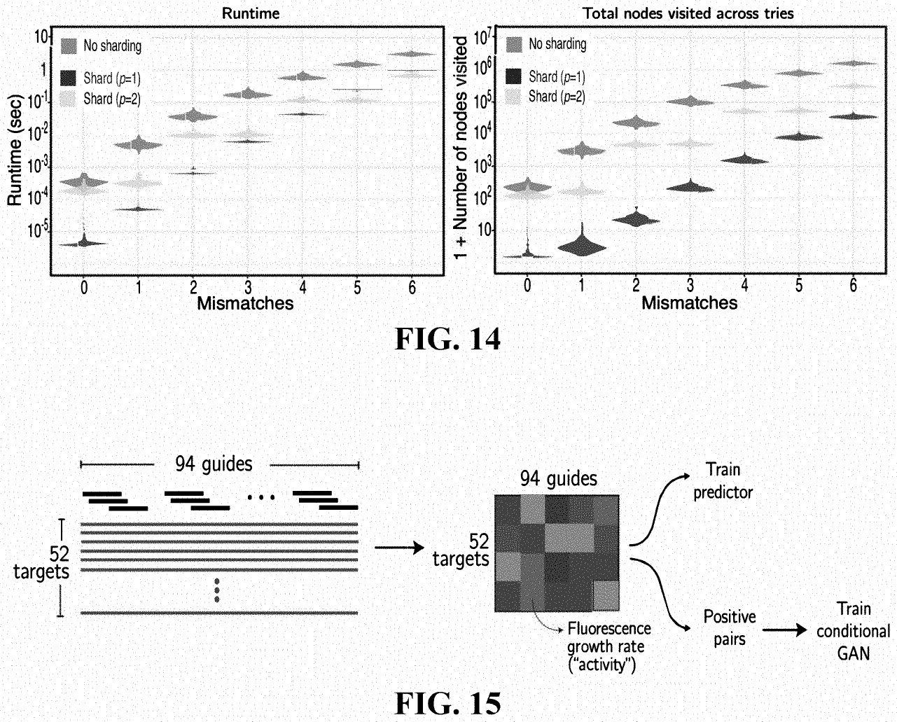

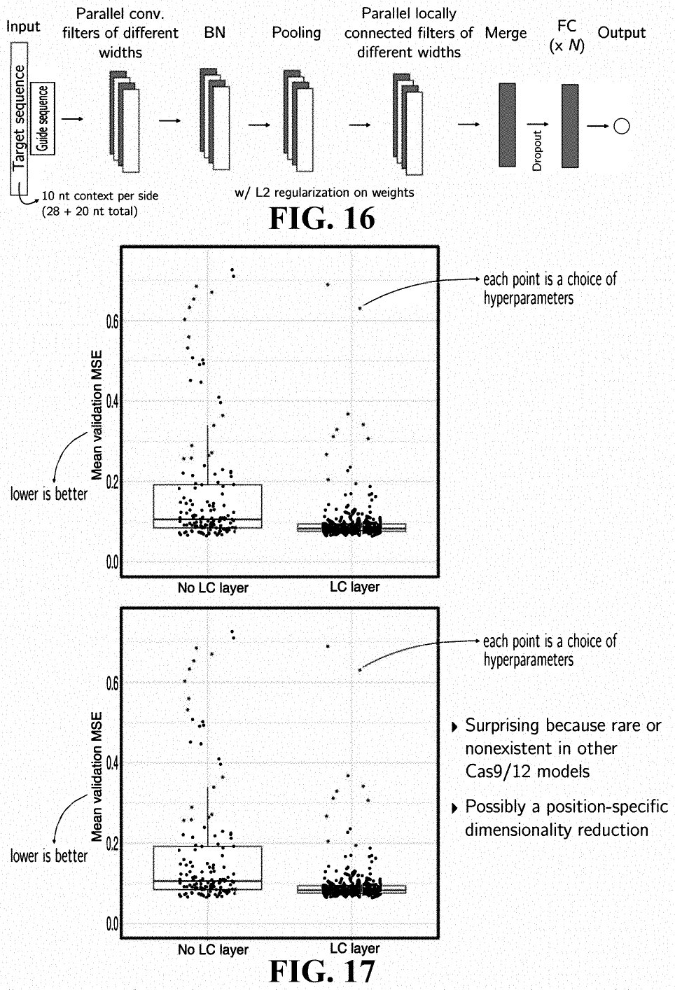

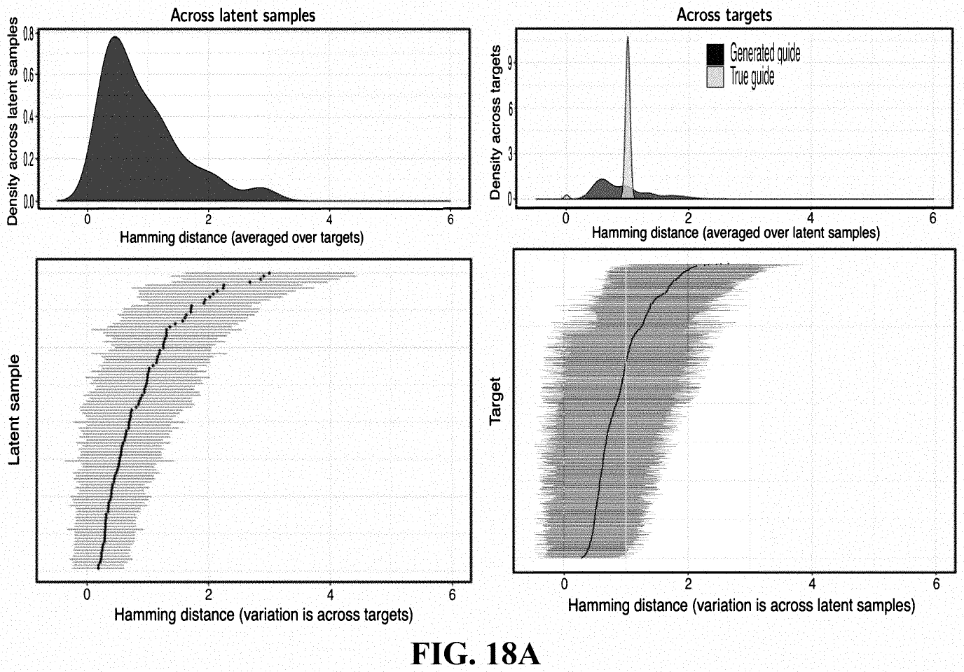

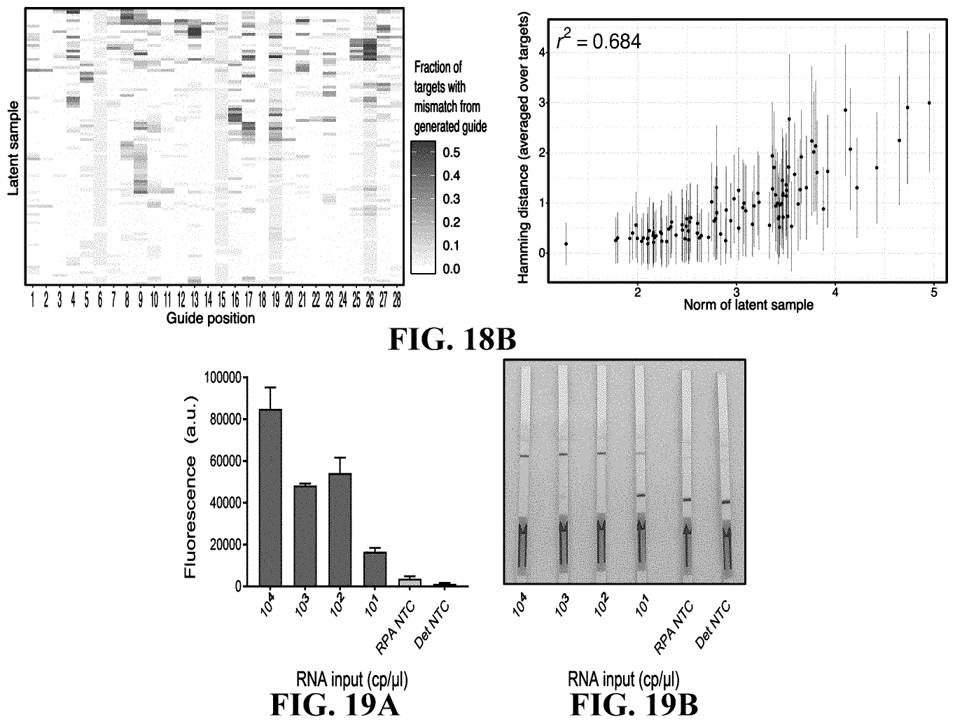

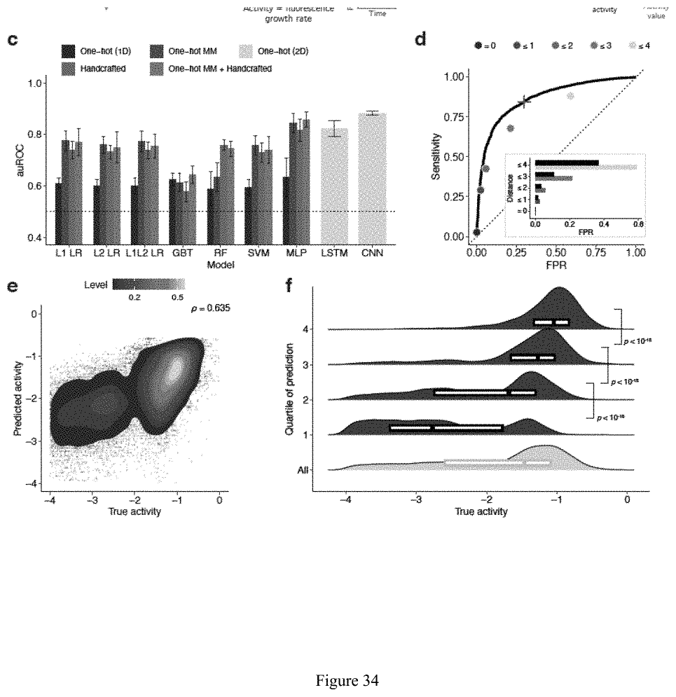

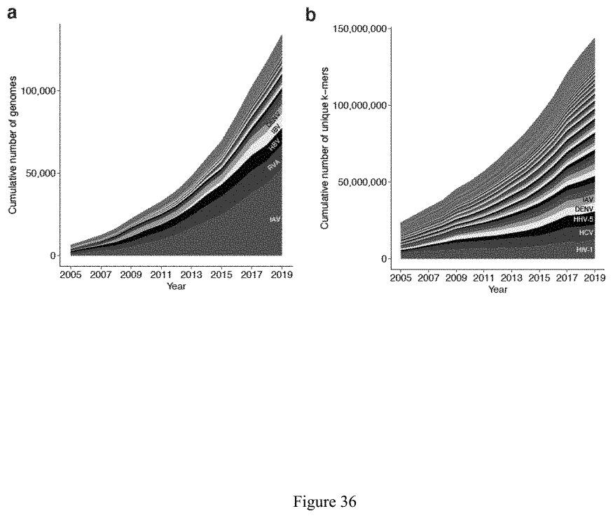

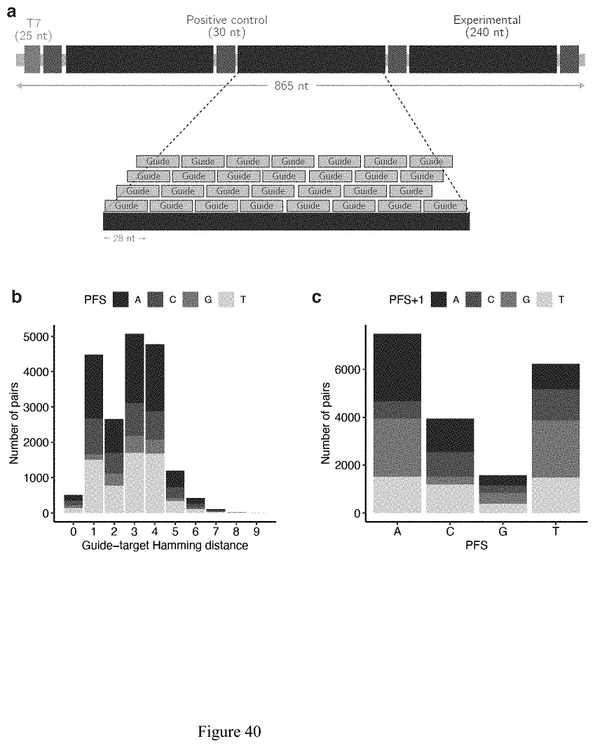

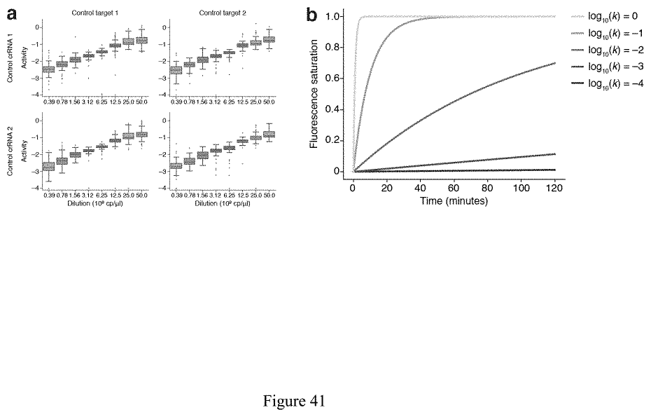

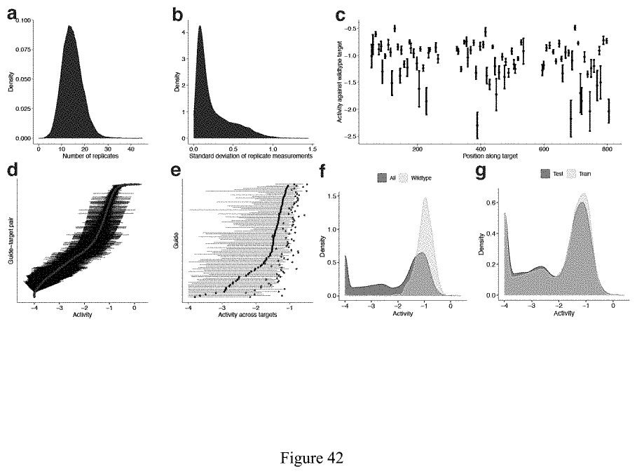

To make Applicants' approach practical, Applicants developed an activity function informed by detection reaction kinetics. Applicants focus on CRISPR-Cas13a technologies16, 17, in which Cas13a enzymes use probes (here, guide RNAs) to detect a target sequence and then exhibit collateral RNase activity that cleaves fluorescent reporters, leading to a readout. Prior studies have established design principles for guide activity—such as the importance of a protospacer flanking site (PFS), RNA secondary structure, and a mismatch-sensitive “seed” region37-39—but have not modeled the activity of Cas13a or other CRISPR enzymes for detection applications. Applicants designed a library of 19,209 unique LwaCas13a guide-target pairs (FIG. 9a, FIG. 40a) having sequence composition representative of viral genomes (Methods), an average of 2.9 mismatches between each guide and target (FIG. 40b), and a representation of the different PFS alleles (FIG. 40c). Applicants model remaining reporter presence during the reaction with an exponential decay and use the negative of the decay to model fluorescence over time (FIG. 34a; Methods). The growth rate of the fluorescence conveys meaningful information about the reaction kinetics because it correlates closely with target concentration and is a metric of the detection performance of a guide-target pair (FIG. 41a,b); Applicants' activity equals the logarithm of this rate (Methods). Applicants used CARMEN-Cas1340, a droplet-based detection platform, to perform the detection reactions and measure fluorescence roughly every 20 minutes, from which Applicants calculate activity for each pair. The data include, on average, about 15 replicate activity measurements per pair (FIG. 42a-d) and exhibit activity differences, spanning several orders of magnitude in fluorescence growth rate, between different guide sequences and across the different targets each guide may detect (FIG. 7e-42g).

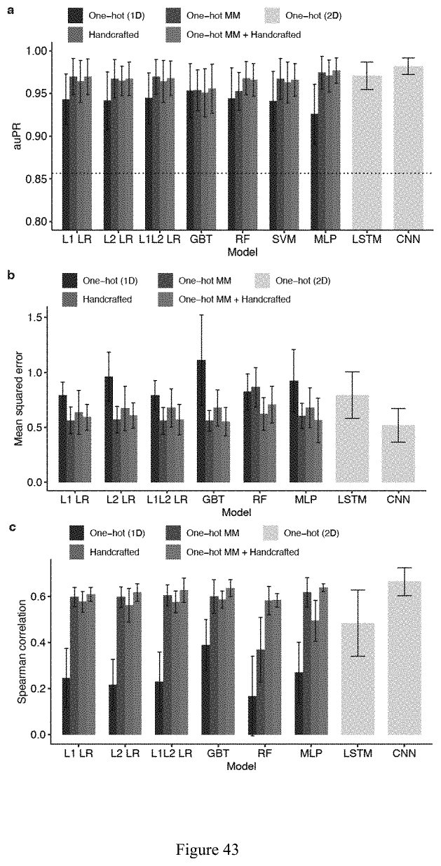

Using Applicants' dataset, Applicants developed a model to predict Cas13a activity of a guide-target pair. Applicants use a two-step hurdle model: classifying a pair as inactive or active, and then regressing activity for active pairs (FIG. 34b, FIG. 41b; 86.8% of the full dataset is labeled active). For classifying guide-target pairs, Applicants first performed nested cross-validation to evaluate Applicants' hyperparameter search and to compare 9 models potentially suited to this task using different inputs, including one-hot encodings and handcrafted features, and found that a convolutional neural network (CNN) outperforms the other models (FIG. 34c, FIG. 43a). Applicants found the same result for regression on active pairs, albeit having a less noticeable improvement over simpler models than with classification (FIGS. 43b,c). CNNs have also performed well for regressing Cas12a guide RNA editing activity on perfectly matching target sequences41, likely because the convolutional layers help to detect motifs in the sequences; here, such layers may also detect types of mismatches. In all model training and testing, Applicants accounted for measurement error (Methods).

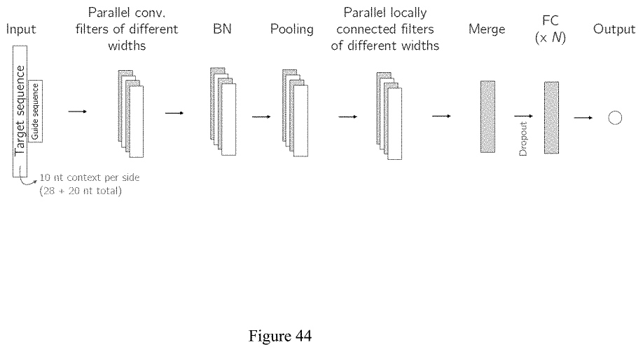

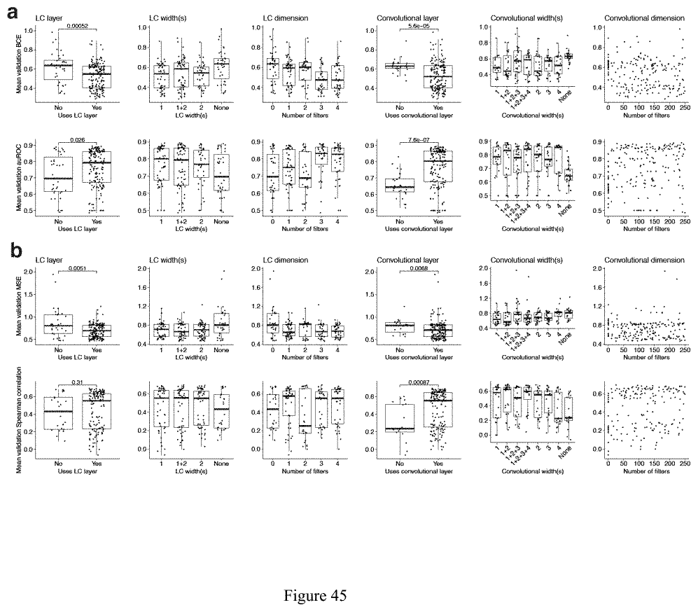

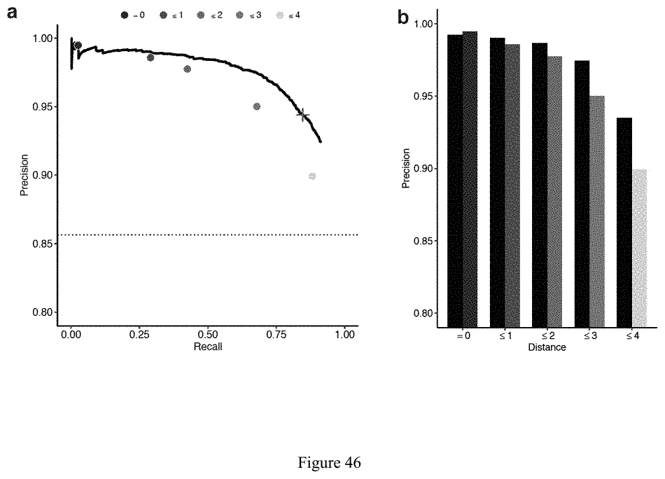

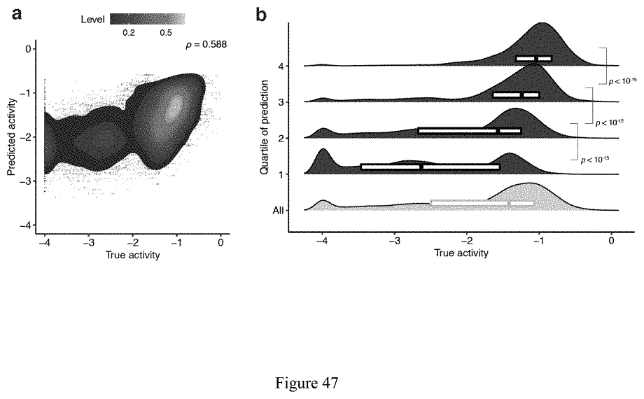

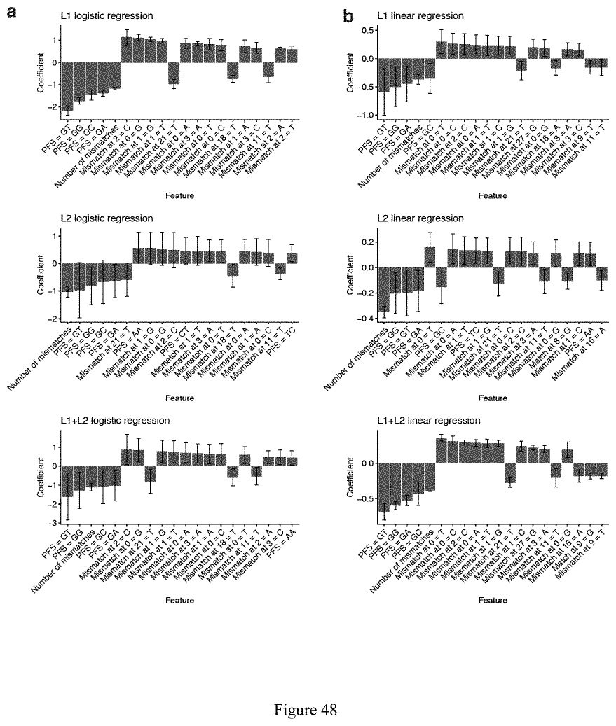

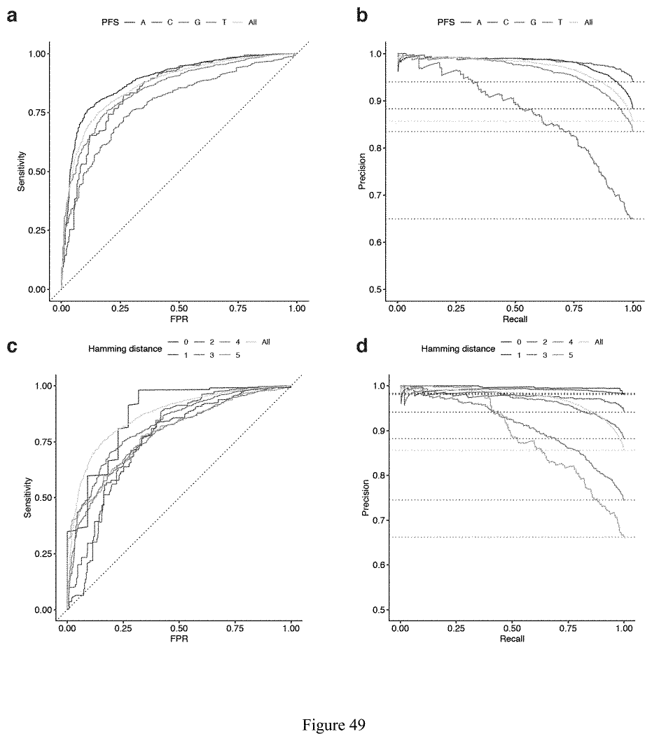

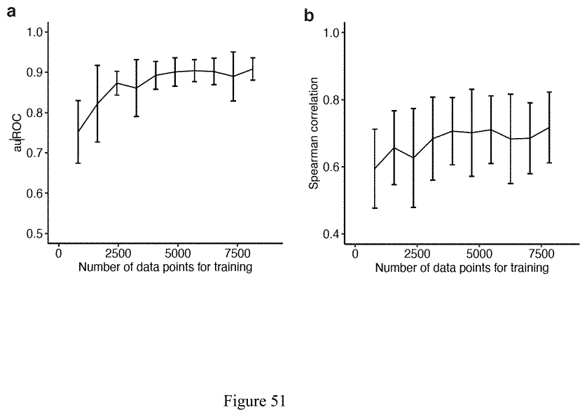

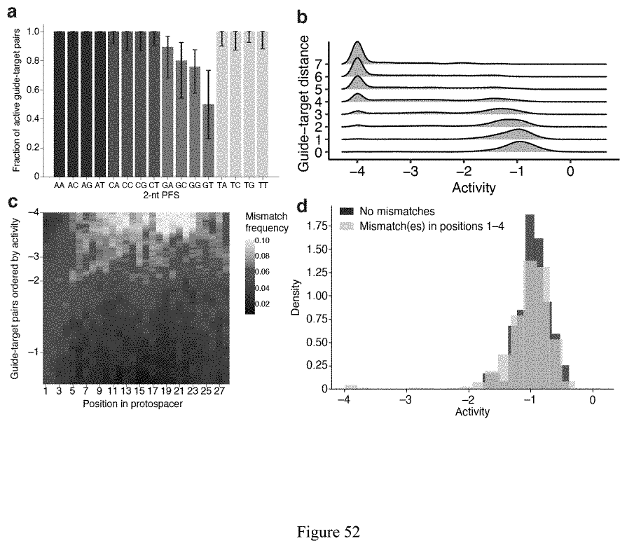

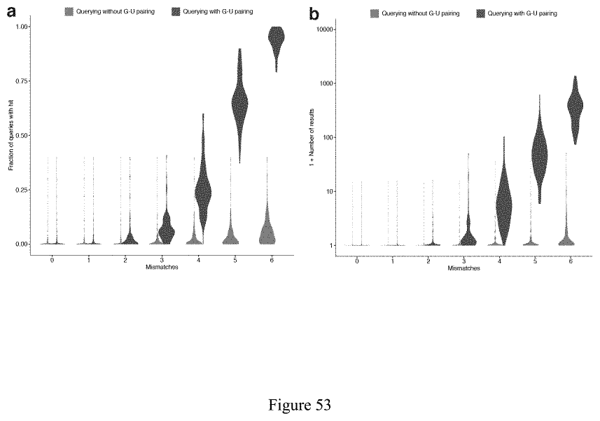

Applicants proceeded with further evaluating CNN models to use in ADAPT. During model selection, Applicants'space of CNN models allows for multiple parallel convolutional and locally-connected filters of different widths (FIG. 44). Using a locally-connected layer significantly improves model performance (FIG. 45), which Applicants hypothesize is because it helps the model to learn strong spatial dependencies in the datafor example, a mismatch-sensitive seed region that may be missed by convolutional layers and difficult for fully connected layers to ascertain. Applicants' classification CNN performs well on a held-out test set (auROC=0.866; auPR=0.972 with 85.6% of the test set being true positive; FIG. 34d, FIG. 46a). When guide and targets are not identical, it also yields a lower false-positive rate and higher precision than a simple method classifying activity according to the PFS and guide-target divergence (FIG. 34d, FIG. 46b). Applicants' regression CNN also predicts the activity of active guide-target pairs well (=0.635; FIG. 34e, FIG. 47a) and accurately separates pairs into quartiles based on predicted activity (FIG. 34f, FIG. 47b). Simple features, such as PFS and number of mismatches (FIG. 48), extracted by the CNN models could explain much of the models' performance, yet they retain high predictive capacity when evaluated individually on different choices of PFS (FIGS. 49a,b and 50a-d) and guide-target divergence (FIGS. 49c,d and 50e-g). To use these models in ADAPT, Applicants chose a decision threshold for the classifier that yields a desired precision (Methods; FIG. 34d, FIG. 46a) and then define a piecewise activity function that is 0 if a guide-target pair is classified as inactive, and the regressed activity if active. A learning curve shows that additional data similar to Applicants' current dataset would not improve model performance (FIG. 51), though this does not preclude the possibility of additional distinct guide sequences improving performance. Beyond modeling activity, Applicants also examined Applicants' dataset to enhance the understanding of LwaCas13a preferences. Prior studies have seen weaker detection activity with a G PFS for LwaCas13a16 and characterized the PFS for other orthologues37, 39 and in other settings38. Applicants also observe reduced activity with a G PFS and, extending to another position, Applicants find that GA, GC, and GG provide higher activity than GT (FIG. 52a), suggesting 3′ HV may be a more stringent preference. The breadth of Applicants' data also clarifies effects of mismatches (FIG. 52b) and the mismatch-sensitive seed region that has previously been characterized, using a limited number of guide-target pairs, for LbuCas13a39 and LshCas13a37. In particular, Applicants find that the worstperforming guide-target pairs are relatively likely to contain mismatches in positions 6-11 of the guide RNA spacer (FIG. 52c), concordant with the known region. Applicants also find nearly full tolerance for multiple mismatches on the 3′ end of the guide RNA spacer (FIG. 52d), a property that ADAPT's predictive model learns and leverages when positioning guides in diverse regions. Coefficients from the regularized linear models included in Applicants' model comparison are consistent with Applicants' findings and also reveal position-specific allele mismatches that affect activity (FIG. 48). A thorough modeling of defined features, as performed for Cas13d42, could highlight additional principles for Cas13a guide design.

Enforcing high taxon-specificity allows designs to accurately differentiate viral species or strains that are genetically similar. Applicants enforce specificity in ADAPT by developing a method to determine whether a probe is specific, which Applicants use to constrain the ground set of probes (Supplementary Note 2a). Determining whether a probe is specific requires a search that tolerates multiple mismatches over a short query length and, when both probe and target are RNA, G-U wobble base pairs. It is central to overcome this latter challenge: Applicants found that ignoring G-U pairing when designing species-specific probes for viruses can result in missing nearly all off-target hits and deciding many probes to be species-specific when they likely are not (FIG. 53). Similar challenges arise when determining off-target effects for small interfering RNA, but prior approaches commonly ignore the G-U pairing problem or use a solution that is inadequate for ADAPT's goals (details in Supplementary Note 2b).

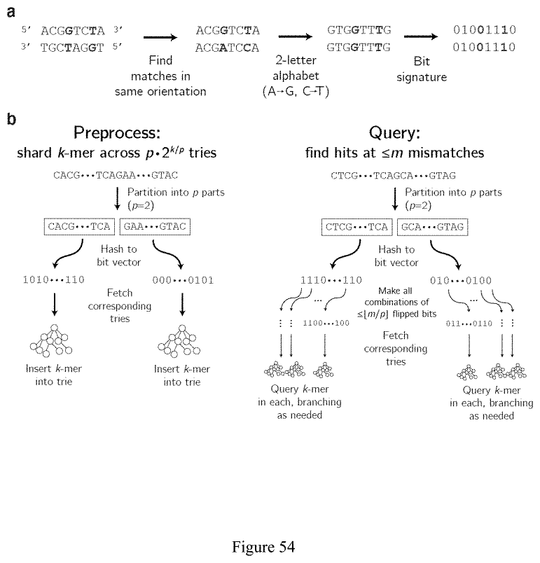

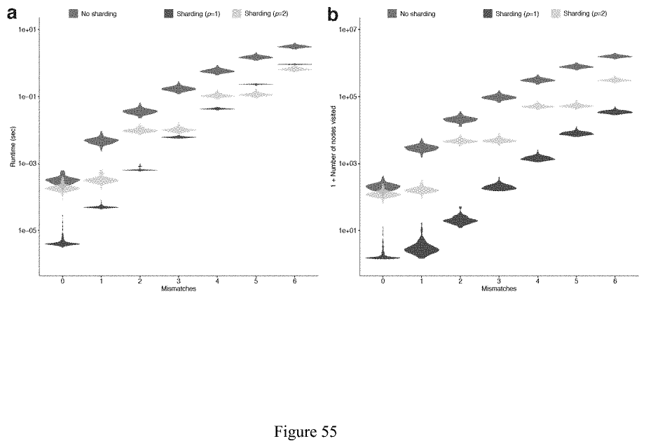

Applicants experimented with two approaches to address these challenges. Applicants first tested a probabilistic near-neighbor query algorithm that has a tunable reporting probability for identifying off-target hits (Supplementary Note 2c). When amplified across many designs, this technique misses many offtarget hits and therefore permits non-specific guides, as expected; raising the reporting probability to be sufficiently high can make this outcome unlikely, but Applicants found doing requires too much memory and makes queries too slow to be practical. Thus, Applicants developed a data structure and query algorithm suited to determining the specificity of a probe, tolerating both high divergence and G-U wobble base pairing. The data structure indexes k-mers from all input taxonomies, in which the k-mers are split across many small tries according to a hash function (FIG. 54 and Supplementary Note 2d). The approach is exact—that is, it finds any non-specificity of a query—and therefore, in theory, guarantees high specificity of ADAPT's designs. The approach runs about 10-100—times faster than a simple data structure with the same capability (FIG. 55).

### End-to-End Design

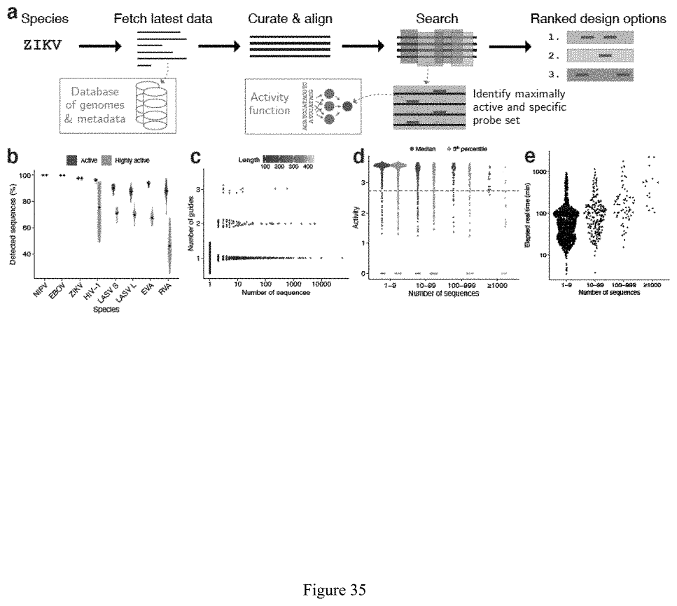

ADAPT links Applicants' methods to enable end-to-end design (FIG. 35a). It performs a search across a viral genome to identify regions to target, scored according to both their amplification potential (e.g., presence of conserved endpoints) and the optimal activity of a probe set within the region (Supplementary Note 3a). The search, which follows the branch and bound paradigm to run efficiently, identifies the best N design options, each containing primers and probes; the design options are non-overlapping to ensure diversity of the output and users preset N, for which smaller

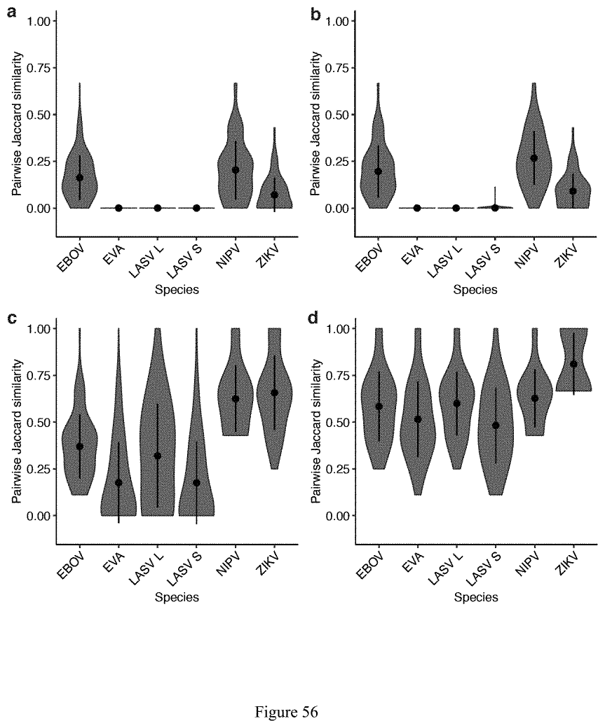

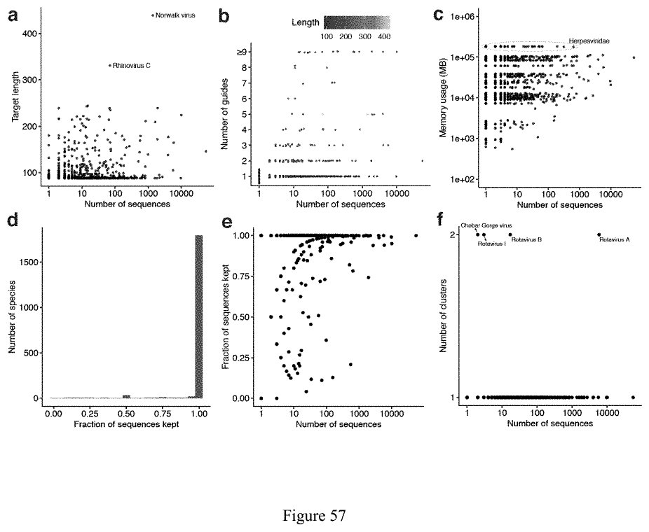

values speed the search. ADAPT also connects directly with viral genome databases to download and curate sequences—usually, all available near-complete or complete genomes—from the targeted taxa, as well as for building the index that enforces specificity (Supplementary Note 3b). For some taxa there are few available genome sequences, notably early in an outbreak, and therefore little data to inform the search. Applicants developed a proactive scheme that uses the GTR model to find relatively likely combinations of substitutions where a probe binds, allowing us to estimate a probability that a probe's activity will degrade over time; for example, degradation may occur owing to transitions at a mismatch-sensitive ref site. Applied to SARS-CoV-2, Applicants found that, for some Cas13a designs, there is a low probability (5%) their predicted activity will degrade over a 5 year timespan (FIG. 56a,b). Applicants report this information in ADAPT because it may be helpful in risk-averse situations, but it has little effect overall on design option rankings (FIG. 56c). Applicants computationally evaluated design options output by ADAPT, accounting together for primers and probes, on 7 RNA viruses that represent different degrees of genomic diversity. Applicants performed designs using Applicants' Cas13a activity function, so the probes are Cas13a guides. Algorithmic randomness and the particular distribution of known sequences may introduce variability; Applicants found that, owing to these variables, designs are rarely exactly the same across runs (FIG. 57a,b), but that they do often target similar genomic regions (FIG. 572c,d). Applicants also performed cross-validation to measure the designs' generalization. Designs are predicted to be active in detecting >85% of held-out genomes for all tested species and are, in all but one species, “highly active” (top 25% of Applicants' dataset) in detecting >65% of held-out genomes (FIG. 35b). The results show that ADAPT's outputs are robust.

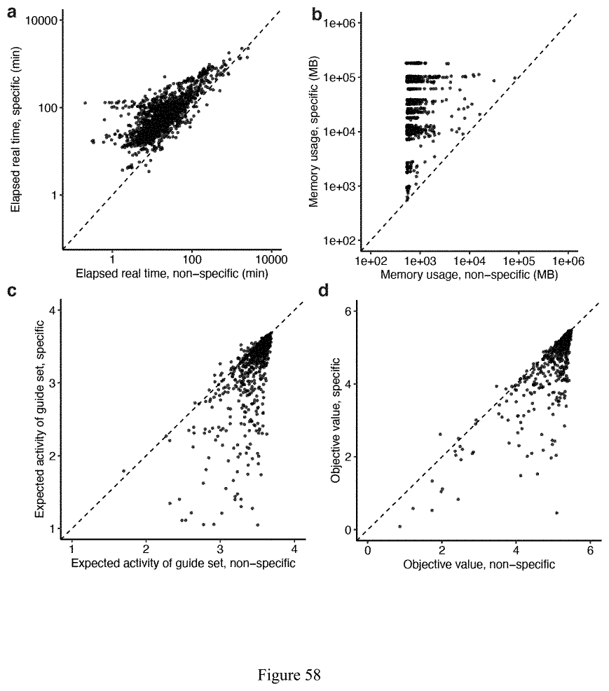

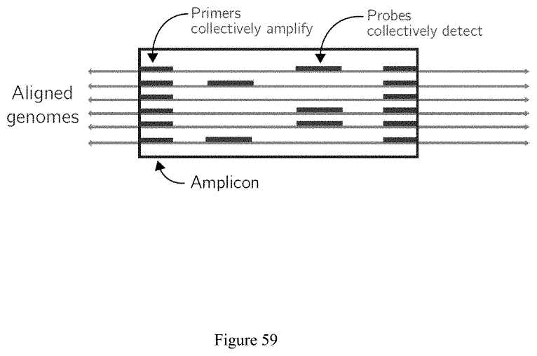

Applicants applied ADAPT to design detection assays, including primers and Cas13a guides, for the 1,933 viral species known to infect vertebrates, enforcing specificity at the species level within each family. The designs generally have short amplicons (<200 nt) and use 3 or fewer guides for all species, including species with thousands of available sequences (FIG. 35c; Supplementary FIG. 58a). For 95% of species, the guides detect most sequences with high activity (FIG. 3d). Using a related objective function in which Applicants minimize the number of guides subject to detecting >98% of sequences with high activity, Applicants similarly obtain few guides for most species although 40 species do require more than 3 guides (FIG. 58b) and, in one extreme case, as many as 73 (Enterovirus B). End-to-end design completed in under 24 hours for all but 3 species (human cytomegalovirus, SARS-related coronavirus, and IAV; under 38 hours for all species and under 2 hours for 80%), with runtime depending in part on the number of sequences for each species (FIG. 35e), and generally used 1-100 GB of memory (Supplementary FIG. 58c). During curation, ADAPT retains almost all sequences for most species (FIGS. 58d-f), including the ones with >1,000 sequences, though it does filter out >50% of sequences for 38 species and further investigation is needed to determine whether this is appropriate. As Applicants expected, enforcing specificity imposes a considerable computational burden, increasing runtime and memory usage (FIGS. 59a,b) while decreasing the solution's activity and objective value (FIGS. 59c,d); however, the effect is tunable by adjusting the specificity's stringency.

### Applicants' Discussion

ADAPT leverages diverse genome data and predictive models to design optimal nucleic acid diagnostic assays for viruses. Early versions of ADAPT have previously been applied to detecting Lassa virus46 and to specific detection of 169 human viruses and influenza subtyping40. Applicants also used ADAPT to quickly design a highly active and specific SARS-CoV-2 assay in January, 2020, as well as assays for 66 related viruses47; though designed from only the 20 genomes available at the time6, Applicants predict the SARS-CoV-2 assay to be active in detecting 99.3% of the 45,000 genomes available in mid-June, 2020 (highly active, 98.6%). Applicants now envision running ADAPT regularly or even continuously for thousands of viruses. Doing so could make it more likely that there exist highly effective assays for many viruses at the start of an outbreak; assays for many strains could even be preemptively validated. Routinely running ADAPT would also provide assay designs that always best reflect the latest diversity. Though there are likely to be regulatory obstacles to updating assays, that could improve because agencies have previously adjusted guidance in response to new bioinformatic and diagnostic technologies that accommodate large diversity48. ADAPT's framework is broadly applicable. While Applicants trained Applicants' activity model on CRISPRCas13a data, one could train similar models for other diagnostic technologies with enzyme-activated signal, given an appropriate dataset. Beyond nucleic acid diagnostics, finding maximally active and specific sequence in viral genomes has other uses, including serology tests, therapies, and vaccines. For example, sequence diversity likely impacts the efficacy of sequence-based siRNA and antibody therapeutics49. CRISPR-based antiviral development also requires deep consideration to sequence diversity, guide activity, and specificity50. Longer term, the framework could help sequence-based vaccine selection51, 52 by proposing antigens that yield high predicted antibody titers across circulating strains.

Applicants are pursuing several directions to advance ADAPT's utility. To obtain even more active probes, Applicants are developing models, trained on Applicants' Cas13a dataset, to generate optimally active Cas13a guides de novo. These methods could replace Applicants' current procedure for constructing the ground set and be useful for other applications in which an optimally active probe does not arise exactly from a known sequence. Applicants are also turning attention toward RT-qPCR, the centerpiece of nucleic acid diagnostics; while there is extensive history on assay design, COVID-19 qPCR diagnostics exhibit different sensitivities and some have mutations against targeted regions53, which suggests room for design improvement.

Applicants see ADAPT as an important component in viral surveillance, helping to translate genomic data—representing new and ever-changing diversity—into effective diagnostic assays and other tools.

### Methods

### ADAPT

Supplementary Notes describe ADAPT's algorithms, data structures, and implementation details. Supplementary Note 1 defines and solves objective functions. Supplementary Note 2 describes how ADAPT enforces specificity. Supplementary Note 3 describes how ADAPT searches for genomic regions to target and links with sequence databases.

Introductory analyses

To illustrate viral database growth, Applicants charted the growth in the number of viral genomes and their unique 31-mers over time (FIG. 37). Applicants first curated a list of viral species from NCBI54 known to infect humans (November 2019). For each, Applicants took all NCBI genome neighbors5 (influenza sequences from the Influenza Virus resource55), which represent near-complete or complete genomes. To assign a date for each, Applicants used the GenBank entry creation date rather than sample collection date for several reasons, including that this date more directly represents Applicants' focus in the analysis (when the sequence becomes present in the database) and that every entry on GenBank contains a value for this field. To control for some viruses having multiple segments (and thus sequences), Applicants only used counts for one segment for each species, namely the segment that has the most number of sequences.

Applicants used influenza A virus subtyping as an example to demonstrate the effect of evolution on diagnostics (FIG. 33a, FIG. 37). Applicants selected the most conserved k-mers—representing probe or guide sequencesfrom the sequences available at different years. Here, for simplicity, Applicants ignored all other constraints, such as detection activity and specificity (the latter of which is critical for subtyping), which would further degrade the temporal performance of the selected k-mers. In particular, for each design year Y, Applicants selected the 15 non-overlapping 30-mers found in the largest number of sequences taken from the two most recent years (Y−1 and Y). Applicants then measured the fraction of sequences in subsequent test years (Y , Y+1, . . . ) that exactly contain each of these k-mers. Applicants performed the design strategy over 10 resamplings of the sequences and use the mean fraction. Applicants repeated this 4 times: for segment 4 sequences of H1 and H3 subtypes, and segment 6 sequences of N1 and N2 subtypes.

To visualize mutations accumulating on a genome during the course of an outbreak (FIG. 33b), Applicants used complete SARS-CoV-2 genomes from GISAID6. Applicants called variants in all genomes, through May 15, 2020, against the reference genome ‘EPI ISL 402119’. For every date d between Feb. 1, 2020 and May 15, 2020, spaced apart by 7 days, at every position Applicants calculated the fraction of all genomes collected up to d that have a variant against the reference. Applicants called all variants present between 0.1% and 1% frequency on some d as “low” frequency and variants at _1% frequency on some d as “high” frequency. Applicants ignored all variants present at _1% frequency on the initial d (ancestral) or that were both low frequency on the initial d and stayed low frequency by the final d—i.e., Applicants keep the variants that transitioned to low or high frequency by the final d. Applicants show the d when the variant first becomes called as low (light purple) or high (dark purple) frequency. If a variant transitions both to low and then to high frequency by the final d, Applicants only show it for the d when it becomes high frequency.

### Cas13a Library Design and Testing

Applicants designed a collection of CRISPR-Cas13a crRNA guides and target molecules to evaluate crRNAtarget activity, focusing on assessing likely-active crRNA-target pairs. First, Applicants designed a target (the wildtype target) that is 865-nt long (design details below). Applicants then created 94 guides (namely, the 28 nt spacers) tiling this target (FIG. 34a, FIG. 40a). The tiling scheme is such that there are 30-nt blocks with 4 guides overlapping, in which the starts of the 3 guides, from the start of the most 5′ guide, are 4 nt, 13 nt, and 23 nt. Of the 94 guides, 87 are considered to be experimental, 3 to be negative controls, and 4 to be positive controls. Applicants created 229 unique target sequences: 1 of them is the wildtype (effectively, a positive control), 225 are experimental (mismatches and varying PFS alleles against the guides), and 3 are negative controls. All guides exactly match the wildtype target and should detect this, except the 3 negative control guides, which are not intended to detect any targets except one of the 3 negative control targets each. The 4 positive control guides target 4 30-nt regions with a perfectly complementary sequence and non-G PFS that are held constant across all targets, with the exception of the 3 negative control targets. Across the experimental targets, the mismatches profile mismatch positions and alleles against the guide. For the experimental targets, Applicants introduced single mismatches evenly spaced every 30 nt along the experimental region such that every guide targeting this region has either a single mismatch or an altered +1 or +2 PFS; Applicants created a total of 45 such targets to probe all 3 possible base mismatches and 15/30 of the possible phasings. In the remainder of the experimental targets, Applicants generated targets with 2, 3, or 4 mismatches per 30 nt block with respect to the guide RNA in phase with the block. Mismatch positions were randomly chosen to uniformly sample (or, when possible, exhaustively enumerate) average mismatch spacing and average mismatch distance to the center of the spacer. The 87 experimental guides may detect up to 226 unique target sequences (the wildtype and 225 experimental targets), providing 19,662 experimental guide-target pairs.

To construct the wildtype target sequence, Applicants aimed to produce a composition spanning viral genomic sequence diversity. In particular, first Applicants took a dataset of genomes from human-infecting viral species56, constructed a vector of dinucleotide frequencies for each species, and performed principal component analysis of the species from these vectors. For each 30-nt block of the wildtype target, Applicants selected a point from the space of the first 3 principal components (uniformly at random), reconstructed a corresponding vector of dinucleotide frequencies (i.e., transformed the point back to the original space), and then iteratively selected every next nucleotide in the block according to the distribution of dinucleotides. In positions that would serve as a PFS site for a guide, Applicants disallowed G, and proportionately adjusted upwards the probability of choosing a G in non-PFS positions to maintain the total dinucleotide frequency in accordance with the randomly selected distribution (mismatches in experimental targets can still introduce a G PFS).

Applicants synthesized the targets as DNA, in vitro transcribed them to RNA, and synthesized the crRNAs as RNA. On all crRNAs, Applicants used the same direct repeat (GAUUUAGACUACCCCAAAAACGAAGGGGACUAAAAC (SEQ ID NO: 44)). To determine a reasonable concentration for measuring fluorescence over time points, Applicants tested 8 concentrations of 2 targets and 2 guides in a pilot experiment (FIG. 41a) and proceeded with 6.25 A 109 cp/μL. Applicants tested the library using CARMEN, a droplet-based Cas13a system; Applicants followed the methodology described in ref. 40, which also contains the protocol. Briefly, a guide-target pair is enclosed in a droplet, together with the Cas13a enzyme, that may result in a detection reaction and thus fluorescence. Applicants took an image of each location of each chip roughly every 20 minutes to measure this fluorescence. To alleviate the presence of microdroplets in the experiment (i.e., an irregular pairing of target and guide; about ⅓ of the droplets), Applicants trained and applied a convolutional neural network on hand-labeled data to identify and remove these.

### Quantifying Activity

In Applicants' Cas13a detection experiments, a fluorescent reporter is cleaved over time and its cleavage follows first-order kinetics:

$$\frac{d [ R ]}{dt} = - \frac{{k}_{cat}}{{K}_{M}} [ E ] [ R ] ⇒ [ R ] = {[ R ]}_{0} {e}^{- \frac{{k}_{cat}}{{K}_{M}} [ E ] t}$$

where [R] is the coucentration of the not-yet-cleaved repeiter, [E] is the concenstration of the Cas13a guide-target complex,

$$\frac{{k}_{cat}}{{k}_{M}}$$

is the catalytic efficiency of the particular guide-target complex, and t is time. The fluorescence measurements that we make, y, are proportional to the quality of cleaved reporter at some time point:

[symbol]

×[R]₀−[R].

Therefore, for each guide-target complex we fit a curve of the form

[symbol]

=C(1−e^{-kt})+B.

Here, C and B represent the saturation point and background fluorescence, respectively, k represents the rate at which the reporter is cleaved, and it is proportional to the catalytic efficiency of the particular guide-target complex:

$$k = \frac{{k}_{cat}}{{K}_{M}} [ E ] .$$

This relationship is validated by the linear relationship between k and [E] (FIG. 41a) when Applicants vary the concentration of target (the limiting component of the complex). In producing Applicants' dataset, Applicants held [E] constant. Applicants used log10(k) as Applicants' measurement of guide activity (FIG. 34, FIG. 41a,b). Intuitively, each step-increase in log10(k) corresponds to a fold-decrease in the half-life of the reporter in the reaction.

Our experimental data incorporates multiple droplets for each guide-target pair (FIG. 42a). Each droplet represents one technical replicate of one of the guide-target pairs. Thus, Applicants have fluorescence values for each replicate at different time points, and in practice Applicants compute the activity log10(k) for each replicate.

Applicants curated the data to obtain a final dataset. Namely, Applicants discarded data from two guides that showed no activity between them and any targets, owing to low concentrations in their synthesis. Applicants also did not use data from positive or negative control guides, nor from the negative control target. Applicants' final dataset contains 19,209 unique guide-target pairs (FIGS. 40b,c), counting 20 nt of sequence context around each protospacer in the target (18,253 unique pairs when not counting context).

Most guide-target pairs show activity (FIG. 52d), as expected. At small values of k on a limited time scale (t up to _120 minutes), Applicants do not observe reporter activation (FIG. 41b). Moreover, the curve becomes approximately linear (first order Maclaurin expansion). At such values of k, Applicants cannot estimate both C and k together; intuitively, this is because there is too little detectable signal. Therefore, there is a cutoff at which Applicants can estimate k; Applicants labeled activities at log(k) >−4 as active, and the others as inactive. This phenomenon also implies that at smaller values of k, including ones Applicants label as active, activity estimates might be less reliable.

### Predicting Detection Activity

### Measurement Error

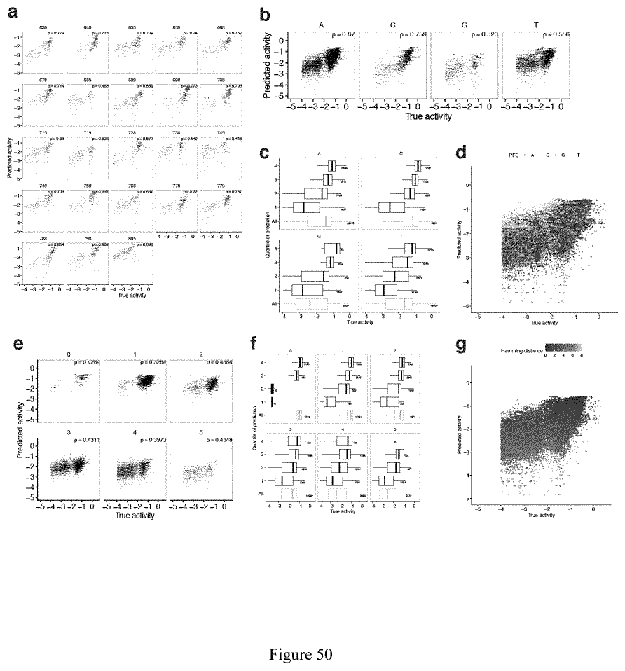

To account for measurement error, Applicants sampled, with replacement, 10 technical replicate measurements of activity for each guide-target pair (FIG. 42a). Applicants used this strategy to ensure that, although there are differing numbers of replicates per guide-target pair, each pair would be represented in the dataset with the same number of replicates. There are 19,209×10=192,090 points in total in Applicants' dataset that Applicants use for training and testing. When plotting regression results with guide-target pairs (FIG. 34e, FIG. 50, FIG. 47a), Applicants use each point to represent a replicate of a guide-target pair; replicates of the same pair yield the same predicted activity but different true activities owing to measurement error, and thus appear on a horizontal line when the vertical axis is predicted activity.

### Model and Input Descriptions

Applicants explored multiple models for classification (FIG. 34c and FIG. 43a), each with a space of hyperparameters:

L1 logistic regression: regularization strength (logarithmic in [10⁻⁴, 10⁴])

L2 logistic regression: regularization strength (logarithmic in [10⁻⁴, 10⁴])

L1+L2 logistic regression (elastic net): regularization strength (logarithmic in [10⁻⁴, 104]), L1/L2 mixing ratio (1.0−2^{x}+2⁻⁵ for x uniform in [−5, 0])

Gradient-boosted trees (GBT): learning rate (logarithmic in [10⁻², 1]), number of trees (logarithmic in [1, 2⁸], integral), minimum number of samples for splitting a node (logarithmic in [2, 2³], integral), minimum number of samples at a leaf node (logarithmic in [1, 2²], integral), maximum depth of a tree (logarithmic in [2, 2³], integral), number of features to consider when splitting a node (for n features, chosen uniform among considering all, 0.1n, p n, and log2 n)

Random forest (RF): number of trees (logarithmic in [1, 2⁸], integral), minimum number of samples for splitting a node (logarithmic in [2, 2³], integral), minimum number of samples at a leaf node (logarithmic in [1, 2²], integral), maximum depth of a tree (chosen uniformly among not restricting the depth or restricting the depth to a value picked logarithmically from [2, 2⁴] and made integral), number of features to consider when splitting a node (for n features, chosen uniform among considering all, 0.1n, p n, and log2 n)

Support vector machine (SVM; linear): regularization strength (logarithmic in [10⁻⁸, 10⁸]), penalty type (chosen uniformly along L1 and L2)

Multilayer perceptron (MLP): number of layers excluding the output layer (uniform in [1, 3]), dimensionality of each layer excluding the output layer (each chosen uniformly in [4, 127]), dropout rate in front of each layer (uniform in [0, 0.5]), activation function (chosen uniformly among ReLU and ELU), batch size always 16

Long short-term memory recurrent neural network (LSTM): dimensionality of the output vector (logarithmic in [2, 2⁸], integral), whether to be bidirectional (chosen uniformly among unidirectional and bidirectional), dropout rate in front of the final layer (uniform in [0, 0.5]), whether to perform an embedding of the one-hot encoded nucleotides and the dimensionality if so (chosen with ⅓ chance to not perform an embedding, and with ⅔ chance to perform an embedding with dimensionality chosen uniformly in [1, 8]), batch size is always 16

Convolutional neural network (CNN): number of parallel convolutional filters and their widths (chosen uniformly among not having a convolutional layer, 1 filter of width 1, 1 filter of width 2, 1 filter of width 3, 1 filter of width 4, 2 filters of widths {1, 2}, 3 filters of widths {1, 2, 3}, and 4 filters of widths {1, 2, 3, 4}), convolutional dimension (uniform in [10, 249]), pooling layer width (uniform in [1, 3]), pooling layer computation (chosen uniformly among maximum, average, and both), number of parallel locally-connected layers and their widths (chosen uniformly among not having a locally-connected layer, 1 filter of width 1, 1 filter of width 2, and 2 filters of widths {1, 2}), locally-connected filter dimension (uniform in [1, 4]), number of fully-connected layers and their dimensions (chosen uniformly among 1 layer with dimension uniform in [25, 74] and 2 layers each with dimension uniform in [25, 74]), whether to perform batch normalization in between the convolutional and pooling layers (uniform among yes and no), activation function (chosen uniformly among ReLU and ELU), dropout rate in front of the fully-connected layers (uniform in [0, 0.5]), L2 regularization coefficient (lognormal with mean μ=−13, _=4), batch size (uniform in [32, 255]), learning rate (logarithmic in [10⁻⁶, 10⁻¹])

Similarly, for regression Applicants explored multiple models (FIG. 43b,c), each with a space of hyperparameters:

L1 linear regression: regularization strength (logarithmic in [10⁻⁸, 10⁸])

L2 linear regression: regularization strength (logarithmic in [10⁻⁸, 10⁸])

L1+L2 linear regression (elastic net): regularization strength (logarithmic in [10⁻⁸, 10⁸]),

L1/L2 mixing ratio (1.0−2^{x}+2⁻⁵ for x uniform in [−5, 0])

Gradient-boosted trees (GBT): same hyperparameter space as for classification

Random forest (RF): same hyperparameter space as for classification

Multilayer perceptron (MLP): same hyperparameter space as for classification

Long short-term memory recurrent neural network (LSTM): same hyperparameter space as for classification

Convolutional neural network (CNN): same hyperparameter space as for classification

When training and testing the models, Applicants used 28 nt guide and target sequence, and include 10 nt of context in the target sequence on each side of the protospacer. Applicants tested the following different inputs:

‘One-hot (1D)’: vector containing 4 bits to encode the nucleotide at each target position and 4 bits similarly for each guide position; with a 28 nt guide and 10 nt of context in the target around the protospacer, there are (10+28+10+28) Å ˜4=304 bits

‘One-hot MM’: similar to ‘One-hot (1D)’ except explicitly encoding mismatches between the guide and target—i.e., vector containing 4 bits to encode the nucleotide at each target position and 4 bits, at each guide position, encoding whether there is a mismatch (if not, all 0) and, if so, the guide allele; same length as ‘One-hot (1D)’

‘Handcrafted’: features are count of each nucleotide in the guide, count of each dinucleotide in the guide, GC count in the guide, total number of mismatches between the guide and target sequence, and a one-hot encoding of the 2-nt PFS (coupling the 2 nucleotides); the number of features are 4+16+1+1+16=38

‘One-hot MM+Handcrafted’: concatenation of features from ‘One-hot MM’ and ‘Handcrafted’, except removing from ‘One-hot MM’ the bits encoding the 2-nt PFS because these are included in ‘Handcrafted’ Applicants used these inputs for all models except the LSTM and CNN. For these two models, which can capture and extract spatial relationships in the input, Applicants used an alternative input (labeled ‘One-hot (2D)’ in figures. Here, the input dimensionality is (48, 8) and consists of a concatenated one-hot encoding of the target and guide sequence. Namely, each element xi (i2 {1 . . . 48}) is a vector [xi,t, xi,g]. Target context corresponds to i 2 {1 . . . 10} (5′ end) and i 2 {39 . . . 48} (3′ end); for these i, xi,t is a one-hot encoding of the target sequence and xi,g is all 0. The guide binds to the target at i 2 {11 . . . 38} and, for these i, xi,t is a one-hot encoding of the target sequence protospacer at position i 10 of where the guide is designed to bind, while xi,g is a one-hot encoding of the guide at position i-10.

Applicants evaluated all models, except the MLP, LSTM, and CNN, in scikit-learn 0.2257. Applicants implemented and evaluated the MLP, LSTM, and CNN models in TensorFlow 2.1.058.

For the MLP, LSTM, and CNN models, Applicants used binary cross-entropy as the loss function for classification and mean squared error for regression. For these 3 models, Applicants used the Adam optimizer59 and performed early stopping during training (maximum of 1,000 epochs) with a held-out portion of the training data. Additionally, for the CNN Applicants regularized the weights (L2). When training all classification models, Applicants weighted the active and inactive classes equally.

### Data Splits and Test Set

In evaluating Applicants' models, Applicants must determine folds of the data and pick a held-out test set. One challenge with this is that, in Applicants' design, guides overlap according the position against which they were designed along the wildtype target. Although effects on activity might be position-dependent within the guide, this overlap can cause guides to have similar sequence composition or to be in regions of the target sequence with similar structure. To remove this possibility of leakage between a data split, after making a split of X into Xtrain and Xtest, Applicants remove all guide-target pairs from Xtest for which the guide has any overlap, in target sequence they are designed to detect, with a guide in Xtrain. Applicants perform this strategy during all cross-validated analyses. Applicants also use it to choose a test set that Applicants hold out from all analyses and use only for evaluating the final CNNs. This test set consists of 30% of all guides, taken from the far 3′ end of the target.

### Model Evaluation

Applicants performed nested cross-validation to select models—both for classification and regression—and evaluate Applicants'selection of them (FIG. 34c, FIG. 43). Applicants used 5 outer folds of the data. For each outer fold, Applicants searched for hyperparameters using a cross-validated (5 inner folds) random search over the space defined in Model and input descriptions; Applicants scored using the mean auROC (classification) or Spearman correlation (regression) over the inner folds. In each random search, Applicants used 100 hyperparameter choices for all models, except for the LSTM and CNN models (50), which Applicants found slower to train. The CNN models outperformed others in the above analysis, so Applicants selected a final CNN model for classification and another for regression. For each of classification and regression, Applicants performed a random search across 5 folds of the data using 200 random samples. Applicants selected the model with the highest auROC (classification) or Spearman correlation (regression) averaged over the folds. Applicants' evaluations of these two models used the held-out test set.

Incorporating into ADAPT

Applicants integrated the CNN models into ADAPT. First, Applicants set the decision threshold on the classifier's output to be 0.577467. Precision matters greatly in Applicants' context because Applicants would like confidence that designs determined to be active are indeed active. Applicants chose the threshold, via cross-validation, to achieve a desired precision of 0.975. In particular, Applicants took 5 folds of out data (excluding test data) and, for each fold, Applicants calculated the threshold that achieves a precision of 0.975 on the validation data. Applicants' decision threshold is the mean across the folds.

Applicants then defined a piecewise function, incorporating the classification and regression models, as:

$$d ( p , s ) = \{ \begin{matrix}0 & if C ( p , s ) < t \\ max ( 0 , r + R ( p , s ) ) & else \end{matrix}$$

where d(p, s) is the predicted detection activity between a probe p and target sequence s (s includes 10 nt of context). C(p, s) is the output of the classifier, t is the classification decision threshold, and R(p, s) is the output of the regression model. r is a shift that Applicants add to regression outputs to ensure d(p, s) is non-negative; though a nice property, it is not strictly needed as long as Applicants constrain the ground set as described in Supplementary Note 1a. The choice of r should depend on the range of activity values in the dataset; here, r=4.

### ADAPT Analyses

### Comparing Algorithms for Submodular Maximization

To compare the canonical greedy algorithm for constrained monotone submodular maximization with the fast randomized combinatorial algorithm35 (FIG. 38), Applicants ran ADAPT 5 times under each choice of parameter settings and species and, for each run, considered the mean final objective value taken across the best 5 design options. Applicants used the arguments ‘-pm3-pp 0.9-primer-gc-content-bounds 0.3 0.7-max-primersat-site 10-gl 28-max-target-len 250’ with Applicants' Cas13a activity model. Applicants used the default objective function in ADAPT: 4+A−0.5P−0.25 L, where A is the expected activity of the guide set, P is the number of primers, and L is the target length.

### Benchmarking Comprehensiveness

To benchmark comprehensiveness (FIG. 33d,e and FIG. 39), Applicants ran ADAPT with three approaches. In all approaches, Applicants decided that a probe detects a target sequence if and only if they are within 1 mismatch, counting G-U wobble pairs as matches; used a sliding window of 200 nt and a probe length of 30 nt; and used, as input, all NCBI genome neighbors5 for each species. In the first approach, Applicants used its ‘design naively.py’ program to select a single probe within each window via two strategies: (1) the consensus probe, computed at every site within the window, that detects the most number of sequences (‘consensus’); and (2) the most common probe sequence, determined at every site within the window, that detects the most number of sequences (‘mode’). In the second approach, Applicants maximized expected activity across the target sequences with different numbers of probes (hard constraints) using a penalty strength of 0 (i.e., no soft constraint). Here, Applicants defined the activity to be binary: 1 for detection, and 0 otherwise; this has the property that expected activity is equivalent to the fraction of sequences detected. In the third approach, Applicants use the objective function in ADAPT that minimizes the number of probes subject to constraints on the fraction of sequences detected (specified via ‘-gp’; 0.9, 0.95, and 0.99).

### Evaluating Dispersion and Generalization

Applicantsevaluated the dispersion, owing to randomness and sampling, in ADAPT's designs (FIG. 57). In all cases, used all NCBI genome neighbors5 for each species and used the following arguments with ADAPT: ‘-obj maximize-activity -soft-guide-constraint 1-hard-guide-constraint 5-penalty-strength 0.25-gl 28-p130-pm 3-pp 0.98-primer-gc-content-bounds 0.35 0.65-max-primers-at-site 10-max-target-length 500-obj-fn-weights 0.50 0.25’, with a cluster threshold such that there is only 1 cluster, and used Applicants' Cas13a activity model. Applicants ran ADAPT in two ways: 20 times without changing the input (output differences are owing to randomness) and 20 times with resampled input (output differences are owing to randomness and input sampling). Then, Applicants measured dispersion by treating the 5 highest-ranked design options from each run as a set and computing pairwise Jaccard similarities across the 20 runs. This computation requires us to evaluate overlap between two sets: in one comparison, Applicants consider a design option x to be in another set if x is present exactly in that other set (same primers and probes) and, in the other comparison, Applicants consider a design option x to be in another set if that other set has some design option with both endpoints within 40 nt of x's endpoints.

To evaluate the generalization of ADAPT's designs (FIG. 35b), Applicants performed cross-validation via repeated random subsampling. For each species, Applicants took all NCBI genome neighbors5 and, 20 times, randomly selected 80% of them to use as input for design and the remaining 20% to test against. For each split, Applicants used the same arguments with ADAPT as when evaluating dispersion: ‘-obj maximize-activity-soft-guide-constraint 1-hard-guide-constraint 5-penalty-strength 0.25-gl 28-p130-pm 3-pp 0.98-primer-gc-content-bounds 0.35 0.65-max-primers-at-site 10-max-target-length 500-obj-fn-weights 0.50 0.25’, with a cluster threshold such that there is only 1 cluster, and used Applicants' Cas13a activity model. When computing the fraction of sequences in the test set that are detected, Applicants required the sequence to be detected by a primer on the 5′ and 3′ ends of a region (within 3 mismatches) and a probe (here, guide) to detect the region; Applicants used the ‘analyze coverage.py’ program in ADAPT for this computation. Applicants labeled detection of a sequence as ‘active’ if a guide in the guide set is decided by Applicants' Cas13a classification model to be active against the target. Applicants labeled the detection as “highly active” if a guide in the guide set is both decided to be active by the Cas13a classification model and its predicted activity, according to the Cas13a regression model, is _2.7198637 (4 added to the output of the model, −1.2801363). This threshold corresponds to the top 25% of predicted values on the subset of Applicants' held-out test set that is classified as active.

### Benchmarking Trie-Based Specificity Queries

Applicants benchmarked the approach described in Supplementary Note 2d against a single, large trie (FIG. 55). For this, Applicants sampled 1.28% of all 28-mers from 570 viral species (78.7 million 28-mers in total), and built data structures indexing these. Applicants then randomly selected 100 species (here, counting each segment of a segmented genome as a separate species), and queried 100 randomly selected 28-mers from each of these for hits against the other 569 species. Applicants performed this for varying choices of mismatches. Applicants used the same approach to generate results in FIG. 53, there comparing queries with and without tolerance of G-U base pairing.

### Designs Across Vertebrate-Infecting Species

Applicants found all viral species in NCBI's viral genomes resources that have a vertebrate as a host, as of April, 2020. Applicants added to this list others that may have been incorrectly labeled, as well as influenza viruses, which are separate from the resource. There were 1,933 species in total and ADAPT was used to design primers and Cas13a guides to detect them. As input, Applicants used all genome neighbors from NCBI's viral genomes resources (influenza database for influenza species55). Applicants constrained primers to have a length and GC content that are recommended for use with RPA60, and thus are suitable for use with SHERLOCK16 detection.

Applicants used the following arguments when running ADAPT to maximize expected activity:

Initial clustering: clustered with a maximum distance of 30% (-cluster-threshold 0.3)

Primers and amplicons: primer length of 30, primers must have GC content between 35% and 65%, at most 10 primers at a sitel, up to 3 mismatches between primers and target sequence for hybridization, primers must hybridize to _98% of sequences, and length of a targeted genome region (amplicon) must be _250 nt (-pl 30-primer-gc-content-bounds 0.35 0.65-max-primers-at-site 10-pm 3-pp 0.98-max-target-length 250)

Guides: Cas13a guide length of 28 nt, together with Applicants' Cas13a predictive model (-gl 28-predict-activity-model-path models/classify/model-51373185 model s/regress/model-f8b6fd5d)

Guide activity objective: soft constraint of 1 guide, hard constraint of 5 guides, guide penalty (J of 0.25, using the randomized greedy algorithm (-obj maximize-activity-soft-guide-constraint 1-hard-guide-constraint 5-penalty-strength 0.25-maximization-algorithm random-greedy)

Specificity: query up to 4 mismatches counting G-U pairs as matches, calling a guide nonspecific if it hits 1% of sequences in another taxon (-id-method shard -id-m 4-idfrac

0.01)

Objective function and search: weights _A=0.5 and _L=0.25 in the objective function (defined in Supplementary Note 3a) and finding the best 20 design options (-obj-fn-weights 0.5 0.25-best-n-targets 20)

Applicants made some species-specific adjustments. For influenza A and dengue viruses, two especially diverse species, Applicants decreased the number of tolerated primer mismatches to 2 and allowed at most 5 primers at a site (-pm 2-max-primers-at-site 5); while these further constrain the design, they decrease runtime. For Norwalk virus and Rhinovirus C, Applicants relaxed the number of primers at a site and the maximum region length to identify designs (-max-primers-at-site 20-maxtarget-length 500). For Cervid alphaherpesvirus 2, which has a short genome, Applicants changed the GC-content bounds on primers to be 20%-80% (-primer-gc-content-bounds 0.2 0.8) to allow more potential amplicons. For 42 species, Applicants relaxed specificity constraints to identify designs (list and details in code).

Of the 1,933 species, 7 could not produce a design while maximizing activity and enforcing specificity, even with species-specific adjustments. They are: Bat mastadenovirus, Bovine associated cyclovirus 1, Chiropteran bocaparvovirus 4, Cyclovirus PKgoat21/PAK/2009, Finkel-Biskis-Jinkins murine sarcoma virus, Panine gammaherpesvirus 1, and Squirrel fibroma virus. Each of these 7 species has just one genome sequence and ADAPT could not identify a guide set satisfying specificity constraints; it is possible they are misclassified or have very high genetic similarity to other species. When showing results for this objective, Applicants report on 1,926 species.

In addition to using the above settings, which maximizes activity and enforces specificity, Applicants ran ADAPT with 3 other approaches: Applicants minimized the number of guides while enforcing specificity, requiring that guides be predicted to be highly active (as defined in Evaluating dispersion and generalization) in detecting 98% of sequences. Applicants also ran the objectives to maximize activity and minimize guides without enforcing specificity. 67 of the 1,933 species did not yield a design when minimizing the number of guides and enforcing specificity, owing to the constraints with this objective: ADAPT could not identify a guide set that is predicted to be highly active and achieves the desired coverage and specificity.

For species with segmented genomes, Applicants ran ADAPT and produced designs separately for each segment. Applicants then selected the segment whose highest-ranked design option has the best objective value (if multiple clusters, according to the largest cluster). Applicants expect the selected segment to generally be the most conserved one.

In all analyses showing results of the designs (e.g., number of guides, guide activity, and target

length), Applicants used the highest-ranked design option output by ADAPT. For the species with more than one cluster, Applicants report the mean across clusters from the highest-ranked design option in each cluster.

### Testing Activity and Specificity

### ADAPT Design Parameters

To generate designs with ADAPT for experimental testing, Applicants used the following arguments unless otherwise noted:

Initial clustering: force a single cluster (-cluster-threshold 1.0)

Primers and amplicons: primer length of 30, primers must have GC content between 35% and 65%, at most 5 primers at a site, up to 1 mismatch between primers and target sequence for hybridization, primers must hybridize to 98% of sequences, and length of a targeted genome region (amplicon) must be 250 nt (-pl 30-primer-gc-content-bounds 0.35 0.65-max-primers-at-site 5-pm 1-pp 0.98-max-target-length 250)

Guides: Cas13a guide length of 28 nt, together with Applicants' Cas13a predictive model (-gl 28-predict-activity-model-path models/classify/model-51373185 model s/regress/model-f8b6fd5 d)

Guide activity objective: soft constraint of 1 guide, hard constraint of 5 guides, guide penalty (J of 0.25, using the randomized greedy algorithm (-obj maximize-activity-soft-guide-constraint 1-hard-guide-constraint 5-penalty-strength 0.25-maximization-algorithm random-greedy)

Specificity: query up to 4 mismatches counting G-U pairs as matches, calling a guide nonspecific if it hits 1% of sequences in another taxon (-id-method shard-id-m 4 idfrac 0.01)

Objective function and search: weights _A=0.5 and _L=0.25 in the objective function (defined in Supplementary Note 3a) (-obj-fn-weights 0.5 0.25)

For SARS-CoV-2 input sequences, Applicants used the 9,054 complete genomes available on GISAID6 as of Apr. 28, 2020. Applicants also used genomes from GISAID for pangolin SARS-like CoV input sequences (isolates from Guangxi, China and Guandong, China). For all other input sequences—SARS-like CoV isolates RaTG13, ZC45, and ZXC21; other SARS-like CoVs; SARS-CoV; and other Coronaviridae species—Applicants used all genome neighbors from NCBI from each species5.

### Generating Test Target Sequences

Experimentally testing design options output by ADAPT also requires generating representative target sequences. Applicants found representative sequences for a design option, using a collection of genomes spanning diversity of a taxon, as follows: (1) Applicants extracted the amplicon (according to provided positions, e.g., from primer sequences), while extending outward to achieve a minimum length (usually 500 nt). (2) Applicants removed sequences that are too short, e.g., owing to gaps in the alignment. (3) Applicants computed pairwise Mash distances61 and performed hierarchical clustering (average linkage) to achieve a desired number of clusters or a maximum inter-cluster distance. (4) To avoid outliers, Applicants greedily selected (in order of descending size) clusters that include a desired total fraction of sequences, a particular number of targets, or ones representing particular taxa (specifics below). (5) Applicants computed the medoid of each cluster—i.e., the sequence with minimal total distance to all other sequences in the cluster. (6) Applicants used the medoids of each clusters as represented target sequences. The pick test targets.py program in ADAPT implements the procedure and Applicants used this program.

### Baseline Distribution of Activity

Applicants established a baseline distribution of activity using Cas13a guides, to detect SARS-CoV-2, selected from the genomic regions targeted by the United States Centers for Disease Control and Prevention (US CDC) RT-qPCR assays62. In particular, Applicants picked 10 random 28-mers from within the US CDC N1 amplicon that would have a non-G PFS and used these as Cas13a guides, according to the hCoV-19/Wuhan/IVDC-HB-01/2019 genome6. Applicants also chose another Cas13a guide at the site of the TaqMan probe. Applicants did the same from the US CDC N2 amplicon. In addition, in the N1 and N2 amplicons, Applicants used ADAPT to design a single guide with maximal activity (ignoring specificity) from within the amplicon. This provides 24 guides in total.

Experimental Designs with ADAPT

To evaluate the activity and lineage-level specificity of SARS-CoV-2 designs, Applicants used ADAPT to produce 10 design options for detecting SARS-CoV-2. Applicants increased the specificity in ADAPT to call a guide non-specific if it hits any sequence outside SARS-CoV-2 and also use the greedy maximization to obtain more intuitive outputs because, in this case, Applicants expect only a single Cas13a guide for each design option (-id-frac 0-maximization-algorithm greedy). Applicants enforced specificity to not detect any sequences outside of SARS-CoV-2 from the SARS-related CoV species (including related bat and pangolin coronavirus isolates) and also to not detect sequences from the other 43 species in the Coronaviridae family. Owing to experimental constraints, Applicants tested the highest-ranked 5. Applicants generated targets for each design option against which to test, using the ones representative of SARS-CoV-2; pangolin SARS-like CoVs (isolates from Guangxi, China); bat SARS-like CoV isolates ZC45 and RaTG13; and SARS-CoV (i.e., SARS-CoV-1).

To further evaluate activity and subspecies-comprehensiveness, Applicants used ADAPT to produce 10 design options for detecting the SARS-CoV-2-related taxon. In referring to SARS-CoV-2-related, Applicants use the definition given in FIG. 1b of ref 44; it encompasses SARS-CoV-2 and several related bat and pangolin SARS-like coronaviruses. To correct for sampling biases, Applicants used 10 sampled SARS-CoV-2 genomes as input so that they make up roughly half of sequences in the SARS-CoV-2-related taxon. Applicants used the same adjusted arguments in ADAPT as used for the SARS-CoV-2 designs (-id-frac 0-maximization-algorithm greedy). Applicants enforced specificity to not detect any sequences outside of SARS-CoV-2-related from the SARS-related CoV species (including other bat SARS-like coronaviruses) and also to not detect sequences from the other 43 species in the Coronaviridae family. For each design option, Applicants generated targets, and used the ones representative of SARS-CoV-2; pangolin SARS-like CoVs (isolates from Guangxi, China and Guangdong, China); bat SARS-like CoV isolates ZC45, ZXC21, and RaTG13; and SARS-CoV (i.e., SARS-CoV-1). For this experiment, the SARS-CoV target allows us to evaluate specificity, while the others allow us to evaluate activity and subspecies-comprehensiveness.

Applicants used ADAPT to produce 10 design options to detect the SARS-related coronavirus species, and Applicants used these to evaluate activity, species-comprehensiveness, and specificity. To correct for sampling biases, Applicants used 300 sampled SARS-CoV-2 genomes as input so that they make up roughly half of sequences in the species. Applicants enforced specificity to not detect sequences from the other 43 species in the Coronaviridae family. For each design option, Applicants generated representative targets that encompass SARS-CoV-2, SARS-CoV (i.e., SARS-CoV-1), bat SARS-like CoVs, pangolin SARS-like CoVs, MERS-CoV, Human coronavirus OC43, and Human coronavirus HKU1 . There were 8 or 9 representative targets in total for each design option. To evaluate species-comprehensiveness, Applicants focused on Enterovirus B and used ADAPT to produce 10 design options. Owing to its extensive diversity, Applicants made several adjustments to arguments, which help to increase the space to potential design options (-primer-gc-content-bounds 0.30 0.70-pm 4-pp 0.80-max-primers-at-site 10-id-frac 0.10-penalty-strength 0.15). Applicants enforced specificity to not detect the 18 other species in the Enterovirus genus. For each design option, Applicants generated representative targets from clusters that encompass at least 90% of all sequences. There were between 1 and 15 targets for each design option (the precise number depends heavily on the location of the design option amplicon in the genome). Applicants additionally tested specificity within the Enterovirus genus by generating a single representative target for each of Enterovirus A, Enterovirus C, and Enterovirus D.

Applicants built a positive control into each target. In particular, Applicants added the sequence 5′-CACTATAGGGGCTCTAGCGACTTCTTTAAATAGTGGCTTAAAATAAC-3′ (SEQ ID NO: 45) to the 5′ end of each target and included in Applicants' tests of every target a guide with spacer sequence 5′-GCTCTAGCGACTTCTTTAAATAGTGGC-3′ (SEQ ID NO: 46).

### Data Availability

Data is available in several repositories:

The CRISPR-Cas13a library and activity dataset is available at: https://github.com/broadinstitute/adapt-seq-design/tree/master/data/CCF-curated

Serialized trained models are available at:

https://github.com/broadinstitute/adapt-seq-design/tree/master/models/cas13

Experimentally tested designs, as output by ADAPT, are available at:

https://github.com/broadinstitute/adapt-designs

### Code Availability

Code is available in several repositories:

ADAPT is freely available under the MIT license at:

https://github.com/broadinstitute/adapt

Code to replicate the predictive modeling and analyses is available at:

https://github.com/broadinstitute/adapt-seq-design

Code to replicate the designs across the vertebrate-infecting viral species is available at:

https://github.com/broadinstitute/adapt-designs-continuous

Code to replicate the other analyses in this paper is available at:

https://github.com/broadinstitute/adapt-analyses

### Supplementary Note 1

This note describes two formulations for objective functions implemented in ADAPT. Unless otherwise noted, for designs and analyses in the paper, Applicants use the formulation in Design formulation #1: maximizing expected activity.

1a Design formulation #1: maximizing expected activity

### Objective

Let S be an alignment of sequences from species t in a genomic region. Applicants wish to find a set P of one or more probes that maximizes detection activity over these sequences. This objective bears some similarity to the problem of designing PCR primers that match a maximum number of sequences23, though solutions generally binarize decisions about detection rather than accommodate continuous predictions. As described in Methods, Applicants have a model to predict a measurement of detection activity between one probe p and one sequence s 2 S, which Applicants represent by d(p, s). While more than one probe in the set P may be able to detect s, Applicants use the best probe against s to measure P′s detection activity for s; that is, the predicted detection activity is

$$d ( P , s ) = {max}_{p ∈ P} d ( p , s ) .$$

(In FIG. 1c we use A(P, s) to represent this quantity.) We represent the predicted activity by P, against all sequences in S, with the function F(P). F(P) is the expected value of d(P,s) taken over all the s ∈ S; the weight wₛ for each s can reflect a prior probability on applying the detection in practice to targeting a sequence like s (e.g., based on the genome's date or geographic location). Thus we define

$$F ( P ) = {\sum }_{s ∈ S} {w}_{s} \cdot d ( P , s ) .$$

We currently set a uniform over targeting the genones in S, with the effect being that all wₛ are equal.

We must introduce penalties for the number probes. Striving for a small number of probes is important because (a) if used in separate reactions, this adds time and labor; (b) if multiplexed in one reaction, there is generally competition in hybriclizion to a target, and having more in a reactian may reduce the resulting derection signal; and (c) they require time and money to synthesize, and to experimentally validate. For this penalty, we use a soft constraint mₚ and hard constraint m̅ₚ̅ on the number of probes. We wish to solve

$${max}_{p} \{ F ( P ) - λ \cdot max ( 0 , \vert P \vert - {m}_{p} ) \text{:} \vert P \vert \leq \overline{{m}_{p}} \}$$

where λ>0 serves as a weight on the penalty. λreflects a tolerance for higher F(P) at expense of more probes.

Submodularity of the objective

Let \tilde{F}(P)=F(P)−λ·max(0, |P|−mₚ). We wamt to prove that \tilde{F}(P) is submodular.

We start by showing first that d(P,s) is submodular. For ease of notation, we drop s, referfring to d(P,s) as d(P) and d(p,s) as d(p). Consider probe sets A and B, with A⊆B, and some possible probe x∉B. Note that

$$\begin{matrix}d ( A ⋃ \{ x \} ) = {max}_{p ∈ A ⋃ \{ x \}} d ( p ) \\ = max ( {max}_{p ∈ A} d ( p ) , d ( x ) ) \\ = max ( d ( A ) , d ( x ) )\end{matrix}$$

and therefore d(A∪{x})−d(A)≥0. Likewise, d(B∪{x})=max(d(B), d(x)), and d(B∪{x})−d(B)≥0. Also, since A⊆B, we have

$$\begin{matrix}d ( B ) = {max}_{p ∈ B} d ( p ) \\ = max ( {max}_{p ∈ A} d ( p ) , {max}_{p ∈ B \backslash A} d ( p ) ) \\ = max ( d ( A ) , {max}_{p ∈ B \backslash A} d ( p ) ) \geq d ( A ) .\end{matrix}$$

We now consider two cases:
Assumed d(B)≥d(x). Then, d(B∪{x})−d(B)=max(d(B), d(x))−d(B)=0. Therefore, d(A∪{x})−d(A)≥d(B∪{x})−d(B).
Assume d(B)<d(x). It follows from d(B)≥d(A) tjhat d(x)≥d(A) and that d(x)−d(A)≥d(x)−d(B). Since max(d(A), d(x))=d(x) and max(d(B), d(x))=d(x), we have max(d(A), d(x))−d(A)≥max(d(B), d(x))−d(B). Therefore, in this case as well, d(A∪{x})−d(A)≥d(B∪{x})−d(B).

Hence, d(G, s) is submodular. Since F(P) is a non-negative linear combination of d(P, s), it follows that F(P) is submodular.

Using the above result, we show that \tilde{F}(P) is submodular. Again, consider probe A and B, with A⊆B, and some possible probe x∉B. We want to show that \tilde{F}(A∪{x})−\tilde{F}(A)≥\tilde{F}(B∪{x})−\tilde{F}(B). We have that

$$\begin{matrix}\tilde{F} ( A ⋃ \{ x \} ) - \tilde{F} ( A ) = & F ( A ⋃ \{ x \} ) - λ \cdot max ( 0 , \vert A \vert + 1 - {m}_{p} ) - \\ & F ( A ) + λ \cdot max ( 0 , \vert A \vert - {m}_{p} ) \\ = & F ( A ⋃ \{ x \} ) - F ( A ) - λ ( max ( 0 , \vert A \vert + \\ & 1 - {m}_{p} ) - max ( 0 , \vert A \vert - {m}_{p} ) ) \geq \\ & F ( B ⋃ \{ x \} ) - F ( B ) - λ ( max ( 0 , \vert A \vert + \\ & 1 - {m}_{p} ) - max ( 0 , \vert A \vert - {m}_{p} ) ) (* )\end{matrix}$$

where the last step follows from submodularity of F(P). We new consider two cases.
Assume |A|≥mₚ. Since A⊆B, |B|≥mₚ. Continuing from (+), we have

$$\begin{matrix}\tilde{F} ( A ⋃ \{ x \} ) - \tilde{F} ( A ) \geq & F ( B ⋃ \{ x \} ) - F ( B ) - λ ( \vert A \vert + 1 - {m}_{p} - \\ & ( \vert A \vert - {m}_{p} ) ) \\ = & F ( B ⋃ \{ x \} ) - F ( B ) - λ \\ = & F ( B ⋃ \{ x \} ) - F ( B ) - λ ( \vert B \vert + 1 - {m}_{p} - \\ & ( \vert B \vert - {m}_{p} ) ) \\ = & F ( B ⋃ \{ x \} ) - λ ( \vert B \vert + 1 - {m}_{p} ) - \\ & [ F ( B ) - λ ( \vert B \vert - {m}_{p} ) ] \\ = & F ( B ⋃ \{ x \} ) - λ \cdot max ( 0 , \vert B ⋃ \{ x \} \vert - {m}_{p} ) - \\ & [ F ( B ) - λ \cdot max ( 0 , \vert B \vert - {m}_{p} ) ] \\ = & \tilde{F} ( B ⋃ \{ x \} ) - \tilde{F} ( B ) .\end{matrix}$$

Assume |A|<mₚ. Continuing from (x) in this case, we now have

$$\begin{matrix}\tilde{F} ( A ⋃ \{ x \} ) - \tilde{F} ( A ) \geq & F ( B ⋃ \{ x \} ) - F ( B ) \\ \geq & F ( B ⋃ \{ x \} ) - F ( B ) - λ [ max ( 0 , \vert B \vert + 1 - \\ & {m}_{p} ) - max ( 0 , \vert B \vert , - {m}_{p} ) ] \\ = & F ( B ⋃ \{ x \} ) - λ \cdot max ( 0 , \vert B \vert + 1 - {m}_{p} ) - \\ & [ F ( B ) - λ \cdot max ( 0 , \vert B \vert - {m}_{p} ) ] \\ = & F ( B ⋃ \{ x \} ) - λ \cdot max ( 0 , \vert B ⋃ \{ x \} \vert - {m}_{p} ) - \\ & [ F ( B ) - λ \cdot max ( 0 , \vert B \vert - {m}_{p} ) ] \\ = & \tilde{F} ( B ⋃ \{ x \} ) - \tilde{F} ( B ) .\end{matrix}$$

Hence, \tilde{F}(P) submodular.

Now we show how to ensure that \tilde{F}(P) is non-negative. Let P only contain probes p such that F({P})≥λ·(m̅ₚ̅−mₚ). Since F is monotonically increasing, F(P)≥λ·(m̅ₚ̅−mₚ). Thus,

\tilde{F}(P)=F(P)−λ·max(0, |P|−mₚ)≥λ·(m̅ₚ−mₚ)−λ·max (0, |P|−mₚ).

If |P|≤mₚ, then

\tilde{F}(P)≥λ·(m̅ₚ−mₚ)−0≥0

where the last inequality follows from m̅ₚ≥mₚ. If |P|>mₚ, then

$$\begin{matrix}\tilde{F} ( P ) \geq λ \cdot ( \overline{{m}_{p}} - {m}_{p} ) - λ \cdot ( \vert P \vert - {m}_{p} ) \\ = λ \cdot ( \overline{{m}_{p}} - {m}_{p} - \vert P \vert + {m}_{p} ) \\ = λ \cdot ( \overline{{m}_{p}} - \vert P \vert ) \geq 0\end{matrix}$$

where the last inequality follows from m̅ₚ̅≥|P|. Therefore, \tilde{F}(P)≥0 always. Let our ground set Q be the set of probes from which we seleet P—i.e., P⊆Q. To enforce non-negativity we restrict Q to only constain probes p such that F({p})≥λ·(m̅ₚ̅−mₚ). In other words, every probe has to be sufficientiy good. In practice, given our activity function, λ∈[0.1, 0.51] is a reasonable choice and the constraint on F({p}) is generally met; for example with λ=0.25, m̅ₚ=5, and mₚ=1, then we require F({p})≥1.

Solving for P

Recall we want to solve

$${max}_{P} \{ \tilde{F} ( P ) \text{:} \vert P \vert \leq \overline{{m}_{p}} \}$$

where \tilde{F}(P)=F(P)−λmax(0, |P|−mₚ). We need to maximize a non-negative and non-monotone submodular function subject a cardinality constraint. The classical discrete greedy

algorithm36 can provide poor results because it assumes a monotone function. Applicants apply the recently developed discrete randomized greedy algorithms in ref. 35, namely Algorithm 1, which provides a 1/e-approximation for non-monotone functions. (Algorithm 5, which provides a better approximation ratio, is likely to not be much better in Applicants' case because the constraint mp is small compared to the size of the ground set.)

Based on the work in ref 35, the function Determine-Probe-Set (Algorithm 1) shows how P was computed to detect a particular genomic window of an alignment S. Applicants use locality-sensitive hashing to rapidly cluster potential probe sequences2 throughout the window, and these form the ground set Q of probes (line 3). Then, Applicants require that probes in Q be specific to the taxon to which S belongs, using the methods in Supplementary Note 2d). Applicants add to the ground set “dummy” elements that provide a marginal contribution of 0 to any set input to Fe (line 6), as required by an assumption of the algorithm (Reduction 1 in ref. 35). Then, Applicants greedily choose_mp probes, at each iteration selecting one randomly from a set of not-yet-chosen probes that maximize marginal contributions to Fe (lines 10-11).

Algorithm 1
Construct set of probes P to maximize \tilde{F}(P) subject to hard constraint.

Input

| | |
|---|---|
| T | alignment of sequences extracted from a widow of S, from taxon t |
| l_{ε} | probe length |
| F | function of probe set, including soft constraint |
| m̅ₚ̅ | hard constraint on number of probes |

Output

| | |
|---|---|
| P | collection of probes |

```
  1  function DETERMINE-PROBE-SET (T, lₚ, \tilde{F}, m̅ₚ̅)
  2  C ← clusters of lₚ-mers at and across each postition of T
  3  Q ← representative of each cluster in C [symbol]    ground set
  4  Q ← Q\ {lₚ-mers in Q not meeting non-negativity constraint }
  5  Q ← Q\ {lₚ-mers in Q not specific to t} [symbol]    enforce specificity
  6  Q ← Q ∪ {2 · m̅ₚ̅ “dummy” element that contribute 0 to \tilde{F}}
  7
  8  P ← { }
  9  for j ← 1 to m̅ₚ̅do
10  Mⱼ ← m̅ₚ̅ elements from Q \ P that maximize ΣuϵMj (\tilde{F}(P ∪{u}) − \tilde{F}(P))
11  _{p*} ← elements from Mⱼ chosen uniformly at random
12  P ← P ∪ {p*}
13  P ← P \ {“dummy” elements}
14  return P
```

The runtime to design probes is practical in the typiczd case. Here we ignore the runtime of evaluating specificity (line 5), which is given in 2d. Let L be the window length and n be the number of sequences. There are O(nL) probes in the gronnd set in the worst-case, and they taken O(nL) time to construct (line 3). Finding the m̅ₚ̅ elements that maximize marginal contributions (line 10) takes O(nL) time, and we do this O(m̅ₚ) times. Thus, the runtime in the worst-case is O(nLm̅ₚ). In a typical case, the number of clusters at a position in the window is a small constant (≤n) owing to sequence homology in the alignimmt; thus, the size of the gronnd set is O(L), although it still takes O(nL) time to construct. Now finding the m̅ₚ elements that maximize marginal contributions takes O(L) time, and we do this O(m̅ₚ) times. So the runtime in a typical case is O(nL+Lm̅ₚ). Note that, in general, m̅ₚ≤L and m̅ₚ≤n.

1b Design formulation #2: minimizing the number of probes

Objective

As in the above objective, let S be an alignment of sequences from species t in a genomic region and let d(p,s) be a predicted detection aetivity between one probe p and one sequence s∈S. We wish to find a set P of the proves with minimal |P| that satisfies constraints on detection activity across these sequences. In particular, we introduce a fixed detection activity m_{d} and say that p is highly active in detecting s if d(p,s)≥m_{d}. To define whether P detects a sequence with high activity, let

$$d ( P , s ) = \{ \begin{matrix}1 & if \vert \{ p \text{:} p ∈ P , d ( p , s ) \geq {m}_{d} \} \vert \geq 1 \\ 0 & otherwise\end{matrix}$$

We additionally introduce a lower bound fₛ on the minimal fraction of sequences in S that must be detected with high activity. Then, we wish solve

$${min}_{P} \{ \vert P \vert \text{:} \vert \{ s \text{:} s ∈ S , d ( P , s ) = 1 \} \vert / \vert S \vert \geq {f}_{S} \} .$$

That is, we want to find the smallest probe set that detects, with high predicted activity, at least a fraction f_{S} of all sequences.

### Solving for P

To approximate the optimal P, ADAPT follows the canonical greedy solution to the set cover problem63, 64 in which the universe consists of the sequences in S and each possible probe covers a subset of sequences in S. Similar approaches have been used for PCR primer selection25, 28, 33, 65, 66; in contrast to prior approaches, rather than starting with a collection of candidate probes (i.e., the sets), Applicants construct them on-the-fly.

Iteratively, Applicants approximate a probes that covers the most number of sequences that still need to be covered. Here, a probe p covers a sequence s if d(p, s)_md. Find-Optimal-Probe, shown in Algorithm 2, implements a heuristic. Briefly, at each position Find-Optimal-Probe

rapidly clusters k-mers in the input sequences (k is the probe length) by sampling nucleotides—i.e., concatenating locality-sensitive hash functions drawn from a Hamming distance family—and uses each of these clusters to propose a probe. It iterates through the clusters in decreasing order of score, stopping early (line 11) if it is unlikely that remaining clusters will provide a probe that achieves more coverage than the current best. This procedure relies on two subroutines, Score-Cluster and Num-Detect, that are described below.

Using this procedure, it is straightforward to construct a set of probes in the window (region) that achieve the desired coverage by repeatedly calling Find-Optimal-Probe. This is shown concretely

Algorithm 2
Construct probe p* with highest coverage

Input

| | |
|---|---|
| U | sequences in S to cover, from taxon t |
| k | probe length |

Output

| | |
|---|---|
| p* | probe in window |

```
  1 function FIND-OPTIMAL-PROBE(U, K)
  2  Initialize p*
  3  for each length k sub-window ω in U do
  4  clusts ← Cluster all k-mers of U in ω
  5  clusts ← Sort clusts, desceding, according to SCORE-CLUSTER(clust)
  6  repeat
  7  p ← Consensus of k-mers in next best cluster in clusts
  8  if p is specfic to taxon t then [symbol]   enforce specificity
  9  if NUM-DETECT(p, U) > NUM-DETECT(p*, U) then
10  p* ← p
11  until early stopping criterion is met
12  return p*
```

by DETERMINE-PROBE-SET, in ALgorithm 3. In other words, the output probes collectively detect, with high activity, the sequences in the region.

Algorithm 3
Construct minimal collection of probes in window that collectively
achieve desired detection coverage

Input

| | |
|---|---|
| T | alignment of sequences extracted from a |
| | window of S, from taxon t |
| k | probe length |
| fs | fraction of sequences in T to detect |

Output

| | |
|---|---|
| C | collection of probes |

1 function DETERMINE-PROBE-SET(T, k, fs)
2 C ← { }
3 while |{s : s ϵ T, d(C,s) = 1}|/|T| < fs do
4 U ← Sequences s ϵ T such that d(C,s) = 0
5 p* ← FIND-OPTIMAL-PROBE(U,k)
6 C ← C ∪ {p*}
7 return C

This approach, with on-the-fly construction of probes, is similar to a reduction to an instance of the set cover problem, the solution to which is essentially the best achievable approximation67, 68. In such a reduction, each set would represent one of the 4k possible probes, consisting of the sequences that it would detect with high activity. Then, each iteration would identify the probe that detects, with high activity, the most not-yet-covered sequences. Here, rather than starting with such a large space, Applicants use a heuristic to approximate the probe at each iteration.

The runtime to design probes in a window is practical in the typical case. Let n be the number of sequences in the alignment and L be length of the window. In the worst-case, Applicants choose n different probes in the window. Each choice requires iterating over O(L) positions, and at each one Applicants iterate through O(n) clusters, taking O(n) time to evaluate the probe proposed by each cluster with Num-Detect. Thus, this is O(n3L) time. In a typical case, there are a small number of clusters owing to sequence homology across the alignment, and the number of probes needed to achieve the constraint is also a small constant. Selecting each probes requires iterating over O(L) positions, and at each one Applicants consider O(L) clusters, taking O(n) time again to evaluate the probe proposed. So the runtime is O(nL) with these assumptions.

### Scoring Clusters and Detection Across Sequences

Sequences from S can be grouped according to metadata such that each group receives a particular desired coverage (fSg). For example, in ADAPT they can be grouped according to year (each group contains sequences from one year), with a desired coverage that decays for each year going back in time, so that ADAPT weights more recent sequences more heavily in the design.

There are two subroutines in Algorithm 2 that Applicants consider here: scoring a cluster and computing he number of sequences detected by a probe. These must account for groupings. First, on line 5 of Find-Optimal-Probe, the function Score-Cluster(clust) computes the number of sequences dust contains that are needed to achieve the desired coverage across all the groups. That is, it calculates

$${\sum }_{x ∈ X} min ( {n}_{x} , \vert ⋂ {U}_{x} \vert )$$

where X is the collection of sequence groups, nx is the number of sequences from group x that must still be covered to achieve x′s desired coverage, dust gives the sequences of U from which the k-mers in dust originated, and Ux consists of the sequences in U that are in group x. In essence, it computes a contribution of each cluster toward achieving the needed coverage of each group, summed over the groups. Similarly, on line 9 of Find-Optimal-Probe, the function Num-Detect(p, U) is the etection coverage provided by probe p across the groups. In particular, its value is

$${\sum }_{x ∈ X} min ( {n}_{x} , \vert B ⋂ {U}_{x} \vert )$$

where B is the set of sequences U that p covers—i.e., B={s: s ∈U, d(p,s)≥m_{d}}.

These subroutines are intuitive in the case where sequences are not grouped. Equivalently, consider a single group x₀. Here SCORE-CLUSTER(clust) is min(nₓ_{₀}. [symbol] ∩Uₓ_{₀}|). Since [symbol] ⊥Uₓ_{₀}=U, this is min(nₓ_{₀}, [symbol] ). Thus, the score is simply the size of the cluster (larger clusters are preferred), or nₓ_{₀} for clusters large enough so as to provide more than sufficient coverage. Similarly, NUM-DETECT(g, U) is min(nₓ_{₀}, |B∩Uₓ_{₀}|). Because B⊆Uₓ_{₀}=U, this is min(nₓ_{₀}, |B|). So NUM-DETECT is effectively the number of sequences covered by that must still be covered to achieve the coverage constraint.

Furthermore, if sequences are grouped, note that line 3 of Algorithm 3 instead iterates until achieving the desired coverage for each group.

A recent paper69 on submodular optimization looks at a similar problem; it refers to the groupings in this problem as “ground sets,” and provides an approximation ratio given by the greedy algorithm.

### Supplementary Note 2

This note describes an overview of the challenge of evaluating specificity and two formulations, implemented in ADAPT, for doing so. Unless otherwise noted, for designs and analyses in the paper, Applicants use the formulation in Exact trie-based search for probe near neighbors.

### 2a Overview

In applications where differentially identifying a taxonomy is important, ADAPT ensures that the probes it constructs are specific to the taxonomy they are designed to detect. In general, the probes directly perform detection; thus, their specificity is ADAPT's focus, rather than other aspects of a design, such as primers.

The framework for this is as follows. Initially, ADAPT constructs an index of probes across all input taxonomies, which includes the taxonomies and particular sequences containing each probes. This index could also include background sequence to avoid, such as the human transcriptome, although Applicants generally do not include non-viral background sequence. Then, when designing a probe for a taxonomy ti with genomes Si, ADAPT queries this index to determine its specificity against all sequences from any Sj for j 6=i. The results inform whether the probe might detect some fraction of sequence diversity from some other taxon. ADAPT performs this query while constructing the ground set, as described in Supplementary Note 1.

This problem is computationally challenging. When querying, Applicants generally wish to tolerate a high divergence within a relatively short query to be conservative in finding potential non-specific hits—e.g., up to _5 mismatches within 28 nt. Also, G-U wobble base pairing (described below) generalizes the usual alphabet of matching nucleotides. Together, these challenges mean that popular existing approaches, including seed/MEM techniques, are not fully adequate for performing queries.

### 2b G-U Wobble Base Pairing

Some detection applications (e.g., CRISPR-Cas13) rely on RNA-RNA binding. That is, the probe Applicants design is synthesized as RNA and the target is RNA as well. RNA-RNA base pairing allows for more pairing possibilities than with DNA-DNA. In particular, G may bind with U, forming a G-U wobble base pair. It has similar thermodynamic stability to the usual Watson-Crick base pairs70. Its effect on an enzymatic process may differ from other base pairs, but in some of ADAPT's applications it is comparable to Watson-Crick base pairs.

In ADAPT, Applicants wish to treat G-U base pairs as matching when querying for a probe's specificity. For simplicity, here Applicants will use T instead of U (the RNA nucleobase U replaces the DNA nucleobase T), and thus Applicants consider G-T base pairing. In particular, Applicants consider a base g[i] in a probe to match a base s[i] in a target sequence if either (a) g[i]=s[i], (b) g[i]=A and s[i]=G, or (c) g[i]=C and s[i]=T3. Note that activity models in ADAPT that are trained for a particular assay technology can prune the query results if the effect is different in some application.

Tolerating G-U base pairing considerably complicates the problem for several reasons. The addition f G-U base pairing raises the probability of a matching hit between a 28-mer and an arbitrary target, thereby expanding the space of potential query results. It also means the Hamming distance between a query and valid hit (considered in the same frame) can often exceed 50% and be as high as 100%. FIG. 53 illustrates the challenge in practice on viral genome data. A similar challenge arises in determining off-target effects when designing small interfering RNA (siRNA)71, 72. It is common to ignore the problem (e.g., using BLAST to query for off-targets)73-76. Other approaches do address it. One is to treat G-U pairs like a mismatch, albeit not as heavily enalized as a Watson-Crick mismatch77; however, with this approach, searching for candidate hits may fail to find valid hits if the Hamming distance between the query and hit is sufficiently high owing to G-U pairs. Another approach uses the seed-and-extend technique where the seed is in a well-defined “seed region” that requires an exact match, tolerating G-U pairs in the seed78; although applicable to siRNA, a seed-based approach may fail to generalize if there is no seed region, if it is too short, or if it is not consistent or is tolerant of mismatches. For some RNA interference applications, G-U pairs may be detrimental to the activity of an enzyme complex79, and therefore it may not be necessary to fully account for it when determining specificity. None of these approaches are fully satisfying in ADAPT. To approach the challenge of G-U wobble base pairing, at several points in the algorithms below using a transformed sequence (FIG. 54a). Applicants transform a probe g into g0 by changing A to G and changing C to T; in g0, the only bases are G and T. Likewise, Applicants do this for a target sequence s. This is useful because any G-T matching between s and the complement of g is not reflected by different letters between g0 and s0—i.e., if the reverse complement of g (what Applicants synthesize) matches with s up to G-U base pairing, then g0 and s0 are equal strings.

### 2c Probabilistic Search for Probe Near Neighbors

To permit queries for specificity, Applicants first experimented with performing an approximate near neighbor lookup similar to the description in ref 80 for points under the Hamming distance. Here, Applicants wish to find probes that are _m mismatches from a query.

The approach precomputes a data structure H={H₁, H₂. . . H_{L}} where each Hₛ is a hash table that has a corresponding locality-sensitive hash function hᵢ, which samples b positions of a probe. The h₂s bear siomilarity to the concept of spaced seeds^{/}. It chooses L to achieve a desired reporting probability r:

|L=[log_{1 . . . p6}(1−r)],

where P^{b}=(1−m/k)^{b} is a lower bound on the probability of (for a single hᵢ) for nearby probes. In ADAPT, we have used r=0.95 and b=22. For all probes g across all sequences in all taxa tⱼ, each Hᵢ[hᵢ(g′)] stores {(g,j)} where j is an identifier of a taxon from which g arises and g′ is g in the two-letter alhabet described above. Additionally, data structure holds a hash table G where G[(g,j)] sotres identifiers of the sequencesin j that contain g. From these data structures, queries are straightforward. For a probe q to query, the query algorithm looks up q′ in each Hₛ and check if it detects (is within m mismatches) each resulting g. For the ones that it does detect, G provides the fraetion of sequences in each taxon containing g and therefore provides the fraction of sequences in each taxon that q detects. The algorithm deems q specific iff this fraction is sufficiently

small. Note that, when designing probes for a taxon tj, it is straightforward to mask j from each Hi; this is important for query runtime because most near neighbors would be from j.

This approach would be suitable if Applicants were to not have to consider G-U base pairing, but this consideration makes it too slow for many applications. To accommodate G-U base pairs, it stores wo-letter transformed probes (g0) and likewise queries transformed probes (q0). The dimensionality reduction enables finding hits within _m mismatches of a query q, sensitive to G-U base pairs, but t also means that most results in each Hi[hi(q0)] are far from q. As a result, the algorithm spends ost of its time validating each of these results by comparing it to q. A higher choice of b can counteract this issue, but results in higher L and thus requires more memory. Also, the approach is probabilistic and may fail to detect non-specificity; while the reporting probability might be high per-taxon, if Applicants use ADAPT to design across many taxonomies it becomes more likely to output a non-specific assay. Thus, below, Applicants develop an alternative approach that is more tailored to the particular challenges Applicants face.

### 2d Exact Trie-Based Search for Probe Near Neighbors

Here Applicants describe a data structure and query algorithm that permits fully accurate queries for non-specific hits of a probe. Unlike the probabilistic approach above, this will always detect nonspecificity if present, and Applicants show it is fast compared to a baseline. Having one trie containing all he indexed probes would satisfy the goal of being fully accurate because Applicants could branch, during a query, for mismatches and G-U base pairs; however, the extensive branching involved means that query time would depend on the size of the trie and may be slow. To alleviate this, Applicants place (or shard) the probes across many smaller tries.

Briefly, the data structure stores an index of all probes across the input sequences from all taxa. Let k be the probe length (e.g., 28). The data structure splits each probe into p partitions (without loss of generality, assume p divides k). Each partition maps to a k p -bit signature such that any two matching strings map to the same signature, tolerating G-U base pairing; each bit corresponds to a letter from the two-letter alphabet described in G-U wobble base pairing. There are p·2k/p tries in total, each associated with a signature and a partition, and every probe is inserted into p tries according to the signatures of its p partitions.

To query a probe q, the algorithm relies on the pigeonhole principle; tolerating up to m mismatches across all of q, there will be at least one partition with ≤[m/p] mismatches against each vaiid hit. For each partition of q, the query algorithm produces all combinations of signatures within [m/p] mismatches—there are

$${\sum }_{i = 0}^{⌊ m / p ⌋} ( \begin{matrix}k / p \\ i\end{matrix} )$$

of them—and looks up q in the tries with these signatures for the partition. During each lookup, it branches to accommodate G-U base pairing and up to m mismatches. Note that the bit signatiure is sensitive to G-U base pairing—i.e., two positions have the same bit if they might be a match, including owing to G-U pairing—so the algorithm finds all hits, even if the query and hit strings diverge due a G-U pairing.

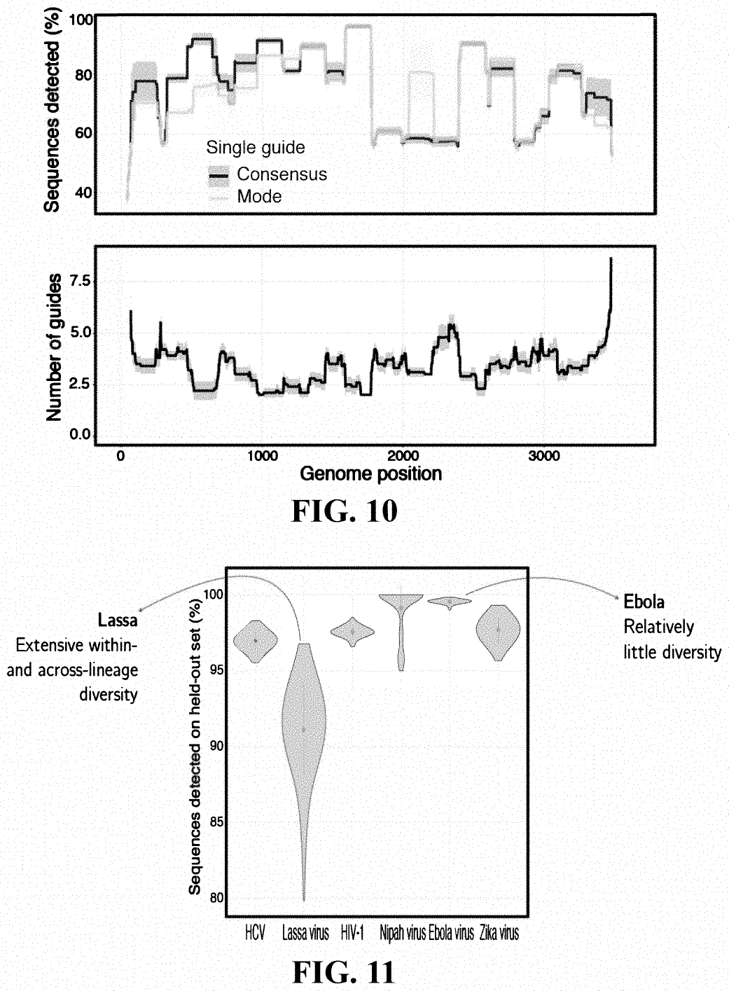

Supplementary FIG. 10 provides a visual depiction of building the data structure and performing queries and Algorithms 4 and 5 provide pseudocode.

A loose bound on the runtime of a query is

$$O ( p \cdot \frac{n}{{2}^{k / p}} \cdot {\sum }_{i = 0}^{⌊ m / p ⌋} ( \begin{matrix}k / p \\ i\end{matrix} ) )$$

Algorithm 4
Build data structure of tries to support specificity queries.

Input

| | |
|---|---|
| {S} | collection of sequences across taxonomies |
| k | probe length |
| p | number of partitions |

Output

| | |
|---|---|
| [symbol] | space of tries indexing probes |

```
  1 function BUILD-TRIES({S}, k, p)
  2  Initialize [symbol] [symbol]   contains p · 2^{k/p} tries, one per pair of partition and bit vector
  3  for each taxonomy tᵢ do
  4  sᵢ ← Sequences for tᵢ
  5  for each probe g in Sᵢ do
  6  for r = 1 to p do
  7  gᵣ ← Partition r of g
  8  g′ᵣ ← Hash of gr: A → 0, G → 0, C → 1, T → 1 [symbol]    bit vector
  9  T ← Trie in [symbol] corresponding to partition r and bit vector g′ᵣ
10  Insert g into T [symbol]    include tᵢ and sequence identifier in leaf node
11  return [symbol]
```

Algorithm 5
Query tries to find non-specific hits

Input

| | |
|---|---|
| q | probe to query for specificity to taxon tᵢ |
| m | number of mismatched to tolerate |
| p | number of partitions |
| Requires: | [symbol] from BUILD-TRIES |
| requires: | taxon tᵢ is masked from [symbol] |

Output

| | |
|---|---|
| G | taxon/sequence identifiers of non-specific hits |

```
  1 function QUERY(q, m, p)
  2  Initialize set G
  3  for r = 1 to p do
  4  qᵣ ← Partition r of q
  5  q′ᵣ ← Hash of qᵣ: A → 0, G → 0, C → 1, T → 1 [symbol]    bit vector
  6  for each variant (q′ᵣ)′ of q′ᵣ with ≤ └m/p┘ flipped bits do
  7  T ← Trie in [symbol]  corresponding to partition r and bit vector (q′ᵣ)′
  8  g ← Query results for q in T, branching always for G-U
  9  pairing and for up to m mismatches
10  Add g to G
11  return G
```

where n be the total number of probes indexed in the data structure. The query algorithm performs a search for p partitions of a query q, For each partition, it considers

$${\sum }_{i = 0}^{⌊ m / p ⌋} ( \begin{matrix}k / p \\ i\end{matrix} )$$

tries, one for each combination of [m/p] bit flips. The size of each trie is a loose upper bound on the query time within it; assuming uniform sharding, the size of each is

$$O ( \frac{n}{{2}^{k / p}} ) .$$

Multiplying the size of each trie by the number of them considered during a query provides the stated runtime. Adjusting p, a small constant, allows us to tune the runtime, higher choices reduce the number of bit flips, and thus the number of tries to search, but yield larger tries and thus requires more time searching within each of them. The runtime does not scale well with our choice of m, but this is generally a small constant (up to ˜5). Because the data structure stores each probe in p seperate tries, the required memory is O(np). Although this scales reasonably with n, it involves large constant factors and is memory-intensive in practive; one future direction would be to comprises the tries.

### Supplementary Note 3

This note describes how Applicants link methods, from Supplementary Notes 1 and 2, in ADAPT to form an end-to-end system for designing assays. In particular, this involves searching across genomic regions and connecting with publicly available genome databases.

### 3a Branch and Bound Search for Genomic Regions

In many diagnostic applications, Applicants must amplify a genomic region to obtain enough material for etection. The probes in a probe set P ought to be within a genomic region of the alignment S that s bound by conserved sequence so that Applicants can design primers to amplify the region; as with probes, Applicants want to penalize the number of primers because they can interfere with each other or require multiple reactions. Similarly, Applicants wish to penalize the length of the region because longer regions are less efficient to amplify; penalizing the logarithm of length approximates the length-dependence of amplification efficiency. When first walk through the search using the objective that maximizes expected activity (Supplementary Note 1a). For this, Applicants now perform a search for a genomic region R that encompasses P and solve

$${max}_{P , R} \{ \tilde{F} ( P ) - {λ}_{A} \vert {R}_{A} \vert - {λ}_{L} log ( {R}_{L} ) \text{:} \vert P \vert \leq \overline{{m}_{p}} \}$$

where \tilde{F}(P) defined in Supplementary Note 1a, R_{A} gives the set of primers bounding the region, R_{L} gives the mucleotide length of the region, aud λ_{A} and λ_{L} give weights on the penalties. Note that λ_{A} and λ_{L} can optionally be set to 0, removing the requirement that a region be bound by conserved seguence and represent an amplicon.

To solve this, we use algorithm in which we search over options for R and prune unnecessary ones. Rather than finding a single maximum, we wish to compute the highest N solutions—i.e., N regions, each containing a probe set—to the objective. This is important so that milltiple design options can be tested and compared experimentally. Note also that the value of \tilde{F} has an upper-bound, which we call \tilde{F}ₕ₁, calculated from F(P)'s highest value (predicted activities are bounded): and |P|=1.

Applicants maintain a min heap h of the N designs with the highest value of the objective. First, at every position of the alignment S, Applicants approximate a minimal set of primers that achieves a desired coverage over the genomes; Applicants use Algorithm 3 in Supplementary Note 1, except parameterized for primers. Then, Applicants search over pairs of positions in the alignment, considering the regions that would be bound by primers at each pair. Although the number of such regions is quadratic in the alignment length, Applicants can effectively prune regions based on IRA1 and RL. Applicants calculate, with these values, the objective value using Fehi in place of Fe(P); if this value falls below that of the minimum in h, the region cannot be in the top N. For regions that could be in the best N, Applicants approximate the probe set P with the maximal Fe(P) as described in Supplementary Note la) (Solving for P).

If the objective value for the design given by (R, P) is greater than the minimum in h, Applicants pop from h and push the design to it. This search is guaranteed to identify the top N regions according to the objective, up to Applicants' approximation of Fe(P). Applicants typically add a fixed constant (4) to the objective values before reporting them to the user, which Applicants find makes the values more interpretable to users because it makes them more likely to be non-negative. This shift has no impact on the design options or their rankings.

This search follows the branch and bound paradigm in which the candidate solutions (R, P) make up a 3-level tree, excluding the root. The levels represent the left primers, right primers, and the probe set. Applicants can prune the choice of right primers based on the length of the amplicon they would form. Exploring the final level in particular—determining the probe set—is the slow step. Since an upper bound can be quickly constructed on the candidate solution for nodes in the final level, which Applicants compare to the minimum in h, nodes can be discarded and thus one can avoid having to compute P for many candidate solutions.

It is also important that the design options are diverse, i.e., reflect meaningfully different regions rather than being simple shifts of one another. To account for this, Applicants implement the following: if a design to push to h has a region overlapping that of an existing design in h, it must replace that existing design (and only does so if the new one has a higher objective value). dditionally, during Applicants'search many of the computations—particularly when computing probe sets—would be performed repeatedly from the same input, owing to overlap between different regions across the search. As a result, Applicants memoize results of these probe set computations according to genome position. A branch and bound implementation, as described above, might start with all the left primers (first level) and then select the best 2nd primers (second level), before advancing to computing probe sets. However, this could force each successive probe set computation to jump to different region in the genome, according to the primer pairs: Applicants would not be able to efficiently cleanup memoizations for these computations and memory would grow throughout the search. To avoid this issue, Applicants scan linearly along the genome and, each time Applicants advance the left primer position, can determine probe set memoizations that Applicants no longer need to store. The above description applies to maximizing expected activity, but it is straightforward to adjust the strategy when minimizing the number of guides (Supplementary Note lb). In this case, the objective is changed to solve for

$${max}_{P , R} \{ ( P ) + {λ}_{A} \vert {R}_{A} \vert + {λ}_{L} log ( {R}_{L} ) \}$$

where Applicants also impose the constraint on coverage described in Supplementary Note 1b. The search now stores a max heap h of the designs with the smallest values of the objective. For pruning, compute a lower bound on the candidate solution by letting |P|=1, and compare this bound to the maximum in h.

The search is embarrassingly parallel (FIG. 59). One future direction is to parallelize the search across genomic egions, in which Applicants perform it separately for contiguous parts of the genome and then merge the resulting heaps. The primary challenge is likely to be handling shared memory, in particular for the large index used to enforce specificity.

### 3b Collecting Sequences to Target

ADAPT accepts a collection of taxonomies provided by a user: {t1, t2, . . . }. It can either design for one ti or for all ti, in either case ensuring designs are specific accounting for all tj where j 6=i. Each ti generally represents a species, but can also be a higher-level classification or a subtype5. In NCBI's databases, each taxonomy has a unique identifier54 and ADAPT accepts these identifiers. ADAPT then downloads all near-complete and complete genomes for each ti from NCBI's genome neighbors database, but uses its Influenza Virus Resource database55 for influenza viruses. It also fetches metadata for these genomes (e.g., date of sample collection), which some downstream design tasks process.

Applicants must then prepare these genomes for design. Briefly, for each ti Applicants curate the genomes by aligning each one to one or more reference sequences6 for ti and remove genomes that align very poorly to all references, as measured by several heuristics: by default, Applicants remove a genome that as <50% identity to all references or that have <60% identity to all references after collapsing consecutive gaps to a single gap. This process prunes genomes that are misclassified, have genes entered in an atypical sense, or are highly divergent for some other reason. Then, Applicants cluster the genomes for ti with an alignment-free approach by computing a MinHash signature for each enome, rapidly estimating pairwise distances from these signatures (namely, the Mash distance61), and performing hierarchical clustering using the distance matrix. The default maximum intercluster distance (approximate average nucleotide dissimilarity) for clustering is 20%. In general, pplicants have a single cluster for a species. This provides another curation mechanism, because it can discard clusters that are too small (by default, just one sequence). Finally, ADAPT aligns the genomes within each cluster using MAFFT82. This yields a collection of alignments, where each is for a cluster of genomes from taxon ti.

Many of these computations—such as curation, clustering, and alignment—are slow and might be repeated on successive runs. ADAPT memoizes results of the above computations to reuse on future runs when the input permits it. Memoization considerably improves runtime for routine use of ADAPT.

One technicality: many species have segmented genomes. For these, ADAPT also needs the label of the segment. ADAPT effectively treats each segment as a separate taxonomy—i.e., for species that are segmented, the tis are actually pairs of taxonomic ID and segment.

The “reference” sequences are determined by NCBI, but can also be provided by the user. They are manually curated, high-quality genomes and encompass important strains. References

[1] Bedford, T. et al. Integrating influenza antigenic dynamics with molecular evolution. eLife 3, e01914 (2014).

[2] Zhou, P. et al. A pneumonia outbreak associated with a new coronavirus of probable bat origin. Nature (2020).

[3] Metsky, H. C. et al. Zika virus evolution and spread in the americas. Nature 546, 411-415 (2017).

[4] Roux, S. et al. Minimum information about an uncultivated virus genome (MIUViG). Nature biotechnology 37,29-37 (2019).

[5] Brister, J. R., Ako-Adjei, D., Bao, Y. & Blinkova, O. NCBI viral genomes resource. Nucleic acids research 43, D571-7 (2015).

[6] Shu, Y. & McCauley, J. GISAID: Global initiative on sharing all influenza data—from vision to reality. Euro surveillance: bulletin Europeen sur les maladies transmissibles =European communicable disease bulletin 22 (2017).

[7] Stellrecht, K. A. The drift in molecular testing for influenza: Mutations affecting assay performance. Journal of clinical microbiology 56 (2018).

[8] Overmeire, Y. et al. Severe sensitivity loss in an influenza a molecular assay due to antigenic drift variants during the 2014/15 influenza season. Diagnostic microbiology and infectious disease 85,42-46 (2016).

[9] Klungthong, C. et al. The impact of primer and probe-template mismatches on the sensitivity of pandemic influenza A/H1N1/2009 virus detection by real-time RT-PCR. Journal of clinical virology: the official publication of the Pan American Society for Clinical Virology 48, 91-95 (2010).

[10] Brault, A. C., Fang, Y., Dannen, M., Anishchenko, M. & Reisen, W. K. A naturally occurring mutation within the probe-binding region compromises a molecular-based west nile virus surveillance assay for mosquito pools (diptera: Culicidae). Journal of medical entomology 49,939-941 (2012).

[11] Lee, H. K. et al. Missed diagnosis of influenza B virus due to nucleoprotein sequence mutations, singapore, april 2011. Euro surveillance: bulletin Europeen sur les maladies transmissibles =European communicable disease bulletin 16 (2011).

[12] Cattoli, G. et al. False-negative results of a validated real-time PCR protocol for diagnosis of newcastle disease due to genetic variability of the matrix gene. Journal of clinical microbiology 47,3791-3792 (2009).

[13] Lengerova, M. et al. Real-time PCR diagnostics failure caused by nucleotide variability within exon 4 of the human cytomegalovirus major immediate-early gene. Journal of clinical microbiology 45,1042-1044 (2007).

[14] Stevenson, J., Hymas, W. & Hillyard, D. Effect of sequence polymorphisms on performance of two real-time PCR assays for detection of herpes simplex virus. Journal of clinical microbiology 43,2391-2398 (2005).

[15] Barrat-Charlaix, P., Huddleston, J., Bedford, T. & Neher, R. A. Limited predictability of amino acid substitutions in seasonal influenza viruses (2020).

[16] Gootenberg, J. S. et al. Nucleic acid detection with CRISPR-Cas13a/C2c2. Science 356,438-442 (2017).

[17] Gootenberg, J. S. et al. Multiplexed and portable nucleic acid detection platform with cas13, cas12a, and csm6. Science 360,439-444 (2018).

[18] Myhrvold, C. et al. Field-deployable viral diagnostics using CRISPR-Cas13. Science 360,444-448 (2018).

[19] Chen, J. S. et al. CRISPR-Cas12a target binding unleashes indiscriminate single-stranded DNase activity. Science 360,436-439 (2018).

[20] Pardee, K. et al. Paper-based synthetic gene networks. Cell 159,940-954 (2014).

[21] Pardee, K. et al. Rapid, Low-Cost detection of zika virus using programmable biomolecular components. Cell 165,1255-1266 (2016).

[22] Chiu, C. Cutting-Edge infectious disease diagnostics with CRISPR. Cell host & microbe 23,702-704 (2018).

[23] Linhart, C. & Shamir, R. The degenerate primer design problem. Bioinformatics 18 Suppl 1, S172-81 (2002).

[24] Fitch, J. P. et al. Rapid development of nucleic acid diagnostics. Proceedings of the IEEE 90, 1708-1721 (2002).

[25] Jabado, O. J. et al. Greene SCPrimer: a rapid comprehensive tool for designing degenerate primers from multiple sequence alignments. Nucleic acids research 34, 6605-6611 (2006).

[26] Vijaya Satya, R., Kumar, K., Zavaljevski, N. & Reifman, J. A high-throughput pipeline for the design of real-time PCR signatures. BMC bioinformatics 11, 340 (2010).

[27] Karim, S. et al. Development of the automated primer design workflow uniqprimer and diagnostic primers for the Broad-Host-Range plant pathogen dickeya dianthicola. Plant disease 103, 2893-2902 (2019).

[28] Duitama, J. et al. PrimerHunter: a primer design tool for PCR-based virus subtype identification. Nucleic acids research 37, 2483-2492 (2009).

[29] Zheng, J. et al. OligoSpawn: a software tool for the design of overgo probes from large unigene datasets. BMC bioinformatics 7, 7 (2006).

[30] Brodin, J. et al. A multiple-alignment based primer design algorithm for genetically highly variable DNA targets. BMC bioinformatics 14, 255 (2013).

[31] Wright, E. S. & Vetsigian, K. H. DesignSignatures: a tool for designing primers that yields amplicons with distinct signatures. Bioinformatics 32, 1565-1567 (2016).

[32] Wright, E. S. et al. Exploiting extension bias in polymerase chain reaction to improve primer specificity in ensembles of nearly identical DNA templates. Environmental microbiology 16, 1354-1365 (2014).

[33] Kreer, C. et al. openPrimeR for multiplex amplification of highly diverse templates. Journal of immunological methods 480, 112752 (2020).

[34] Indyk, P. & Motwani, R. Approximate nearest neighbors: Towards removing the curse of dimensionality. In Proceedings of the Thirtieth Annual ACM Symposium on Theory of Computing, STOC '98, 604-613 (ACM, New York, NY, USA, 1998).

[35] Buchbinder, N., Feldman, M., Naor, J. s. & Schwartz, R. Submodular maximization with cardinality constraints. In Proceedings of the twenty-fifth annual ACM-SIAM symposium on Discrete algorithms, SODA '14, 1433-1452 (Society for Industrial and Applied Mathematics, SA, 2014).

[36] Nemhauser, G. L., Wolsey, L. A. & Fisher, M. L. An analysis of approximations for maximizing submodular set functions. Mathematical Programming. A Publication of the Mathematical Programming Society 14, 265-294 (1978).

[37] Abudayyeh, O. O. et al. C2c2 is a single-component programmable RNA-guided RNA-targeting CRISPR effector. Science 353, aaf5573 (2016).

[38] Abudayyeh, O. O. et al. RNA targeting with CRISPR-Cas13. Nature 550, 280-284 (2017).

[39] Tambe, A., East-Seletsky, A., Knott, G. J., Doudna, J. A. & O'Connell, M. R. RNA binding and HEPN-Nuclease activation are decoupled in CRISPR-Cas13a. Cell reports 24, 1025-1036 (2018).

[40] Ackerman, C. M. et al. Massively multiplexed nucleic acid detection using cas13. Nature 2020).

[41] Kim, H. K. et al. Deep learning improves prediction of CRISPR-Cpf1 guide RNA activity. Nature biotechnology 36, 239-241 (2018).

[42] Wessels, H.-H. et al. Massively parallel cas13 screens reveal principles for guide RNA design. Nature biotechnology 38, 722-727 (2020).

[43] Tavar'e, S. Some probabilistic and statistical problems in the analysis of DNA sequences. Lectures on mathematics in the life sciences (1986).

[44] Lam, T. T.-Y. et al. Identifying SARS-CoV-2-related coronaviruses in malayan pangolins. Nature (2020).

[45] Broughton, J. P. et al. CRISPR-Cas12-based detection of SARS-CoV-2. Nature biotechnology (2020).

[46] Barnes, K. G. et al. Deployable CRISPR-Cas13a diagnostic tools to detect and report ebola and lassa virus cases in real-time (2020).

[47] Metsky, H. C., Freije, C. A., Kosoko-Thoroddsen, T.-S. F., Sabeti, P. C. & Myhrvold, C. CRISPR-based surveillance for COVID-19 using genomically-comprehensive machine learning design (2020).

[48] U.S. Food and Drug Administration. Infectious disease next generation sequencing based diagnostic devices. Tech. Rep. (2016).

[49] Kugelman, J. R. et al. Evaluation of the potential impact of ebola virus genomic drift on the efficacy of sequence-based candidate therapeutics. mBio 6 (2015).

[50] Freije, C. A. et al. Programmable inhibition and detection of RNA viruses using cas13. Molecular cell 76, 826-837.el 1 (2019).

[51] Plotkin, J. B., Dushoff, J. & Levin, S. A. Hemagglutinin sequence clusters and the antigenic evolution of influenza a virus. Proceedings of the National Academy of Sciences of the United States of America 99, 6263-6268 (2002).

[52] Langat, P. et al. Genome-wide evolutionary dynamics of influenza B viruses on a global scale. PLoS pathogens 13, e1006749 (2017).

[53] Vogels, C. B. F. et al. Analytical sensitivity and efficiency comparisons of SARS-COV-2 qRTPCR primer-probe sets (2020).

[54] Federhen, S. The NCBI taxonomy database. Nucleic acids research 40, D136-43 (2012).

[55] Bao, Y. et al. The influenza virus resource at the national center for biotechnology information. Journal of virology 82, 596-601 (2008).

[56] Metsky, H. C. et al. Capturing sequence diversity in metagenomes with comprehensive and scalable probe design. Nature biotechnology 37, 160-168 (2019).

[57] Pedregosa, F. et al. Scikit-learn: Machine learning in python. Journal of machine learning research: JMLR 12, 2825-2830 (2011).

[58] Martin Abadi et al. TensorFlow: Large-Scale machine learning on heterogeneous systems (2015).

[59] Kingma, D. P. & Ba, J. Adam: A method for stochastic optimization (2014). 1412.6980.

[60] Daher, R. K., Stewart, G., Boissinot, M. & Bergeron, M. G. Recombinase polymerase amplification for diagnostic applications. Clinical chemistry 62, 947-958 (2016).

[61] Ondov, B. D. et al. Mash: fast genome and metagenome distance estimation using MinHash. Genome biology 17, 132 (2016).

[62] United States Centers for Disease Control and Prevention. Research use only 2019-novel coronavirus (2019-nCoV) real-time RT-PCR primers and probes. https://www.cdc.gov/coronavirus/2019-ncov/lab/rt-per-panel-primer-probes.html .

[63] Chvatal, V. A greedy heuristic for the Set-Covering problem. Mathematics of Operations Research 4, 233-235 (1979).

[64] Johnson, D. S. Approximation algorithms for combinatorial problems. Journal of Computer and System Sciences 9, 256-278 (1974).

[65] Pearson, W. R., Robins, G., Wrege, D. E. & Zhang, T. On the primer selection problem in polymerase chain reaction experiments. Discrete applied mathematics 71, 231-246 (1996).

[66] Huang, Y.-C. et al. Integrated minimum-set primers and unique probe design algorithms for differential detection on symptom-related pathogens. Bioinformatics 21, 4330-4337 (2005).

[67] Feige, U. A threshold of 1n n for approximating set cover. Journal of the ACM 45, 634-652 (1998).

[68] Moshkovitz, D. The projection games conjecture and the NP-Hardness of 1n n-Approximating Set-Cover. Theory of Computing 11, 221-235 (2015).

[69] Har-Peled, S. & Jones, M. Few cuts meet many point sets (2018). 1808.03260.

[70] Varani, G. & McClain, W. H. The G x U wobble base pair. a fundamental building block of RNA structure crucial to RNA function in diverse biological systems. EMBO reports 1, 18-23 (2000).

[71] Saxena, S., J'onsson, Z. O. & Dutta, A. Small RNAs with imperfect match to endogenous mRNA repress translation. implications for off-target activity of small inhibitory RNA in mammalian cells. The Journal of biological chemistry 278, 44312-44319 (2003).

[72] Du, Q., Thonberg, H., Wang, J., Wahlestedt, C. & Liang, Z. A systematic analysis of the silencing effects of an active siRNA at all single-nucleotide mismatched target sites. Nucleic acids research 33, 1671-1677 (2005).

[73] Snove, O., Jr & Holen, T. Many commonly used siRNAs risk off-target activity. Biochemical and biophysical research communications 319, 256-263 (2004).

[74] Naito, Y., Yamada, T., Ui-Tei, K., Morishita, S. & Saigo, K. sidirect: highly effective, targetspecific siRNA design software for mammalian RNA interference. Nucleic acids research 32, W124-9 (2004).

[75] Qiu, S., Adema, C. M. & Lane, T. A computational study of off-target effects of RNA interference. Nucleic acids research 33, 1834-1847 (2005).

[76] Yamada, T. & Morishita, S. Accelerated off-target search algorithm for siRNA. Bioinformatics 21, 1316-1324 (2005).

[77] Zhao, W. & Lane, T. siRNA off-target search: A hybrid q-gram based filtering approach. In Proceedings of the 5th International Workshop on Bioinformatics, BIOKDD '05, 54-60 (ACM, New York, NY, USA, 2005).

[78] Alkan, F. et al. Research2: suffix array-based large-scale prediction of RNA-RNA interactions and siRNA off-targets. Nucleic acids research 45, e60 (2017).

[79] Doench, J. G. & Sharp, P. A. Specificity of microRNA target selection in translational repression. Genes & development 18, 504-511 (2004).

[80] Andoni, A. & Indyk, P. Near-optimal hashing algorithms for approximate nearest neighbor in high dimensions. of Computer Science, 2006. FOCS'06. 47th . . . (2006).

[81] Wrinda, K., Sykulski, M. & Kucherov, G. Spaced seeds improve k-mer-based metagenomic classification. Bioinformatics 31, 3584-3592 (2015).

[82] Katoh, K. & Standley, D. M. MAFFT multiple sequence alignment software version 7: improvements in performance and usability. Molecular biology and evolution 30, 772-780 (2013)._

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A computer-implemented method to design sensitive and specific binding molecules, comprising, by, one or more computing devices:
identifying binding molecules with maximal activity across a diverse set of genomes by:
identifying all known sequences within a region,
constructing a ground set of possible binding molecules by finding representative subsequences across the set using locality sensitive hashing,
identifying a function that quantifies detection activity between a binding molecule and a targeting sequence, and
identifying a set of binding molecules within the ground set that maximizes a function of the expected activity,
constructing an activity function by:
creating a data base of unique guide-target pairs having sequence composition representative of viral genomes,
training a classifier on all pairs, and
creating a regressing model for active pairs;
developing an exact query algorithm to enforce specificity by:
splitting sequences into a configured number of components,
hashing each component to a bit vector,
constructing all combinations of flipped bits,
fetching corresponding tries, and
querying k-mers in each of the tries, and
performing a branch and bound search to identify a ranked list of binding molecules.

2. The computer-implemented method of claim 1, wherein the database is updated periodically, continually, or in real time.

3. The computer-implemented method of claim 1, wherein identifying the set of binding molecules within the ground set that maximizes a function of the expected activity is performed by an algorithm for maximizing a non-negative and non-monotone submodular function.

4. The computer-implemented method of claim 1, wherein the classifier is a convolutional neural network.

5. The computer-implemented method of claim 1, wherein the regressing model is created via a convolutional neural network.

6. The computer-implemented method of claim 5, wherein the convolutional neural network uses multiple parallel convolutional and locally-connected filters of different widths.

7. The computer-implemented method of claim 1, wherein the branch and bound search is performed over viral genomes from a viral genome database.

8. The computer-implement method of claim 1, wherein the binding molecule is an oligonucleotide binding molecule, optionally wherein the oligonucleotide binding molecule is an amplification primer, hybridization probe, toehold switch, or guide molecule.

9. (canceled)

10. The computer-implemented method of claim 1, wherein the analysis of each cluster is based on a consensus of the target sequences, or on a mode of the target sequences.

11. (canceled)

12. The computer-implemented method of claim 1, wherein the identification of each cluster is based on a generated sequence.

13. The computer-implemented method of claim 1, further comprising, identifying, by one or more computing devices, a set of windows of a configured nt length in a set of target sequences of the target sample.

14. A computer-implemented method to design sensitive and specific binding molecules, comprising, by, one or more computing devices:
identifying binding molecules with maximal activity across a diverse set of genomes by:
identifying all known sequences within a region,
construct a ground set of possible binding molecules by finding representative subsequences across the set using locality sensitive hashing,
identifying a function that quantifies detection activity between a binding molecule and a targeting sequence, and
identifying a set of binding molecules within the ground set that maximizes a function of the expected activity,
inputting a particular target sequence into the machine-learning algorithm;
receiving a binding molecule generated by the machine-learning algorithm based on the inputted particular target sequence, the generated binding molecule being optimally active for the particular target sequence.

15. A nucleic acid detection system for detecting the presence of a target molecule in a sample comprising: one or more binding molecules according to the method of claim 1.

16. The nucleic acid detection system of claim 15, wherein the binding molecule is an amplification primer, hybridization probe, toehold switch, or guide molecule.

17. The nucleic acid detection system of claim 15, wherein the target molecule is a virus, optionally wherein the target molecule comprises a coronavirus.

18. (canceled)

19. The nucleic acid detection system of claim 17, wherein the coronavirus is SARS -CoV-2.

20. The nucleic acid detection system of claim 15 wherein the binding molecule comprises an amplification primer, or guide molecule from Table 4.

21. The nucleic acid detection system of claim 15, comprising one or more CRISPR systems comprising:
one or more Cas proteins;
one or more guide molecules made according to a computer-implemented method to design sensitive and specific guide molecules, comprising, by, one or more computing devices:
identifying guide molecules with maximal activity across a diverse set of genomes by:
identifying all known sequences within a region,
constructing a ground set of possible guide molecules by finding representative subsequences across the set using locality sensitive hashing,
identifying a function that quantifies detection activity between a guide molecule and a targeting sequence, and
identifying a set of guide molecules within the ground set that maximizes a function of the expected activity,
constructing an activity function by:
creating a data base of unique guide-target pairs having sequence composition representative of viral genomes,
training a classifier on all pairs, and
creating a regressing model for active pairs;
developing an exact query algorithm to enforce specificity by:
splitting sequences into a configured number of components,
hashing each component to a bit vector,
constructing all combinations of flipped bits,
fetching corresponding tries, and
querying k-mers in each of the tries, and
performing a branch and bound search to identify a ranked list of guide molecules and designed to bind to one or more corresponding target sequences of one or more viral species or subspecies; and
a detection construct.

22. The system of claim 21, wherein the one or more viral species comprise Coronavirus, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, human parainfluenza virus, Hepatitis D virus influenza, Enterovirus, human metapneumovirus, optionally wherein the coronavirus comprises SARS-CoV-2.

23. (canceled)

24. The system of claim 21, wherein the one or more Cas proteins is a Class 1 or Class 2 CRISPR protein, optionally wherein the one or more Cas proteins is one or more Type II, one or more Type V Cas protein, one or more Type VI Cas proteins, or a combination of one or more Type V and Type VI proteins.

25. (canceled)

26. The system of claim 21, wherein the one or more Cas proteins comprises two HEPN domains, optionally wherein the one or more HEPN domains comprise a RxxxxH motif sequence, optionally wherein the RxxxxH motif comprises a R[N/H/K]X₁X₂X₃H (SEQ ID NO: 1-3) sequence, wherein X₁ is R, S, D, E, Q, N, G, or Y, and X₂ is independently I, S, T, V, or L, and X₃ is independently L, F, N, Y, V, I, S, D, E, or A.

27. (canceled)

28. The system of claim 26, wherein the one or more Cas proteins is a Cas13, optionally wherein the Cas13 is Cas13a, Cas13b, or Cas13c.

29. (canceled)

30. The system of claim 24, wherein the Type V Cas is a Cas12a, a Cas12b, a Cas12c, a Cas12d, or a Cas12e.

31. The system of claim 21, wherein the detection construct suppresses generation of a detectable positive signal until cleaved or deactivated, or masks a detectable positive signal, or generates a detectable negative signal until the detection construct is deactivated or cleaved.

32. The system of claim 21, further comprising reagents to amplify target sequences comprising reagents for nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).

33. The system of claim 21, further comprising nuclease inhibitors, tris(2-carboxyethyl)phosphine hydrochloride (TCEP) and Ethylenediaminetetraacetic acid (EDTA).

34. A method for detecting target nucleic acids in samples comprising:
contacting one or more samples with the system of claim 21, the system further comprising a polynucleotide-based masking construct comprising a non-target sequence; and
heating the sample for 5 to 10 minutes,
wherein the Cas protein exhibits collateral nuclease activity and cleaves the non-target sequence of the nuclease-based masking construct once activated by the target sequence; and detecting a signal from cleavage of the non-target sequence, thereby detecting the one or more target sequences in the sample.

35. The method of claim 34, wherein heating the sample comprises heating the sample at two different temperatures, a first temperature of about 40° C. for 5 minutes and a second temperature of about 70° C. for 5 minutes.

36. A diagnostic device comprising one or more individual discrete volumes, each individual discrete volume comprising a CRISPR system of claim 21.

37. The device of claim 36, wherein the individual discrete volumes are droplets or microwells, or are defined on a solid substrate, or are spots defined on the solid substrate.

38. (canceled)

39. (canceled)

40. The device of claim 37, further comprising a mobile phone readout of the detectable signal.

41. A kit for detecting viral nucleic acids in a sample comprising
nucleic acid amplification reagents; and
a CRISPR system of claim 21.

42. A method for developing or designing a therapy or therapeutic, comprising
optimizing a binding molecule for the therapy or therapeutic according to claim 1, wherein specificity and sensitivity are optimized, optionally wherein the binding molecule is an antisense RNA, microRNA or guide molecule.

43. A method of modifying a target locus of interest, comprising delivering to the target a binding molecule designed according to claim 1.

44. (canceled)

45. The method of claim 42, comprising delivering to the target a CRISPR system comprising one or more Cas proteins and wherein the binding molecule designed is one or more guide molecules.

46. A composition for modifying a target molecule, the composition comprising a binding molecule designed according to claim 1, optionally wherein the binding molecule is an antisense RNA, microRNA or guide molecule.

47. (canceled)

48. The composition of claim 46, wherein the composition comprises a CRISPR system, the CRISPR system comprising one or more Cas proteins and one or more guide molecules, and wherein the binding molecule is one or more guide molecules.

49. The composition of claim 48, wherein the Cas protein is a Cas protein from a Class 1 or Class 2 CRISPR-Cas system.

50. The composition of claim 48, wherein the Cas protein is a Cas protein from a Class 2 Type II, Type V or Type VI CRISRP-Cas system, optionally wherein the Cas protein is a Cas9, Cas12 or Cas13.

51. (canceled)

52. The composition of claim 50, wherein the target is associated with a disease, virus, is expressed in cancer cells, or is expressed in pathogen-infected cells,. optionally wherein the target is associated with SARS-CoV-2.

53. (canceled)


SEQUENCE LISTING (46 sequences)

SEQ ID NO: 1 - protein, 6 aa, Artificial Sequence
  Synthetic
  x (3)..(3): R, S, D, E, Q, N, G, or Y
  x (4)..(4): I, S, T, V, or L
  x (5)..(5): L, F, N, Y, V, I, S, D, E, or A
RNXXXH

SEQ ID NO: 2 - protein, 6 aa, Artificial Sequence
  Synthetic
  x (3)..(3): R, S, D, E, Q, N, G, or Y
  x (4)..(4): I, S, T, V, or L
  x (5)..(5): L, F, N, Y, V, I, S, D, E, or A
RHXXXH

SEQ ID NO: 3 - protein, 6 aa, Artificial Sequence
  Synthetic
  x (3)..(3): R, S, D, E, Q, N, G, or Y
  x (4)..(4): I, S, T, V, or L
  x (5)..(5): L, F, N, Y, V, I, S, D, E, or A
RKXXXH

SEQ ID NO: 4 - protein, 6 aa, Artificial Sequence
  Synthetic
  x (3)..(3): R, S, D, E, Q, N, G, Y, or H
  x (4)..(4): I, S, T, V, or L
  x (5)..(5): L, F, N, Y, V, I, S, D, E, or A
RNXXXH

SEQ ID NO: 5 - protein, 6 aa, Artificial Sequence
  Synthetic
  x (3)..(3): R, S, D, E, Q, N, G, Y, or H
  x (4)..(4): I, S, T, V, or L
  x (5)..(5): L, F, N, Y, V, I, S, D, E, or A
RHXXXH

SEQ ID NO: 6 - protein, 6 aa, Artificial Sequence
  Synthetic
  x (3)..(3): R, S, D, E, Q, N, G, Y, or H
  x (4)..(4): I, S, T, V, or L
  x (5)..(5): L, F, N, Y, V, I, S, D, E, or A
RKXXXH

SEQ ID NO: 7 - protein, 6 aa, Artificial Sequence
  Synthetic
  x (3)..(3): R, S, D, E, Q, N, G, Y, or H
  x (4)..(4): I, S, T, V, or L
  x (5)..(5): L, F, N, Y, V, I, S, D, E, or A
RNXXXH

SEQ ID NO: 8 - protein, 6 aa, Artificial Sequences
  Synthetic
  x (3)..(3): R, S, D, E, Q, N, G, or Y
  x (4)..(4): I, S, T, V, or L
  x (5)..(5): L, F, N, Y, V, I, S, D, E, or A
RHXXXH

SEQ ID NO: 9 - protein, 6 aa, Artificial Sequence
  Synthetic
  x (3)..(3): R, S, D, E, Q, N, G, or Y
  x (4)..(4): I, S, T, V, or L
  x (5)..(5): L, F, N, Y, V, I, S, D, E, or A
RNXXXH

SEQ ID NO: 10 - protein, 6 aa, Artificial Sequence
  Synthetic
  x (3)..(3): R, S, D, E, Q, N, G, or Y
  x (4)..(4): I, S, T, V, or L
  x (5)..(5): L, F, N, Y, V, I, S, D, E, or A
RKXXXH

SEQ ID NO: 11 - protein, 5 aa, Artificial Sequence
  Synthetic
NGRRT

SEQ ID NO: 12 - protein, 5 aa, Artificial Sequence
  Synthetic
NGRRN

SEQ ID NO: 13 - protein, 8 aa, Artificial Sequence
  Synthetic
NNNNGATT

SEQ ID NO: 14 - protein, 8 aa, Artificial Sequence
  Synthetic
NNNNRYAC

SEQ ID NO: 15 - protein, 7 aa, Artificial Sequence
  Synthetic
NNAGAAW

SEQ ID NO: 16 - protein, 4 aa, Artificial Sequence
  Synthetic
TTTV

SEQ ID NO: 17 - protein, 4 aa, Artificial Sequence
  Synthetic
TTCN

SEQ ID NO: 18 - protein, 7 aa, Artificial Sequence
  Synthetic
ENLYFQG

SEQ ID NO: 19 - protein, 8 aa, Artificial Sequence
  Synthetic
GPLGIAGQ

SEQ ID NO: 20 - protein, 5 aa, Artificial Sequence
  Synthetic
RGDYC

SEQ ID NO: 21 - protein, 5 aa, Artificial Sequence
  Synthetic
ASSHN

SEQ ID NO: 22 - protein, 6 aa, Artificial Sequence
  Synthetic
KCCYSL

SEQ ID NO: 23 - protein, 7 aa, Artificial Sequence
  Synthetic
YEVGHRC

SEQ ID NO: 24 - DNA, 55 bp, Artificial Sequence
  Synthetic
gaaattaatacgactcactatagggccaaggtaaacctttggaatttggtgccac

SEQ ID NO: 25 - DNA, 30 bp, Artificial Sequence
  Synthetic
actatcatcatctaaccaatcttcttcttg

SEQ ID NO: 26 - DNA, 500 bp, Artificial Sequence
  Synthetic
gaaattaatacgactcactataggggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggagg

SEQ ID NO: 27 - RNA, 64 nt, Artificial Sequence
  Synthetic
gauuuagacuaccccaaaaacgaaggggacuaaaaccucuucuucagguugaagagcagcagaa

SEQ ID NO: 28 - RNA, 28 nt, Artificial Sequence
  Synthetic
  misc_feature (1)..(1): /56-FAM/=isomer derivative of fluorescein attachment for oligonucleotides
  misc_feature (28)..(28): Biotin Tag=/3Bio/
rurururururururururururururu

SEQ ID NO: 29 - DNA, 20 bp, Artificial Sequence
  Synthetic
gaccccaaaatcagcgaaat

SEQ ID NO: 30 - DNA, 24 bp, Artificial Sequence
  Synthetic
tctggttactgccagttgaatctg

SEQ ID NO: 31 - DNA, 24 bp, Artificial Sequence
  Synthetic
  misc_feature (1)..(1): FAM=6-carboxyfluorescein
  misc_feature (24)..(24): BHQ1=Black Hole Quencher-1
accccgcattacgtttggtggacc

SEQ ID NO: 32 - DNA, 25 bp, Artificial Sequence
  Synthetic
gaaattaatacgactcactataggg

SEQ ID NO: 33 - DNA, 25 bp, Artificial Sequence
  Synthetic
gaaattaatacgactcactataggg

SEQ ID NO: 34 - RNA, 28 nt, Artificial Sequence
  Synthetic
cucuucuucagguugaagagcagcagaa

SEQ ID NO: 35 - DNA, 20 bp, Artificial Sequence
  Synthetic
gaccccaaaatcagcgaaat

SEQ ID NO: 36 - DNA, 24 bp, Artificial Sequence
  Synthetic
tctggttactgccagttgaatctg

SEQ ID NO: 37 - DNA, 24 bp, Artificial Sequence
  Synthetic
  misc_feature (1)..(1): /FAM/=6-carboxyfluorescein
  misc_feature (24)..(24): BHQ1=Black Hole Quencher-1
accccgcattacgtttggtggacc

SEQ ID NO: 38 - DNA, 30 bp, Artificial Sequence
  Synthetic
ccaaggtaaacctttggaatttggtgccac

SEQ ID NO: 39 - DNA, 30 bp, Artificial Sequence; identical to SEQ ID NO: 25
  Synthetic

SEQ ID NO: 40 - DNA, 475 bp, Artificial Sequence
  Synthetic
gtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggagg

SEQ ID NO: 41 - DNA, 25 bp, Artificial Sequence
  Synthetic
gaaattaatacgactcactataggg

SEQ ID NO: 42 - RNA, 28 nt, Artificial Sequence
  Synthetic
  misc_feature (1)..(1): /56-FAM/=isomer derivative of fluorescein attachment for oligonucleotides
  misc_feature (28)..(28): Biotin Tag = /3Bio/
rurururururururururururururu

SEQ ID NO: 43 - RNA, 14 nt, Artificial Sequence
  Synthetic
  misc_feature (1)..(1): /56-FAM/=isomer derivative of fluorescein attachment for oligonucleotides
  misc_feature (14)..(14): /3IABkFQ/=Iowa Black quencher ideal for use with fluorescein
rururururururu

SEQ ID NO: 44 - RNA, 36 nt, Artificial Sequence
  Synthetic
gauuuagacuaccccaaaaacgaaggggacuaaaac

SEQ ID NO: 45 - DNA, 47 bp, Artificial Sequence
  Synthetic
cactataggggctctagcgacttctttaaatagtggcttaaaataac

SEQ ID NO: 46 - DNA, 27 bp, Artificial Sequence
  Synthetic
gctctagcgacttctttaaatagtggc